(19) United States
(12) Patent Application Publication
(10) Pub. No.: US 2024/0166612 A1
(43) Pub. Date: May 23, 2024

(54) ARYLTHIOETHER ACETAMIDE AND RELATED COMPOUNDS AND THEIR USE IN TREATING MEDICAL CONDITIONS

(71) Applicant: X-Biotix Therapeutics, Inc., Waltham, MA (US)

(72) Inventors: Michael Dominic RYAN, Littleton, MA (US); Alastair PARKES, Abingdon (GB); Olivier Rémi BARBEAU, Abingdon (GB); Adele FAULKNER, Abingdon (GB); Maisie HOLBROW-WILSHAW, Abingdon (GB); Michelle SOUTHEY, Abingdon (GB); Angelo SANZONE, Abingdon (GB); Christophe BOLDRON, Toulouse (FR); Elise GADOULEAU, Abingdon (GB); Timothy GORMAN, Abingdon (GB); Kate SPEAR, Abingdon (GB); Thomas Martin KRULLE, Abingdon (GB); Vasileios ROUMPELAKIS, Abingdon (GB); Spencer Charles Robert NAPIER, Abingdon (GB)

(21) Appl. No.: 18/275,880

(22) PCT Filed: Feb. 18, 2022
(86) PCT No.: PCT/US2022/016941

§ 371 Date: Aug. 4, 2023

Related U.S. Application Data
(60) Provisional application No. 63/150,617, filed on Feb. 18, 2021.

(51) Int. Cl. C07D 271/113; A61P 31/04; C07C 323/41; C07D 205/04; C07D 211/58; C07D 231/12; C07D 231/56; C07D 233/64; C07D 249/14; C07D 257/04; C07D 263/32; C07D 265/30; C07D 271/06; C07D 285/135; C07D 295/185; C07D 413/12; C07D 413/14

(52) U.S. Cl. CPC C07D 271/113; A61P 31/04; C07C 323/41; C07D 205/04; C07D 211/58; C07D 231/12; C07D 231/56; C07D 233/64; C07D 249/14; C07D 257/04; C07D 263/32; C07D 265/30; C07D 271/06; C07D 285/135; C07D 295/185; C07D 413/12; C07D 413/14; C07B 2200/05

(57) ABSTRACT

The invention provides arylthioether acetamide and related compounds, pharmaceutical compositions, and their use in the treatment of medical conditions, such as bacterial infections, and in inhibiting LpxA activity.

### CROSS REFERENCE TO RELATED APPLICATIONS

This application is the national stage application of International (PCT) Patent Application Serial No. PCT/US2022/016941, filed Feb. 18, 2022, which claims the benefit of and priority to U.S. Provisional Patent Application Ser. No. 63/150,617, filed Feb. 18, 2021, the contents of each of which are hereby incorporated by reference.

### FIELD OF THE INVENTION

The invention provides arylthioether acetamide and related compounds, pharmaceutical compositions, and their use in the treatment of medical conditions, such as bacterial infections, and in inhibiting LpxA activity.

### BACKGROUND

Bacterial infection continues to be a serious health problem despite the substantial research efforts and scientific advances reported in the literature. Difficulties in treating bacterial infections are exacerbated by the development of bacteria with resistance to one or more antibiotics, particularly those that are used routinely.

Without effective treatment, bacterial infections can cause significant health problems and even result in death. For example, Escherichia coli can infect the urinary tract, lungs, and gastrointestinal system. Severe and bloody diarrhea from gastrointestinal E. coli infection can lead to serious complications. Hemolytic uremic syndrome, which can induce life-threatening kidney failure, can also be caused by E. coli infection.

A frequent hospital-acquired infection is Pseudomonas aeruginosa, which can be particularly dangerous to immunocompromised patients or patients recovering from major surgery. According to the United States Centers for Disease Control and Prevention, in the United States each year, more than 50,000 healthcare-associated P. aeruginosa infections are estimated to occur, with over 6,000 cases being multidrug-resistant, and approximately 400 deaths being attributed to P. aeruginosa infections.

Accordingly, a need exists for improved treatments for bacterial infections. The present invention addresses this need and provides other related advantages.

### SUMMARY

The invention provides arylthioether acetamide and related compounds, pharmaceutical compositions, and their use in the treatment of medical conditions, such as bacterial infections, and in inhibiting LpxA activity. In particular, one aspect of the invention provides a collection of arylthioether acetamide and related compounds, such as a compound represented by Formula I.

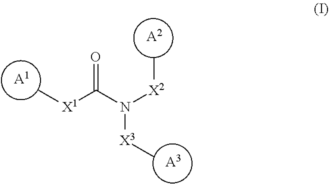

or a pharmaceutically acceptable salt thereof, wherein the variables are as defined in the detailed description. Further description of additional collections of arylthioether acetamide and related compounds are described in the detailed description. The compounds may be part of a pharmaceutical composition comprising a pharmaceutically acceptable carrier.

Another aspect of the invention provides a method of treating a bacterial infection in a patient. The method comprises administering to the patient a therapeutically effective amount of an arylthioether acetamide or related compound described herein, e.g., a compound of Formula I or I-A, to treat the bacterial infection. In certain embodiments, the bacterial infection is an infection by Escherichia coli, Pseudomonas aeruginosa, or a combination thereof. The compound may be used as monotherapy, or as part of a combination therapy, to treat the bacterial infection.

Another aspect of the invention provides a method of inducing death of a bacterial cell. The method comprises exposing the bacterial cell to an effective amount of an arylthioether acetamide or related compound described herein, e.g., a compound of Formula I or I-A, to induce death of the bacterial cell. In certain embodiments, the bacterial cell is an Escherichia coli or Pseudomonas aeruginosa bacterium.

Another aspect of the invention provides a method of inhibiting the activity of LpxA. The method comprises exposing a LpxA to an effective amount of an arylthioether acetamide or related compound described herein, e.g., a compound of Formula I or I-A, to inhibit the activity of LpxA.

### DETAILED DESCRIPTION

The invention provides arylthioether acetamide and related compounds, pharmaceutical compositions, and their use in the treatment of medical conditions, such as bacterial infections, and in inhibiting LpxA activity. The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name, and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “—O-alkyl” etc.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “alkylene” refers to a diradical of an alkyl group. Exemplary alkylene groups include —CH₂—, —CH₂CH₂—, and —CH₂C(H)(CH₃)CH₂—. The term “—(C₀ alkylene)-” refers to a bond. Accordingly, the term “—(C₀₋₃ alkylene)-” encompasses a bond (i.e., C₀) and a —(C₁₋₃ alkylene) group.

The term “heteroalkyl” refers to an alkyl group in which one or more carbon atoms has been replaced by a heteroatom (e.g., N, O, or S). Exemplary heteroalkyl groups include —CH₂O—, —CH₂OCH₂—, and —CH₂CH₂O—. The heteroalkyl group may contain, for example, from 2-4, 2-6, or 2-8 atoms selected from the group consisting of carbon and a heteroatom (e.g., N, O, or S). The phrase 2-3 membered heteroalkyl refers to a heteroalkyl group having from 2 to 3 atoms selected from the group consisting of carbon and a heteroatom.

The term “heteroalkylene” refers to a diradical of a heteroalkyl group. The term “heterohaloalkylene” refers to a diradical of a heteroalkyl group, wherein one or more carbon atoms are substituted with at least one halogen.

The term “cycloalkyl” refers to a monovalent saturated cyclic, bicyclic, bridged cyclic (e.g., adamantyl), or spirocyclic hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl.

The term “cycloalkylene” refers to a diradical of a cycloalkyl group. Exemplary cycloalkylene groups include

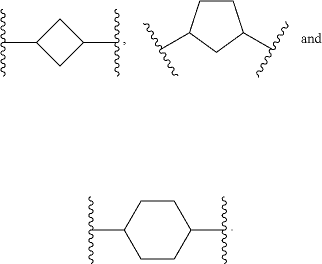

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like. In certain embodiments, the haloalkyl contains 1-10, 1-6, or 1-3 carbon atoms. The term “haloalkylene” refers to a diradical of a haloalkyl group.

The term “deuteroalkyl” refers to an alkyl group that is substituted with at least one deuterium. Exemplary deuteroalkyl groups include —CH₂D, —CHD₂, —CD₃, —CH₂CD₃, —CD₂CD₃, and the like. In certain embodiments, the deuteroalkyl contains 1-10, 1-6, or 1-3 carbon atoms. In certain embodiments, the deuteroalkyl contains 1, 2, or 3 deuterium atoms. In certain embodiments, the deuteroalkyl is a C₁₋₆ deuteroalkyl containing 1, 2, 3, 4, 5, or 6 deuterium atoms. In certain embodiments, the deuteroalkyl is a C₂₋₃ deuteroalkyl containing 1, 2, 3, 4, or 5 deuterium atoms.

The term “hydroxyalkyl” refers to an alkyl group that is substituted with at least one hydroxyl. Exemplary hydroxyalkyl groups include —CH₂CH₂OH, —C(H)(OH)CH₃, —CH₂C(H)(OH)CH₂CH₂OH, and the like.

The term “aralkyl” refers to an alkyl group substituted with an aryl group. Exemplary aralkyl groups include

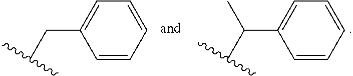

In certain embodiments, the aralkyl contains 7-15, 7-11, or 7-9 carbon atoms.

The term “heteroaralkyl” refers to an alkyl group substituted with a heteroaryl group.

The terms “alkenyl” and “alkynyl” refer to unsaturated aliphatic groups analogous in length and possible substitution to the alkyls described above, but that contain at least one double or triple bond respectively.

The term “carbocyclyl” refers to a mono-radical of a saturated, partially unsaturated, or aromatic carbocyclic ring (e.g., a monocyclic, bicyclic, bridged (e.g., adamantyl), or spirocyclic ring). In certain embodiments, the carbocyclyl contains 3-10, 4-8, or 5-6 carbons, referred to herein, e.g., as “C₅-C₆ carbocyclyl”. Unless specified otherwise, the carbocyclic ring may be substituted at one or more ring positions with, for example, halogen, azide, alkyl, aralkyl, alkenyl, alkynyl, cycloalkyl, hydroxyl, alkoxyl, hydroxyalkyl, alkoxyalkyl, oxo, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the carbocyclyl is substituted at one or more ring positions. In certain embodiments, the carbocyclyl is not substituted.

The term “carbocyclylene” refers to a multivalent radical (e.g., a divalent or trivalent radical) of a saturated, partially unsaturated, or aromatic carbocyclic ring (e.g., a monocyclic, bicyclic, bridged (e.g., adamantyl), or spirocyclic ring). In certain embodiments, the carbocyclylene contains 3-10, 4-8, or 5-6 carbons, referred to herein, e.g., as “C₅-C₆ carbocyclylene”. Unless specified otherwise, the carbocyclic ring may be substituted at one or more ring positions with, for example, halogen, azide, alkyl, aralkyl, alkenyl, alkynyl, cycloalkyl, hydroxyl, alkoxyl, hydroxyalkyl, alkoxyalkyl, oxo, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the carbocyclylene is substituted at one or more ring positions. In certain embodiments, the carbocyclylene is not substituted.

The term “aryl” refers to a carbocyclic aromatic group and includes polycyclic aromatic ring systems having two or more carbocyclic rings in which two or more carbons are common to two adjoining rings (the rings are “fused rings”) wherein all of the fused rings are aromatic rings, e.g., in a naphthyl group. Representative aryl groups include phenyl, naphthyl, anthracenyl, and the like. Unless specified otherwise, the aromatic ring may be substituted at one or more ring positions with, for example, halogen, azide, alkyl, aralkyl, alkenyl, alkynyl, cycloalkyl, hydroxyl, alkoxyl, hydroxyalkyl, alkoxyalkyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aryl is a 6-10 membered ring. In certain embodiments, the aryl is a 6 membered ring. In certain embodiments, the aryl is substituted with 1, 2, or 3 substituents. In certain embodiments, the aryl is not substituted.

The terms “heterocyclic” and “heterocyclyl” refer to a saturated, partially unsaturated, or aromatic ring (e.g., a monocyclic, bicyclic ring, bridged, or spirocyclic ring) containing one or more heteroatoms (e.g., 1, 2, 3, or 4 heteroatoms, such as where the heteroatom is selected from oxygen, nitrogen, and sulfur). The heteroatoms can be the same or different from each other. Examples of heteroatoms include, but are not limited to nitrogen, oxygen and sulfur. Aromatic and nonaromatic heterocyclic rings are well-known in the art. Some non-limiting examples of aromatic heterocyclic rings include, but are not limited to, pyridine, pyrimidine, indole, purine, quinoline and isoquinoline. Nonlimiting examples of nonaromatic heterocyclic compounds include, but are not limited to, piperidine, piperazine, morpholine, pyrrolidine and pyrazolidine. Examples of oxygen containing heterocyclic rings include, but are not limited to, furan, oxirane, 2H-pyran, 4H-pyran, 2H-chromene, benzofuran, and 2,3-dihydrobenzo[b][1,4]dioxine. Examples of sulfur-containing heterocyclic rings include, but are not limited to, thiophene, benzothiophene, and parathiazine. Examples of nitrogen containing rings include, but are not limited to, pyrrole, pyrrolidine, pyrazole, pyrazolidine, imidazole, imidazoline, imidazolidine, pyridine, piperidine, pyrazine, piperazine, pyrimidine, indole, purine, benzimidazole, quinoline, isoquinoline, triazole, and triazine. Examples of heterocyclic rings containing two different heteroatoms include, but are not limited to, phenothiazine, morpholine, parathiazine, oxazine, oxazole, thiazine, and thiazole. Unless specified otherwise, the “heterocyclic” and “heterocyclyl” ring is optionally substituted at one or more ring positions with, for example, halogen, azide, alkyl, aralkyl, alkenyl, alkynyl, cycloalkyl, hydroxyl, alkoxyl, hydroxyalkyl, alkoxyalkyl, oxo, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the heterocyclyl group is a 3-10 membered ring that, unless specified otherwise, is substituted or unsubstituted. In certain embodiments, the heterocyclyl group is a 3-7 membered ring that, unless specified otherwise, is substituted or unsubstituted. In certain embodiments, the heterocyclyl group is a 5-6 membered ring that, unless specified otherwise, is substituted or unsubstituted.

The terms “aza-heterocyclic” and “aza-heterocyclyl” refer to a heterocyclic or heterocyclyl group containing at least one ring nitrogen atom.

The term “heterocycloalkyl” refers to a saturated heterocyclyl group having, for example, 3-7 ring atoms selected from carbon and heteroatoms (e.g., O, N, or S).

The term “heteroaryl” refers to aromatic groups that include at least one ring heteroatom. In certain occurrences, a heteroaryl group contains 1, 2, 3, or 4 ring heteroatoms (e.g., O, N, and S). Representative examples of heteroaryl groups include pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring may be substituted at one or more ring positions with, for example, halogen, azide, alkyl, aralkyl, alkenyl, alkynyl, cycloalkyl, hydroxyl, alkoxyl, hydroxyalkyl, alkoxyalkyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “heteroaryl” also includes polycyclic aromatic ring systems having two or more rings in which two or more ring atoms are common to two adjoining rings (the rings are “fused rings”) wherein all of the fused rings are heteroaromatic, e.g., in a naphthyridinyl group. In certain embodiments, the heteroaryl is a 5-6 membered monocylic ring or a 9-10 membered bicyclic ring.

The term “aza-heteroaryl” refers to a heteroaryl containing at least one ring nitrogen atom.

The terms ortho, meta, and para are art-recognized and refer to 1,2-, 1,3- and 1,4-disubstituted benzenes, respectively. For example, the names 1,2-dimethylbenzene and ortho-dimethylbenzene are synonymous.

The terms “amine” and “amino” are art-recognized and refer to both unsubstituted and substituted amines, e.g., a moiety that may be represented by the general formulas:

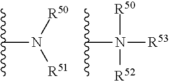

wherein R⁵⁰, R⁵¹, R⁵² and R⁵³ each independently represent a hydrogen, an alkyl, an alkenyl, —(CH₂)ₘ—R⁶¹, or R⁵⁰ and R⁵¹, taken together with the N atom to which they are attached complete a heterocycle having from 4 to 8 atoms in the ring structure; R⁶¹ represents an aryl, a cycloalkyl, a cycloalkenyl, a heterocycle or a polycycle; and m is zero or an integer in the range of 1 to 8. In certain embodiments, only one of R⁵⁰ or R⁵¹ may be a carbonyl, e.g., R⁵⁰, R⁵¹ and the nitrogen together do not form an imide. In other embodiments, R⁵⁰ and R⁵¹ (and optionally R⁵²) each independently represent a hydrogen, an alkyl, an alkenyl, or —(CH₂)ₘ—R⁶¹.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O— alkenyl, —O-alkynyl, and —O—(CH₂)ₘ—R⁶¹, where m and R⁶¹ are described above.

The term “oxo” is art-recognized and refers to a “═O” substituent. For example, a cyclopentane substituted with an oxo group is cyclopentanone.

The symbol “ [symbol] ” indicates a point of attachment.

The term “substituted” means that one or more hydrogens on the atoms of the designated group are replaced with a selection from the indicated group, provided that the atoms' normal valencies under the existing circumstances are not exceeded, and that the substitution results in a stable compound. Combinations of substituents and/or variables are permissible only if such combinations result in stable compounds. The terms “stable compound’ or “stable structure” refer to a compound that is sufficiently robust to survive isolation to a useful degree of purity from a reaction mixture, and formulation into an efficacious therapeutic agent.

When any substituent or variable occurs more than one time in any constituent or the compound of the invention, its definition on each occurrence is independent of its definition at every other occurrence, unless otherwise indicated.

It should also be noted that any carbon as well as heteroatom with unsatisfied valences in the text, schemes, examples and tables herein is assumed to have the sufficient number of hydrogen atom(s) to satisfy the valences.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain occurrences the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

Certain compounds contained in compositions of the present invention may exist in particular geometric or stereoisomeric forms. Further, certain compounds described herein may be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. The compounds may contain one or more stereogenic centers. For example, asymmetric carbon atoms may be present in a substituent such as an alkyl group. All such isomers, as well as mixtures thereof, are intended to be included in this invention, such as, for example, racemic mixtures, single enantiomers, diastereomeric mixtures and individual diastereomers. Additional asymmetric centers may be present depending upon the nature of the various substituents on the molecule. Each such asymmetric center will independently produce two optical isomers, and it is intended that all of the possible optical isomers, diastereomers in mixtures, and pure or partially purified compounds are included within the ambit of this invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as a atropisomer (e.g., substituted biaryls), all forms of such atropisomer are considered part of this invention.

As used herein, the terms “subject” and “patient” are used interchangeably and refer to organisms to be treated by the methods of the present invention. Such organisms preferably include, but are not limited to, mammals (e.g., murines, simians, equines, bovines, porcines, canines, felines, and the like), and most preferably includes humans.

The term “EC₅₀” is art-recognized and refers to the concentration of a compound that is required to achieve 50% of the maximum possible activation of the target.

As used herein, the term “effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₃, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate (also known as toluenesulfonate), undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. Further examples of salts include, but are not limited to: ascorbate, borate, nitrate, phosphate, salicylate, and sulfate. Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference.

Additional exemplary basic salts include, but are not limited to: ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

In addition, when a compound of the invention contains both a basic moiety (such as, but not limited to, a pyridine or imidazole) and an acidic moiety (such as, but not limited to, a carboxylic acid) zwitterions (“inner salts”) may be formed. Such acidic and basic salts used within the scope of the invention are pharmaceutically acceptable (i.e., non-toxic, physiologically acceptable) salts. Such salts of the compounds of the invention may be formed, for example, by reacting a compound of the invention with an amount of acid or base, such as an equivalent amount, in a medium such as one in which the salt precipitates or in an aqueous medium followed by lyophilization.

The present invention includes the compounds of the invention in all their isolated forms (such as any solvates, hydrates, stereoisomers, and tautomers thereof). Further, the invention includes compounds in which one or more of the atoms may be artificially enriched in a particular isotope having the same atomic number, but an atomic mass or mass number different from the atomic mass or mass number predominantly found in nature. The present invention is meant to include all suitable isotopic variations of the compounds of the invention. For example, different isotopic forms of hydrogen (H) include protium (H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds can be prepared without undue experimentation by conventional techniques known to those skilled in the art or by processes analogous to those described in the Schemes and Examples herein using appropriate isotopically-enriched reagents and/or intermediates.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

The terms “a” and “an” as used herein mean “one or more” and include the plural unless the context is inappropriate.

As a general matter, compositions specifying a percentage are by weight unless otherwise specified.

### I. Arylthioether Acetamide and Related Compounds

The invention provides arylthioether acetamide and related compounds. Exemplary compounds are described in the following sections.

One aspect of the invention provides a compound represented by Formula I:

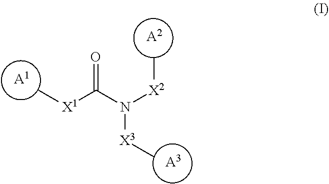

or a pharmaceutically acceptable salt thereof, wherein:
A¹ is a phenyl, 6-10 membered aza-heterocyclyl, or C₃₋₇ cycloalkyl, each of which is substituted with (i) one occurrence of R¹ and (ii) 0, 1, 2, or 3 occurrences of R²;
A² is one of the following:
   a 5-membered heteroaryl containing at least two ring heteroatoms independently selected from the group consisting of nitrogen, oxygen, and sulfur, wherein at least one ring heteroatom is nitrogen, and said heteroaryl is substituted with 0, 1, 2, or 3 occurrences of R³; or
   a 9-10 membered bicyclic aza-heteroaryl or 4-7 membered heterocycloalkyl, each of which is substituted with 0, 1, 2, or 3 occurrences of R³;
A³ is a 3-10 membered carbocyclyl or 3-10 membered heterocyclyl;
X¹ is 2-4 membered heteroalkylene or C₁₋₄ alkylene, each of which is substituted by 0, 1, or 2 occurrences of R⁴ and optionally one or more occurrences of halogen or deuterium;
X² is C₁₋₄ alkylene, —(C₁₋₃ alkylene)-C(O)-ψ, or —(C₁₋₄ alkylene)-C(O)N(R⁷)-ψ, each of which substituted by 0, 1, or 2 occurrences of R⁵ and optionally one or more occurrences of halogen; wherein ψ is a bond to A²; or

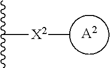

is —(C₁₋₄ alkylene)-C(O)N(R⁷)-(5-membered heteroaryl);
X³ is C₁₋₄ alkylene, C₂₋₄ alkenylene, or C₂₋₄ alkynylene; each of which substituted by 0, 1, or 2 occurrences of R⁶ and optionally one or more occurrences of halogen;
R¹ is halogen, cyano, C₁₋₄ alkyl, C₁₋₄ haloalkyl, C₁₋₄ deuteroalkyl, —O—C₁₋₄ alkyl, —O—C₁₋₄ haloalkyl, —S—C₁₋₄ alkyl, or —S—C₁₋₄ haloalkyl;
R² represents independently for each occurrence halogen, cyano, hydroxyl, —N(R⁷)₂, —CON(R⁷)₂, —SOₜN(R⁷)₂, C₁₋₆ alkyl, —O—C₁₋₆ alkyl, —S—C₁₋₆ alkyl, C₂₋₆ alkenyl, C₂₋₆ alkynyl, C₃₋₆ cycloalkyl, —O—C₃₋₆ cycloalkyl, or 3-6 membered heterocyclyl; wherein the C₃₋₆ cycloalkyl, —O—C₃₋₆ cycloalkyl, and 3-6 membered heterocyclyl are each optionally substituted with 1 or 2 substituents independently selected from C₁₋₆ alkyl, hydroxyl, oxo, —N(R⁴)₂, —CON(R⁴)₂, —SOₜN(R⁶)₂, and halogen; wherein each of the foregoing C₁₋₆ alkyl groups is optionally substituted with —OR⁷, —N(R⁷)₂, —CON(R⁷)₂, —SOₜN(R⁷)₂, or 1-3 halogens;
R³ represents independently for each occurrence —(C₀₋₄ alkylene)-N(R⁷)₂, —(C₀₋₄ alkylene)-OR⁷, C₁₋₄ alkyl, C₁₋₄ haloalkyl, or halogen;
R⁴, R⁵, and R⁶ each represent independently for each occurrence —(C₀₋₄ alkylene)-N(R⁷)₂ or —(C₀₋₄ alkylene)-OR⁷; or two occurrences of R⁴ are taken together with the carbon atom or atoms to which they are attached to form X⁴, two occurrences of R⁵ are taken together with the carbon atom or atoms to which they are attached to form X⁴, and/or two occurrences of R⁶ are taken together with the carbon atom or atoms to which they are attached to form X⁴; wherein X⁴ is a 3-7 membered ring optionally substituted with 1 or 2 substituents independently selected from halogen, C₁₋₄ alkyl, C₁₋₄ haloalkyl, —(C₀₋₄ alkylene)-N(R⁷)₂, and —(C₀₋₄ alkylene)-OR⁷;
R⁷ represents independently for each occurrence hydrogen, C₁₋₆ alkyl, C₁₋₆ haloalkyl, or —(C₀₋₆ alkylene)-(C₃-7 cycloalkyl); or two R⁷ attached to same nitrogen atom are taken together with the nitrogen atom to which they are attached to form a 3-7 membered heterocyclic ring; and
t represents independently for each occurrence 1 or 2.

The definitions of variables in Formula I above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is represented by Formula I.

In certain embodiments, A¹ is phenyl or 6-membered aza-heteroaryl, each of which is substituted with (i) one occurrence of R¹ and (ii) 0, 1, 2, or 3 occurrences of R². In certain embodiments, A¹ is phenyl, pyridinyl, or pyrimidinyl; each of which is substituted with (i) one occurrence of R¹ and (ii) 0, 1, 2, or 3 occurrences of R². In certain embodiments, A¹ is a C₃₋₇ cycloalkyl substituted with (i) one occurrence of R¹ and (ii) 0, 1, 2, or 3 occurrences of R². In certain embodiments, A¹ is an 8-10 membered bicyclic aza-heteroaryl substituted with (i) one occurrence of R¹ and (ii) 0, 1, 2, or 3 occurrences of R².

In certain embodiments, A¹ is

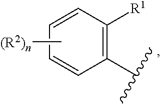

wherein n is 0, 1 or 2. In certain embodiments, A¹ is

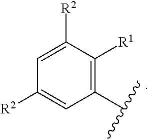

In certain embodiments, A¹ is

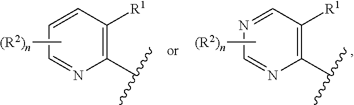

wherein n is 0, 1 or 2. In certain embodiments, n is 0. In certain embodiments, n is 1 or 2. In certain embodiments, n is 1. In certain embodiments, n is 2. In certain embodiments, n is 0 or 1.

In certain embodiments, A¹ is a phenyl substituted with (i) one occurrence of R¹ and (ii) 0, 1, 2, or 3 occurrences of R². In certain embodiments, A¹ is a 6-10 membered aza-heterocyclyl substituted with (i) one occurrence of R¹ and (ii) 0, 1, 2, or 3 occurrences of R².

In certain embodiments, A² is a 5-membered heteroaryl containing at least two ring heteroatoms independently selected from the group consisting of nitrogen, oxygen, and sulfur, wherein at least one ring heteroatom is nitrogen, and said heteroaryl is substituted with 0, 1, 2, or 3 occurrences of R³. In certain embodiments, A² is a 9-10 membered bicyclic aza-heteroaryl substituted with 0, 1, 2, or 3 occurrences of R³. In certain embodiments, A² is a 4-7 membered heterocycloalkyl substituted with 0, 1, 2, or 3 occurrences of R³.

In certain embodiments, A² is a 5-membered heteroaryl containing two or three ring heteroatoms each independently selected from the group consisting of nitrogen, oxygen, and sulfur, wherein at least one ring heteroatom is nitrogen, and said heteroaryl is substituted with 0, 1, 2, or 3 occurrences of R³. In certain embodiments, A² is a 5-membered heteroaryl containing two or three ring heteroatoms each independently selected from the group consisting of nitrogen, oxygen, and sulfur, wherein at least two ring heteroatoms are nitrogen, and said heteroaryl is substituted with 0, 1, 2, or 3 occurrences of R³.

In certain embodiments, A² is oxadiazolyl, thiadiazolyl, oxazolyl, triazolyl, imidazolyl, or pyrazolyl, each of which is optionally substituted with 0, 1, 2, or 3 occurrences of R³. In certain embodiments, A² is oxadiazolyl or thiadiazolyl, each of which is optionally substituted with one occurrence of R³. In certain embodiments, A² is

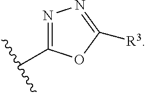

In certain embodiments, A² is

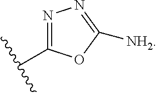 [SMILES: *c1nnc(N)o1]

In certain embodiments, A² is oxazolyl, triazolyl, imidazolyl, or pyrazolyl, each of which is optionally substituted with 1 or 2 occurrences of R³.

In certain embodiments, A³ is a 3-10 membered carbocyclyl or 5-6 membered heteroaryl, each of which is substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, oxo, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀-6 alkylene)-(C₃₋₇ cycloalkyl).

In certain embodiments, A³ is a 3-10 membered carbocyclyl substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, oxo, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl). In certain embodiments, A³ is a 3-10 membered saturated or partially unsaturated carbocyclyl substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, oxo, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀-6 alkylene)-(C₃₋₇ cycloalkyl).

In certain embodiments, A³ is naphthyl substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, and 3-6 membered heterocyclyl, and (ii) 0, 1, 2, or 3 substituents independent selected from the group consisting of halogen, C₁₋₆ alkyl, —O—C₁₋₆ alkyl, and C₁₋₆ haloalkyl; and R⁸ is C₁₋₆ alkyl, C₁₋₆ haloalkyl, or —(C₀₋₆ alkylene)-(C₃-7 cycloalkyl).

In certain embodiments, A³ is phenyl or a 5-6 membered heteroaryl, each of which is substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃-7 cycloalkyl).

In certain embodiments, A³ is phenyl substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀-6 alkylene)-(C₃₋₇ cycloalkyl). In certain embodiments, A³ is phenyl substituted with (i) one substituent selected from the group consisting of halogen, cyano, —SOₜN(H)R⁷, —CON(H)R⁷, —N(H)SOₜR⁸, —N(H)COR⁷, and 3-6 membered heterocyclyl, and (ii) 0, 1, 2, or 3 substituents independent selected from the group consisting of halogen, hydroxyl, cyano, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃-7 cycloalkyl).

In certain embodiments, A³ is a 5-6 membered heteroaryl substituted by (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃-7 cycloalkyl). In certain embodiments, A³ is 5-6 membered heteroaryl substituted with (i) one substituent selected from the group consisting of halogen, cyano, —SOₜN(H)R⁷, —CON(H)R⁷, —N(H)SOₜR⁸, —N(H)COR⁷, and 3-6 membered heterocyclyl, and (ii) 0, 1, 2, or 3 substituents independent selected from the group consisting of halogen, hydroxyl, cyano, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃-7 cycloalkyl).

In certain embodiments, A³ is a 6-membered heteroaryl substituted by (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃-7 cycloalkyl).

In certain embodiments, A³ is

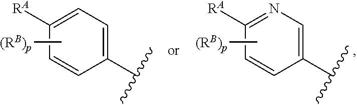

wherein R^{A} is halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), or —N(R⁷)SOₜ-(3-6 membered heterocyclyl); and R^{B} is halogen, hydroxyl, oxo, cyano, —N(R⁷)₂, C₁₋₆ alkyl, C₁₋₆ haloalkyl, and C₁₋₆ hydroxyalkyl, and p is 0, 1, or 2; and R⁸ is C₁₋₆ alkyl, C₁₋₆ haloalkyl, or —(C₀₋₆ alkylene)-(C₃₋₇ cycloalkyl). In certain embodiments, A³ is

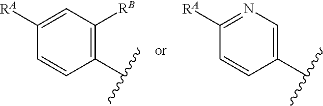

wherein R^{A} is halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), or —N(R⁷)SOₜ-(3-6 membered heterocyclyl); and R^{B} is halogen, hydroxyl, oxo, cyano, —N(R⁷)₂, C₁₋₆ alkyl, C₁₋₆ haloalkyl, or C₁₋₆ hydroxyalkyl; and R⁸ is C₁₋₆ alkyl, C₁₋₆ haloalkyl, or —(C₀₋₆ alkylene)-(C₃-7 cycloalkyl).

In certain embodiments, A³ is a 3-10 membered monocyclic saturated or partially unsaturated heterocyclyl or 5-10 membered bicyclic heterocyclyl, each of which is substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

In certain embodiments, A³ is a 3-10 membered monocyclic saturated or partially unsaturated heterocyclyl substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

In certain embodiments, A³ is a 3-7 membered monocyclic heterocycloalkyl substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

In certain embodiments, A³ is a 3-7 membered monocyclic partially unsaturated heterocyclyl substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

In certain embodiments, A³ is a 6-membered monocyclic partially unsaturated heterocyclyl substituted with (i) one substituent selected from the group consisting of halogen, cyano, —SOₜN(H)R⁷, —CON(H)R⁷, —N(H)SOₜR⁷, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

In certain embodiments, A³ is

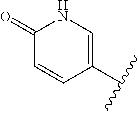 [SMILES: *c1ccc(=O)[nH]c1]

substituted by (i) one substituent selected from the group consisting of cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, or C₁₋₆ hydroxyalkyl; and R⁸ is C₁₋₆ alkyl, C₁₋₆ haloalkyl, and —(C₀₋₆ alkylene)-(C₃₋₇ cycloalkyl); and R⁸ is C₁₋₆ alkyl, C₁₋₆ haloalkyl, or —(C₀₋₆ alkylene)-(C₃-7 cycloalkyl).

In certain embodiments, X¹ is 2-4 membered heteroalkylene or C₁₋₄ alkylene, each of which is substituted by 0, 1, or 2 occurrences of R⁴ and optionally one or more occurrences of halogen. In certain embodiments, X¹ is 2-4 membered heteroalkylene or C₁₋₄ alkylene, each of which is substituted by 0, 1, or 2 occurrences of R⁴. In certain embodiments, X¹ is 2-4 membered heteroalkylene.

In certain embodiments, X¹ is 2-4 membered heteroalkylene or C₁₋₄ alkylene, each of which is optionally substituted with one or more occurrences of halogen. In certain embodiments, X¹ is 2-4 membered heteroalkylene or C₁₋₄ alkylene. In certain embodiments, X¹ is 2-4 membered heteroalkylene optionally substituted with one or more occurrences of halogen.

In certain embodiments, X¹ is —(C₁₋₂ alkylene)-S-ϕ, —(C₁₋₂ alkylene)-O-ϕ, —(C₁₋₂ alkylene)-SOₜ-ϕ, or C₁₋₃ alkylene; each of which is substituted by 0, 1, or 2 occurrences of R⁴; wherein ϕ is a bond to A¹. In certain embodiments, X¹ is —(C₁₋₂ alkylene)-S-ϕ or —(C₁₋₂ alkylene)-O-ϕ, wherein the alkylene portion of each is optionally substituted with 1 or 2 occurrences of R⁴; wherein ϕ is a bond to A¹. In certain embodiments, X¹ is —(C₁₋₂ alkylene)-S-ϕ or —(C₁₋₂ alkylene)-O-ϕ; wherein ϕ is a bond to A¹.

In certain embodiments, X¹ is —(C₁₋₂ alkylene)-S-ϕ optionally substituted with 1 or 2 occurrences of R⁴; wherein 4 is a bond to A¹. In certain embodiments, X¹ is —CH₂—S—; wherein ϕ is a bond to A¹. In certain embodiments, X¹ is —(C₁₋₂ alkylene)-O-ϕ optionally substituted with 1 or 2 occurrences of R⁴; wherein 4 is a bond to A¹. In certain embodiments, X¹ is —CH₂—O-ϕ; wherein ϕ is a bond to A¹. In certain embodiments, X¹ is —(C₁₋₂ alkylene)-SOₜ-ϕ; wherein ϕ is a bond to A¹. In certain embodiments, X¹ is —CH₂— SOₜ-ϕ; wherein ϕ is a bond to A¹.

In certain embodiments, X¹ is C₁₋₃ alkylene optionally substituted with 1 or 2 occurrences of R⁴. In certain embodiments, X¹ is C₁₋₃ alkylene.

In certain embodiments, X² is C₁₋₄ alkylene substituted by 0, 1, or 2 occurrences of R⁵. In certain embodiments, X² is C₁₋₄ alkylene. In certain embodiments, X² is C₁₋₃ alkylene. In certain embodiments, X² is —CH₂—. In certain embodiments, X² is C₁₋₃ alkylene substituted with 1 or 2 occurrences of R⁵.

In certain embodiments, X² is C₁₋₄ alkylene substituted by 0, 1, or 2 occurrences of R⁵ and optionally one or more occurrences of halogen. In certain embodiments, X² is C₁₋₄ alkylene optionally substituted by one or more occurrences of halogen. In certain embodiments, X² is —(C₁₋₃ alkylene)-C(O)-ψ substituted by 0, 1, or 2 occurrences of R⁵ and optionally one or more occurrences of halogen; wherein ψ is a bond to A². In certain embodiments, X² is —(C₁₋₄ alkylene)-C(O)N(R⁷)-ψ substituted by 0, 1, or 2 occurrences of R⁵ and optionally one or more occurrences of halogen; wherein ψ is a bond to A².

In certain embodiments,

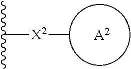

is —(C₁₋₄ alkylene)-C(O)N(R⁷)-(5-membered heteroaryl).

In certain embodiments, X³ is C₁₋₃ alkylene or C₂₋₃ alkenylene, each of which is substituted by 0, 1, or 2 occurrences of R⁶. In certain embodiments, X³ is C₁₋₄ alkylene substituted by 0, 1, or 2 occurrences of R⁶. In certain embodiments, X³ is C₁₋₄ alkylene. In certain embodiments, X³ is C₁₋₃ alkylene optionally substituted with 1 or 2 occurrences of R⁶. In certain embodiments, X³ is C₁₋₃ alkylene. In certain embodiments, X³ is —CH₂—. In certain embodiments, X³ is C₁₋₄ alkylene optionally substituted by one or more occurrences of halogen.

In certain embodiments, X³ is C₂₋₄ alkenylene substituted by 0, 1, or 2 occurrences of R⁶ and optionally one or more occurrences of halogen. In certain embodiments, X³ is C₂₋₃ alkenylene optionally substituted with 1 or 2 occurrences of R⁶. In certain embodiments, X³ is C₂₋₄ alkenylene. In certain embodiments, X³ is C₂₋₃ alkenylene. In certain embodiments, X³ is C₂₋₄ alkynylene substituted by 0, 1, or 2 occurrences of R⁶ and optionally one or more occurrences of halogen. In certain embodiments, X³ is C₂₋₃ alkynylene optionally substituted with 1 or 2 occurrences of R⁶. In certain embodiments, X³ is C₂₋₄ alkynylene. In certain embodiments, X³ is C₂₋₃ alkynylene.

In certain embodiments, X¹ is 2-4 membered heteroalkylene, X² is C₁₋₄ alkylene, and X³ is C₁₋₄ alkylene.

In certain embodiments, R¹ is halogen, cyano, C₁₋₄ alkyl, C₁₋₄ haloalkyl, —O—C₁₋₄ alkyl, —O—C₁₋₄ haloalkyl, —S—C₁₋₄ alkyl, or —S—C₁₋₄ haloalkyl. In certain embodiments, R¹ is halogen, C₁₋₄ alkyl, C₁₋₄ haloalkyl, —O—C₁₋₄ alkyl, or —O—C₁₋₄ haloalkyl. In certain embodiments, R¹ is fluoro, chloro, methyl, or ethyl.

In certain embodiments, R¹ is halogen. In certain embodiments, R¹ is C₁₋₄ alkyl or C₁₋₄ haloalkyl. In certain embodiments, R¹ is C₁₋₄ alkyl. In certain embodiments, R¹ is C₁₋₄ haloalkyl. In certain embodiments, R¹ is —O—C₁₋₄ alkyl or —O—C₁₋₄ haloalkyl.

In certain embodiments, R² represents independently for each occurrence halogen, cyano, hydroxyl, —N(R⁷)₂, —CON(R⁷)₂, —SOₜN(R⁷)₂, C₁₋₆ alkyl, —O—C₁₋₆ alkyl, —S—C₁₋₆ alkyl, C₂₋₆ alkenyl, or C₂₋₆ alkynyl; wherein each of the foregoing C₁₋₆ alkyl groups is optionally substituted with —OR⁷, —N(R⁷)₂, —CON(R⁷)₂, —SOₜN(R⁷)₂, or 1-3 halogens. In certain embodiments, R² represents independently for each occurrence halogen, cyano, hydroxyl, —N(R⁷)₂, —CON(R⁷)₂, —SOₜN(R⁷)₂, C₁₋₆ alkyl, or —O—C₁₋₆ alkyl; wherein each of the foregoing C₁₋₆ alkyl groups is optionally substituted 1-3 halogens. In certain embodiments, R² represents independently for each occurrence halogen, C₁₋₆ alkyl, or —O—C₁₋₆ alkyl; wherein said C₁₋₆ alkyl and —O—C₁₋₆ alkyl are each optionally substituted with —OR⁷, —N(R⁷)₂, or 1-3 halogens. In certain embodiments, R² represents independently for each occurrence fluoro, chloro, methyl, or ethyl.

In certain embodiments, R² represents independently for each occurrence C₃₋₆ cycloalkyl, —O—C₃₋₆ cycloalkyl, or 3-6 membered heterocyclyl; each of which are optionally substituted with 1 or 2 substituents independently selected from C₁₋₆ alkyl, hydroxyl, oxo, —N(R⁷)₂, —CON(R⁷)₂, —SOₜN(R⁷)₂, and halogen; wherein said C₁₋₆ alkyl is optionally substituted with —OR⁷, —N(R⁷)₂, —CON(R⁷)₂, —SOₜN(R⁷)₂, or 1-3 halogens. In certain embodiments, R² represents independently for each occurrence 3-6 membered heterocyclyl optionally substituted with 1 or 2 substituents independently selected from C₁₋₆ alkyl, hydroxyl, oxo, —N(R⁷)₂, and halogen; wherein said C₁₋₆ alkyl is optionally substituted with —OR⁷, —N(R⁷)₂, or 1-3 halogens.

In certain embodiments, R² represents independently for each occurrence halogen. In certain embodiments, R² represents independently for each occurrence fluoro or chloro. In certain embodiments, R² represents independently for each occurrence C₁₋₄alkyl optionally substituted with 1-3 halogens. In certain embodiments, R² represents independently for each occurrence C₁₋₄alkyl. In certain embodiments, R² represents independently for each occurrence methyl or ethyl. In certain embodiments, R² represents independently for each occurrence hydroxyl, —N(R⁷)₂, —CON(R⁷)₂, or —SOₜN(R⁷)₂.

In certain embodiments, R³ represents independently for each occurrence —(C₀₋₄ alkylene)-N(R⁷)₂. In certain embodiments, R³ is —N(R⁷)₂.

In certain embodiments, R³ represents independently for each occurrence —N(R⁷)₂, —OR⁷, C₁₋₄ alkyl, C₁₋₄ haloalkyl, or halogen. In certain embodiments, R³ represents independently for each occurrence —(C₀₋₄ alkylene)-OR⁷. In certain embodiments, R³ represents independently for each —OR⁷. In certain embodiments, R³ represents independently for each occurrence C₁₋₄ alkyl. In certain embodiments, R³ represents independently for each occurrence methyl or ethyl. In certain embodiments, R³ represents independently for each occurrence C₁₋₄ haloalkyl. In certain embodiments, R³ represents independently for each occurrence C₁₋₂ haloalkyl. In certain embodiments, R³ represents independently for each occurrence halogen. In certain embodiments, R³ represents independently for each occurrence fluoro or chloro. In certain embodiments, R³ represents independently for each occurrence —(C₀₋₄ alkylene)-N(R⁷)₂, —(C₀₋₄ alkylene)-OR⁷, C₁₋₄ alkyl, C₁₋₄ haloalkyl, or halogen.

In certain embodiments, two occurrences of R⁴ are taken together with the carbon atom or atoms to which they are attached to form a 3-7 membered ring optionally substituted with 1 or 2 substituents independently selected from halogen, C₁₋₄ alkyl, C₁₋₄ haloalkyl, —(C₀₋₄ alkylene)-N(R⁷)₂, and —(C₀₋₄ alkylene)-OR⁷. In certain embodiments, two occurrences of R⁴ are taken together with the carbon atom or atoms to which they are attached to form a 3-5 membered carbocyclic ring optionally substituted with 1 or 2 substituents independently selected from halogen, C₁₋₄ alkyl, and C₁₋₄ haloalkyl. In certain embodiments, two occurrences of R⁴ are taken together with the carbon atom or atoms to which they are attached to form a saturated 3-membered carbocyclic ring. In certain embodiments, two occurrences of R⁴ are taken together with the carbon atom or atoms to which they are attached to form a 3-5 membered heterocyclic ring optionally substituted with 1 or 2 substituents independently selected from halogen, C₁₋₄ alkyl, and C₁₋₄ haloalkyl.

In certain embodiments, R⁵ represents independently for each occurrence —(C₀₋₄ alkylene)-N(R⁷)₂ or —(C₀₋₄ alkylene)-OR⁷. In certain embodiments, R⁵ represents independently for each occurrence —(C₁₋₄ alkylene)-N(R⁷)₂ or —(C₁₋₄ alkylene)-OR⁷.

In certain embodiments, two occurrences of R⁵ are taken together with the carbon atom or atoms to which they are attached to form a 3-7 membered ring optionally substituted with 1 or 2 substituents independently selected from halogen, C₁₋₄ alkyl, C₁₋₄ haloalkyl, —(C₀₋₄ alkylene)-N(R⁷)₂, and —(C₀₋₄ alkylene)-OR⁷. In certain embodiments, two occurrences of R⁵ are taken together with the carbon atom or atoms to which they are attached to form a 3-5 membered carbocyclic ring optionally substituted with 1 or 2 substituents independently selected from halogen, C₁₋₄ alkyl, and C₁₋₄ haloalkyl. In certain embodiments, two occurrences of R⁵ are taken together with the carbon atom or atoms to which they are attached to form a saturated 3-5 membered carbocyclic ring. In certain embodiments, two occurrences of R⁵ are taken together with the carbon atom or atoms to which they are attached to form a 3-5 membered heterocyclic ring optionally substituted with 1 or 2 substituents independently selected from halogen, C₁₋₄ alkyl, and C₁₋₄ haloalkyl.

In certain embodiments, two occurrences of R⁶ are taken together with the carbon atom or atoms to which they are attached to form a 3-7 membered ring optionally substituted with 1 or 2 substituents independently selected from halogen, C₁₋₄ alkyl, C₁₋₄ haloalkyl, —(C₀₋₄ alkylene)-N(R⁷)₂, and —(C₀₋₄ alkylene)-OR⁷. In certain embodiments, two occurrences of R⁶ are taken together with the carbon atom or atoms to which they are attached to form a 3-5 membered carbocyclic ring optionally substituted with 1 or 2 substituents independently selected from halogen, C₁₋₄ alkyl, and C₁₋₄ haloalkyl. In certain embodiments, two occurrences of R⁶ are taken together with the carbon atom or atoms to which they are attached to form a saturated 3-membered carbocyclic ring. In certain embodiments, two occurrences of R⁶ are taken together with the carbon atom or atoms to which they are attached to form a 3-5 membered heterocyclic ring optionally substituted with 1 or 2 substituents independently selected from halogen, C₁₋₄ alkyl, and C₁₋₄ haloalkyl.

In certain embodiments, R⁷ represents independently for each occurrence hydrogen, C₁₋₆ alkyl, C₁₋₆ haloalkyl, or —(C₀₋₆ alkylene)-(C₃₋₇ cycloalkyl). In certain embodiments, R⁷ represents independently for each occurrence hydrogen or C₁₋₄ alkyl. In certain embodiments, R⁷ represents independently for each occurrence hydrogen or methyl. In certain embodiments, R⁷ is hydrogen. In certain embodiments, R⁷ is methyl.

In certain embodiments, R⁸ is C₁₋₆ alkyl or C₁₋₆ haloalkyl. In certain embodiments, R⁸ is C₁₋₄ alkyl or C₁₋₄ haloalkyl. In certain embodiments, R⁸ is C₁₋₄ alkyl. In certain embodiments, R⁸ is C₁₋₄ haloalkyl.

In certain embodiments, t is 2. In certain embodiments, t is 1.

The description above describes multiple embodiments relating to compounds of Formula I. The patent application specifically contemplates all combinations of the embodiments.

Another aspect of the invention provides a compound represented by Formula I-1:

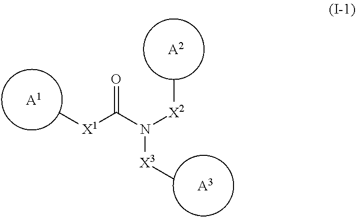

or a pharmaceutically acceptable salt thereof, wherein:
A¹ is a phenyl, 6-10 membered aza-heterocyclyl, or C₃₋₇ cycloalkyl, each of which is substituted with (i) one occurrence of R¹ and (ii) 0, 1, 2, or 3 occurrences of R²;
A² is one of the following:
   a 5-membered heteroaryl containing at least two ring heteroatoms independently selected from the group consisting of nitrogen, oxygen, and sulfur, wherein at least one ring heteroatom is nitrogen, and said heteroaryl is substituted with 0, 1, 2, or 3 occurrences of R³; or
   a 9-10 membered bicyclic aza-heteroaryl or 4-7 membered heterocycloalkyl, each of which is substituted with 0, 1, 2, or 3 occurrences of R³;
A³ is a 3-10 membered carbocyclyl or 3-10 membered heterocyclyl;
X¹ is 2-4 membered heteroalkylene or C₁₋₄ alkylene, each of which is substituted by 0, 1, or 2 occurrences of R⁴ and optionally one or more occurrences of halogen;
X² is C₁₋₄ alkylene, —(C₁₋₃ alkylene)-C(O)-ψ, or —(C₁₋₄ alkylene)-C(O)N(R⁷)-ψ, each of which substituted by 0, 1, or 2 occurrences of R⁵ and optionally one or more occurrences of halogen; wherein ψ is a bond to A²; or

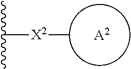

is —(C₁₋₄ alkylene)-C(O)N(R⁷)-(5-membered heteroaryl);
X³ is C₁₋₄ alkylene, C₂₋₄ alkenylene, or C₂₋₄ alkynylene; each of which substituted by 0, 1, or 2 occurrences of R⁶ and optionally one or more occurrences of halogen;
R¹ is halogen, cyano, C₁₋₄ alkyl, C₁₋₄ haloalkyl, —O—C₁₋₄ alkyl, —O—C₁₋₄ haloalkyl, —S—C₁₋₄ alkyl, or —S—C₁₋₄ haloalkyl;
R² represents independently for each occurrence halogen, cyano, hydroxyl, —N(R⁷)₂, —CON(R⁷)₂, —SOₜN(R⁷)₂, C₁₋₆ alkyl, —O—C₁₋₆ alkyl, —S—C₁₋₆ alkyl, C₂₋₆ alkenyl, C₂₋₆ alkynyl, C₃₋₆ cycloalkyl, —O—C₃₋₆ cycloalkyl, or 3-6 membered heterocyclyl; wherein the C₃₋₆ cycloalkyl, —O—C₃₋₆ cycloalkyl, and 3-6 membered heterocyclyl are each optionally substituted with 1 or 2 substituents independently selected from C₁₋₆ alkyl, hydroxyl, oxo, —N(R⁴)₂, —CON(R⁴)₂, —SOₜN(R⁶)₂, and halogen; wherein each of the foregoing C₁₋₆ alkyl groups is optionally substituted with —OR⁷, —N(R⁷)₂, —CON(R⁷)₂, —SOₜN(R⁷)₂, or 1-3 halogens;
R³ represents independently for each occurrence —(C₀₋₄ alkylene)-N(R⁷)₂, —(C₀₋₄ alkylene)-OR⁷, C₁₋₄ alkyl, C₁₋₄ haloalkyl, or halogen;
R⁴, R⁵, and R⁶ each represent independently for each occurrence —(C₀₋₄ alkylene)-N(R⁷)₂ or —(C₀₋₄ alkylene)-OR⁷; or two occurrences of R⁴ are taken together with the carbon atom or atoms to which they are attached to form X⁴, two occurrences of R⁵ are taken together with the carbon atom or atoms to which they are attached to form X⁴, and/or two occurrences of R⁶ are taken together with the carbon atom or atoms to which they are attached to form X⁴; wherein X⁴ is a 3-7 membered ring optionally substituted with 1 or 2 substituents independently selected from halogen, C₁₋₄ alkyl, C₁₋₄ haloalkyl, —(C₀₋₄ alkylene)-N(R⁷)₂, and —(C₀₋₄ alkylene)-OR⁷;
R⁷ represents independently for each occurrence hydrogen, C₁₋₆ alkyl, C₁₋₆ haloalkyl, or —(C₀₋₆ alkylene)-(C₃₋₇ cycloalkyl); or two R⁷ attached to same nitrogen atom are taken together with the nitrogen atom to which they are attached to form a 3-7 membered heterocyclic ring; and
t represents independently for each occurrence 1 or 2.

The definitions of variables in Formula I-1 above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is represented by Formula I-1. In certain embodiments, the definition of one or more of A¹, A², A³, X¹, X², R¹, R², R³, R⁴, R⁵, R⁶, R⁷, and t is as set forth in the embodiments above for Formula I.

The description above describes multiple embodiments relating to compounds of Formula I-1. The patent application specifically contemplates all combinations of the embodiments.

Another aspect of the invention provides a compound represented by Formula I-A:

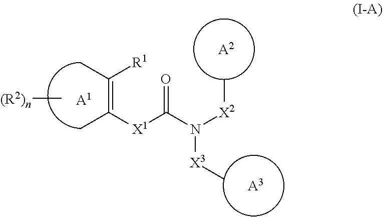

or a pharmaceutically acceptable salt thereof, wherein:
A¹ is phenyl or 6-membered aza-heteroaryl;
A² is a 5-membered heteroaryl containing at least two ring heteroatoms independently selected from the group consisting of nitrogen, oxygen, and sulfur, wherein at least one ring heteroatom is nitrogen, and said heteroaryl is substituted with 0, 1, 2, or 3 occurrences of R³;
A³ is 3-10 membered carbocyclyl or 3-10 membered heterocyclyl;
X¹ is —(C₁₋₂ alkylene)-S-ϕ, —(C₁₋₂ alkylene)-O-ϕ, —(C₁₋₂ alkylene)-SOₜ-ϕ, or C₁₋₃ alkylene; each of which is substituted by 0, 1, or 2 occurrences of R⁴; wherein 4 is a bond to A¹;
X² is C₁₋₃ alkylene optionally substituted with 1 or 2 occurrences of R⁵;
X³ is C₁₋₃ alkylene or C₂₋₃ alkenylene, each of which is substituted by 0, 1, or 2 occurrences of R⁶;
R¹ is halogen, cyano, C₁₋₄ alkyl, C₁₋₄ haloalkyl, —O—C₁₋₄ alkyl, —O—C₁₋₄ haloalkyl, —S—C₁₋₄ alkyl, or —S—C₁₋₄ haloalkyl;
R² represents independently for each occurrence halogen, cyano, hydroxyl, —N(R⁷)₂, —CON(R⁷)₂, —SOₜN(R⁷)₂, C₁₋₆ alkyl, —O—C₁₋₆ alkyl, —S—C₁₋₆ alkyl, C₂₋₆ alkenyl, C₂₋₆ alkynyl, C₃₋₆ cycloalkyl, —O—C₃₋₆ cycloalkyl, or 3-6 membered heterocyclyl; wherein the C₃₋₆ cycloalkyl, —O—C₃₋₆ cycloalkyl, and 3-6 membered heterocyclyl are each optionally substituted with 1 or 2 substituents independently selected from C₁₋₆ alkyl, hydroxyl, oxo, —N(R⁴)₂, —CON(R⁴)₂, —SOₜN(R⁴)₂, and halogen; wherein each of the foregoing C₁₋₆ alkyl groups is optionally substituted with —OR⁷, —N(R⁷)₂, —CON(R⁷)₂, —SOₜN(R⁷)₂, or 1-3 halogens;
R³ represents independently for each occurrence —(C₀₋₄ alkylene)-N(R⁷)₂, —(C₀₋₄ alkylene)-OR⁷, C₁₋₄ alkyl, C₁₋₄ haloalkyl, or halogen;
R⁴, R⁵, and R⁶ each represent independently for each occurrence —(C₀₋₄ alkylene)-N(R⁷)₂ or —(C₀₋₄ alkylene)-OR⁷; or two occurrences of R⁴ are taken together with the carbon atom or atoms to which they are attached to form X⁴, two occurrences of R⁵ are taken together with the carbon atom or atoms to which they are attached to form X⁴, and/or two occurrences of R⁶ are taken together with the carbon atom or atoms to which they are attached to form X⁴; wherein X⁴ is a 3-7 membered ring optionally substituted with 1 or 2 substituents independently selected from halogen, C₁₋₄ alkyl, C₁₋₄ haloalkyl, —(C₀₋₄ alkylene)-N(R⁷)₂, and —(C₀₋₄ alkylene)-OR⁷;
R⁷ represents independently for each occurrence hydrogen, C₁₋₆ alkyl, C₁₋₆ haloalkyl, or —(C₀₋₆ alkylene)-(C₃₋₇ cycloalkyl);
n is 0, 1, or 2; and
t represents independently for each occurrence 1 or 2.

The definitions of variables in Formula I-A above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is represented by Formula I-A.

In certain embodiments, is

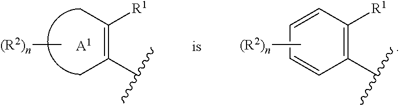

In certain embodiments,

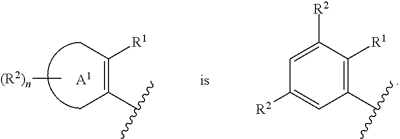

In certain embodiments,

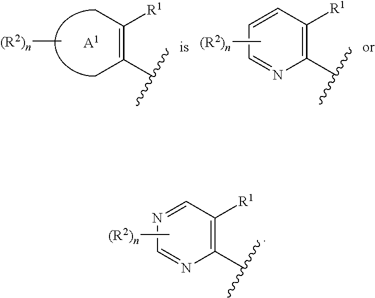

In certain embodiments,

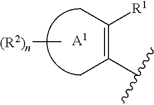

is

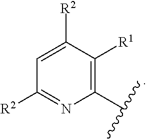

In certain embodiments, R¹ is halogen, C₁₋₄ alkyl, C₁₋₄ haloalkyl, —O—C₁₋₄ alkyl, or —O—C₁₋₄ haloalkyl. In certain embodiments, R¹ is fluoro, chloro, methyl, or ethyl.

In certain embodiments, R¹ is halogen. In certain embodiments, R¹ is C₁₋₄ alkyl or C₁₋₄ haloalkyl. In certain embodiments, R¹ is C₁₋₄ alkyl. In certain embodiments, R¹ is C₁₋₄ haloalkyl. In certain embodiments, R¹ is —O—C₁₋₄ alkyl or —O—C₁₋₄ haloalkyl.

In certain embodiments, R² represents independently for each occurrence halogen, cyano, hydroxyl, —N(R⁷)₂, —CON(R⁷)₂, —SOₜN(R⁷)₂, C₁₋₆ alkyl, —O—C₁₋₆ alkyl, —S—C₁₋₆ alkyl, C₂₋₆ alkenyl, or C₂₋₆ alkynyl; wherein each of the foregoing C₁₋₆ alkyl groups is optionally substituted with —OR⁷, —N(R⁷)₂, —CON(R⁷)₂, —SOₜN(R⁷)₂, or 1-3 halogens. In certain embodiments, R² represents independently for each occurrence halogen, cyano, hydroxyl, —N(R⁷)₂, —CON(R⁷)₂, —SOₜN(R⁷)₂, C₁₋₆ alkyl, or —O—C₁₋₆ alkyl; wherein each of the foregoing C₁₋₆ alkyl groups is optionally substituted 1-3 halogens. In certain embodiments, R² represents independently for each occurrence halogen, C₁₋₆ alkyl, or —O—C₁₋₆ alkyl; wherein said C₁₋₆ alkyl and —O—C₁₋₆ alkyl are each optionally substituted with —OR⁷, —N(R⁷)₂, or 1-3 halogens. In certain embodiments, R² represents independently for each occurrence fluoro, chloro, methyl, or ethyl.

In certain embodiments, R² represents independently for each occurrence C₃₋₆ cycloalkyl, —O—C₃₋₆ cycloalkyl, or 3-6 membered heterocyclyl; each of which are optionally substituted with 1 or 2 substituents independently selected from C₁₋₆ alkyl, hydroxyl, oxo, —N(R⁷)₂, —CON(R⁷)₂, —SOₜN(R⁷)₂, and halogen; wherein said C₁₋₆ alkyl is optionally substituted with —OR⁷, —N(R⁷)₂, —CON(R⁷)₂, —SOₜN(R⁷)₂, or 1-3 halogens. In certain embodiments, R² represents independently for each occurrence 3-6 membered heterocyclyl optionally substituted with 1 or 2 substituents independently selected from C₁₋₆ alkyl, hydroxyl, oxo, —N(R⁷)₂, and halogen; wherein said C₁₋₆ alkyl is optionally substituted with —OR⁷, —N(R⁷)₂, or 1-3 halogens.

In certain embodiments, R² represents independently for each occurrence halogen. In certain embodiments, R² represents independently for each occurrence fluoro or chloro. In certain embodiments, R² represents independently for each occurrence C₁₋₄alkyl optionally substituted with 1-3 halogens. In certain embodiments, R² represents independently for each occurrence C₁₋₄alkyl. In certain embodiments, R² represents independently for each occurrence methyl or ethyl. In certain embodiments, R² represents independently for each occurrence hydroxyl, —N(R⁷)₂, —CON(R⁷)₂, or —SOₜN(R⁷)₂.

In certain embodiments, R³ represents independently for each occurrence —(C₀₋₄ alkylene)-N(R⁷)₂. In certain embodiments, R³ is —N(R⁷)₂.

In certain embodiments, R³ represents independently for each occurrence —N(R⁷)₂, —OR⁷, C₁₋₄ alkyl, C₁₋₄ haloalkyl, or halogen. In certain embodiments, R³ represents independently for each occurrence —(C₀₋₄ alkylene)-OR⁷. In certain embodiments, R³ represents independently for each —OR⁷. In certain embodiments, R³ represents independently for each occurrence C₁₋₄ alkyl. In certain embodiments, R³ represents independently for each occurrence methyl or ethyl. In certain embodiments, R³ represents independently for each occurrence C₁₋₄ haloalkyl. In certain embodiments, R³ represents independently for each occurrence C₁₋₂ haloalkyl. In certain embodiments, R³ represents independently for each occurrence halogen. In certain embodiments, R³ represents independently for each occurrence fluoro or chloro. In certain embodiments, R³ represents independently for each occurrence —(C₀₋₄ alkylene)-N(R⁷)₂, —(C₀₋₄ alkylene)-OR⁷, C₁₋₄ alkyl, C₁₋₄ haloalkyl, or halogen.

In certain embodiments, two occurrences of R⁴ are taken together with the carbon atom or atoms to which they are attached to form a 3-7 membered ring optionally substituted with 1 or 2 substituents independently selected from halogen, C₁₋₄ alkyl, C₁₋₄ haloalkyl, —(C₀₋₄ alkylene)-N(R⁷)₂, and —(C₀₋₄ alkylene)-OR⁷. In certain embodiments, two occurrences of R⁴ are taken together with the carbon atom or atoms to which they are attached to form a 3-5 membered carbocyclic ring optionally substituted with 1 or 2 substituents independently selected from halogen, C₁₋₄ alkyl, and C₁₋₄ haloalkyl. In certain embodiments, two occurrences of R⁴ are taken together with the carbon atom or atoms to which they are attached to form a saturated 3-membered carbocyclic ring. In certain embodiments, two occurrences of R⁴ are taken together with the carbon atom or atoms to which they are attached to form a 3-5 membered heterocyclic ring optionally substituted with 1 or 2 substituents independently selected from halogen, C₁₋₄ alkyl, and C₁₋₄ haloalkyl.

In certain embodiments, R⁵ represents independently for each occurrence —(C₀₋₄ alkylene)-N(R⁷)₂ or —(C₀₋₄ alkylene)-OR⁷. In certain embodiments, R⁵ represents independently for each occurrence —(C₁₋₄ alkylene)-N(R⁷)₂ or —(C₁₋₄ alkylene)-OR⁷.

In certain embodiments, two occurrences of R⁵ are taken together with the carbon atom or atoms to which they are attached to form a 3-7 membered ring optionally substituted with 1 or 2 substituents independently selected from halogen, C₁₋₄ alkyl, C₁₋₄ haloalkyl, —(C₀₋₄ alkylene)-N(R⁷)₂, and —(C₀₋₄ alkylene)-OR⁷. In certain embodiments, two occurrences of R⁵ are taken together with the carbon atom or atoms to which they are attached to form a 3-5 membered carbocyclic ring optionally substituted with 1 or 2 substituents independently selected from halogen, C₁₋₄ alkyl, and C₁₋₄ haloalkyl. In certain embodiments, two occurrences of R⁵ are taken together with the carbon atom or atoms to which they are attached to form a saturated 3-5 membered carbocyclic ring. In certain embodiments, two occurrences of R⁵ are taken together with the carbon atom or atoms to which they are attached to form a 3-5 membered heterocyclic ring optionally substituted with 1 or 2 substituents independently selected from halogen, C₁₋₄ alkyl, and C₁₋₄ haloalkyl.

In certain embodiments, two occurrences of R⁶ are taken together with the carbon atom or atoms to which they are attached to form a 3-7 membered ring optionally substituted with 1 or 2 substituents independently selected from halogen, C₁₋₄ alkyl, C₁₋₄ haloalkyl, —(C₀₋₄ alkylene)-N(R⁷)₂, and —(C₀₋₄ alkylene)-OR⁷. In certain embodiments, two occurrences of R⁶ are taken together with the carbon atom or atoms to which they are attached to form a 3-5 membered carbocyclic ring optionally substituted with 1 or 2 substituents independently selected from halogen, C₁₋₄ alkyl, and C₁₋₄ haloalkyl. In certain embodiments, two occurrences of R⁶ are taken together with the carbon atom or atoms to which they are attached to form a saturated 3-membered carbocyclic ring. In certain embodiments, two occurrences of R⁶ are taken together with the carbon atom or atoms to which they are attached to form a 3-5 membered heterocyclic ring optionally substituted with 1 or 2 substituents independently selected from halogen, C₁₋₄ alkyl, and C₁₋₄ haloalkyl.

In certain embodiments, R⁷ represents independently for each occurrence hydrogen or C₁₋₄ alkyl. In certain embodiments, R⁷ represents independently for each occurrence hydrogen or methyl. In certain embodiments, R⁷ is hydrogen. In certain embodiments, R⁷ is methyl.

In certain embodiments, R⁸ is C₁₋₆ alkyl or C₁₋₆ haloalkyl. In certain embodiments, R⁸ is C₁₋₄ alkyl or C₁₋₄ haloalkyl. In certain embodiments, R⁸ is C₁₋₄ alkyl. In certain embodiments, R⁸ is C₁₋₄ haloalkyl.

In certain embodiments, A² is a 5-membered heteroaryl containing two or three ring heteroatoms each independently selected from the group consisting of nitrogen, oxygen, and sulfur, wherein at least one ring heteroatom is nitrogen, and said heteroaryl is substituted with 0, 1, 2, or 3 occurrences of R³. In certain embodiments, A² is a 5-membered heteroaryl containing two or three ring heteroatoms each independently selected from the group consisting of nitrogen, oxygen, and sulfur, wherein at least two ring heteroatoms are nitrogen, and said heteroaryl is substituted with 0, 1, 2, or 3 occurrences of R³.

In certain embodiments, A² is oxadiazolyl, thiadiazolyl, oxazolyl, triazolyl, imidazolyl, or pyrazolyl, each of which is optionally substituted with 0, 1, 2, or 3 occurrences of R³. In certain embodiments, A² is oxadiazolyl or thiadiazolyl, each of which is optionally substituted with one occurrence of R³. In certain embodiments, A² is

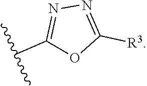

In certain embodiments, A² is

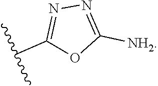 [SMILES: *c1nnc(N)o1]

In certain embodiments, A² is oxazolyl, triazolyl, imidazolyl, or pyrazolyl, each of which is optionally substituted with 1 or 2 occurrences of R³.

In certain embodiments, A³ is a 3-10 membered carbocyclyl or 5-6 membered heteroaryl, each of which is substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, oxo, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀-6 alkylene)-(C₃₋₇ cycloalkyl).

In certain embodiments, A³ is a 3-10 membered carbocyclyl substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, oxo, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl). In certain embodiments, A³ is a 3-10 membered saturated or partially unsaturated carbocyclyl substituted with (i) one substituent selected from the group consisting of halogen, cyano, —SOₜN(H)R⁷, —CON(H)R⁷, —N(H)SOₜR⁷, —N(H)COR⁷, —O—C₁₋₆ alkyl, hydroxyl, oxo, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀-6 alkylene)-(C₃₋₇ cycloalkyl).

In certain embodiments, A³ is naphthyl substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, and 3-6 membered heterocyclyl, and (ii) 0, 1, 2, or 3 substituents independent selected from the group consisting of halogen, C₁₋₆ alkyl, —O—C₁₋₆ alkyl, and C₁₋₆ haloalkyl; and R⁸ is C₁₋₆ alkyl, C₁₋₆ haloalkyl, or —(C₀₋₆ alkylene)-(C₃₋₇ cycloalkyl).

In certain embodiments, A³ is phenyl or a 5-6 membered heteroaryl, each of which is substituted with (i) one substituent selected from the group consisting of halogen, cyano, —SOₜN(H)R⁷, —CON(H)R⁷, —N(H)SOₜR⁷, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

In certain embodiments, A³ is phenyl substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀-6 alkylene)-(C₃₋₇ cycloalkyl). In certain embodiments, A³ is phenyl substituted with (i) one substituent selected from the group consisting of halogen, cyano, —SOₜN(H)R⁷, —CON(H)R⁷, —N(H)SOₜR⁸, —N(H)COR⁷, and 3-6 membered heterocyclyl, and (ii) 0, 1, 2, or 3 substituents independent selected from the group consisting of halogen, hydroxyl, cyano, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

In certain embodiments, A³ is a 5-6 membered heteroaryl substituted by (i) one substituent selected from the group consisting of halogen, cyano, —SOₜN(H)R⁷, —CON(H)R⁷, —N(H)SOₜR⁷, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl). In certain embodiments, A³ is 5-6 membered heteroaryl substituted with (i) one substituent selected from the group consisting of halogen, cyano, —SOₜN(H)R⁷, —CON(H)R⁷, —N(H)SOₜR⁸, —N(H)COR⁷, and 3-6 membered heterocyclyl, and (ii) 0, 1, 2, or 3 substituents independent selected from the group consisting of halogen, hydroxyl, cyano, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

In certain embodiments, A³ is a 6-membered heteroaryl substituted by (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

In certain embodiments, A³ is

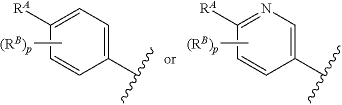

wherein R^{A} is halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), or —N(R⁷)SOₜ-(3-6 membered heterocyclyl); and R^{B} is halogen, hydroxyl, oxo, cyano, —N(R⁷)₂, C₁₋₆ alkyl, C₁₋₆ haloalkyl, and C₁₋₆ hydroxyalkyl, and p is 0, 1, or 2; and R⁸ is C₁₋₆ alkyl, C₁₋₆ haloalkyl, or —(C₀₋₆ alkylene)-(C₃₋₇ cycloalkyl). In certain embodiments, A³ is

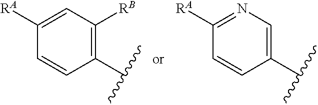

wherein R^{A} is halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), or —N(R⁷)SOₜ-(3-6 membered heterocyclyl); and R^{B} is halogen, hydroxyl, oxo, cyano, —N(R⁷)₂, C₁₋₆ alkyl, C₁₋₆ haloalkyl, or C₁₋₆ hydroxyalkyl; and R⁸ is C₁₋₆ alkyl, C₁₋₆ haloalkyl, or —(C₀₋₆ alkylene)-(C₃₋₇ cycloalkyl).

In certain embodiments, A³ is a 3-10 membered monocyclic saturated or partially unsaturated heterocyclyl or 5-10 membered bicyclic heterocyclyl, each of which is substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

In certain embodiments, A³ is a 3-10 membered monocyclic saturated or partially unsaturated heterocyclyl substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

In certain embodiments, A³ is a 3-7 membered monocyclic heterocycloalkyl substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

In certain embodiments, A³ is a 3-7 membered monocyclic partially unsaturated heterocyclyl substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

In certain embodiments, A³ is a 6-membered monocyclic partially unsaturated heterocyclyl substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

In certain embodiments, A³ is

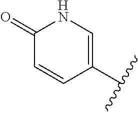 [SMILES: *c1ccc(=O)[nH]c1]

substituted by (i) one substituent selected from the group consisting of cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, or C₁₋₆ hydroxyalkyl; and R⁸ is C₁₋₆ alkyl, C₁₋₆ haloalkyl, and —(C₀₋₆ alkylene)-(C₃₋₇ cycloalkyl); and R⁸ is C₁₋₆ alkyl, C₁₋₆ haloalkyl, or —(C₀₋₆ alkylene)-(C₃₋₇ cycloalkyl).

In certain embodiments, X¹ is —(C₁₋₂ alkylene)-S-ϕ or —(C₁₋₂ alkylene)-O-ϕ, wherein the alkylene portion of each is optionally substituted with 1 or 2 occurrences of R⁴; wherein is a bond to A¹. In certain embodiments, X¹ is —(C₁₋₂ alkylene)-S-ϕ or —(C₁₋₂ alkylene)-O-ϕ; wherein ϕ is a bond to A¹.

In certain embodiments, X¹ is —(C₁₋₂ alkylene)-S-ϕ optionally substituted with 1 or 2 occurrences of R⁴; wherein 4 is a bond to A¹. In certain embodiments, X¹ is —CH₂—S—; wherein ϕ is a bond to A¹. In certain embodiments, X¹ is —(C₁₋₂ alkylene)-O-ϕ optionally substituted with 1 or 2 occurrences of R⁴; wherein 4 is a bond to A¹. In certain embodiments, X¹ is —CH₂—O-ϕ; wherein ϕ is a bond to A¹. In certain embodiments, X¹ is —(C₁₋₂ alkylene)-SOₜ-ϕ; wherein ϕ is a bond to A¹. In certain embodiments, X¹ is —CH₂— SOₜ-ϕ; wherein ϕ is a bond to A¹.

In certain embodiments, X¹ is C₁₋₃ alkylene optionally substituted with 1 or 2 occurrences of R⁴. In certain embodiments, X¹ is C₁₋₃ alkylene.

In certain embodiments, X² is C₁₋₃ alkylene. In certain embodiments, X² is —CH₂—. In certain embodiments, X² is C₁₋₃ alkylene substituted with 1 or 2 occurrences of R⁵.

In certain embodiments, X³ is C₁₋₃ alkylene optionally substituted with 1 or 2 occurrences of R⁶. In certain embodiments, X³ is C₁₋₃ alkylene. In certain embodiments, X³ is —CH₂—. In certain embodiments, X³ is C₂₋₃ alkenylene optionally substituted with 1 or 2 occurrences of R⁶. In certain embodiments, X³ is C₂₋₃ alkenylene.

In certain embodiments, n is 0. In certain embodiments, n is 1 or 2. In certain embodiments, n is 1. In certain embodiments, n is 2. In certain embodiments, n is 0 or 1.

In certain embodiments, t is 2. In certain embodiments, t is 1.

The description above describes multiple embodiments relating to compounds of Formula I-A. The patent application specifically contemplates all combinations of the embodiments.

Another aspect of the invention provides a compound represented by Formula II:

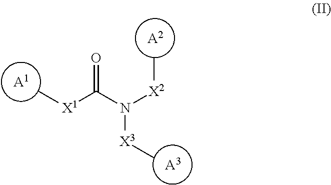

or a pharmaceutically acceptable salt thereof, wherein:
A¹ is a 3-10 membered carbocyclyl or 3-10 membered heterocyclyl;
A² is 5-10 membered heterocyclyl;
A³ is a 3-10 membered carbocyclyl or 3-10 membered heterocyclyl;
X¹ is 2-4 membered heteroalkylene or C₁₋₄ alkylene, each of which is substituted by 0, 1, or 2 occurrences of R⁴ and optionally one or more occurrences of halogen;
X² is C₁₋₄ alkylene, —(C₁₋₃ alkylene)-C(O)-ψ, or —(C₁₋₄ alkylene)-C(O)N(R⁷)-ψ, each of which substituted by 0, 1, or 2 occurrences of R⁵ and optionally one or more occurrences of halogen; wherein ψ is a bond to A²; or

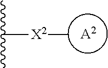

is —(C₁₋₄ alkylene)-C(O)N(R⁷)-(5-membered heteroaryl);
X³ is C₁₋₄ alkylene, C₂₋₄ alkenylene, or C₂₋₄ alkynylene; each of which substituted by 0, 1, or 2 occurrences of R⁶ and optionally one or more occurrences of halogen;
R⁴, R⁵, and R⁶ each represent independently for each occurrence —(C₀₋₄ alkylene)-N(R⁷)₂ or —(C₀₋₄ alkylene)-OR⁷; or two occurrences of R⁴ are taken together with the carbon atom or atoms to which they are attached to form X⁴, two occurrences of R⁵ are taken together with the carbon atom or atoms to which they are attached to form X⁴, and/or two occurrences of R⁶ are taken together with the carbon atom or atoms to which they are attached to form X⁴; wherein X⁴ is a 3-7 membered ring optionally substituted with 1 or 2 substituents independently selected from halogen, C₁₋₄ alkyl, C₁₋₄ haloalkyl, —(C₀₋₄ alkylene)-N(R⁷)₂, and —(C₀₋₄ alkylene)-OR⁷;
R⁷ represents independently for each occurrence hydrogen, C₁₋₆ alkyl, C₁₋₆ haloalkyl, or —(C₀₋₆ alkylene)-(C₃₋₇ cycloalkyl); or two R⁷ attached to same nitrogen atom are taken together with the nitrogen atom to which they are attached to form a 3-7 membered heterocyclic ring; and t represents independently for each occurrence 1 or 2.

The definitions of variables in Formula II above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is represented by Formula II.

In certain embodiments, A¹ is a phenyl, 6-10 membered aza-heterocyclyl, or C₃₋₇ cycloalkyl, each of which is substituted with (i) one occurrence of R¹ and (ii) 0, 1, 2, or 3 occurrences of R²; wherein R¹ is halogen, cyano, C₁₋₄ alkyl, C₁₋₄ haloalkyl, —O—C₁₋₄ alkyl, —O—C₁₋₄ haloalkyl, —S—C₁₋₄ alkyl, or —S—C₁₋₄ haloalkyl; and R² represents independently for each occurrence halogen, cyano, hydroxyl, —N(R⁷)₂, —CON(R⁷)₂, —SOₜN(R⁷)₂, C₁₋₆ alkyl, —O—C₁₋₆ alkyl, —S—C₁₋₆ alkyl, C₂₋₆ alkenyl, C₂₋₆ alkynyl, C₃₋₆ cycloalkyl, —O—C₃₋₆ cycloalkyl, or 3-6 membered heterocyclyl; wherein the C₃₋₆ cycloalkyl, —O—C₃₋₆ cycloalkyl, and 3-6 membered heterocyclyl are each optionally substituted with 1 or 2 substituents independently selected from C₁₋₆ alkyl, hydroxyl, oxo, —N(R⁴)₂, —CON(R⁴)₂, —SOₜN(R⁶)₂, and halogen; wherein each of the foregoing C₁₋₆ alkyl groups is optionally substituted with —OR⁷, —N(R⁷)₂, —CON(R⁷)₂, —SOₜN(R⁷)₂, or 1-3 halogens.

In certain embodiments, A¹ is phenyl or 6-membered aza-heteroaryl, each of which is substituted with (i) one occurrence of R¹ and (ii) 0, 1, 2, or 3 occurrences of R². In certain embodiments, A¹ is phenyl, pyridinyl, or pyrimidinyl; each of which is substituted with (i) one occurrence of R¹ and (ii) 0, 1, 2, or 3 occurrences of R². In certain embodiments, A¹ is a C₃₋₇ cycloalkyl substituted with (i) one occurrence of R¹ and (ii) 0, 1, 2, or 3 occurrences of R². In certain embodiments, A¹ is an 8-10 membered bicyclic aza-heteroaryl substituted with (i) one occurrence of R¹ and (ii) 0, 1, 2, or 3 occurrences of R².

In certain embodiments, A¹ is

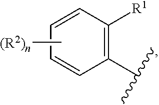

wherein n is 0, 1 or 2. In certain embodiments, A¹ is

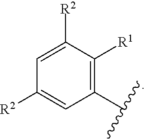

In certain embodiments, A¹ is

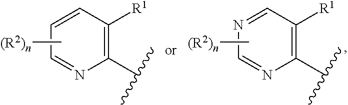

wherein n is 0, 1 or 2. In certain embodiments, n is 0. In certain embodiments, n is 1 or 2. In certain embodiments, n is 1. In certain embodiments, n is 2. In certain embodiments, n is 0 or 1.

In certain embodiments, A¹ is a phenyl substituted with (i) one occurrence of R¹ and (ii) 0, 1, 2, or 3 occurrences of R². In certain embodiments, A¹ is a 6-10 membered aza-heterocyclyl substituted with (i) one occurrence of R¹ and (ii) 0, 1, 2, or 3 occurrences of R².

In certain embodiments, A² is 5-10 membered heterocyclyl substituted with 0, 1, 2, or 3 occurrences of R³; wherein R³ represents independently for each occurrence —(C₀₋₄ alkylene)-N(R⁷)₂, —(C₀₋₄ alkylene)-OR⁷, C₁₋₄ alkyl, C₁₋₄ haloalkyl, or halogen.

In certain embodiments, A² is a 5-membered heteroaryl containing at least two ring heteroatoms independently selected from the group consisting of nitrogen, oxygen, and sulfur, wherein at least one ring heteroatom is nitrogen.

In certain embodiments, A² is a 5-membered heteroaryl containing at least two ring heteroatoms independently selected from the group consisting of nitrogen, oxygen, and sulfur, wherein at least one ring heteroatom is nitrogen, and said heteroaryl is substituted with 0, 1, 2, or 3 occurrences of R³; wherein R³ represents independently for each occurrence —(C₀₋₄ alkylene)-N(R⁷)₂, —(C₀₋₄ alkylene)-OR⁷, C₁₋₄ alkyl, C₁₋₄ haloalkyl, or halogen. In certain embodiments, A² is a 9-10 membered bicyclic aza-heteroaryl substituted with 0, 1, 2, or 3 occurrences of R³. In certain embodiments, A² is a 4-7 membered heterocycloalkyl substituted with 0, 1, 2, or 3 occurrences of R³.

In certain embodiments, A² is a 5-membered heteroaryl containing two or three ring heteroatoms each independently selected from the group consisting of nitrogen, oxygen, and sulfur, wherein at least one ring heteroatom is nitrogen, and said heteroaryl is substituted with 0, 1, 2, or 3 occurrences of R³; wherein R³ represents independently for each occurrence —(C₀₋₄ alkylene)-N(R⁷)₂, —(C₀₋₄ alkylene)-OR⁷, C₁₋₄ alkyl, C₁₋₄ haloalkyl, or halogen. In certain embodiments, A² is a 5-membered heteroaryl containing two or three ring heteroatoms each independently selected from the group consisting of nitrogen, oxygen, and sulfur, wherein at least two ring heteroatoms are nitrogen, and said heteroaryl is substituted with 0, 1, 2, or 3 occurrences of R³.

In certain embodiments, A² is oxadiazolyl, thiadiazolyl, oxazolyl, triazolyl, imidazolyl, or pyrazolyl, each of which is optionally substituted with 0, 1, 2, or 3 occurrences of R³; wherein R³ represents independently for each occurrence —(C₀₋₄ alkylene)-N(R⁷)₂, —(C₀₋₄ alkylene)-OR⁷, C₁₋₄ alkyl, C₁₋₄ haloalkyl, or halogen. In certain embodiments, A² is oxadiazolyl or thiadiazolyl, each of which is optionally substituted with one occurrence of R³. In certain embodiments, A² is

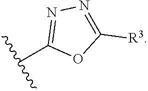

In certain embodiments, A² is

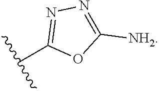 [SMILES: *c1nnc(N)o1]

In certain embodiments, A² is oxazolyl, triazolyl, imidazolyl, or pyrazolyl, each of which is optionally substituted with 1 or 2 occurrences of R³.

In certain embodiments, A³ is a 3-10 membered carbocyclyl or 5-6 membered heteroaryl, each of which is substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, oxo, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

In certain embodiments, A³ is a 3-10 membered carbocyclyl substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, oxo, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl). In certain embodiments, A³ is a 3-10 membered saturated or partially unsaturated carbocyclyl substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, oxo, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀-6 alkylene)-(C₃₋₇ cycloalkyl).

In certain embodiments, A³ is naphthyl substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, and 3-6 membered heterocyclyl, and (ii) 0, 1, 2, or 3 substituents independent selected from the group consisting of halogen, C₁₋₆ alkyl, —O—C₁₋₆ alkyl, and C₁₋₆ haloalkyl; and R⁸ is C₁₋₆ alkyl, C₁₋₆ haloalkyl, or —(C₀₋₆ alkylene)-(C₃₋₇ cycloalkyl).

In certain embodiments, A³ is phenyl or a 5-6 membered heteroaryl, each of which is substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

In certain embodiments, A³ is phenyl substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀-6 alkylene)-(C₃₋₇ cycloalkyl). In certain embodiments, A³ is phenyl substituted with (i) one substituent selected from the group consisting of halogen, cyano, —SOₜN(H)R⁷, —CON(H)R⁷, —N(H)SOₜR⁸, —N(H)COR⁷, and 3-6 membered heterocyclyl, and (ii) 0, 1, 2, or 3 substituents independent selected from the group consisting of halogen, hydroxyl, cyano, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

In certain embodiments, A³ is a 5-6 membered heteroaryl substituted by (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl). In certain embodiments, A³ is 5-6 membered heteroaryl substituted with (i) one substituent selected from the group consisting of halogen, cyano, —SOₜN(H)R⁷, —CON(H)R⁷, —N(H)SOₜR⁸, —N(H)COR⁷, and 3-6 membered heterocyclyl, and (ii) 0, 1, 2, or 3 substituents independent selected from the group consisting of halogen, hydroxyl, cyano, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

In certain embodiments, A³ is a 6-membered heteroaryl substituted by (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

In certain embodiments, A³ is

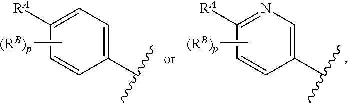

wherein R^{A} is halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), or —N(R⁷)SOₜ-(3-6 membered heterocyclyl); and R^{B} is halogen, hydroxyl, oxo, cyano, —N(R⁷)₂, C₁₋₆ alkyl, C₁₋₆ haloalkyl, and C₁₋₆ hydroxyalkyl, and p is 0, 1, or 2; and R⁸ is C₁₋₆ alkyl, C₁₋₆ haloalkyl, or —(C₀₋₆ alkylene)-(C₃₋₇ cycloalkyl). In certain embodiments, A³ is

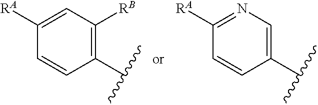

wherein R^{A} is halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), or —N(R⁷)SOₜ-(3-6 membered heterocyclyl); and R^{B} is halogen, hydroxyl, oxo, cyano, —N(R⁷)₂, C₁₋₆ alkyl, C₁₋₆ haloalkyl, or C₁₋₆ hydroxyalkyl; and R⁸ is C₁₋₆ alkyl, C₁₋₆ haloalkyl, or —(C₀₋₆ alkylene)-(C₃₋₇ cycloalkyl).

In certain embodiments, A³ is a 3-10 membered monocyclic saturated or partially unsaturated heterocyclyl or 5-10 membered bicyclic heterocyclyl, each of which is substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

In certain embodiments, A³ is a 3-10 membered monocyclic saturated or partially unsaturated heterocyclyl substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

In certain embodiments, A³ is a 3-7 membered monocyclic heterocycloalkyl substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

In certain embodiments, A³ is a 3-7 membered monocyclic partially unsaturated heterocyclyl substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

In certain embodiments, A³ is a 6-membered monocyclic partially unsaturated heterocyclyl substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

In certain embodiments, A³ is

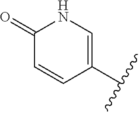 [SMILES: *c1ccc(=O)[nH]c1]

substituted by (i) one substituent selected from the group consisting of cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, or C₁₋₆ hydroxyalkyl; and R⁸ is C₁₋₆ alkyl, C₁₋₆ haloalkyl, and —(C₀₋₆ alkylene)-(C₃₋₇ cycloalkyl); and R⁸ is C₁₋₆ alkyl, C₁₋₆ haloalkyl, or —(C₀₋₆ alkylene)-(C₃₋₇ cycloalkyl).

In certain embodiments, X¹ is 2-4 membered heteroalkylene or C₁₋₄ alkylene, each of which is substituted by 0, 1, or 2 occurrences of R⁴. In certain embodiments, X¹ is 2-4 membered heteroalkylene.

In certain embodiments, X¹ is 2-4 membered heteroalkylene or C₁₋₄ alkylene, each of which is optionally substituted with one or more occurrences of halogen. In certain embodiments, X¹ is 2-4 membered heteroalkylene or C₁₋₄ alkylene. In certain embodiments, X¹ is 2-4 membered heteroalkylene optionally substituted with one or more occurrences of halogen.

In certain embodiments, X¹ is —(C₁₋₂ alkylene)-S-ϕ, —(C₁₋₂ alkylene)-O-ϕ, —(C₁₋₂ alkylene)-SOₜ-ϕ, or C₁₋₃ alkylene; each of which is substituted by 0, 1, or 2 occurrences of R⁴; wherein ϕ is a bond to A¹. In certain embodiments, X¹ is —(C₁₋₂ alkylene)-S-ϕ or —(C₁₋₂ alkylene)-O-ϕ, wherein the alkylene portion of each is optionally substituted with 1 or 2 occurrences of R⁴; wherein 4 is a bond to A¹. In certain embodiments, X¹ is —(C₁₋₂ alkylene)-S— or —(C₁₋₂ alkylene)-O-ϕ; wherein ϕ is a bond to A¹.

In certain embodiments, X¹ is —(C₁₋₂ alkylene)-S-ϕ optionally substituted with 1 or 2 occurrences of R⁴; wherein 4 is a bond to A¹. In certain embodiments, X¹ is —CH₂—S—; wherein ϕ is a bond to A¹. In certain embodiments, X¹ is —(C₁₋₂ alkylene)-O-ϕ optionally substituted with 1 or 2 occurrences of R⁴; wherein 4 is a bond to A¹. In certain embodiments, X¹ is —CH₂—O-ϕ; wherein ϕ is a bond to A¹. In certain embodiments, X¹ is —(C₁₋₂ alkylene)-SOₜ-ϕ; wherein ϕ is a bond to A¹. In certain embodiments, X¹ is —CH₂— SOₜ-ϕ; wherein ϕ is a bond to A¹.

In certain embodiments, X¹ is C₁₋₃ alkylene optionally substituted with 1 or 2 occurrences of R⁴. In certain embodiments, X¹ is C₁₋₃ alkylene.

In certain embodiments, X² is C₁₋₄ alkylene substituted by 0, 1, or 2 occurrences of R⁵. In certain embodiments, X² is C₁₋₄ alkylene. In certain embodiments, X² is C₁₋₃ alkylene. In certain embodiments, X² is —CH₂—. In certain embodiments, X² is C₁₋₃ alkylene substituted with 1 or 2 occurrences of R⁵.

In certain embodiments, X² is C₁₋₄ alkylene substituted by 0, 1, or 2 occurrences of R⁵ and optionally one or more occurrences of halogen. In certain embodiments, X² is C₁₋₄ alkylene optionally substituted by one or more occurrences of halogen. In certain embodiments, X² is —(C₁₋₃ alkylene)-C(O)-ψ substituted by 0, 1, or 2 occurrences of R⁵ and optionally one or more occurrences of halogen; wherein ψ is a bond to A². In certain embodiments, X² is —(C₁₋₄ alkylene)-C(O)N(R⁷)-ψ substituted by 0, 1, or 2 occurrences of R⁵ and optionally one or more occurrences of halogen; wherein ψ is a bond to A².

In certain embodiments,

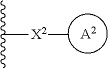

is —(C₁₋₄ alkylene)-C(O)N(R⁷)-(5-membered heteroaryl).

In certain embodiments, X³ is C₁₋₃ alkylene or C₂₋₃ alkenylene, each of which is substituted by 0, 1, or 2 occurrences of R⁶. In certain embodiments, X³ is C₁₋₄ alkylene substituted by 0, 1, or 2 occurrences of R⁶. In certain embodiments, X³ is C₁₋₄ alkylene. In certain embodiments, X³ is C₁₋₃ alkylene optionally substituted with 1 or 2 occurrences of R⁶. In certain embodiments, X³ is C₁₋₃ alkylene. In certain embodiments, X³ is —CH₂—. In certain embodiments, X³ is C₁₋₄ alkylene optionally substituted by one or more occurrences of halogen.

In certain embodiments, X³ is C₂₋₄ alkenylene substituted by 0, 1, or 2 occurrences of R⁶ and optionally one or more occurrences of halogen. In certain embodiments, X³ is C₂₋₃ alkenylene optionally substituted with 1 or 2 occurrences of R⁶. In certain embodiments, X³ is C₂₋₄ alkenylene. In certain embodiments, X³ is C₂₋₃ alkenylene. In certain embodiments, X³ is C₂₋₄ alkynylene substituted by 0, 1, or 2 occurrences of R⁶ and optionally one or more occurrences of halogen. In certain embodiments, X³ is C₂₋₃ alkynylene optionally substituted with 1 or 2 occurrences of R⁶. In certain embodiments, X³ is C₂₋₄ alkynylene. In certain embodiments, X³ is C₂₋₃ alkynylene.

In certain embodiments, X¹ is 2-4 membered heteroalkylene, X² is C₁₋₄ alkylene, and X³ is C₁₋₄ alkylene.

In certain embodiments, R¹ is halogen, C₁₋₄ alkyl, C₁₋₄ haloalkyl, —O—C₁₋₄ alkyl, or —O—C₁₋₄ haloalkyl. In certain embodiments, R¹ is fluoro, chloro, methyl, or ethyl.

In certain embodiments, R¹ is halogen. In certain embodiments, R¹ is C₁₋₄ alkyl or C₁₋₄ haloalkyl. In certain embodiments, R¹ is C₁₋₄ alkyl. In certain embodiments, R¹ is C₁₋₄ haloalkyl. In certain embodiments, R¹ is —O—C₁₋₄ alkyl or —O—C₁₋₄ haloalkyl.

In certain embodiments, R² represents independently for each occurrence halogen, cyano, hydroxyl, —N(R⁷)₂, —CON(R⁷)₂, —SOₜN(R⁷)₂, C₁₋₆ alkyl, —O—C₁₋₆ alkyl, —S—C₁₋₆ alkyl, C₂₋₆ alkenyl, or C₂₋₆ alkynyl; wherein each of the foregoing C₁₋₆ alkyl groups is optionally substituted with —OR⁷, —N(R⁷)₂, —CON(R⁷)₂, —SOₜN(R⁷)₂, or 1-3 halogens. In certain embodiments, R² represents independently for each occurrence halogen, cyano, hydroxyl, —N(R⁷)₂, —CON(R⁷)₂, —SOₜN(R⁷)₂, C₁₋₆ alkyl, or —O—C₁₋₆ alkyl; wherein each of the foregoing C₁₋₆ alkyl groups is optionally substituted 1-3 halogens. In certain embodiments, R² represents independently for each occurrence halogen, C₁₋₆ alkyl, or —O—C₁₋₆ alkyl; wherein said C₁₋₆ alkyl and —O—C₁₋₆ alkyl are each optionally substituted with —OR⁷, —N(R⁷)₂, or 1-3 halogens. In certain embodiments, R² represents independently for each occurrence fluoro, chloro, methyl, or ethyl.

In certain embodiments, R² represents independently for each occurrence C₃₋₆ cycloalkyl, —O—C₃₋₆ cycloalkyl, or 3-6 membered heterocyclyl; each of which are optionally substituted with 1 or 2 substituents independently selected from C₁₋₆ alkyl, hydroxyl, oxo, —N(R⁷)₂, —CON(R⁷)₂, —SOₜN(R⁷)₂, and halogen; wherein said C₁₋₆ alkyl is optionally substituted with —OR⁷, —N(R⁷)₂, —CON(R⁷)₂, —SOₜN(R⁷)₂, or 1-3 halogens. In certain embodiments, R² represents independently for each occurrence 3-6 membered heterocyclyl optionally substituted with 1 or 2 substituents independently selected from C₁₋₆ alkyl, hydroxyl, oxo, —N(R⁷)₂, and halogen; wherein said C₁₋₆ alkyl is optionally substituted with —OR⁷, —N(R⁷)₂, or 1-3 halogens.

In certain embodiments, R² represents independently for each occurrence halogen. In certain embodiments, R² represents independently for each occurrence fluoro or chloro. In certain embodiments, R² represents independently for each occurrence C₁₋₄alkyl optionally substituted with 1-3 halogens. In certain embodiments, R² represents independently for each occurrence C₁₋₄alkyl. In certain embodiments, R² represents independently for each occurrence methyl or ethyl. In certain embodiments, R² represents independently for each occurrence hydroxyl, —N(R⁷)₂, —CON(R⁷)₂, or —SOₜN(R⁷)₂.

In certain embodiments, R³ represents independently for each occurrence —(C₀₋₄ alkylene)-N(R⁷)₂. In certain embodiments, R³ is —N(R⁷)₂.

In certain embodiments, R³ represents independently for each occurrence —N(R⁷)₂, —OR⁷, C₁₋₄ alkyl, C₁₋₄ haloalkyl, or halogen. In certain embodiments, R³ represents independently for each occurrence —(C₀₋₄ alkylene)-OR⁷. In certain embodiments, R³ represents independently for each —OR⁷. In certain embodiments, R³ represents independently for each occurrence C₁₋₄ alkyl. In certain embodiments, R³ represents independently for each occurrence methyl or ethyl. In certain embodiments, R³ represents independently for each occurrence C₁₋₄ haloalkyl. In certain embodiments, R³ represents independently for each occurrence C₁₋₂ haloalkyl. In certain embodiments, R³ represents independently for each occurrence halogen. In certain embodiments, R³ represents independently for each occurrence fluoro or chloro. In certain embodiments, R³ represents independently for each occurrence —(C₀₋₄ alkylene)-N(R⁷)₂, —(C₀₋₄ alkylene)-OR⁷, C₁₋₄ alkyl, C₁₋₄ haloalkyl, or halogen.

In certain embodiments, two occurrences of R⁴ are taken together with the carbon atom or atoms to which they are attached to form a 3-7 membered ring optionally substituted with 1 or 2 substituents independently selected from halogen, C₁₋₄ alkyl, C₁₋₄ haloalkyl, —(C₀₋₄ alkylene)-N(R⁷)₂, and —(C₀₋₄ alkylene)-OR⁷. In certain embodiments, two occurrences of R⁴ are taken together with the carbon atom or atoms to which they are attached to form a 3-5 membered carbocyclic ring optionally substituted with 1 or 2 substituents independently selected from halogen, C₁₋₄ alkyl, and C₁₋₄ haloalkyl. In certain embodiments, two occurrences of R⁴ are taken together with the carbon atom or atoms to which they are attached to form a saturated 3-membered carbocyclic ring. In certain embodiments, two occurrences of R⁴ are taken together with the carbon atom or atoms to which they are attached to form a 3-5 membered heterocyclic ring optionally substituted with 1 or 2 substituents independently selected from halogen, C₁₋₄ alkyl, and C₁₋₄ haloalkyl.

In certain embodiments, R⁵ represents independently for each occurrence —(C₀₋₄ alkylene)-N(R⁷)₂ or —(C₀₋₄ alkylene)-OR⁷. In certain embodiments, R⁵ represents independently for each occurrence —(C₁₋₄ alkylene)-N(R⁷)₂ or —(C₁₋₄ alkylene)-OR⁷.

In certain embodiments, two occurrences of R⁵ are taken together with the carbon atom or atoms to which they are attached to form a 3-7 membered ring optionally substituted with 1 or 2 substituents independently selected from halogen, C₁₋₄ alkyl, C₁₋₄ haloalkyl, —(C₀₋₄ alkylene)-N(R⁷)₂, and —(C₀₋₄ alkylene)-OR⁷. In certain embodiments, two occurrences of R⁵ are taken together with the carbon atom or atoms to which they are attached to form a 3-5 membered carbocyclic ring optionally substituted with 1 or 2 substituents independently selected from halogen, C₁₋₄ alkyl, and C₁₋₄ haloalkyl. In certain embodiments, two occurrences of R⁵ are taken together with the carbon atom or atoms to which they are attached to form a saturated 3-5 membered carbocyclic ring. In certain embodiments, two occurrences of R⁵ are taken together with the carbon atom or atoms to which they are attached to form a 3-5 membered heterocyclic ring optionally substituted with 1 or 2 substituents independently selected from halogen, C₁₋₄ alkyl, and C₁₋₄ haloalkyl.

In certain embodiments, two occurrences of R⁶ are taken together with the carbon atom or atoms to which they are attached to form a 3-7 membered ring optionally substituted with 1 or 2 substituents independently selected from halogen, C₁₋₄ alkyl, C₁₋₄ haloalkyl, —(C₀₋₄ alkylene)-N(R⁷)₂, and —(C₀₋₄ alkylene)-OR⁷. In certain embodiments, two occurrences of R⁶ are taken together with the carbon atom or atoms to which they are attached to form a 3-5 membered carbocyclic ring optionally substituted with 1 or 2 substituents independently selected from halogen, C₁₋₄ alkyl, and C₁₋₄ haloalkyl. In certain embodiments, two occurrences of R⁶ are taken together with the carbon atom or atoms to which they are attached to form a saturated 3-membered carbocyclic ring. In certain embodiments, two occurrences of R⁶ are taken together with the carbon atom or atoms to which they are attached to form a 3-5 membered heterocyclic ring optionally substituted with 1 or 2 substituents independently selected from halogen, C₁₋₄ alkyl, and C₁₋₄ haloalkyl.

In certain embodiments, R⁷ represents independently for each occurrence hydrogen, C₁₋₆ alkyl, C₁₋₆ haloalkyl, or —(C₀₋₆ alkylene)-(C₃-7 cycloalkyl). In certain embodiments, R⁷ represents independently for each occurrence hydrogen or C₁₋₄ alkyl. In certain embodiments, R⁷ represents independently for each occurrence hydrogen or methyl. In certain embodiments, R⁷ is hydrogen. In certain embodiments, R⁷ is methyl.

In certain embodiments, R⁸ is C₁₋₆ alkyl or C₁₋₆ haloalkyl. In certain embodiments, R⁸ is C₁₋₄ alkyl or C₁₋₄ haloalkyl. In certain embodiments, R⁸ is C₁₋₄ alkyl. In certain embodiments, R⁸ is C₁₋₄ haloalkyl.

In certain embodiments, t is 2. In certain embodiments, t is 1.

The description above describes multiple embodiments relating to compounds of Formula II. The patent application specifically contemplates all combinations of the embodiments.

In certain other embodiments, the compound is one of the compounds listed in Table 1 or 9 below, or a pharmaceutically acceptable salt thereof. In certain embodiments, the compound is one of the compounds listed in Table 1 or 9 below. In certain other embodiments, the compound is one of the compounds listed in Table 1 below, or a pharmaceutically acceptable salt thereof. In certain embodiments, the compound is one of the compounds listed in Table 1 below. In certain other embodiments, the compound is one of the compounds listed in Table 9 below, or a pharmaceutically acceptable salt thereof. In certain embodiments, the compound is one of the compounds listed in Table 9 below.

TABLE 1

| Com- | |
|---|---|
| pound | |
| No. | Chemical Structure |
| I-1 | 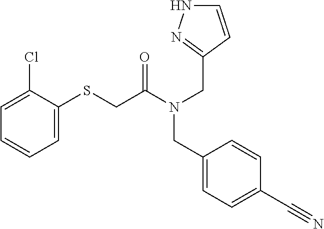 [SMILES: N#Cc1ccc(CN(Cc2cc[nH]n2)C(=O)CSc2ccccc2Cl)cc1] |
| I-2 | 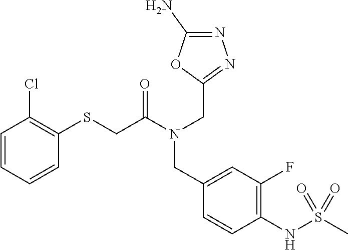 [SMILES: CS(=O)(=O)Nc1ccc(CN(Cc2nnc(N)o2)C(=O)CSc2ccccc2Cl)cc1F] |
| I-3 | 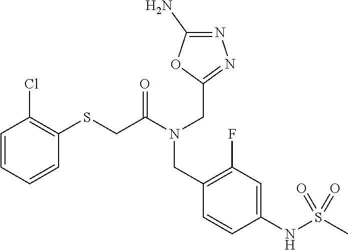 [SMILES: CS(=O)(=O)Nc1ccc(CN(Cc2nnc(N)o2)C(=O)CSc2ccccc2Cl)c(F)c1] |
| I-4 | 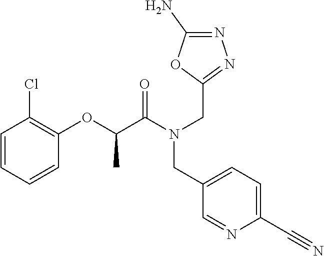 [SMILES: C[C@@H](Oc1ccccc1Cl)C(=O)N(Cc1ccc(C#N)nc1)Cc1nnc(N)o1] |
| I-5 | 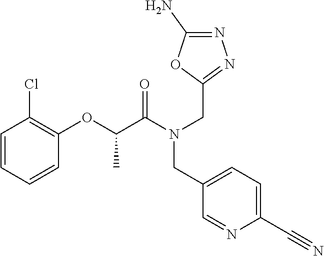 [SMILES: C[C@H](Oc1ccccc1Cl)C(=O)N(Cc1ccc(C#N)nc1)Cc1nnc(N)o1] |
| I-6 | 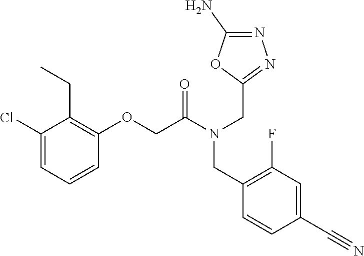 [SMILES: CCc1c(Cl)cccc1OCC(=O)N(Cc1nnc(N)o1)Cc1ccc(C#N)cc1F] |
| I-7 | 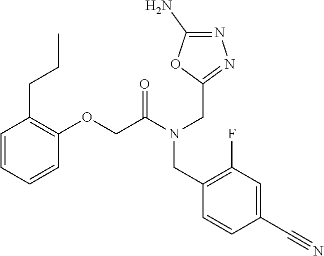 [SMILES: CCCc1ccccc1OCC(=O)N(Cc1nnc(N)o1)Cc1ccc(C#N)cc1F] |
| I-8 | 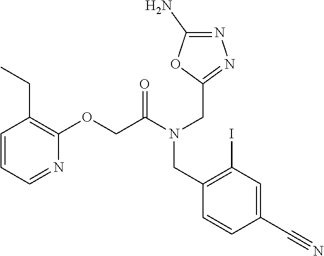 [SMILES: CCc1cccnc1OCC(=O)N(Cc1nnc(N)o1)Cc1ccc(C#N)cc1I] |
| I-9 | 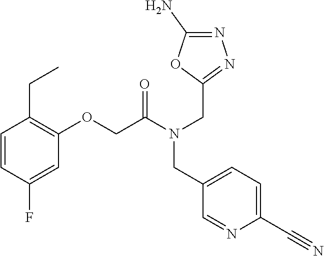 [SMILES: CCc1ccc(F)cc1OCC(=O)N(Cc1ccc(C#N)nc1)Cc1nnc(N)o1] |
| I-10 | 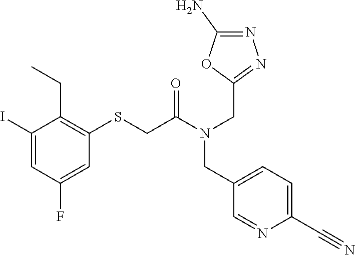 [SMILES: CCc1c(I)cc(F)cc1SCC(=O)N(Cc1ccc(C#N)nc1)Cc1nnc(N)o1] |
| I-11 | 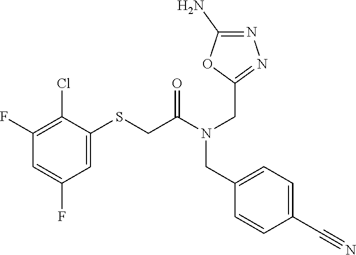 [SMILES: N#Cc1ccc(CN(Cc2nnc(N)o2)C(=O)CSc2cc(F)cc(F)c2Cl)cc1] |
| I-12 | 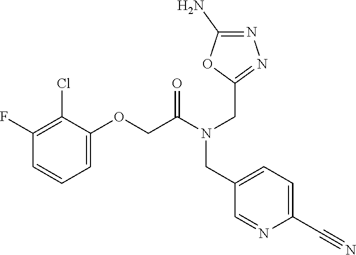 [SMILES: N#Cc1ccc(CN(Cc2nnc(N)o2)C(=O)COc2cccc(F)c2Cl)cn1] |
| I-13 | 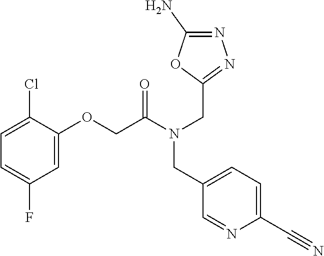 [SMILES: N#Cc1ccc(CN(Cc2nnc(N)o2)C(=O)COc2cc(F)ccc2Cl)cn1] |
| I-14 | 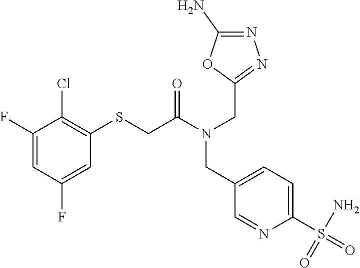 [SMILES: Nc1nnc(CN(Cc2ccc(S(N)(=O)=O)nc2)C(=O)CSc2cc(F)cc(F)c2Cl)o1] |
| I-15 | 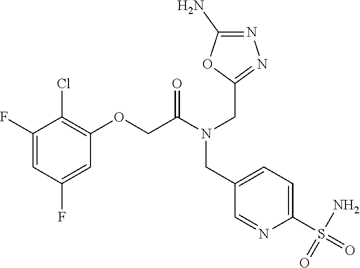 [SMILES: Nc1nnc(CN(Cc2ccc(S(N)(=O)=O)nc2)C(=O)COc2cc(F)cc(F)c2Cl)o1] |
| I-16 | 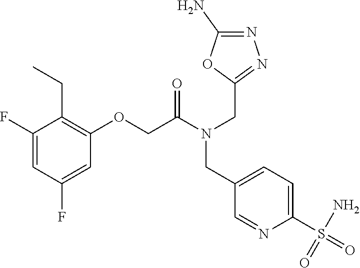 [SMILES: CCc1c(F)cc(F)cc1OCC(=O)N(Cc1ccc(S(N)(=O)=O)nc1)Cc1nnc(N)o1] |
| I-17 | 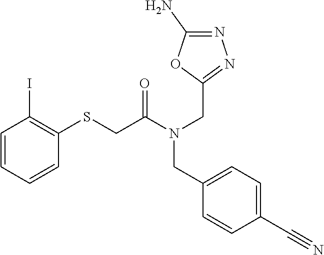 [SMILES: N#Cc1ccc(CN(Cc2nnc(N)o2)C(=O)CSc2ccccc2I)cc1] |
| I-18 | 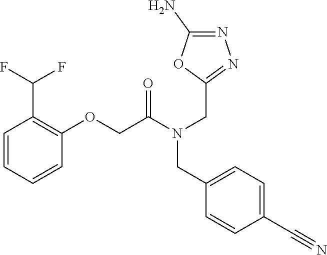 [SMILES: N#Cc1ccc(CN(Cc2nnc(N)o2)C(=O)COc2ccccc2C(F)F)cc1] |
| I-19 | 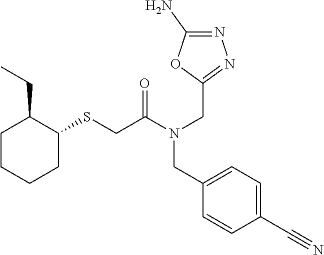 [SMILES: CC[C@@H]1CCCC[C@H]1SCC(=O)N(Cc1ccc(C#N)cc1)Cc1nnc(N)o1] |
| I-20 | 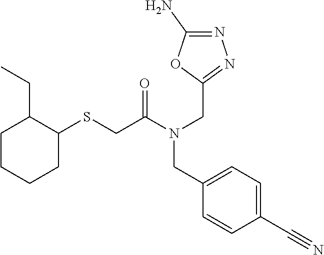 [SMILES: CCC1CCCCC1SCC(=O)N(Cc1ccc(C#N)cc1)Cc1nnc(N)o1] |
| I-21 | 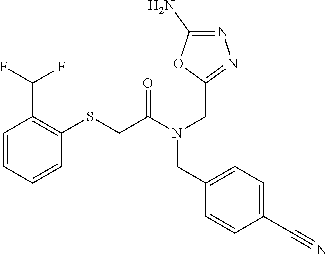 [SMILES: N#Cc1ccc(CN(Cc2nnc(N)o2)C(=O)CSc2ccccc2C(F)F)cc1] |
| I-22 | 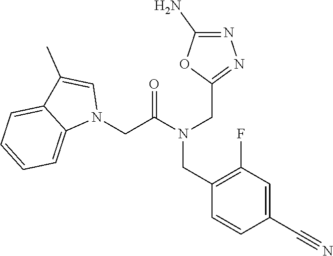 [SMILES: Cc1cn(CC(=O)N(Cc2nnc(N)o2)Cc2ccc(C#N)cc2F)c2ccccc12] |
| I-23 | 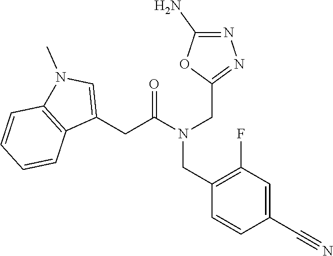 [SMILES: Cn1cc(CC(=O)N(Cc2nnc(N)o2)Cc2ccc(C#N)cc2F)c2ccccc21] |
| I-24 | 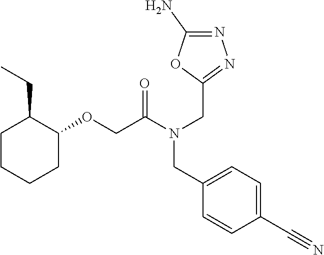 [SMILES: CC[C@@H]1CCCC[C@H]1OCC(=O)N(Cc1ccc(C#N)cc1)Cc1nnc(N)o1] |
| I-25 | 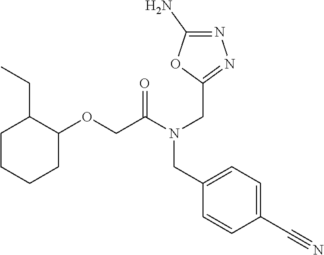 [SMILES: CCC1CCCCC1OCC(=O)N(Cc1ccc(C#N)cc1)Cc1nnc(N)o1] |
| I-26 | 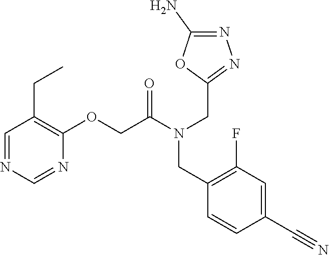 [SMILES: CCc1cncnc1OCC(=O)N(Cc1nnc(N)o1)Cc1ccc(C#N)cc1F] |
| I-27 | 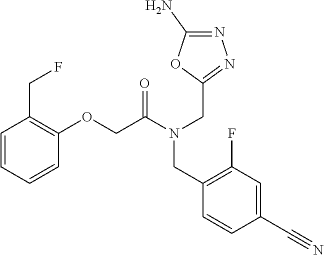 [SMILES: N#Cc1ccc(CN(Cc2nnc(N)o2)C(=O)COc2ccccc2CF)c(F)c1] |
| I-28 | 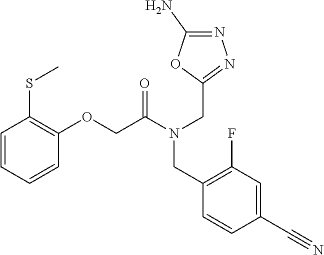 [SMILES: CSc1ccccc1OCC(=O)N(Cc1nnc(N)o1)Cc1ccc(C#N)cc1F] |
| I-29 | 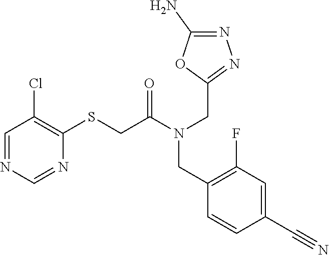 [SMILES: N#Cc1ccc(CN(Cc2nnc(N)o2)C(=O)CSc2ncncc2Cl)c(F)c1] |
| I-30 | 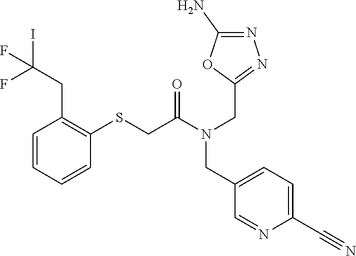 [SMILES: N#Cc1ccc(CN(Cc2nnc(N)o2)C(=O)CSc2ccccc2CC(F)(F)I)cn1] |
| I-31 | 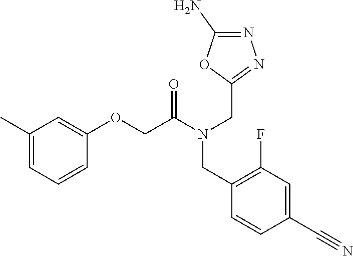 [SMILES: Cc1cccc(OCC(=O)N(Cc2nnc(N)o2)Cc2ccc(C#N)cc2F)c1] |
| I-32 | 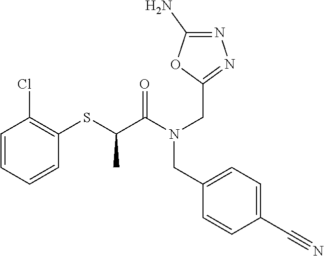 [SMILES: C[C@@H](Sc1ccccc1Cl)C(=O)N(Cc1ccc(C#N)cc1)Cc1nnc(N)o1] |
| I-33 | 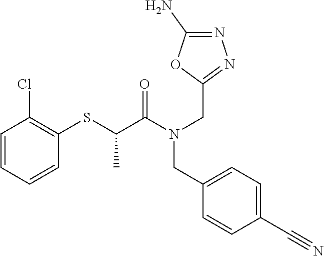 [SMILES: C[C@H](Sc1ccccc1Cl)C(=O)N(Cc1ccc(C#N)cc1)Cc1nnc(N)o1] |
| I-34 | [symbol] |
| I-35 | 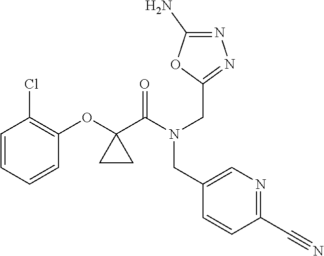 [SMILES: N#Cc1ccc(CN(Cc2nnc(N)o2)C(=O)C2(Oc3ccccc3Cl)CC2)cn1] |
| I-36 | 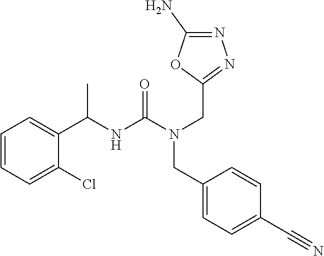 [SMILES: CC(NC(=O)N(Cc1ccc(C#N)cc1)Cc1nnc(N)o1)c1ccccc1Cl] |
| I-37 | 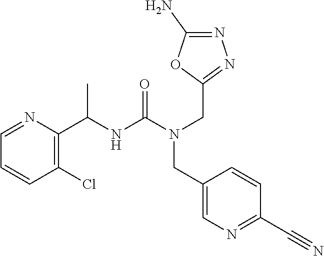 [SMILES: CC(NC(=O)N(Cc1ccc(C#N)nc1)Cc1nnc(N)o1)c1ncccc1Cl] |
| I-38 | 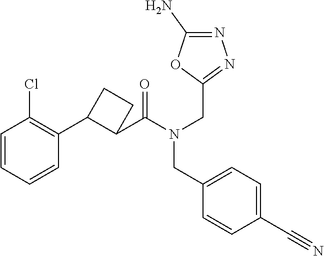 [SMILES: N#Cc1ccc(CN(Cc2nnc(N)o2)C(=O)C2CCC2c2ccccc2Cl)cc1] |
| I-39 | 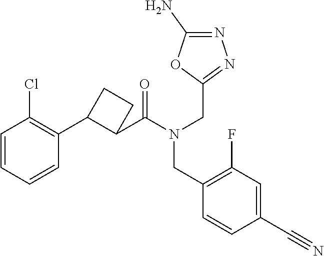 [SMILES: N#Cc1ccc(CN(Cc2nnc(N)o2)C(=O)C2CCC2c2ccccc2Cl)c(F)c1] |
| I-40 | 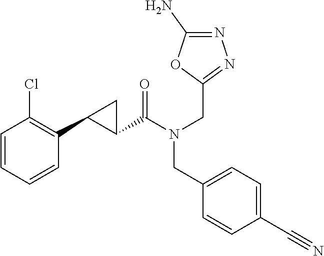 [SMILES: N#Cc1ccc(CN(Cc2nnc(N)o2)C(=O)[C@H]2C[C@@H]2c2ccccc2Cl)cc1] |
| I-41 | 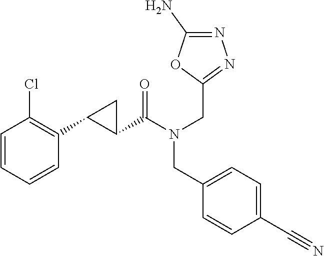 [SMILES: N#Cc1ccc(CN(Cc2nnc(N)o2)C(=O)[C@H]2C[C@H]2c2ccccc2Cl)cc1] |
| I-42 | 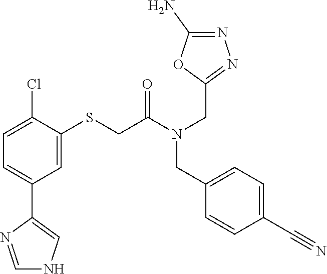 [SMILES: N#Cc1ccc(CN(Cc2nnc(N)o2)C(=O)CSc2cc(-c3c[nH]cn3)ccc2Cl)cc1] |
| I-43 | 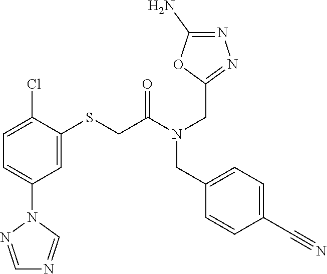 [SMILES: N#Cc1ccc(CN(Cc2nnc(N)o2)C(=O)CSc2cc(-n3cncn3)ccc2Cl)cc1] |
| I-44 | 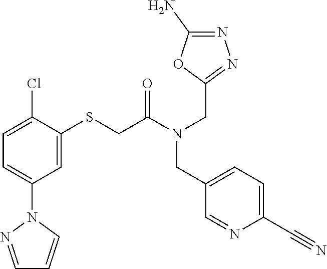 [SMILES: N#Cc1ccc(CN(Cc2nnc(N)o2)C(=O)CSc2cc(-n3cccn3)ccc2Cl)cn1] |
| I-45 | 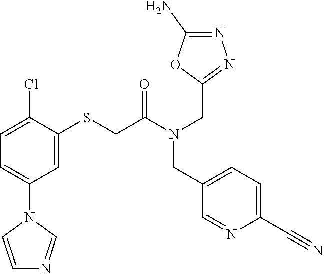 [SMILES: N#Cc1ccc(CN(Cc2nnc(N)o2)C(=O)CSc2cc(-n3ccnc3)ccc2Cl)cn1] |
| I-46 | 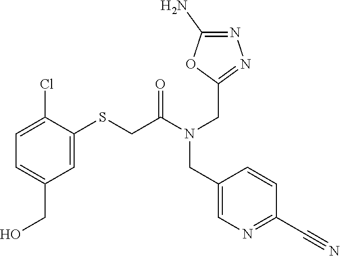 [SMILES: N#Cc1ccc(CN(Cc2nnc(N)o2)C(=O)CSc2cc(CO)ccc2Cl)cn1] |
| I-47 | 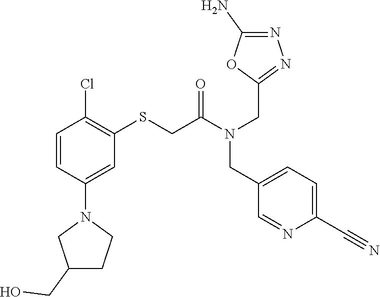 [SMILES: N#Cc1ccc(CN(Cc2nnc(N)o2)C(=O)CSc2cc(N3CCC(CO)C3)ccc2Cl)cn1] |
| I-48 | 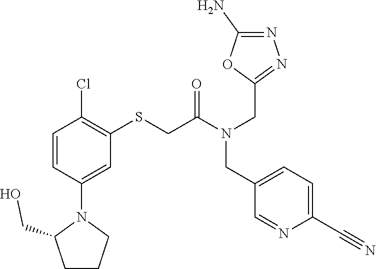 [SMILES: N#Cc1ccc(CN(Cc2nnc(N)o2)C(=O)CSc2cc(N3CCC[C@@H]3CO)ccc2Cl)cn1] |
| I-49 | 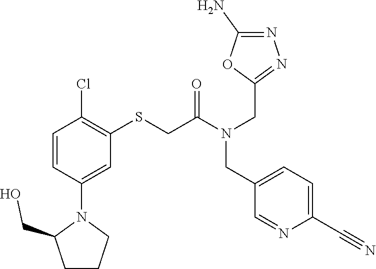 [SMILES: N#Cc1ccc(CN(Cc2nnc(N)o2)C(=O)CSc2cc(N3CCC[C@H]3CO)ccc2Cl)cn1] |
| I-50 | 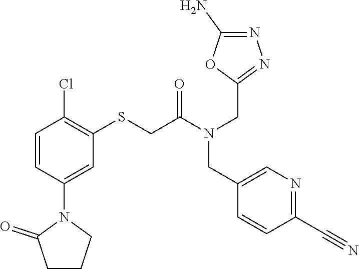 [SMILES: N#Cc1ccc(CN(Cc2nnc(N)o2)C(=O)CSc2cc(N3CCCC3=O)ccc2Cl)cn1] |
| I-51 | 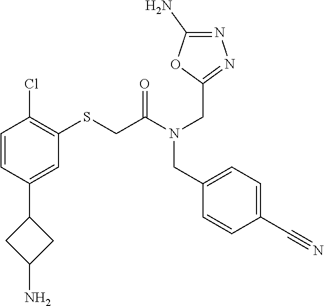 [SMILES: N#Cc1ccc(CN(Cc2nnc(N)o2)C(=O)CSc2cc(C3CC(N)C3)ccc2Cl)cc1] |
| I-52 | 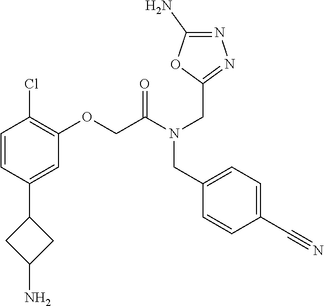 [SMILES: N#Cc1ccc(CN(Cc2nnc(N)o2)C(=O)COc2cc(C3CC(N)C3)ccc2Cl)cc1] |
| I-53 | 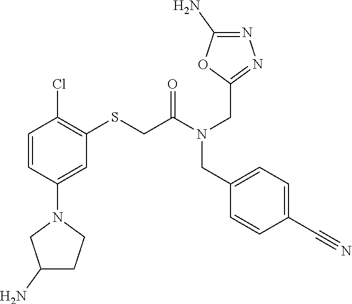 [SMILES: N#Cc1ccc(CN(Cc2nnc(N)o2)C(=O)CSc2cc(N3CCC(N)C3)ccc2Cl)cc1] |
| I-54 | 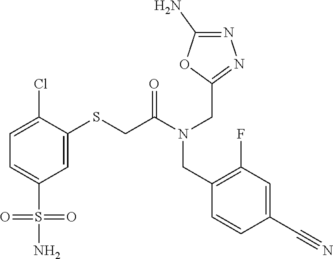 [SMILES: N#Cc1ccc(CN(Cc2nnc(N)o2)C(=O)CSc2cc(S(N)(=O)=O)ccc2Cl)c(F)c1] |
| I-55 | 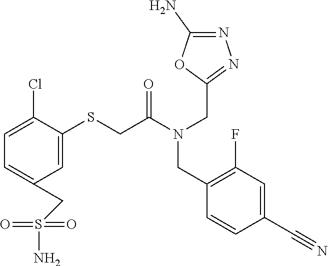 [SMILES: N#Cc1ccc(CN(Cc2nnc(N)o2)C(=O)CSc2cc(CS(N)(=O)=O)ccc2Cl)c(F)c1] |
| I-56 | 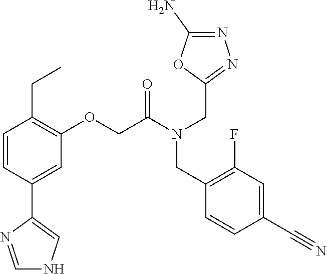 [SMILES: CCc1ccc(-c2c[nH]cn2)cc1OCC(=O)N(Cc1nnc(N)o1)Cc1ccc(C#N)cc1F] |
| I-57 | 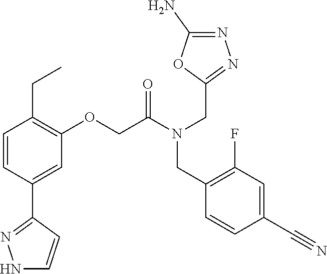 [SMILES: CCc1ccc(-c2cc[nH]n2)cc1OCC(=O)N(Cc1nnc(N)o1)Cc1ccc(C#N)cc1F] |
| I-58 | 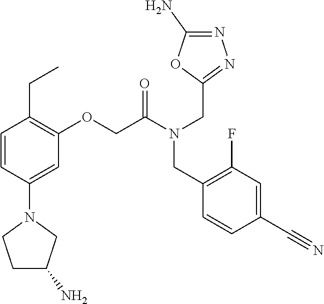 [SMILES: CCc1ccc(N2CC[C@@H](N)C2)cc1OCC(=O)N(Cc1nnc(N)o1)Cc1ccc(C#N)cc1F] |
| I-59 | 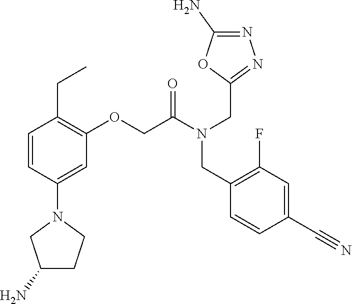 [SMILES: CCc1ccc(N2CC[C@H](N)C2)cc1OCC(=O)N(Cc1nnc(N)o1)Cc1ccc(C#N)cc1F] |
| I-60 | 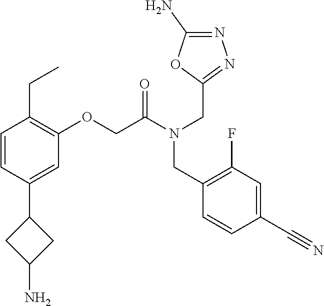 [SMILES: CCc1ccc(C2CC(N)C2)cc1OCC(=O)N(Cc1nnc(N)o1)Cc1ccc(C#N)cc1F] |
| I-61 | 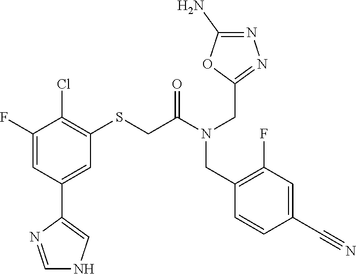 [SMILES: N#Cc1ccc(CN(Cc2nnc(N)o2)C(=O)CSc2cc(-c3c[nH]cn3)cc(F)c2Cl)c(F)c1] |
| I-62 | 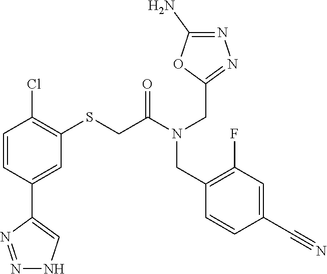 [SMILES: N#Cc1ccc(CN(Cc2nnc(N)o2)C(=O)CSc2cc(-c3c[nH]nn3)ccc2Cl)c(F)c1] |
| I-63 | 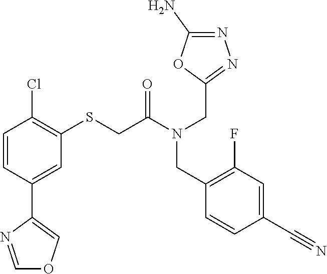 [SMILES: N#Cc1ccc(CN(Cc2nnc(N)o2)C(=O)CSc2cc(-c3cocn3)ccc2Cl)c(F)c1] |
| I-64 | 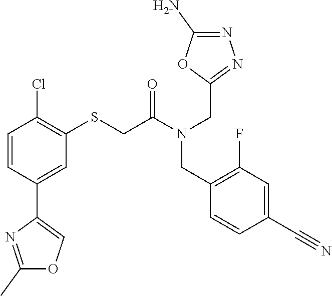 [SMILES: Cc1nc(-c2ccc(Cl)c(SCC(=O)N(Cc3nnc(N)o3)Cc3ccc(C#N)cc3F)c2)co1] |
| I-65 | 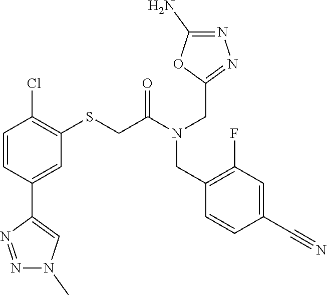 [SMILES: Cn1cc(-c2ccc(Cl)c(SCC(=O)N(Cc3nnc(N)o3)Cc3ccc(C#N)cc3F)c2)nn1] |
| I-66 | 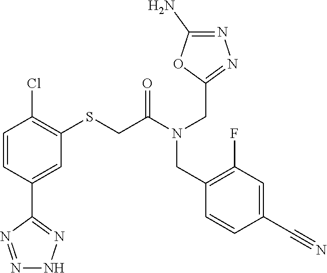 [SMILES: N#Cc1ccc(CN(Cc2nnc(N)o2)C(=O)CSc2cc(-c3nn[nH]n3)ccc2Cl)c(F)c1] |
| I-67 | 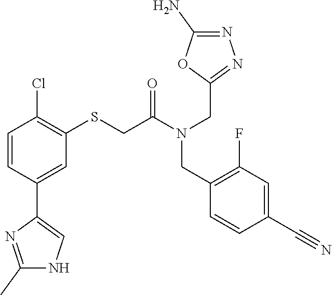 [SMILES: Cc1nc(-c2ccc(Cl)c(SCC(=O)N(Cc3nnc(N)o3)Cc3ccc(C#N)cc3F)c2)c[nH]1] |
| I-68 | 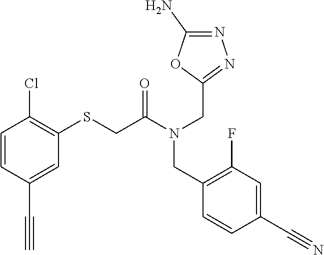 [SMILES: C#Cc1ccc(Cl)c(SCC(=O)N(Cc2nnc(N)o2)Cc2ccc(C#N)cc2F)c1] |
| I-69 | 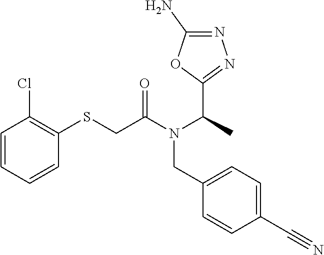 [SMILES: C[C@H](c1nnc(N)o1)N(Cc1ccc(C#N)cc1)C(=O)CSc1ccccc1Cl] |
| I-70 | 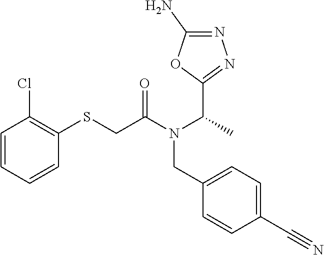 [SMILES: C[C@@H](c1nnc(N)o1)N(Cc1ccc(C#N)cc1)C(=O)CSc1ccccc1Cl] |
| I-71 | 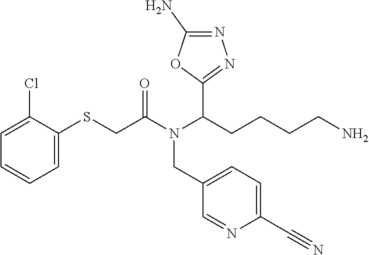 [SMILES: N#Cc1ccc(CN(C(=O)CSc2ccccc2Cl)C(CCCCN)c2nnc(N)o2)cn1] |
| I-72 | 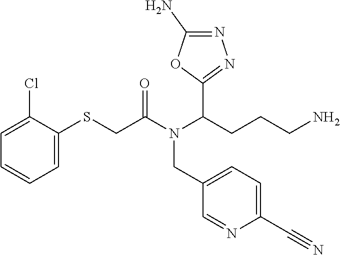 [SMILES: N#Cc1ccc(CN(C(=O)CSc2ccccc2Cl)C(CCCN)c2nnc(N)o2)cn1] |
| I-73 | 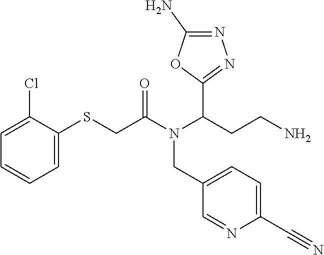 [SMILES: N#Cc1ccc(CN(C(=O)CSc2ccccc2Cl)C(CCN)c2nnc(N)o2)cn1] |
| I-74 | 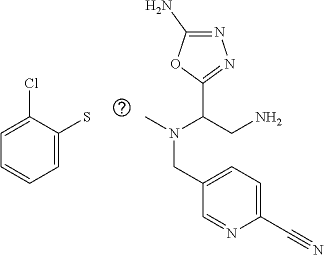 [SMILES: Sc1ccccc1Cl ; CN(Cc1ccc(C#N)nc1)C(CN)c1nnc(N)o1] |
| I-75 | 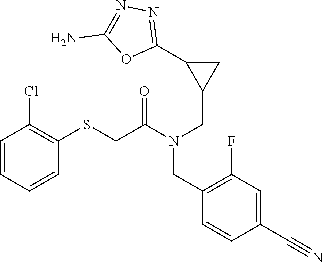 [SMILES: N#Cc1ccc(CN(CC2CC2c2nnc(N)o2)C(=O)CSc2ccccc2Cl)c(F)c1] |
| I-76 | 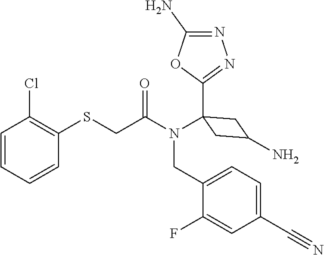 [SMILES: N#Cc1ccc(CN(C(=O)CSc2ccccc2Cl)C2(c3nnc(N)o3)CC(N)C2)c(F)c1] |
| I-77 | 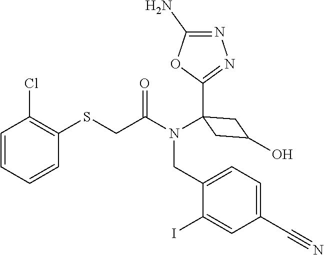 [SMILES: N#Cc1ccc(CN(C(=O)CSc2ccccc2Cl)C2(c3nnc(N)o3)CC(O)C2)c(I)c1] |
| I-78 | 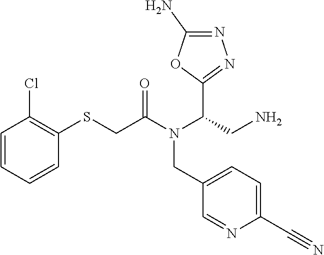 [SMILES: N#Cc1ccc(CN(C(=O)CSc2ccccc2Cl)[C@@H](CN)c2nnc(N)o2)cn1] |
| I-79 | 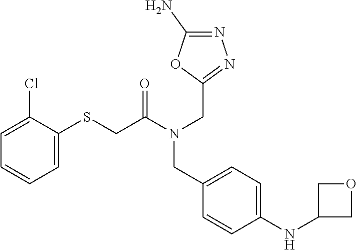 [SMILES: Nc1nnc(CN(Cc2ccc(NC3COC3)cc2)C(=O)CSc2ccccc2Cl)o1] |
| I-80 | 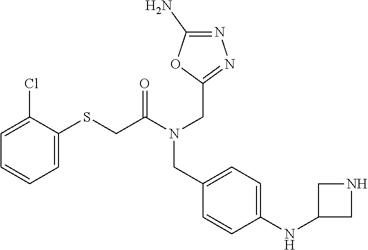 [SMILES: Nc1nnc(CN(Cc2ccc(NC3CNC3)cc2)C(=O)CSc2ccccc2Cl)o1] |
| I-81 | 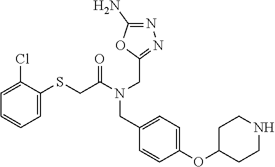 [SMILES: Nc1nnc(CN(Cc2ccc(OC3CCNCC3)cc2)C(=O)CSc2ccccc2Cl)o1] |
| I-82 | 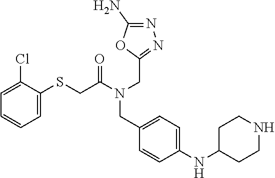 [SMILES: Nc1nnc(CN(Cc2ccc(NC3CCNCC3)cc2)C(=O)CSc2ccccc2Cl)o1] |
| I-83 | 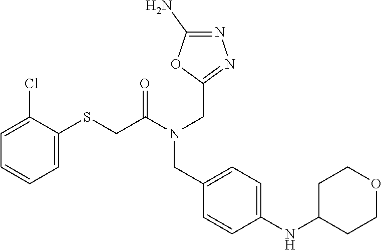 [SMILES: Nc1nnc(CN(Cc2ccc(NC3CCOCC3)cc2)C(=O)CSc2ccccc2Cl)o1] |
| I-84 | 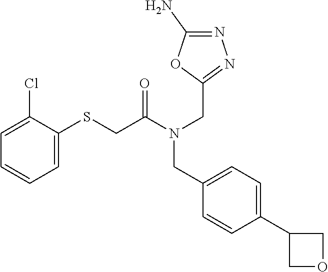 [SMILES: Nc1nnc(CN(Cc2ccc(C3COC3)cc2)C(=O)CSc2ccccc2Cl)o1] |
| I-85 | 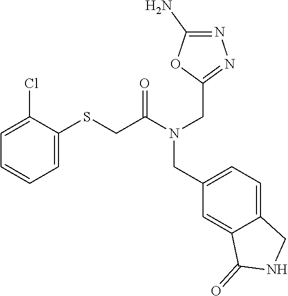 [SMILES: Nc1nnc(CN(Cc2ccc3c(c2)C(=O)NC3)C(=O)CSc2ccccc2Cl)o1] |
| I-86 | 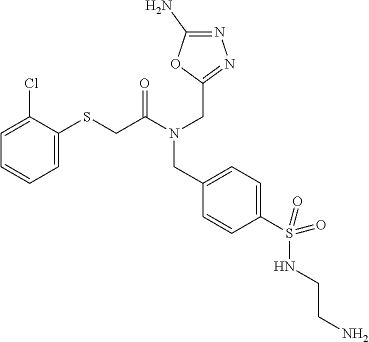 [SMILES: NCCNS(=O)(=O)c1ccc(CN(Cc2nnc(N)o2)C(=O)CSc2ccccc2Cl)cc1] |
| I-87 | 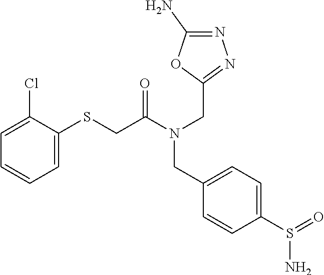 [SMILES: Nc1nnc(CN(Cc2ccc(S(N)=O)cc2)C(=O)CSc2ccccc2Cl)o1] |
| I-88 | 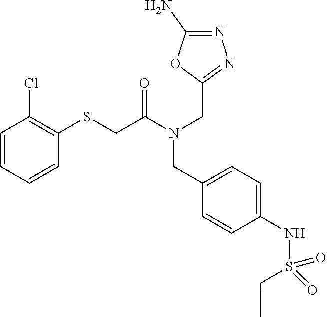 [SMILES: CCS(=O)(=O)Nc1ccc(CN(Cc2nnc(N)o2)C(=O)CSc2ccccc2Cl)cc1] |
| I-89 | 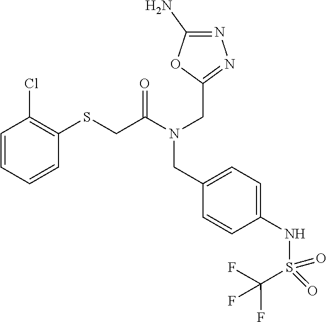 [SMILES: Nc1nnc(CN(Cc2ccc(NS(=O)(=O)C(F)(F)F)cc2)C(=O)CSc2ccccc2Cl)o1] |
| I-90 | 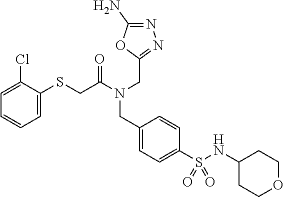 [SMILES: Nc1nnc(CN(Cc2ccc(S(=O)(=O)NC3CCOCC3)cc2)C(=O)CSc2ccccc2Cl)o1] |
| I-91 | 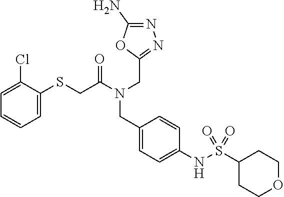 [SMILES: Nc1nnc(CN(Cc2ccc(NS(=O)(=O)C3CCOCC3)cc2)C(=O)CSc2ccccc2Cl)o1] |
| I-92 | 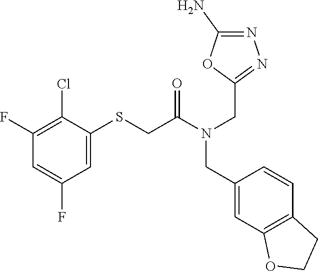 [SMILES: Nc1nnc(CN(Cc2ccc3c(c2)OCC3)C(=O)CSc2cc(F)cc(F)c2Cl)o1] |
| I-93 | 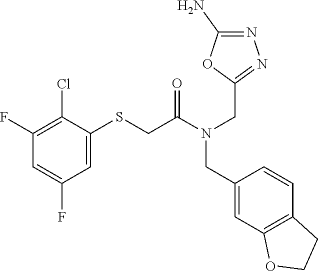 [SMILES: Nc1nnc(CN(Cc2ccc3c(c2)OCC3)C(=O)CSc2cc(F)cc(F)c2Cl)o1] |
| I-94 | 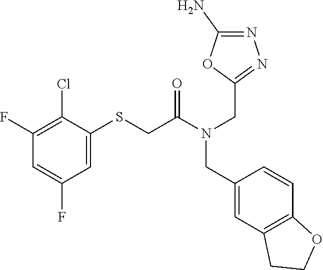 [SMILES: Nc1nnc(CN(Cc2ccc3c(c2)CCO3)C(=O)CSc2cc(F)cc(F)c2Cl)o1] |
| I-95 | 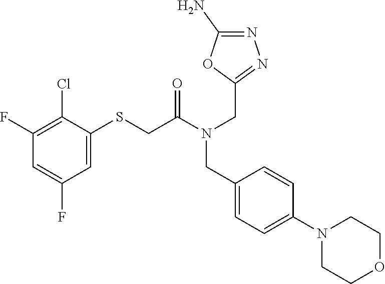 [SMILES: Nc1nnc(CN(Cc2ccc(N3CCOCC3)cc2)C(=O)CSc2cc(F)cc(F)c2Cl)o1] |
| I-96 | 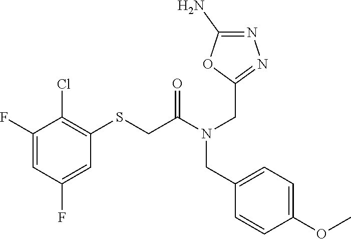 [SMILES: COc1ccc(CN(Cc2nnc(N)o2)C(=O)CSc2cc(F)cc(F)c2Cl)cc1] |
| I-97 | 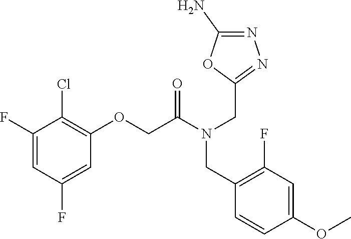 [SMILES: COc1ccc(CN(Cc2nnc(N)o2)C(=O)COc2cc(F)cc(F)c2Cl)c(F)c1] |
| I-98 | 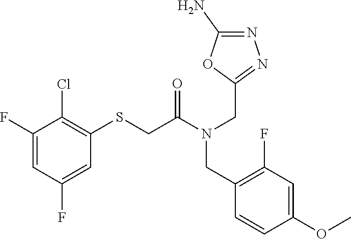 [SMILES: COc1ccc(CN(Cc2nnc(N)o2)C(=O)CSc2cc(F)cc(F)c2Cl)c(F)c1] |
| I-99 | 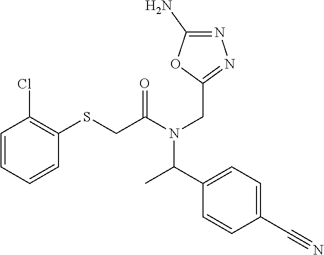 [SMILES: CC(c1ccc(C#N)cc1)N(Cc1nnc(N)o1)C(=O)CSc1ccccc1Cl] |
| I-100 | 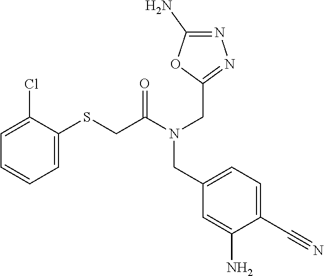 [SMILES: N#Cc1ccc(CN(Cc2nnc(N)o2)C(=O)CSc2ccccc2Cl)cc1N] |
| I-101 | 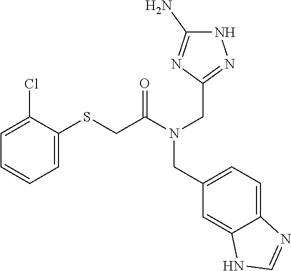 [SMILES: Nc1nc(CN(Cc2ccc3nc[nH]c3c2)C(=O)CSc2ccccc2Cl)n[nH]1] |
| I-102 | 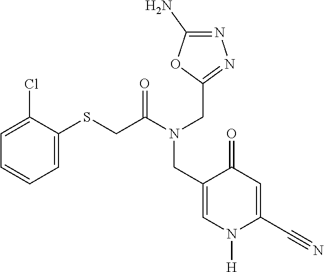 [SMILES: N#Cc1cc(=O)c(CN(Cc2nnc(N)o2)C(=O)CSc2ccccc2Cl)c[nH]1] |
| I-103 | 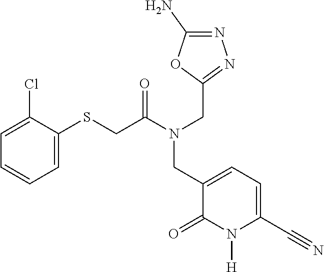 [SMILES: N#Cc1ccc(CN(Cc2nnc(N)o2)C(=O)CSc2ccccc2Cl)c(=O)[nH]1] |
| I-104 | 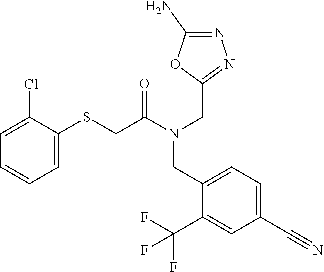 [SMILES: N#Cc1ccc(CN(Cc2nnc(N)o2)C(=O)CSc2ccccc2Cl)c(C(F)(F)F)c1] |
| I-105 | 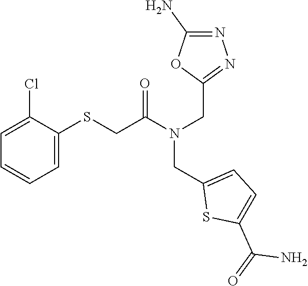 [SMILES: NC(=O)c1ccc(CN(Cc2nnc(N)o2)C(=O)CSc2ccccc2Cl)s1] |
| I-106 | 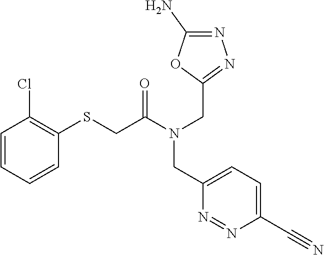 [SMILES: N#Cc1ccc(CN(Cc2nnc(N)o2)C(=O)CSc2ccccc2Cl)nn1] |
| I-107 | 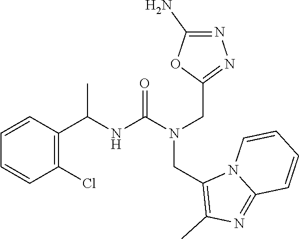 [SMILES: Cc1nc2ccccn2c1CN(Cc1nnc(N)o1)C(=O)NC(C)c1ccccc1Cl] |
| I-108 | 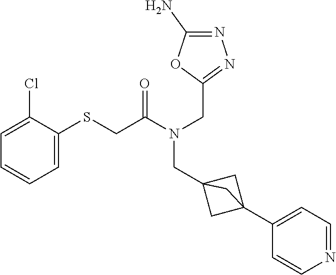 [SMILES: Nc1nnc(CN(CC23CC(c4ccncc4)(C2)C3)C(=O)CSc2ccccc2Cl)o1] |
| I-109 | 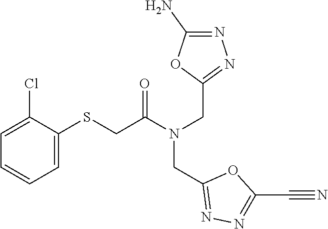 [SMILES: N#Cc1nnc(CN(Cc2nnc(N)o2)C(=O)CSc2ccccc2Cl)o1] |
| I-110 | 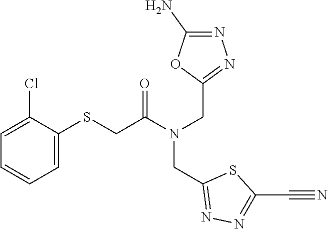 [SMILES: N#Cc1nnc(CN(Cc2nnc(N)o2)C(=O)CSc2ccccc2Cl)s1] |
| I-111 | 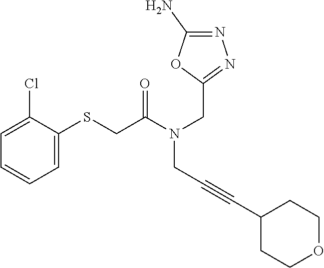 [SMILES: Nc1nnc(CN(CC#CC2CCOCC2)C(=O)CSc2ccccc2Cl)o1] |
| I-112 | 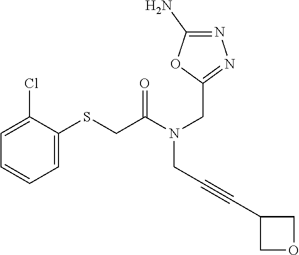 [SMILES: Nc1nnc(CN(CC#CC2COC2)C(=O)CSc2ccccc2Cl)o1] |
| I-113 | 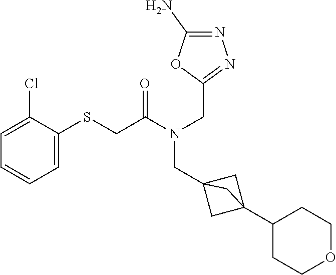 [SMILES: Nc1nnc(CN(CC23CC(C4CCOCC4)(C2)C3)C(=O)CSc2ccccc2Cl)o1] |
| I-114 | 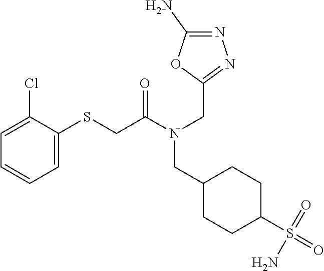 [SMILES: Nc1nnc(CN(CC2CCC(S(N)(=O)=O)CC2)C(=O)CSc2ccccc2Cl)o1] |
| I-115 | 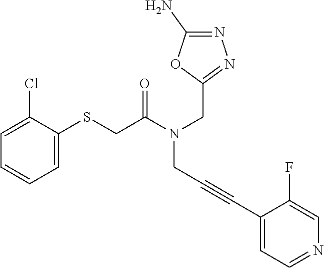 [SMILES: Nc1nnc(CN(CC#Cc2ccncc2F)C(=O)CSc2ccccc2Cl)o1] |
| I-116 | 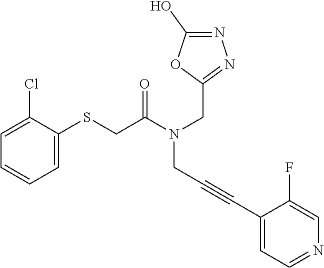 [SMILES: O=C(CSc1ccccc1Cl)N(CC#Cc1ccncc1F)Cc1nnc(O)o1] |

[symbol]

indicates data missing or illegible when filed

Methods for preparing compounds described herein are illustrated in the following synthetic Schemes. The Schemes are given for the purpose of illustrating the invention, and are not intended to limit the scope or spirit of the invention. Starting materials shown in the Schemes can be obtained from commercial sources or be prepared based on procedures described in the literature.

Scheme 1 illustrates a general method for preparing compounds of the type 1F and 1H. Aldehydes 1A are commercially available, known in the literature, and/or are readily prepared, for example, from the corresponding acid or alcohol. Reductive amination of aldehyde 1A with t-butyl 2-aminoacetate hydrochloride, for example, in the presence of acid and a reductant (e.g., sodium triacetoxyborohydride) affords secondary amine 1B. Carboxylic acids 1C are commercially available, known in the literature, and/or are or readily prepared by a wide variety of known methods. Condensation of secondary amine 1B and carboxylic acid 1C using standard amide coupling conditions (such as HATU or T₃P®) affords amide 1D. Deprotection of the t-butyl ester in 1D, for example, under acidic conditions (such as TFA in DCM), affords carboxylic acid 1E. Condensation of carboxylic acid 1E and an amine, for example, a primary amine (R³—NH₂, as depicted in Scheme 1) or a secondary amine (R³—N(H)—R³, including cyclic secondary amines, such as morpholine), using standard amide coupling conditions (such as HATU or T₃P®) affords amide 1F.

Alternatively, carboxylic acid 1E may be elaborated to a wide variety of cyclic groups, for example, heteroaryl and heterocycloalkyl groups, by a wide variety of known methods. For example, as depicted in Scheme 1, condensation of carboxylic acid 1E with t-butyl N-aminocarbamate using standard amide coupling conditions (such as HATU or T₃P®) affords hydrazide 1G, after deprotection of the Boc protecting group (for example, under acidic conditions, such as TFA). Condensation of hydrazide 1G affords heteroaryl compounds, such as amino-oxadiazole 1H, as depicted in Scheme 1, when hydrazide 1G is condensed with, for example, carbononitridic bromide and a base (such as NaHCO₃). Alternatively, hydrazide 1G may be condensed, for example, with a carboxylic acid (R⁴—CO₂H, using standard amide coupling conditions, such as HATU or T₃P®), and the resulting bis-acyl hydrazine can be cyclocondensed, for example with bis(trichloromethyl)carbonate, to afford an R⁴-substituted 1,3,4-oxadiazole.

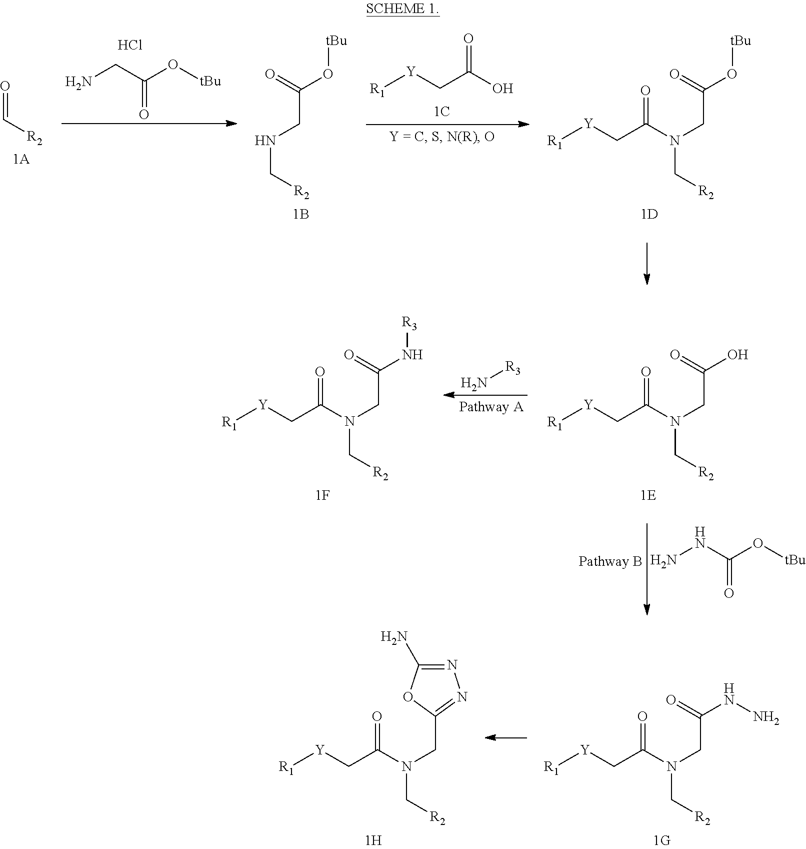

Scheme 2 illustrates a general method for preparing compounds of the type 2D. Aminomethyl compounds 2A are commercially available, known in the literature, and/or are readily prepared. Primary amines 2A may be elaborated to secondary amines 2B by a wide variety of known methods, for example, by reductive amination (as described above for conversion of aldehyde 1A to secondary amine 1B) or alkylation (with a reagent of the type R²—CH₂-LG, where LG is a leaving group, such as a halide, sulfonate, etc.). Condensation of secondary amine 2B and carboxylic acid 2C using standard amide coupling conditions (such as HATU or T3P®) affords amide 2D.

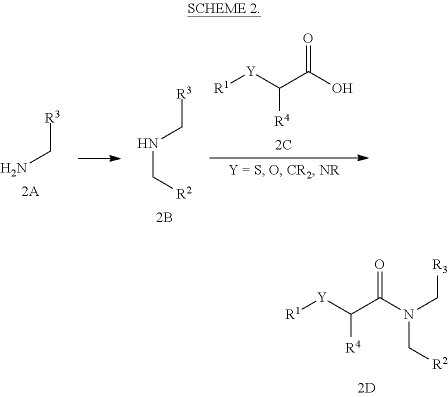

If a functional group is not amenable to a reaction condition, it is envisioned that the functional group can first be protected under standard conditions and then the protecting group removed after completing the transformation.

### II. Therapeutic Applications of Arylthioether Acetamide and Related Compounds

The arylthioether acetamide and related compounds described herein, such as a compound of Formula I, I-A, or other compounds in Section I, provide therapeutic benefits to patients suffering from a bacterial infection. Accordingly, one aspect of the invention provides a method of treating a bacterial infection in a patient. The method comprises administering a therapeutically effective amount of an arylthioether acetamide or related compound described herein, such as a compound of Formula I, I-A, or other compounds in Section I, to a patient in need thereof to treat the bacterial infection. In certain embodiments, the particular compound of Formula I or I-A is a compound defined by one of the embodiments described above. In certain embodiments, the compound is a compound of Formula II.

In certain embodiments, the bacterial infection is an infection by a gram-negative bacteria. In certain embodiments, the bacterial infection is an infection by a gram-positive bacteria. In certain embodiments, the bacterial infection is an anaerobic bacterial infection. In certain other embodiments, the bacterial infection is an aerobic bacterial infection.

In certain embodiments, the bacterial infection is an infection by a Staphylococcus, Streptococcus, Enterococcus, Pseudomonas, Escherichia, Fusobacterium, Klebsiella, Haemophilus, Bordetella, Serratia, Proteus, Enterobacter, Campylobacter, Citrobacter, Vibrio, Morganella, Salmonella, Shigella, Acinetobacter, Legionella, Bacteroides, Neisseria, Moraxella, Chlamydia, Helicobacter, Prevotella, Porphyromonas, Veillonella, Bilophila, Centipeda, Leptotrichia, Selenomonas, or Sutterella bacterium, or a combination thereof. In certain embodiments, the bacterial infection is an infection by a Staphylococcus, Streptococcus, Enterococcus, Pseudomonas, Escherichia, Fusobacterium, Klebsiella, Haemophilus, Bordetella, Serratia, Proteus, Enterobacter, Campylobacter, Citrobacter, Vibrio, Morganella, Salmonella, Shigella, Acinetobacter, Legionella, Bacteroides, Neisseria, Moraxella, Chlamydia, or Helicobacter bacterium, or a combination thereof. In certain embodiments, the bacterial infection is an infection by a Bacteroides, Prevotella, Fusobacterium, Porphyromonas, Veillonella, Bilophila, Centipeda, Leptotrichia, Selenomonas, or Sutterella bacterium, or a combination thereof. In certain embodiments, the bacterial infection is an infection by a Pseudomonas, Escherichia, or Fusobacterium bacterium, or a combination thereof. In certain embodiments, the bacterial infection is an infection by a Pseudomonas or Escherichia bacterium, or a combination thereof.

In certain embodiments, the bacterial infection is an infection by Staphylococcus aureus, Staphylococcus epidermidis, Streptococcus pyogenes, Streptococcus agalactiae, Streptococcus pneumoniae, Enterococcus faecalis, Enterococcus faecium, Pseudomonas aeruginosa, Escherichia coli, Fusobacterium nucleatum, Fusobacterium polymorphum, Fusobacterium vincentii, Fusobacterium animalis, Fusobacterium fusiforme, Fusobacterium canifelium, Fusobacterium necrophorum, Fusobacterium funduliforme, Fusobacterium ulcerans, Fusobacterium gonidiaformans, Fusobacterium mortiferum, Fusobacterium naviforme, Fusobacterium necrogenes, Fusobacterium russii, Fusobacterium varium, Klebsiella pneumoniae, Klebsiella oxytoca, Haemophilus influenzae, Haemophilus parainfluenzae, Bordetella pertussis, Bordetella bronchiseptica, Serratia marcescens, Proteus mirabilis, Enterobacter cloacae, Campylobacter jejuni, Vibrio parahemolyticus, Vibrio cholerae, Morganella morganii, Salmonella typhi, Salmonella paratyphi, Shigella dysenteriae, Acinetobacter baumannii, Acinetobacter calcoaceticus, Legionella pneumophila, Bacteroides fragilis, Bacteroides thetaiotaomicron, Bacteroides vulgatus, Bacteroides distasonis, Bacteroides ovatus, Bacteroides uniformis, Bacteroides caccae, Neisseria gonorrhoeae, Neisseria meningitides, Moraxella catarrhalis, Chlamydia trachomatis, Chlamydia psittaci, Helicobacter pylori, Prevotella intermedia, Prevotella melaninogenica, Prevotella bivia, Prevotella nigrescens, Prevotella disiens, Porphyromonas gingivalis, Veillonella atypica, Veillonella caviae, Veillonella criceti, Veillonella denticariosi, Veillonella dispar, Veillonella magna, Veillonella montpellierensis, Veillonella parvula, Veillonella ratti, Veillonella rodentium, Veillonella rogosae, Veillonella seminalis, Veillonella tobetsuensis Bilophila wadsworthia, Centipeda periodontii, Leptotrichia buccalis, Leptotrichia goodfellowii, Leptotrichia hofstadii, Leptotrichia honkongensis, Leptotrichia shahii, Leptotrichia trevisanii, Leptotrichia wadei, Selenomonas sputigena, Sutterella wadsworthensis, Sutterella parvirubra, Sutterella stercoricanis, or a combination thereof.

In certain embodiments, the bacterial infection is an infection by Staphylococcus aureus, Staphylococcus epidermidis, Streptococcus pyogenes, Streptococcus agalactiae, Streptococcus pneumoniae, Enterococcus faecalis, Enterococcus faecium, Pseudomonas aeruginosa, Escherichia coli, Fusobacterium nucleatum, Klebsiella pneumoniae, Klebsiella oxytoca, Haemophilus influenzae, Haemophilus parainfluenzae, Bordetella pertussis, Bordetella bronchiseptica, Serratia marcescens, Proteus mirabilis, Enterobacter cloacae, Campylobacter jejuni, Vibrio parahemolyticus, Vibrio cholerae, Morganella morganii, Salmonella typhi, Salmonella paratyphi, Shigella dysenteriae, Acinetobacter baumannii, Acinetobacter calcoaceticus, Legionella pneumophila, Bacteroides fragilis, Neisseria gonorrhoeae, Neisseria meningitides, Moraxella catarrhalis, Chlamydia trachomatis, Chlamydia psittaci, Helicobacter pylori, or a combination thereof. In certain embodiments, the bacterial infection is an infection by Pseudomonas aeruginosa, Escherichia coli, Fusobacterium nucleatum, or a combination thereof. In certain embodiments, the bacterial infection is an infection by Pseudomonas aeruginosa, Escherichia coli, or a combination thereof. In certain embodiments, the bacterial infection is an infection by Pseudomonas aeruginosa.

In certain embodiments, the bacterial infection is an infection of a bacteria selected from the group consisting of Enterobacteriaceae, Acinetobacter, Stenotrophomonas, Burkholderia, Pseudomonas, Alcaligenes, Haemophilus, Franciscellaceae and Neisseria species. In certain embodiments, the bacteria is Enterobacteriaceae or Acinetobacter. In certain embodiments, the Enterobacteriaceae is selected from the group consisting of Serratia, Proteus, Klebsiella, Enterobacter, Citrobacter, Salmonella, Providencia, Yersinia, Morganella, Cedecea, Edwardsiella species, and Escherichia coli. In certain embodiments, the Burkholderia is Burkholderia cepacia, Burkholderia pseudomallei or Burkholderia mallei. In certain embodiments, the Burkholderia is Burkholderia pseudomallei, Burkholderia mallei, or Burkholderia cepacia. In some embodiments, the Pseudomonas is Pseudomonas aeruginosa. In another embodiment, the Stenotrophomonas is Stenotrophomonas maltophila. In yet another embodiment, the Alcaligenes is Alcaligenes xylosoxidans.

In certain embodiments, the Acinetobacter is selected from the group consisting of Acinetobacter baumannii, Acinetobacter lwoffi, Acinetobacter albensis, Acinetobacter apis, Acinetobacter beijerinckii, Acinetobacter bereziniae, Acinetobacter bohemicus, Acinetobacter boissieri, Acinetobacter bouvetii, Acinetobacter brisouii, Acinetobacter calcoaceticus, Acinetobacter courvalinii, Acinetobacter dispersus, Acinetobacter equi, Acinetobacter gandensis, Acinetobacter gerneri, Acinetobacter guangdongensis, Acinetobacter guillouiae, Acinetobacter gyllenbergii, Acinetobacter haemolyticus, Acinetobacter harbinensis, Acinetobacter indicus, Acinetobacter junii, Acinetobacter kookii, Acinetobacter modestus, Acinetobacter nectaris, Acinetobacter nosocomialis, Acinetobacter parvus, Acinetobacter pakistanensis, Acinetobacter populi, Acinetobacter proteolyticus, Acinetobacter pittii, Acinetobacter puyangensis, Acinetobacter qingfengensis, Acinetobacter radioresistens, Acinetobacter rudis, Acinetobacter schindleri, Acinetobacter seifertii, Acinetobacter soli, Acinetobacter tandoii, Acinetobacter tjernbergiae, Acinetobacter towneri, Acinetobacter ursingii, Acinetobacter variabilis, Acinetobacter venetianus, and Acinetobacter vivianii.

In certain embodiments, the patient is a human.

Another aspect of the invention provides for the use of a compound described herein (such as a compound of Formula I, I-A, or other compounds in Section I) in the manufacture of a medicament. In certain embodiments, the medicament is for treating a bacterial infection described herein.

Another aspect of the invention provides for the use of a compound described herein (such as a compound of Formula I, I-A, or other compounds in Section I) for treating a bacterial infection described herein.

Further, the arylthioether acetamide and related compounds described herein, such as a compound of Formula I, I-A, or other compounds in Section I, can induce death of a bacterial cell. Accordingly, one aspect of the invention provides a method of inducing death of a bacterial cell. The method comprises exposing a bacterial cell to an effective amount of an arylthioether acetamide or related compound described herein, such as a compound of Formula I, I-A, or other compounds in Section I, to induce death of the bacterial cell. In certain embodiments, the particular compound of Formula I or I-A is a compound defined by one of the embodiments described above. In certain embodiments, the compound is a compound of Formula II.

In certain embodiments, the bacterial cell is one of those described above as being the bacterial species causing a bacterial infection. For example, in certain embodiments, the bacterial cell is a gram-negative bacteria. In certain embodiments, the bacterial cell is a gram-positive bacteria. In certain embodiments, the bacterial cell is an anaerobic bacteria. In certain other embodiments, the bacterial cell is an aerobic bacteria.

In certain embodiments, the bacterial cell is a Staphylococcus, Streptococcus, Enterococcus, Pseudomonas, Escherichia, Fusobacterium, Klebsiella, Haemophilus, Bordetella, Serratia, Proteus, Enterobacter, Campylobacter, Citrobacter, Vibrio, Morganella, Salmonella, Shigella, Acinetobacter, Legionella, Bacteroides, Neisseria, Moraxella, Chlamydia, Helicobacter, Prevotella, Porphyromonas, Veillonella, Bilophila, Centipeda, Leptotrichia, Selenomonas, or Sutterella bacterium, or a combination thereof. In certain embodiments, the bacterial cell is a Staphylococcus, Streptococcus, Enterococcus, Pseudomonas, Escherichia, Fusobacterium, Klebsiella, Haemophilus, Bordetella, Serratia, Proteus, Enterobacter, Campylobacter, Citrobacter, Vibrio, Morganella, Salmonella, Shigella, Acinetobacter, Legionella, Bacteroides, Neisseria, Moraxella, Chlamydia, or Helicobacter bacterium. In certain embodiments, the bacterial cell is a Bacteroides, Prevotella, Fusobacterium, Porphyromonas, Veillonella, Bilophila, Centipeda, Leptotrichia, Selenomonas, or Sutterella bacterium, or a combination thereof. In certain embodiments, the bacterial cell is a Pseudomonas, Escherichia, or Fusobacterium bacterium. In certain embodiments, the bacterial cell is a Pseudomonas or Escherichia bacterium.

In certain embodiments, the bacterial cell is a Staphylococcus aureus, Staphylococcus epidermidis, Streptococcus pyogenes, Streptococcus agalactiae, Streptococcus pneumoniae, Enterococcus faecalis, Enterococcus faecium, Pseudomonas aeruginosa, Escherichia coli, Fusobacterium nucleatum, Fusobacterium polymorphum, Fusobacterium vincentii, Fusobacterium animalis, Fusobacterium fusiforme, Fusobacterium canifelium, Fusobacterium necrophorum, Fusobacterium funduliforme, Fusobacterium ulcerans, Fusobacterium gonidiaformans, Fusobacterium mortiferum, Fusobacterium naviforme, Fusobacterium necrogenes, Fusobacterium russii, Fusobacterium varium, Klebsiella pneumoniae, Klebsiella oxytoca, Haemophilus influenzae, Haemophilus parainfluenzae, Bordetella pertussis, Bordetella bronchiseptica, Serratia marcescens, Proteus mirabilis, Enterobacter cloacae, Campylobacter jejuni, Vibrio parahemolyticus, Vibrio cholerae, Morganella morganii, Salmonella typhi, Salmonella paratyphi, Shigella dysenteriae, Acinetobacter baumannii, Acinetobacter calcoaceticus, Legionella pneumophila, Bacteroides fragilis, Bacteroides thetaiotaomicron, Bacteroides vulgatus, Bacteroides distasonis, Bacteroides ovatus, Bacteroides uniformis, Bacteroides caccae, Neisseria gonorrhoeae, Neisseria meningitides, Moraxella catarrhalis, Chlamydia trachomatis, Chlamydia psittaci, Helicobacter pylori, Prevotella intermedia, Prevotella melaninogenica, Prevotella bivia, Prevotella nigrescens, Prevotella disiens, Porphyromonas gingivalis, Veillonella atypica, Veillonella caviae, Veillonella criceti, Veillonella denticariosi, Veillonella dispar, Veillonella magna, Veillonella montpellierensis, Veillonella parvula, Veillonella ratti, Veillonella rodentium, Veillonella rogosae, Veillonella seminalis, Veillonella tobetsuensis Bilophila wadsworthia, Centipeda periodontii, Leptotrichia buccalis, Leptotrichia goodfellowii, Leptotrichia hofstadii, Leptotrichia honkongensis, Leptotrichia shahii, Leptotrichia trevisanii, Leptotrichia wadei, Selenomonas sputigena, Sutterella wadsworthensis, Sutterella parvirubra, Sutterella stercoricanis, or a combination thereof. In certain embodiments, the bacterial cell is a Staphylococcus aureus, Staphylococcus epidermidis, Streptococcus pyogenes, Streptococcus agalactiae, Streptococcus pneumoniae, Enterococcus faecalis, Enterococcus faecium, Pseudomonas aeruginosa, Escherichia coli, Fusobacterium nucleatum, Klebsiella pneumoniae, Klebsiella oxytoca, Haemophilus influenzae, Haemophilus parainfluenzae, Bordetella pertussis, Bordetella bronchiseptica, Serratia marcescens, Proteus mirabilis, Enterobacter cloacae, Campylobacter jejuni, Vibrio parahemolyticus, Vibrio cholerae, Morganella morganii, Salmonella typhi, Salmonella paratyphi, Shigella dysenteriae, Acinetobacter baumannii, Acinetobacter calcoaceticus, Legionella pneumophila, Bacteroides fragilis, Neisseria gonorrhoeae, Neisseria meningitides, Moraxella catarrhalis, Chlamydia trachomatis, Chlamydia psittaci, Helicobacter pylori bacterium. In certain embodiments, the bacterial cell is a Pseudomonas aeruginosa, Escherichia coli, or Fusobacterium nucleatum bacterium. In certain embodiments, the bacterial cell is a Pseudomonas aeruginosa or Escherichia coli bacterium. In certain embodiments, the bacterial cell is a Pseudomonas aeruginosa.

In certain embodiments, the bacterial cell is a bacteria selected from the group consisting of Enterobacteriaceae, Acinetobacter, Stenotrophomonas, Burkholderia, Pseudomonas, Alcaligenes, Haemophilus, Franciscellaceae and Neisseria species. In certain embodiments, the bacteria is Enterobacteriaceae or Acinetobacter. In certain embodiments, the Enterobacteriaceae is selected from the group consisting of Serratia, Proteus, Klebsiella, Enterobacter, Citrobacter, Salmonella, Providencia, Yersinia, Morganella, Cedecea, Edwardsiella species, and Escherichia coli. In certain embodiments, the Burkholderia is Burkholderia cepacia, Burkholderia pseudomallei or Burkholderia mallei. In certain embodiments, the Burkholderia is Burkholderia pseudomallei, Burkholderia mallei, or Burkholderia cepacia. In some embodiments, the Pseudomonas is Pseudomonas aeruginosa. In another embodiment, the Stenotrophomonas is Stenotrophomonas maltophila. In yet another embodiment, the Alcaligenes is Alcaligenes xylosoxidans.

In certain embodiments, the Acinetobacter is selected from the group consisting of Acinetobacter baumannii, Acinetobacter lwoffi, Acinetobacter albensis, Acinetobacter apis, Acinetobacter beijerinckii, Acinetobacter bereziniae, Acinetobacter bohemicus, Acinetobacter boissieri, Acinetobacter bouvetii, Acinetobacter brisouii, Acinetobacter calcoaceticus, Acinetobacter courvalinii, Acinetobacter dispersus, Acinetobacter equi, Acinetobacter gandensis, Acinetobacter gerneri, Acinetobacter guangdongensis, Acinetobacter guillouiae, Acinetobacter gyllenbergii, Acinetobacter haemolyticus, Acinetobacter harbinensis, Acinetobacter indicus, Acinetobacter junii, Acinetobacter kookii, Acinetobacter modestus, Acinetobacter nectaris, Acinetobacter nosocomialis, Acinetobacter parvus, Acinetobacter pakistanensis, Acinetobacter populi, Acinetobacter proteolyticus, Acinetobacter pittii, Acinetobacter puyangensis, Acinetobacter qingfengensis, Acinetobacter radioresistens, Acinetobacter rudis, Acinetobacter schindleri, Acinetobacter seifertii, Acinetobacter soli, Acinetobacter tandoii, Acinetobacter tjernbergiae, Acinetobacter towneri, Acinetobacter ursingii, Acinetobacter variabilis, Acinetobacter venetianus, and Acinetobacter vivianii.

Further, the arylthioether acetamide and related compounds described herein, such as a compound of Formula I, I-A, or other compounds in Section I, can kill a bacterial cell. Accordingly, one aspect of the invention provides a method of killing a bacterial cell. The method comprises exposing a bacterial cell to an effective amount of an arylthioether acetamide or related compound described herein, such as a compound of Formula I, I-A, or other compounds in Section I, to kill the bacterial cell. In certain embodiments, the particular compound of Formula I or I-A is a compound defined by one of the embodiments described above. In certain embodiments, the compound is a compound of Formula II.

In certain embodiments, the bacterial cell is one of those described above as being the bacterial species causing a bacterial infection. For example, in certain embodiments, the bacterial cell is a gram-negative bacteria. In certain embodiments, the bacterial cell is a gram-positive bacteria. In certain embodiments, the bacterial cell is an anaerobic bacteria. In certain other embodiments, the bacterial cell is an aerobic bacteria.

In certain embodiments, the bacterial cell is a Staphylococcus, Streptococcus, Enterococcus, Pseudomonas, Escherichia, Fusobacterium, Klebsiella, Haemophilus, Bordetella, Serratia, Proteus, Enterobacter, Campylobacter, Citrobacter, Vibrio, Morganella, Salmonella, Shigella, Acinetobacter, Legionella, Bacteroides, Neisseria, Moraxella, Chlamydia, Helicobacter, Prevotella, Porphyromonas, Veillonella, Bilophila, Centipeda, Leptotrichia, Selenomonas, or Sutterella bacterium, or a combination thereof. In certain embodiments, the bacterial cell is a Staphylococcus, Streptococcus, Enterococcus, Pseudomonas, Escherichia, Fusobacterium, Klebsiella, Haemophilus, Bordetella, Serratia, Proteus, Enterobacter, Campylobacter, Citrobacter, Vibrio, Morganella, Salmonella, Shigella, Acinetobacter, Legionella, Bacteroides, Neisseria, Moraxella, Chlamydia, or Helicobacter bacterium. In certain embodiments, the bacterial cell is a Staphylococcus, Streptococcus, Enterococcus, Pseudomonas, Escherichia, Fusobacterium, Klebsiella, Haemophilus, Bordetella, Serratia, Proteus, Enterobacter, Campylobacter, Citrobacter, Vibrio, Morganella, Salmonella, Shigella, Acinetobacter, Legionella, Bacteroides, Neisseria, Moraxella, Chlamydia, or Helicobacter bacterium. In certain embodiments, the bacterial cell is a Pseudomonas, Escherichia, or Fusobacterium bacterium. In certain embodiments, the bacterial cell is a Pseudomonas or Escherichia bacterium.

In certain embodiments, the bacterial cell is a Staphylococcus aureus, Staphylococcus epidermidis, Streptococcus pyogenes, Streptococcus agalactiae, Streptococcus pneumoniae, Enterococcus faecalis, Enterococcus faecium, Pseudomonas aeruginosa, Escherichia coli, Fusobacterium nucleatum, Fusobacterium polymorphum, Fusobacterium vincentii, Fusobacterium animalis, Fusobacterium fusiforme, Fusobacterium canifelium, Fusobacterium necrophorum, Fusobacterium funduliforme, Fusobacterium ulcerans, Fusobacterium gonidiaformans, Fusobacterium mortiferum, Fusobacterium naviforme, Fusobacterium necrogenes, Fusobacterium russii, Fusobacterium varium, Klebsiella pneumoniae, Klebsiella oxytoca, Haemophilus influenzae, Haemophilus parainfluenzae, Bordetella pertussis, Bordetella bronchiseptica, Serratia marcescens, Proteus mirabilis, Enterobacter cloacae, Campylobacter jejuni, Vibrio parahemolyticus, Vibrio cholerae, Morganella morganii, Salmonella typhi, Salmonella paratyphi, Shigella dysenteriae, Acinetobacter baumannii, Acinetobacter calcoaceticus, Legionella pneumophila, Bacteroides fragilis, Bacteroides thetaiotaomicron, Bacteroides vulgatus, Bacteroides distasonis, Bacteroides ovatus, Bacteroides uniformis, Bacteroides caccae, Neisseria gonorrhoeae, Neisseria meningitides, Moraxella catarrhalis, Chlamydia trachomatis, Chlamydia psittaci, Helicobacter pylori, Prevotella intermedia, Prevotella melaninogenica, Prevotella bivia, Prevotella nigrescens, Prevotella disiens, Porphyromonas gingivalis, Veillonella atypica, Veillonella caviae, Veillonella criceti, Veillonella denticariosi, Veillonella dispar, Veillonella magna, Veillonella montpellierensis, Veillonella parvula, Veillonella ratti, Veillonella rodentium, Veillonella rogosae, Veillonella seminalis, Veillonella tobetsuensis Bilophila wadsworthia, Centipeda periodontii, Leptotrichia buccalis, Leptotrichia goodfellowii, Leptotrichia hofstadii, Leptotrichia honkongensis, Leptotrichia shahii, Leptotrichia trevisanii, Leptotrichia wadei, Selenomonas sputigena, Sutterella wadsworthensis, Sutterella parvirubra, Sutterella stercoricanis, or a combination thereof. In certain embodiments, the bacterial cell is a Staphylococcus aureus, Staphylococcus epidermidis, Streptococcus pyogenes, Streptococcus agalactiae, Streptococcus pneumoniae, Enterococcus faecalis, Enterococcus faecium, Pseudomonas aeruginosa, Escherichia coli, Fusobacterium nucleatum, Klebsiella pneumoniae, Klebsiella oxytoca, Haemophilus influenzae, Haemophilus parainfluenzae, Bordetella pertussis, Bordetella bronchiseptica, Serratia marcescens, Proteus mirabilis, Enterobacter cloacae, Campylobacter jejuni, Vibrio parahemolyticus, Vibrio cholerae, Morganella morganii, Salmonella typhi, Salmonella paratyphi, Shigella dysenteriae, Acinetobacter baumannii, Acinetobacter calcoaceticus, Legionella pneumophila, Bacteroides fragilis, Neisseria gonorrhoeae, Neisseria meningitides, Moraxella catarrhalis, Chlamydia trachomatis, Chlamydia psittaci, Helicobacter pylori bacterium. In certain embodiments, the bacterial cell is a Pseudomonas aeruginosa, Escherichia coli, or Fusobacterium nucleatum bacterium. In certain embodiments, the bacterial cell is a Pseudomonas aeruginosa or Escherichia coli bacterium. In certain embodiments, the bacterial cell is a Pseudomonas aeruginosa.

In certain embodiments, the bacterial cell is a bacteria selected from the group consisting of Enterobacteriaceae, Acinetobacter, Stenotrophomonas, Burkholderia, Pseudomonas, Alcaligenes, Haemophilus, Franciscellaceae and Neisseria species. In certain embodiments, the bacteria is Enterobacteriaceae or Acinetobacter. In certain embodiments, the Enterobacteriaceae is selected from the group consisting of Serratia, Proteus, Klebsiella, Enterobacter, Citrobacter, Salmonella, Providencia, Yersinia, Morganella, Cedecea, Edwardsiella species, and Escherichia coli. In certain embodiments, the Burkholderia is Burkholderia cepacia, Burkholderia pseudomallei or Burkholderia mallei. In certain embodiments, the Burkholderia is Burkholderia pseudomallei, Burkholderia mallei, or Burkholderia cepacia. In some embodiments, the Pseudomonas is Pseudomonas aeruginosa. In another embodiment, the Stenotrophomonas is Stenotrophomonas maltophila. In yet another embodiment, the Alcaligenes is Alcaligenes xylosoxidans.

In certain embodiments, the Acinetobacter is selected from the group consisting of Acinetobacter baumannii, Acinetobacter lwoffi, Acinetobacter albensis, Acinetobacter apis, Acinetobacter beijerinckii, Acinetobacter bereziniae, Acinetobacter bohemicus, Acinetobacter boissieri, Acinetobacter bouvetii, Acinetobacter brisouii, Acinetobacter calcoaceticus, Acinetobacter courvalinii, Acinetobacter dispersus, Acinetobacter equi, Acinetobacter gandensis, Acinetobacter gerneri, Acinetobacter guangdongensis, Acinetobacter guillouiae, Acinetobacter gyllenbergii, Acinetobacter haemolyticus, Acinetobacter harbinensis, Acinetobacter indicus, Acinetobacter junii, Acinetobacter kookii, Acinetobacter modestus, Acinetobacter nectaris, Acinetobacter nosocomialis, Acinetobacter parvus, Acinetobacter pakistanensis, Acinetobacter populi, Acinetobacter proteolyticus, Acinetobacter pittii, Acinetobacter puyangensis, Acinetobacter qingfengensis, Acinetobacter radioresistens, Acinetobacter rudis, Acinetobacter schindleri, Acinetobacter seifertii, Acinetobacter soli, Acinetobacter tandoii, Acinetobacter tjernbergiae, Acinetobacter towneri, Acinetobacter ursingii, Acinetobacter variabilis, Acinetobacter venetianus, and Acinetobacter vivianii.

Further, the arylthioether acetamide and related compounds described herein, such as a compound of Formula I, I-A, or other compounds in Section I, inhibit the activity of LpxA. Accordingly, one aspect of the invention provides a method of inhibiting the activity of LpxA. The method comprises exposing an LpxA to an effective amount of an arylthioether acetamide or related compound described herein, such as a compound of Formula I, I-A, or other compounds in Section I, to inhibit the activity of LpxA. In certain embodiments, the particular compound of Formula I or I-A is a compound defined by one of the embodiments described above.

Another aspect of the invention provides a method of inhibiting the activity of LpxD. The method comprises exposing an LpxD to an effective amount of an arylthioether acetamide or related compound described herein, such as a compound of Formula I, I-A, or other compounds in Section I, to inhibit the activity of LpxD. In certain embodiments, the particular compound of Formula I or I-A is a compound defined by one of the embodiments described above.

### Analysis of Ability to Inhibit LpxA or LpxD Activity

Ability of compounds to inhibit LpxA or LpxD activity may be evaluated, for example, using an in vitro substrate acylation assay with RapidFire™ MS analysis, according to the procedures described in Example 20 and Example 21.

Ability of Compounds to Inhibit Growth of Bacteria (e.g. Escherichia and Pseudomonas)

Ability of compounds to inhibit the growth of Escherichia, Pseudomonas, and other bacteria can be evaluated using broth microdilution methods, for example, according to the procedures described in Example 22.

### III. Combination Therapy

Another aspect of the invention provides for combination therapy. Arylthioether acetamide and related compounds (e.g., a compound of Formula I, I-A, or other compounds in Section I) or their pharmaceutically acceptable salts may be used in combination with additional therapeutic agents to treat a bacterial infection.

The amount of arylthioether acetamide or related compound (e.g., a compound of Formula I, I-A, or other compounds in Section I) and additional therapeutic agent and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, an arylthioether acetamide or related compound (e.g., a compound of any one of Formula I, I-A, or other compounds in Section I) may be administered during a time when the additional therapeutic agent(s) exerts its prophylactic or therapeutic effect, or vice versa.

The doses and dosage regimen of the active ingredients used in the combination therapy may be determined by an attending clinician. In certain embodiments, the arylthioether acetamide or related compound (e.g., a compound of any one of Formula I, I-A, or other compounds in Section I) and the additional therapeutic agent(s) are administered in doses commonly employed when such agents are used as monotherapy for treating the bacterial infection. In other embodiments, the arylthioether acetamide or related compound (e.g., a compound of any one of Formula I, I-A, or other compounds in Section I) and the additional therapeutic agent(s) are administered in doses lower than the doses commonly employed when such agents are used as monotherapy for treating the bacterial infection. In certain embodiments, the arylthioether acetamide or related compound (e.g., a compound of any one of Formula I, I-A, or other compounds in Section I) and the additional therapeutic agent(s) are present in the same composition, which is suitable for oral administration.

In certain embodiments, the arylthioether acetamide or related compound (e.g., a compound of any one of Formula I, I-A, or other compounds in Section I) and the additional therapeutic agent(s) may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

In certain embodiments, the additional therapeutic agent is selected from the group consisting of amikacin, arbekacin, dibekacin, gentamicin, isepamicin, kanamycin a, neomycin, netilmicin, paromomycin, sisomicin, streptomycin, tobramycin, chloramphenicol, loracarbef, ertapenem, imipenem, meropenem, R-119699, ceflaclor, cefadroxil, cefamandole, cefazolin, cefdinir, cefditoren, cefepime, cefetamet, cefixime, cefmetazole, cefotaxime, cefoxitin, ceftibuten, cefoperazone, cefotetan, cefpirome, cefpodoxime, cefprozil, ceftazidime, ceftizoxime, ceftobiprole, ceftriaxone, cefuroxime, cephalexin, cephalothin, cephradine, T-91929, iclaprim, trimethoprim, ciprofloxacin, ABT-492, clinafloxacin, danofloxacin, difloxacin, DX-619, enoxacin, fleroxacin, garenoxacin, gatifloxacin, gemifloxacin, grepafloxacin, levofloxacin, lomefloxacin, moxifloxacin, nadifloxacin, nalidixic acid, norfiloxacin, pefloxacin, rufloxacin, sitafloxacin, sparfloxacin, temafloxacin, trovafloxacin, dalbavancin, oritavancin, teicoplanin, telavancin, vancomycin, chlorobiocin, novobiocin, pseudomonic acid A, clindamycin, lincomycin, daptomycin, azithromycin, cethromycin, clarithromycin, dirithromycin, EP-13420, erythromycin, roxithromycin, telithromycin, aztreonam, fosfomycin, linezolid, ranbezolid, doripenem, faropenem, amoxicillin, ampicillin, azlocillin, carenicllin, cloxacillin, dicloxacillin, methicillin, mezlocillin, nafcillin, oxacillin, penicillin G, penicillin V, piperacillin, ticarcillin, rifalazil, rifampin, dalfopristin, quinupristin, sulfabenzamide, sulfacetamide, sulfachlorpyridazine, sulfadiazine, sulfadimethoxine, sulfaguanidine, sulfamerazine, sulfameter, sulfamethazine, sulfamethizole, sulfamethoxazole, sulfamethoxypyridazine, sulfamonomethoxine, sulfanitran, sulfaphenazole, sulfapyridine, sulfaquinoxaline, sulfathiazole, sulfisoxazole, gramicidin, polymyxin B1, triclosan, chlortetracycline, demeclocycline, doxycycline, meclocycline, methacycline, minocycline, oxytetracycline, PTK-0796, tetracycline, tigecycline, fusidic acid, and combinations thereof.

Another aspect of this invention is a kit comprising a therapeutically effective amount of the arylthioether acetamide or related compound (e.g., a compound of any one of Formula I, I-A, or other compounds in Section I), a pharmaceutically acceptable carrier, vehicle or diluent, and optionally at least one additional therapeutic agent listed above.

### IV. Pharmaceutical Compositions and Dosing Considerations

As indicated above, the invention provides pharmaceutical compositions. In one embodiment, the pharmaceutical composition comprises a compound described herein and a pharmaceutically acceptable carrier. In a more specific embodiment, the pharmaceutical composition comprises a therapeutically-effective amount of a compound described above, formulated together with a pharmaceutically acceptable carrier. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (9) sublingually; (6) ocularly; (7) transdermally; or (9) nasally.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 9 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (9) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (9) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.9 mg/kg to about 90 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising an arylthioether acetamide or related compound described herein in a therapeutically effective amount for the treatment of a bacterial infection described herein.

### EXAMPLES

The invention now being generally described, will be more readily understood by reference to the following examples, which are included merely for purposes of illustration of certain aspects and embodiments of the present invention, and are not intended to limit the invention. Starting materials described herein can be obtained from commercial sources or may be readily prepared from commercially available materials using transformations known to those of skill in the art.

### General Experimental Details

Abbreviations used include: aq.=aqueous; DCE=dichloroethane; DCM=dichloromethane; DIAD=Di-isopropyl azodicarboxylate; DIPEA=diisopropylethylamine; DMF=N,N-dimethylformamide; DMSO=dimethyl sulfoxide; EtOAc=ethyl acetate; h=hour; HATU=N-[(Dimethylamino)-1H-1,2,3-triazolo-[4,5-b]pyridin-1-ylmethylene]-N-methylmethanaminium hexafluorophosphate N-oxide; HPLC=high performance liquid chromatography; LCMS=liquid chromatography mass spectrometry; Me=methyl; min=minutes; NMR=nuclear magnetic resonance; RT=room temperature; rt=retention time; sat.=saturated; SCX-2=strong cation exchange chromatography; TBAF=tetrabutylammonium fluoride; TFA=trifluoroacetic acid; TFAA=Trifluoroacetic anhydride; THE=Tetrahydrofuran; T3P=propylphosphonic anhydride.

All solvents and commercial reagents were used as received, unless otherwise specified.

NMR spectra were obtained on a Bruker Avance III HD 500 MHz NMR or a Bruker Avance III HD 250 MHz NMR. Shifts are given in ppm relative to tetramethylsilane (δ=0 ppm). J values are given in Hz throughout.

Flash column chromatography was performed in a Biotage Isolera system using either a reverse phase column (C18) or a normal phase column (SiO₂). Methods employed, and the corresponding eluents, were:
High pH Reverse Phase—Water+0.1% NH₄OH:MeCN+0.1% NH₄OH; 90:10 to 0:100.
Low pH Reverse Phase—Water+0.1% HCO₂H:MeCN+0.1% HCO₂H; 90:10 to 0:100.
Normal Phase Method 1—Heptane:EtOAc; 100:0 to 0:100, or until product eluted.
Normal Phase Method 2—CH₂Cl₂:MeOH; 100:0 to 90:10, or until product eluted.

Preparative HPLC purification was performed with a flow rate of 40 mL/min, UV detection at 215 nm, and a Waters Prep. C18 10 μm OBD™, 30×100 mm column (XBridge™ for High pH, and Sunfire™ for Low pH). Methods employed, and corresponding eluents, were:
High pH Standard—MeCN+0.2% NH₄OH:Water+0.2% NH₄OH; 30:70 to 95:5 over 14 min.
High pH Early Elute—MeCN+0.2% NH₄OH:Water+0.2% NH₄OH; 10:90 to 95:5 over 17 min.
Low pH Standard—MeCN+0.1% HCO₂H:Water+0.1% HCO₂H; 30:70 to 95:5 over 14 min.
Low pH Early Elute—MeCN+0.1% HCO₂H H₄OH:Water+0.1% HCO₂H; 10:90 to 95:5 over 17 min.

Analytical liquid chromatography mass spectroscopy (LCMS) was conducted at 40° C. with the following methods:
LCMS Method A—Mobile phase: 5-100% MeCN in water (both with 0.1% HCO₂H) over 5.8 min at a flow rate of 0.6 mL/min; Column: Phenomenex Kinetex-XB C18 column (Part No. 00D-4498-AN), 2.1×100 mm, 1.7 μm.
LCMS Method B—Mobile phase: 5-100% MeCN in water (both with 0.1% HCO₂H) over 1.3 min at a flow rate of 1.2 mL/min; Column: Kinetex Core-Shell C18, (Part No. 00B-4608-AN), 2.1 mm×50 mm, 5 μm.
LCMS Method C—Mobile phase: 5-100% MeCN in water (both with 0.1% HCO₂H) over 5.4 min at a flow rate of 0.6 mL/min; Column: Waters Atlantis dC18, (Part No. 186001295), 2.1×100 mm, 3 μm.
LCMS Method D—Mobile phase: 1-100% MeCN in water (both with 2 mM ammonium bicarbonate, buffered to pH 10) over 1.8 min at a flow rate of 1.0 mL/min; Column: Phenomenex Gemini-NX C18, (Part No. 00B-4453B0), 2.0×50 mm, 3 μm.
LCMS Method E—Mobile phase: 5-100% MeCN in water (both with 0.1% HCO₂H) over 1.1 min at a flow rate of 0.9 mL/min; Column: Waters UPLC® CSH™ C18 (Part No. 186005297), 2.1×100 mm, 1.7 μm.
LCMS Method F—Mobile phase: 5-100% MeCN in water (both with 0.1% HCO₂H) over 1.8 min at a flow rate of 1.2 mL/min; Column: Kinetex Core-Shell C₈, (Part No. 00B-4608-AN), 2.1×50 mm, 5 μm.
LCMS Method G—Mobile phase: 5-100% MeCN in water (both with 0.1% HCO₂H) over 2.7 min at a flow rate of 1.0 mL/min; Column: Waters Atlantis dC18, (Part No. 186001291), 2.1×50 mm, 3 μm.
LCMS Method H—Mobile phase: 1-100% MeCN in water (both with 2 mM ammonium bicarbonate, buffered to pH 10) over 5.9 min at a flow rate of 0.5 mL/min; Column: Phenomenex Gemini-NX C18, (Part No. 00B-4453B0), 2.0×50 mm, 3 μm.

### Example 1—Synthesis of 2-[[2-(2-Chlorophenyl)sulfanylacetyl]-[(4-cyanophenyl)methyl]amino]-N-(oxetan-3-yl)acetamide (Compound No. 1)

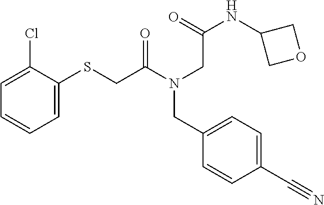 [SMILES: N#Cc1ccc(CN(CC(=O)NC2COC2)C(=O)CSc2ccccc2Cl)cc1]

### tert-Butyl (4-cyanobenzyl)glycinate

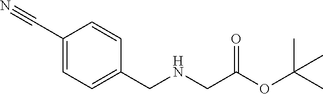 [SMILES: CC(C)(C)OC(=O)CNCc1ccc(C#N)cc1]

To a solution of tert-butyl 2-aminoacetate hydrochloride (3.84 g, 22.9 mmol) in Methanol (95.0 mL), 12 g of MP-carbonate was added and the reaction stirred for 3 hours to remove the HCl salt. The resultant solution was filtered and to the filtrate was added 4-formylbenzonitrile (1.9 mL, 15.3 mmol) and 10 drops of acetic acid. The reaction was stirred for 15 minutes before sodium triacetoxyborahydride (7.11 g, 33.6 mmol) was added and the reaction was stirred for a further 24 hours. Further portions of sodium triacetoxyborahydride were added until consumption of the 4-formylbenzonitrile was observed. The reaction was concentrated in vacuo to remove the methanol and diluted with Na₂CO₃ (150 mL). The aqueous layer was extracted with ethyl acetate (3×70 mL) and the combined organic extracts were dried over Na₂SO₄, filtered and concentrated in vacuo. The residue was purified by Normal Phase column chromatography Method 2 to afford the title compound (2.68 g, 10.9 mmol, 71% yield) as a pale yellow oil. 1H NMR (400 MHz, Methanol-d4) δ 7.73-7.67 (m, 2H), 7.57-7.51 (m, 2H), 3.86 (s, 2H), 3.29 (s, 2H), 1.47 (s, 9H). (ESI⁺)—LCMS Method D, rt=1.57 min, [M+H]⁺=247.2.

### Tert-butyl 2-[[2-(2-chlorophenyl)sulfanylacetyl]-[(4-cyanophenyl)methyl]amino]acetate

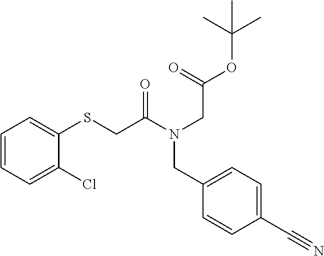 [SMILES: CC(C)(C)OC(=O)CN(Cc1ccc(C#N)cc1)C(=O)CSc1ccccc1Cl]

To a solution of 2-(2-chlorophenyl)sulfanylacetic acid (98%, 1.00 g, 4.84 mmol) and N-ethyl-N-isopropyl-propan-2-amine (1.3 mL, 7.25 mmol) in DMF (30 mL) was added tert-butyl (4-cyanobenzyl)glycinate (100%, 1310 mg, 5.32 mmol) and 1-[bis(dimethylamino) methylidene]-1H-[1,2,3]triazolo[4,5-b]pyridin-1-ium 3-oxide hexafluorophosphate (2.21 g, 5.80 mmol) and the reaction stirred to RT for 17 hours. The reaction was concentrated in vacuo to remove the DMF and the residue taken up in ethyl acetate (50 mL). The organic layer was washed with sat. aqueous NaHCO₃ (2×25 mL) followed by brine (2×25 mL) then dried over Na₂SO₄, filtered and concentrated in vacuo. The residue was purified by Normal phase column chromatography Method 1 to afford the title compound (2.08 g, 4.54 mmol, 94% yield) as a very pale yellow oil. 1H NMR (400 MHz, DMSO-d6) δ 7.86-7.76 (m, 2H), 7.57-7.16 (m, 6H), 4.89-4.58 (m, 2H), 4.33-4.09 (m, 2H), 4.08-3.90 (m, 2H), 1.37-1.34 (m, 9H). (ESI⁺)—LCMS Method D, rt=1.85 mins, [M+H]⁺=431.1.

### 2-[[2-(2-Chlorophenyl)sulfanylacetyl]-[(4-cyanophenyl)methyl]amino]acetic acid

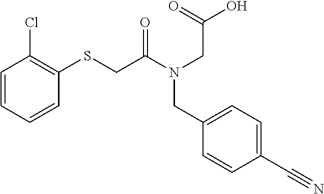 [SMILES: N#Cc1ccc(CN(CC(=O)O)C(=O)CSc2ccccc2Cl)cc1]

To a solution of tert-butyl 2-[[2-(2-chlorophenyl)sulfanylacetyl]-[(4-cyanophenyl)methyl]amino]acetate (2.08 g, 4.83 mmol) in DCM (48.0 mL) was added 2,2,2-trifluoroacetic acid (1.8 mL, 24.1 mmol) and the reaction left to stir at RT for 42 hours. The reaction was concentrated in vacuo and the crude residue was azeotroped with DCM (2×40 mL) to afford 2-[[2-(2-chlorophenyl)sulfanylacetyl]-[(4-cyanophenyl)methyl]amino]acetic acid (1.88 mg, 4.67 mmol, 97% yield) as a viscous oil which was used without any further purification. 1H NMR (500 MHz, DMSO-d6) δ 7.86-7.76 (m, 2H), 7.59-7.13 (m, 6H), 4.92-4.56 (m, 2H), 4.31-3.93 (m, 4H). (ESI⁺)—LCMS Method D, rt=1.13 mins, [M+H]⁺=375.1.

### 2-[[2-(2-Chlorophenyl)sulfanylacetyl]-[(4-cyanophenyl)methyl]amino]-N-(oxetan-3-yl)acetamide (Compound No. 1)

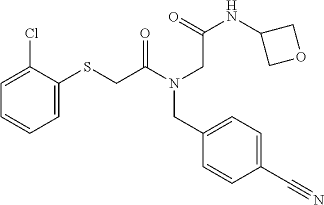 [SMILES: N#Cc1ccc(CN(CC(=O)NC2COC2)C(=O)CSc2ccccc2Cl)cc1]

2-[[2-(2-Chlorophenyl)sulfanylacetyl]-[(4-cyanophenyl)methyl]amino]acetic acid (0.050 mL, 0.133 mmol), N-ethyl-N-isopropyl-propan-2-amine (0.026 mL, 0.147 mmol) and oxetan-3-amine (0.019 mL, 0.267 mmol) were dissolved in anhydrous DMF (1.5 mL). Without delay, 1-[bis(dimethylamino)methylidene]-1H-[1,2,3]triazolo[4,5-b]pyridin-1-ium 3-oxide hexafluorophosphate (56 mg, 0.147 mmol) was added and the reaction was stirred at room temperature for 1 hour. The reaction was purified by Low pH preparative HPLC (Early elute method) and the fraction were concentrated in vacuo and dissolved in MeCN:H2O 1:1 and freeze dried to afford 2-[[2-(2-chlorophenyl)sulfanylacetyl]-[(4-cyanophenyl)methyl]amino]-N-(oxetan-3-yl)acetamide (31 mg, 0.0721 mmol, 54% yield) as a white powder. 1H NMR (500 MHz, Methanol-d4) δ 7.75-7.63 (m, 2H), 7.52-7.35 (m, 4H), 7.30-7.16 (m, 2H), 4.94-4.87 (m, 2H), 4.84-4.81 (m, 2H), 4.65 (s, 1H), 4.54-4.46 (m, 2H), 4.22-3.98 (in, 4H). (ESI⁺)—LCMS Method C, rt=3.64 mins, [M+H]⁺=430.05.

The compounds in Table 2 below were prepared based on experimental procedures described above in this Example 1.

TABLE 2

| | | Mass | |
|---|---|---|---|
| Compound | | Spec. | |
| No. | Chemical Structure | (ES, m/z) | ¹H NMR Spectrum |
| 2 | 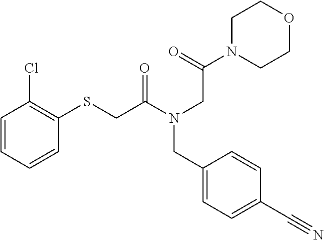 [SMILES: N#Cc1ccc(CN(CC(=O)N2CCOCC2)C(=O)CSc2ccccc2Cl)cc1] | 466.1 [M + H]+ | (500 MHz, Methanol-d4) δ 7.75-7.70 (m, 1H), 7.68- 7.63 (m, 1H), 7.50-7.35 (m, 4H), 7.30-7.15 (m, 2H), 4.92-4.62 (m, 2H), 4.47-4.21 (m, 2H), 4.03- 3.89 (m, 2H), 3.67-3.60 (m, 4H), 3.58-3.53 (m, 2H), 3.46-3.40 (m, 2H). |
| 3 | 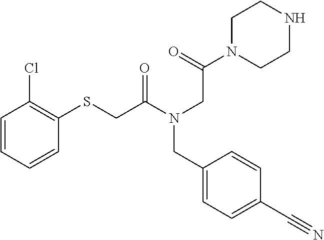 [SMILES: N#Cc1ccc(CN(CC(=O)N2CCNCC2)C(=O)CSc2ccccc2Cl)cc1] | 443.0 [M + H]+ | (500 MHz, Methanol-d4) δ 7.76-7.65 (m, 2H), 7.52- 7.37 (m, 4H), 7.32-7.17 (m, 2H), 4.67-4.55 (m, 2H), 4.49-4.23 (m, 2H), 4.04-3.90 (m, 2H), 3.61- 3.54 (m, 2H), 3.48-3.40 (m, 2H), 2.88-2.80 (m, 4H). |
| 4 | 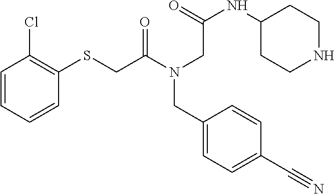 [SMILES: N#Cc1ccc(CN(CC(=O)NC2CCNCC2)C(=O)CSc2ccccc2Cl)cc1] | 457.2 [M + H]+ | (500 MHz, DMSO-d6) δ 8.05-7.72 (m, 3H), 7.52- 7.15 (m, 6H), 4.89-4.50 (m, 2H), 4.17-3.81 (m, 4H), 3.64-3.54 (m, 1H), 2.94-2.85 (m, 2H), 2.48- 2.42 (m, 2H), 1.67-1.57 (m, 2H), 1.27-1.14 (m, 2H). |
| 5 | 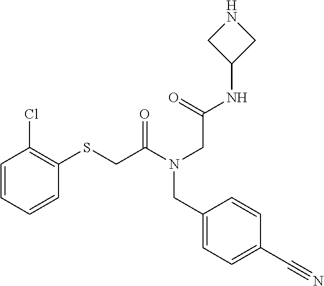 [SMILES: N#Cc1ccc(CN(CC(=O)NC2CNC2)C(=O)CSc2ccccc2Cl)cc1] | 429.2 [M + H]+ | (400 MHz, Methanol-d4) δ 7.76-7.62 (m, 2H), 7.53- 7.16 (m, 6H), 4.92-4.89 (m, 1H), 4.66-4.58 (m, 2H), 4.22-3.88 (m, 6H), 3.87-3.70 (m, 2H). |
| 6 | 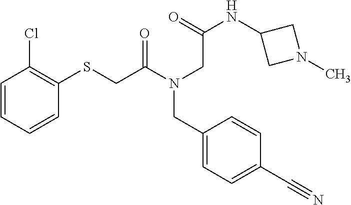 [SMILES: CN1CC(NC(=O)CN(Cc2ccc(C#N)cc2)C(=O)CSc2ccccc2Cl)C1] | 443.1 [M + H]+ | (500 MHz, Methanol-d4) δ 7.75-7.62 (m, 2H), 7.51- 7.36 (m, 4H), 7.31-7.16 (m, 2H), 4.89 (s, 1H), 4.64 (s, 1H), 4.41-4.30 (m, 1H), 4.20-3.93 (m, 4H), 3.67-3.60 (m, 2H), 2.99- 2.91 (m, 2H), 2.34 (s, 3H). |

### Example 2—Synthesis of N-[(5-Amino-1,3,4-oxadiazol-2-yl)methyl]-N-[(6-cyano-3-pyridyl)methyl]-2-(2-ethylphenyl)sulfanyl-acetamide (Compound No. 7)

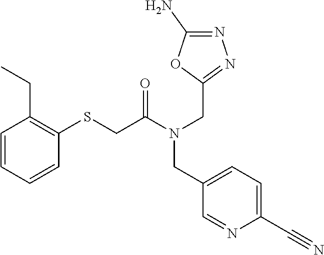 [SMILES: CCc1ccccc1SCC(=O)N(Cc1ccc(C#N)nc1)Cc1nnc(N)o1]

### N-[(6-Cyano-3-pyridyl)methyl]-2-(2-ethylphenyl)sulfanyl-N-(2-hydrazino-2-oxo-ethyl)acetamide

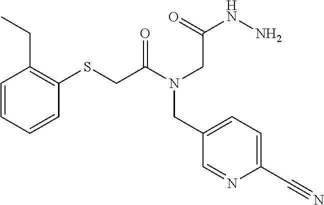 [SMILES: CCc1ccccc1SCC(=O)N(CC(=O)NN)Cc1ccc(C#N)nc1]

The corresponding acid, 2-[(6-cyano-3-pyridyl)methyl-[2-(2-ethylphenyl) sulfanylacetyl]amino]acetic acid, was synthesised by analogous procedures as in Example 1.

To a solution of 2-[(6-cyano-3-pyridyl)methyl-[2-(2-ethylphenyl)sulfanylacetyl]amino]acetic acid (160 mg, 0.433 mmol) and N-ethyl-N-isopropyl-propan-2-amine (0.15 mL, 0.866 mmol) in DMF (2.5432 mL) was added tert-butyl N-aminocarbamate (86 mg, 0.650 mmol) and 1-[bis(dimethylamino)methylidene]-1H-[1,2,3]triazolo[4,5-b]pyridin-1-ium 3-oxide hexafluorophosphate (198 mg, 0.520 mmol) and the reaction stirred for 18 hours. The reaction was purified by Normal phase column chromatography Method 1 and the desired fractions were concentrated in vacuo to afford the BOC protected intermediate. This intermediate was dissolved in DCM (2 mL) and 2,2,2-trifluoroacetic acid (0.32 mL, 4.33 mmol) was added and the reaction stirred at RT for 24 hours. The reaction was concentrated in vacuo and the crude residue was dissolved in MeOH (10 mL) and purified by SCX-2 cartridge (5 g), eluting with MeOH (10 mL) and then 2M NH₃ in MeOH (15 mL). The ammonia solution was concentrated in vacuo to afford the title compound (100 mg, 0.203 mmol, 47% yield) as a yellow oil. 1H NMR (500 MHz, DMSO-d6) δ 9.29-9.05 (m, 1H), 8.71-8.60 (m, 1H), 8.03-7.96 (m, 1H), 7.84-7.80 (m, 1H), 7.36-7.11 (m, 4H), 6.95-6.72 (m, 2H), 4.88-4.56 (m, 2H), 4.11-3.85 (m, 4H), 2.71-2.59 (m, 2H), 1.21-1.06 (m, 3H). (ESI⁺)—LCMS Method B, rt=1.00 mins, [M+H]⁺=383.95.

### N-[(5-Amino-1,3,4-oxadiazol-2-yl)methyl]-N-[(6-cyano-3-pyridyl)methyl]-2-(2-ethylphenyl)sulfanyl-acetamide (Compound No. 7)

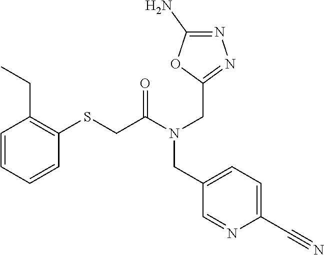 [SMILES: CCc1ccccc1SCC(=O)N(Cc1ccc(C#N)nc1)Cc1nnc(N)o1]

To a solution of N-[(6-cyano-3-pyridyl)methyl]-2-(2-ethylphenyl)sulfanyl-N-(2-hydrazino-2-oxo-ethyl)acetamide (78%, 50 mg, 0.102 mmol) and sodium hydrogenocarbonate (8.5 mg, 0.102 mmol) in anhydrous 1,4-Dioxane (1.8924 mL) was added carbononitridic bromide (12 mg, 0.112 mmol) and the reaction stirred at room temperature for 15 hours. The reaction was concentrated in vacuo to remove the solvent and was diluted with water (5 mL) and extracted with EtOAc (2×10 mL). The organic fractions were combined and concentrated in vacuo and then purified by High pH preparative HPLC (Early Elute method) and the desired fractions were freeze-dried to afford the title compound (12 mg, 0.0288 mmol, 28% yield) as a white solid. 1H NMR (500 MHz, Methanol-d4) δ 8.63-8.52 (m, 1H), 7.85-7.68 (m, 2H), 7.44-7.10 (m, 4H), 4.82-4.78 (m, 2H), 4.73-4.60 (m, 2H), 4.06-3.85 (m, 2H), 2.83-2.71 (m, 2H), 1.22-1.14 (m, 3H). (ESI⁺)—LCMS Method C, rt=4.09 mins, [M+H]⁺=364.0.

### Example 3—Synthesis of 2-(2-Chlorophenyl)sulfanyl-N-[(6-cyano-3-pyridyl)methyl]-N-[(1S)-1-(5-amino-1,3,4-oxadiazol-2-yl)-2-hydroxy-ethyl]acetamide (Compound No. 8)

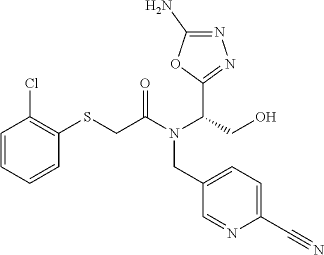 [SMILES: N#Cc1ccc(CN(C(=O)CSc2ccccc2Cl)[C@@H](CO)c2nnc(N)o2)cn1]

2-(2-Chlorophenyl)sulfanyl-N-[(6-cyano-3-pyridyl)methyl]-N-[(1S)-1-(5-amino-1,3,4-oxadiazol-2-yl)-2-tert-butoxy-ethyl]acetamide was synthesised by analogous procedures as in Example 1.

To a solution of 2-(2-chlorophenyl)sulfanyl-N-[(6-cyano-3-pyridyl)methyl]-N-[(1S)-1-(5-amino-1,3,4-oxadiazol-2-yl)-2-tert-butoxy-ethyl]acetamide (97%, 20 mg, 0.0387 mmol) in DCM (0.5 mL) was added 2,2,2-trifluoroacetic acid (0.030 mL, 0.387 mmol) and the reaction stirred at RT for 6 hours. The reaction was concentrated in vacuo and was purified by High pH preparative HPLC (Early Elute method) to afford the title compound (7.7 mg, 0.0173 mmol, 45% Yield) as a white solid. 1H NMR (500 MHz, DMSO-d6) δ 8.67-8.45 (m, 1H), 7.99-7.69 (m, 2H), 7.48-7.16 (m, 4H), 7.03-6.96 (m, 2H), 5.64-5.54 (m, 1H), 5.50-5.18 (m, 1H), 5.00-4.30 (m, 3H), 4.11-3.92 (m, 3H). (ESI⁺)—LCMS Method A, rt=2.29 mins, [M+H]⁺=445.2.

The compounds in Table 3 below were prepared based on experimental procedures described herein above and in Example 2.

TABLE 3

| | | Mass | |
|---|---|---|---|
| Compound | | Spec. | |
| No. | Chemical Structure | (ES, m/z) | ¹H NMR Spectrum |
| 9 | 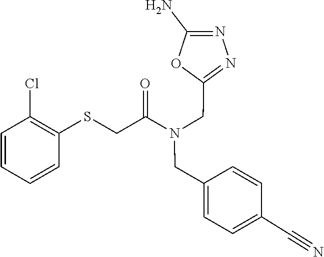 [SMILES: N#Cc1ccc(CN(Cc2nnc(N)o2)C(=O)CSc2ccccc2Cl)cc1] | 414.1 [M + H]+ | (400 MHz, Methanol-d4) δ 7.75-7.60 (m, 2H), 7.52- 7.17 (m, 6H), 4.94-4.58 (m, 4H), 4.21-3.95 (m, 2H). |
| 10 | 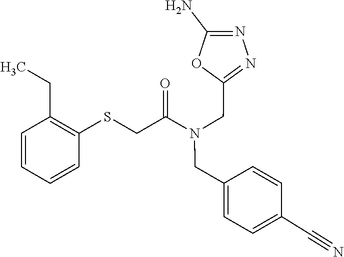 [SMILES: CCc1ccccc1SCC(=O)N(Cc1ccc(C#N)cc1)Cc1nnc(N)o1] | 408.0 [M + H]+ | (400 MHz, Chloroform-d) δ 7.40-7.34 (m, 1H), 7.23- 7.12 (m, 3H), 3.55 (s, 2H), 2.81 (q, J = 7.5 Hz, 2H), 1.39 (s, 9H), 1.24 (t, J = 7.5 Hz, 3H). |
| 11 | 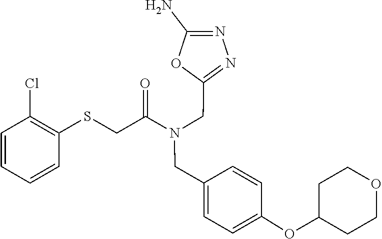 [SMILES: Nc1nnc(CN(Cc2ccc(OC3CCOCC3)cc2)C(=O)CSc2ccccc2Cl)o1] | 489.1 [M + H]+ | (500 MHz, DMSO-d6) δ 7.48-6.87 (m, 10H), 4.72- 4.43 (m, 5H), 4.28-4.14 (m, 2H), 3.88-3.78 (m, 2H), 3.51-3.42 (m, 2H), 1.98- 1.90 (m, 2H), 1.60-1.51 (m, 2H). |
| 12 | 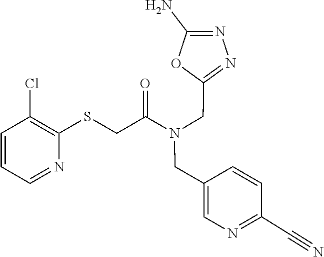 [SMILES: N#Cc1ccc(CN(Cc2nnc(N)o2)C(=O)CSc2ncccc2Cl)cn1] | 416.1 [M + H]+ | (500 MHz, Methanol-d4) δ 8.84-8.58 (m, 1H), 8.36- 8.26 (m, 1H), 8.10-7.65 (m, 3H), 7.15-7.09 (m, 1H), 5.15-4.95 (m, 2H), 4.83- 4.65 (m, 2H), 4.37-4.21 (m, 2H). |
| 13 | 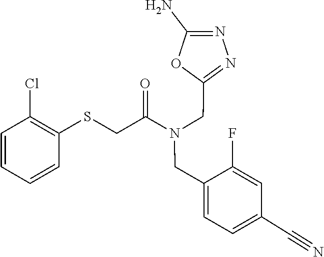 [SMILES: N#Cc1ccc(CN(Cc2nnc(N)o2)C(=O)CSc2ccccc2Cl)c(F)c1] | 432.0 [M + H]+ | (500 MHz, Methanol-d4) δ 7.61-7.18 (m, 7H), 4.96- 4.86 (m, 2H), 4.76-4.60 (m, 2H), 4.18-4.02 (m, 2H). |
| 14 | 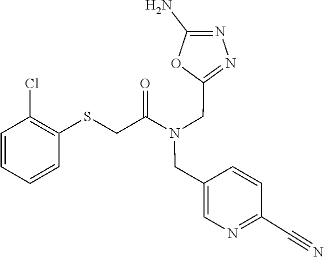 [SMILES: N#Cc1ccc(CN(Cc2nnc(N)o2)C(=O)CSc2ccccc2Cl)cn1] | 414.9 [M + H]+ | (500 MHz, Methanol-d4) δ 8.66-8.53 (m, 1H), 7.91- 7.72 (m, 2H), 7.49-7.38 (m, 2H), 7.29-7.19 (m, 2H), 4.99-4.88 (m, 2H), 4.74- 4.62 (m, 2H), 4.17-4.02 (m, 2H). |
| 15 | 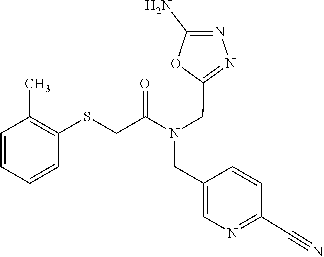 [SMILES: Cc1ccccc1SCC(=O)N(Cc1ccc(C#N)nc1)Cc1nnc(N)o1] | 395.0 [M + H]+ | (400 MHz, Methanol-d4) δ 8.63-8.51 (m, 1H), 7.84- 7.68 (m, 2H), 7.40-7.09 (m, 4H), 4.83-4.56 (m, 4H), 4.04-3.89 (m, 2H), 2.42- 2.32 (m, 3H). |
| 16 | 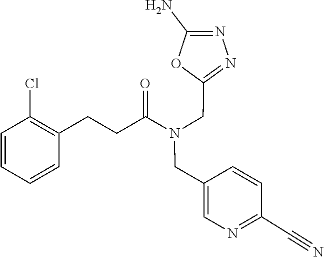 [SMILES: N#Cc1ccc(CN(Cc2nnc(N)o2)C(=O)CCc2ccccc2Cl)cn1] | 397.0 [M + H]+ | (400 MHz, DMSO-d6) δ 8.60 (d, J = 1.7 Hz, 1H), 8.01- 7.79 (m, 2H), 7.43-7.20 (m, 4H), 7.05-6.96 (m, 2H), 4.83-4.59 (m, 4H), 3.00- 2.90 (m, 2H), 2.86-2.65 (m, 2H) |
| 17 | 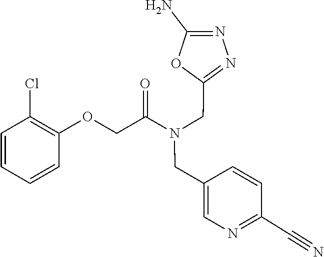 [SMILES: N#Cc1ccc(CN(Cc2nnc(N)o2)C(=O)COc2ccccc2Cl)cn1] | 399.2 [M + H]+ | (500 MHz, DMSO-d6) δ 8.72-8.61 (m, 1H), 8.04- 7.85 (m, 2H), 7.47-6.89 (m, 6H), 5.21-5.03 (m, 2H), 4.89-4.51 (m, 4H). |

### Example 4—Synthesis of N-((5-amino-1,3,4-oxadiazol-2-yl)methyl)-2-(2-chloro-3,5-difluorophenoxy)-N-((6-cyanopyridin-3-yl)methyl)acetamide (Compound No. 18)

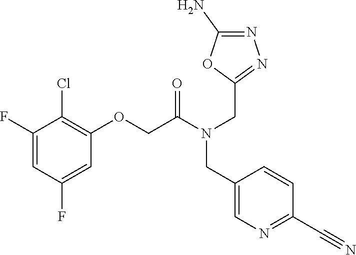 [SMILES: N#Cc1ccc(CN(Cc2nnc(N)o2)C(=O)COc2cc(F)cc(F)c2Cl)cn1]

### Methyl 2-(2-chloro-3,5-difluoro-phenoxy)acetate

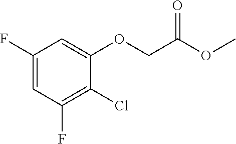

Methyl bromoacetate (1.3 mL, 9.12 mmol), 2-chloro-3,5-difluoro-phenol (1.00 g, 6.08 mmol), potassium carbonate (1.68 g, 12.2 mmol) and DMF (20.258 mL) were heated to 80° C. and stirred for 18 hours. The reaction was then cooled, dissolved in water and the product extracted with EtOAc (100 mL) and the organic layer washed with brine (3×100 mL), then dried with MgSO₄ and filtered. The solvent was then evaporated to give an oily yellow residue. The crude product was purified by Normal phase column chromatography Method 1. The desired fractions were concentrated in vacuo to give methyl 2-(2-chloro-3,5-difluoro-phenoxy)acetate (100.0%) (1.28 g, 5.41 mmol, 89% Yield). 1HNMR (500 MHz, Chloroform-d) δ 6.62 (td, J=8.7, 2.7 Hz, 1H), 6.40 (dt, J=9.9, 2.3 Hz, 1H), 4.71 (s, 2H), 3.83 (s, 3H). LCMS—LCMS Method A rt.=3.15 min (ESI+) 254.1, 256.1.

### 2-(2-Chloro-3,5-difluoro-phenoxy)acetic acid

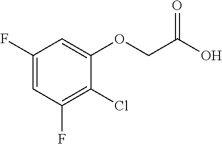 [SMILES: O=C(O)COc1cc(F)cc(F)c1Cl]

A solution of methyl 2-(2-chloro-3,5-difluoro-phenoxy)acetate (557 mg, 2.36 mmol) and hydroxylithium hydrate (109 mg, 2.59 mmol) in Water (2.4 mL)/Methanol (2.4 mL)/THF (2.4 mL) was stirred at RT over 2 hours. The solvent was then evaporated, the solid was then dissolved in water (50 mL). The basic aqueous phase was extracted with diethyl ether and the organic layer found to contain only impurities. The pH was then adjusted from pH 10 to 1 by adding HCl 6M solution and the product was extracted with EtOAc (3×50 mL), then dried with MgSO₄, filtered and concentrated to obtain 2-(2-chloro-3,5-difluoro-phenoxy)acetic acid (100.0%) (428 mg, 1.92 mmol, 82% Yield) as a white solid. ¹H NMR (400 MHz, Chloroform-d) δ 6.65 (td, J=8.6, 2.7 Hz, 1H), 6.45 (dt, J=9.7, 2.3 Hz, 1H), 4.76 (s, 2H). (ESI⁻)—LCMS Method B rt.=0.⁷² min (M−H)⁻ 221.0, 223.0.

### Methyl ((6-cyanopyridin-3-yl)methyl)glycinate

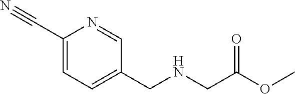

To a solution of methyl glycinate hydrochloride (1:1) (3.80 g, 30.3 mmol) in Methanol (120 mL), 4 g of MP-carbonate was added and the reaction stirred for 3 hours to free base the amine. The resultant solution was filtered and to the filtrate was added 5-formylpyridine-2-carbonitrile (1.9 mL, 15.1 mmol) and 10 drops of acetic acid. The reaction was stirred for 15 minutes before sodium triacetoxyborohydride (7.06 g, 33.3 mmol) was added. After stirring for 6 h the product could be observed and after 16 hours an extra equivalent of sodium triacetoxyborohydride (3.21 g, 15.1 mmol) was added and the reaction was stirred for 3 hours. The reaction was directly purified by SCX column (50 g) then eluted with 2M NH₃/MeOH solution. The reaction was concentrated to dryness and then purified by High pH preparative HPLC. The desired fractions were concentrated to dryness in vacuo to afford methyl ((6-cyanopyridin-3-yl)methyl)glycinate (1.27 g, 6.19 mmol, 41% yield) as a purple liquid. ¹H NMR (400 MHz, DMSO-d₆) δ 8.70 (s, 1H), 7.98 (d, J=1.1 Hz, 2H), 3.85 (s, 2H), 3.62 (s, 3H), 3.36 (s, 2H), 3.17 (s, 1H). (ESI⁺)—LCMS Method D rt=2.50 min (M+H)⁺ 206.5

### Methyl 2-[[2-(2-chloro-3,5-difluoro-phenoxy)acetyl]-[(6-cyano-3-pyridyl)methyl]amino]acetate

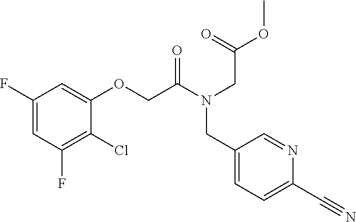 [SMILES: COC(=O)CN(Cc1ccc(C#N)nc1)C(=O)COc1cc(F)cc(F)c1Cl]

A stirred solution of 2-(2-chloro-3,5-difluoro-phenoxy)acetic acid (80 mg, 0.359 mmol), 1-[bis(dimethylamino)methylidene]-1H-[1,2,3]triazolo[4,5-b]pyridin-1-ium 3-oxide hexafluorophosphate (164 mg, 0.431 mmol) DMF-Anhydrous (1.8 mL), N-ethyl-N-isopropyl-propan-2-amine (0.13 mL, 0.719 mmol) and methyl 2-[(6-cyano-3-pyridyl)methylamino]acetate (74 mg, 0.359 mmol) was prepared without stepwise or sequential addition. The reaction was then stirred 16 hours before assessing reaction progress by LCMS. The reaction was diluted in 50 mL EtOAc and 50 mL water, separated and the organics washed with 3×50 ml saturated brine solution. The organics were then separated and dried using MgSO₄ before concentration to dryness to provide methyl 2-[[2-(2-chloro-3,5-difluoro-phenoxy)acetyl]-[(6-cyano-3-pyridyl)methyl]amino]acetate (95.0%) (140 mg, 0.325 mmol, 90% Yield) as a purple liquid. (ESI⁺)—LCMS Method A rt=3.15 min (M+H)⁺ 410.1 and 412.1

### N-((5-amino-1,3,4-oxadiazol-2-yl)methyl)-2-(2-chloro-3,5-difluorophenoxy)-N-((6-cyanopyridin-3-yl)methyl)acetamide (Compound No. 18)

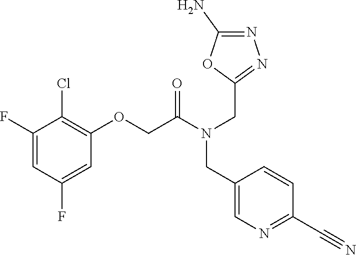 [SMILES: N#Cc1ccc(CN(Cc2nnc(N)o2)C(=O)COc2cc(F)cc(F)c2Cl)cn1]

Methyl 2-[[2-(2-chloro-3,5-difluoro-phenoxy)acetyl]-[(6-cyano-3-pyridyl)methyl]amino]acetate (140 mg, 0.342 mmol) was dissolved in ethanol (3.4 mL) then hydrazine hydrate (98%, 0.086 mL, 1.71 mmol) was added. After stirring for 4 hours at room temperature the resultant suspension was filter, and the resultant filter cake was washed with more EtOH (5 mL).

The resulting solid was then transferred and the remaining volatiles removed under vacuum, providing an off-white solid that was then dissolved in 1,4-Dioxane (3.9 mL) to which was added sodium hydrogen carbonate (144 mg, 1.71 mmol) then cyanic bromide (40 mg, 0.376 mmol). The reaction mixture was stirred at room temperature under nitrogen for 16 hours then filtered through cotton wool then the filter cake was washed with MeOH. The filtered and quenched reaction mixture was then concentrated and purified directly by High pH preparative HPLC. The desired fractions were then concentrated to provide 2-(2-chloro-3,5-difluoro-phenoxy)-N-[(6-cyano-3-pyridyl)methyl]-N-(2-hydrazino-2-oxo-ethyl)acetamide (60.0%) (80 mg, 0.117 mmol, 34% Yield) as an off-white solid. ¹H-NMR (400 MHz, DMSO-d6) δ 8.77-8.57 (m, 1H), 8.09-7.83 (m, 2H), 7.20-6.86 (m, 4H), 5.30-4.51 (m, 6H). (ESI⁺)—LCMS Method A rt=2.53 min (M+H)⁺ 435.1 and 437.1.

The compounds in Table 4 below were prepared based on experimental procedures described herein above.

TABLE 4

| | | Mass | |
|---|---|---|---|
| Compound | | Spec. | |
| No. | Chemical Structure | (ES, m/z) | ¹H NMR Spectrum |
| 19 | 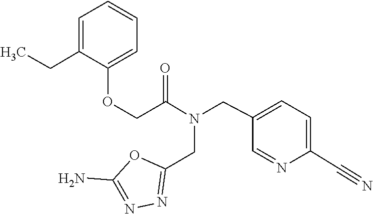 [SMILES: CCc1ccccc1OCC(=O)N(Cc1ccc(C#N)nc1)Cc1nnc(N)o1] | 393.3 [M + H]+ | (400 MHz, DMSO-d6) δ 8.60 (s, 1H), 7.90-7.72 (m, 2H), 7.25-7.03 (m, 2H), 6.98- 6.77 (m, 2H), 5.10-4.51 (m, 6H), 2.75-2.36 (m, 2H), 1.25-0.98 (m, 3H). |
| 20 | 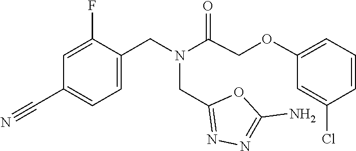 [SMILES: N#Cc1ccc(CN(Cc2nnc(N)o2)C(=O)COc2cccc(Cl)c2)c(F)c1] | 416.2 [M + H]⁺ | (400 MHz, Methanol-d4) δ 7.63-7.37 (m, 3H), 7.30- 7.18 (m, 1H), 7.04-6.77 (m, 5H), 5.09-4.95 (m, 2H), 4.91-4.78 (m, 2H), 4.78- 4.66 (m, 2H). |

### Example 5—Synthesis of N-[(5-Amino-1,3,4-oxadiazol-2-yl)methyl]-2-(2-chloro-3,5-difluoro-phenyl)sulfanyl-N-[(6-cyano-3-pyridyl)methyl]acetamide (Compound No. 21)

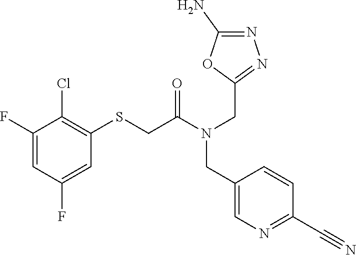 [SMILES: N#Cc1ccc(CN(Cc2nnc(N)o2)C(=O)CSc2cc(F)cc(F)c2Cl)cn1]

### Methyl 2-(2-chloro-3,5-difluoro-phenyl)sulfanylacetate

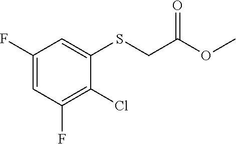

To a pressure vial was added 1-bromo-2-chloro-3,5-difluoro-benzene (1.00 g, 4.40 mmol), methyl sulfanylacetate (0.48 mL, 5.28 mmol), disodium carbonate (1.17 g, 11.0 mmol) and 1,4-dioxane (13.3 mL). The reaction vial was purged with nitrogen for 5 minutes and then XantPhos Pd G3 (417 mg, 0.440 mmol) was added and the reaction was purged with nitrogen for 1 further minute. The reaction was placed in a pre-heated hot plate at 100° C. and stirred for 16 h before LCMS showed consumption of SM. The crude reaction mixture was concentrated directly onto silica to dryness. The crude was then purified by Normal phase column chromatography Method 1. The desired fractions were concentrated to dryness in vacuo to provide methyl 2-(2-chloro-3,5-difluoro-phenyl)sulfanylacetate (100.0%) (1.04 g, 4.12 mmol, 94% Yield) as a yellow solid. ¹H NMR (400 MHz, DMSO-d₆) δ 7.35 (ddd, J=9.1, 2.7 Hz, 1H), 7.15 (ddd, J=9.5, 2.7, 1.7 Hz, 1H), 4.18 (s, 2H), 3.68 (s, 3H). (ESI⁺)—LCMS Method A rt=3.45 min (M+H)⁺ 253.0.

### 2-(2-Chloro-3,5-difluoro-phenyl)sulfanylacetic acid

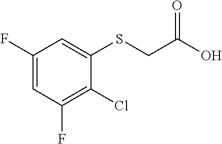 [SMILES: O=C(O)CSc1cc(F)cc(F)c1Cl]

2-(2-Chloro-3,5-difluoro-phenyl)sulfanylacetic acid was prepared using an experimental procedure similar to that for 2-(2-chloro-3,5-difluoro-phenoxy)acetic acid in Example 4, above. ¹H-NMR (500 MHz, DMSO-d₆) δ 13.09 (s, 1H), 7.42-7.27 (m, 1H), 7.20-7.09 (m, 1H), 4.07 (s, 2H). (ESI⁺)—LCMS Method A: rt=2.86 min (M+H)⁺ 236.9.

### Methyl 2-[[2-(2-chloro-3,5-difluoro-phenyl)sulfanylacetyl]-[(6-cyano-3-pyridyl)methyl]amino]acetate

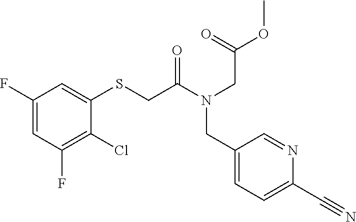 [SMILES: COC(=O)CN(Cc1ccc(C#N)nc1)C(=O)CSc1cc(F)cc(F)c1Cl]

Methyl 2-[[2-(2-chloro-3,5-difluoro-phenyl)sulfanylacetyl]-[(6-cyano-3-pyridyl)methyl]amino]acetate was prepared using an experimental procedure similar to that for methyl 2-[[2-(2-chloro-3,5-difluoro-phenoxy)acetyl]-[(6-cyano-3-pyridyl)methyl]amino]acetate in Example 4, above. ¹H-NMR (500 MHz, Chloroform-d) δ 8.68-8.53 (m, 1H), 7.85-7.61 (m, 2H), 7.13-6.99 (m, 1H), 6.84-6.74 (m, 1H), 4.92-4.62 (m, 2H), 4.19-4.01 (m, 2H), 3.89-3.65 (m, 4H), 2.80 (s, 3H). (ESI⁺)—LCMS Method A rt.=3.33 min (M+H)⁺ 426.1 and 428.1

### N-[(5-Amino-1,3,4-oxadiazol-2-yl)methyl]-2-(2-chloro-3,5-difluoro-phenyl)sulfanyl-N-[(6-cyano-3-pyridyl)methyl]acetamide (Compound No. 21)

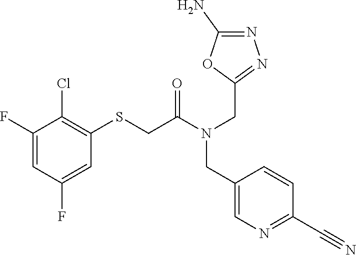 [SMILES: N#Cc1ccc(CN(Cc2nnc(N)o2)C(=O)CSc2cc(F)cc(F)c2Cl)cn1]

N-[(5-amino-1,3,4-oxadiazol-2-yl)methyl]-2-(2-chloro-3,5-difluoro-phenyl)sulfanyl-N-[(6-cyano-3-pyridyl)methyl]acetamide was prepared using an experimental procedure similar to that for N-((5-amino-1,3,4-oxadiazol-2-yl)methyl)-2-(2-chloro-3,5-difluorophenoxy)-N-((6-cyanopyridin-3-yl)methyl)acetamide in Example 4, above. ¹H-NMR (400 MHz, DMSO-d6) δ 8.75-8.57 (m, 1H), 8.07-7.81 (m, 2H), 7.36-7.16 (m, 2H), 7.09-6.91 (m, 2H), 5.00-4.82 (m, 2H), 4.74-74.51 (m, 2H), 4.48-0.4.33 (m, 2H). (ESI⁺)—LCMS Method A rt=2.70 min (M+H)⁺ 451.1 and 453.1.

The compounds in Table 5 below were prepared based on experimental procedures described herein above.

TABLE 5

| | | Mass | |
|---|---|---|---|
| Compound | | Spec. | |
| No. | Chemical Structure | (ES, m/z) | ¹H NMR Spectrum |
| 22 | 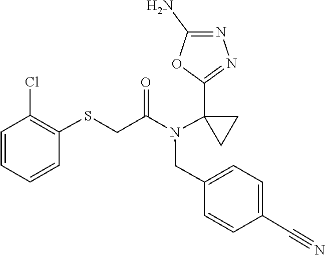 [SMILES: N#Cc1ccc(CN(C(=O)CSc2ccccc2Cl)C2(c3nnc(N)o3)CC2)cc1] | 440.1 [M + H]+ | (400 MHz, DMSO-d6 ) δ 7.84-7.71 (m, 2H), 7.53- 7.41 (m, 1H), 7.40-7.33 (m, 3H), 7.30 (td, J = 7.6, 1.3 Hz, 1H), 7.18 (td, J = 7.9, 1.6 Hz, 1H), 7.05-6.83 (m, 2H), 5.04-4.87 (m, 1H), 4.62- 4.41 (m, 2H), 4.28-4.15 (m, 1H), 2.07 (s, 1H), 1.81- 1.66 (m, 1H), 1.44-1.34 (m, 1H), 1.30-1.14 (m, 1H). |
| 23 | 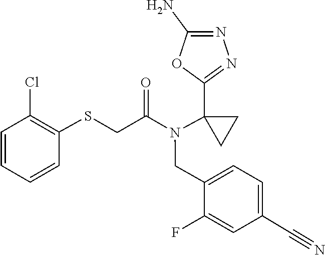 [SMILES: N#Cc1ccc(CN(C(=O)CSc2ccccc2Cl)C2(c3nnc(N)o3)CC2)c(F)c1] | 458.1 [M + H]+ | (400 MHz, DMSO-d6) δ 7.79 (dd, J = 10.0, 1.5 Hz, 1H), 7.63 (dd, J = 8.0, 1.5 Hz, 1H), 7.48-7.26 (m, 4H), 7.23-7.14 (m, 1H), 7.01 (s, 2H), 4.82 (d, J = 16.4 Hz, 1H), 4.64 (d, J = 16.5 Hz, 1H), 4.46 (d, J = 16.1 Hz, 1H), 4.21 (d, J = 16.2 Hz, 1H), 2.17-2.08 (m, 1H), 1.81-1.70 (m, 1H), 1.47-1.29 (m, 2H). |
| 24 | 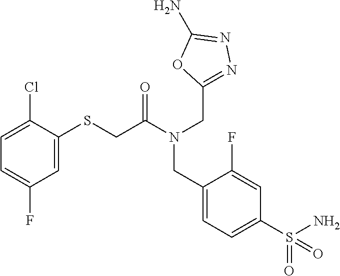 [SMILES: Nc1nnc(CN(Cc2ccc(S(N)(=O)=O)cc2F)C(=O)CSc2cc(F)ccc2Cl)o1] | 504.1 [M + H]+ | (500 MHz, DMSO-d6) δ 7.68-7.54 (m, 2H), 7.54- 7.40 (m, 4H), 7.37-7.30 (m, 1H), 7.13-6.96 (m, 3H), 4.98-4.82 (m, 2H), 4.67- 4.50 (m, 2H), 4.43-4.25 (m, 2H). |
| 25 | 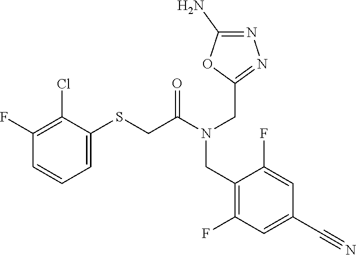 [SMILES: N#Cc1cc(F)c(CN(Cc2nnc(N)o2)C(=O)CSc2cccc(F)c2Cl)c(F)c1] | 468.1 [M + H]+ | (500 MHz, DMSO-d6) δ 7.92-7.65 (m, 2H), 7.46- 7.09 (m, 3H), 7.11-6.87 (m, 2H), 4.99-4.22 (m, 6H). |
| 26 | 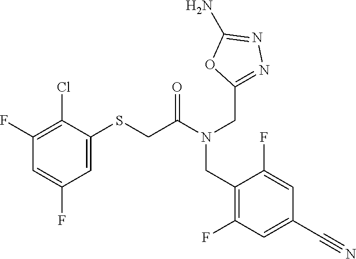 [SMILES: N#Cc1cc(F)c(CN(Cc2nnc(N)o2)C(=O)CSc2cc(F)cc(F)c2Cl)c(F)c1] | 486.2 [M + H]+ | (400 MHz, DMSO-d6) δ 7.66 (m, 2H), 7.36-7.25 (m, 1H), 7.19-6.87 (m, 3H), 5.08-4.74 (m, 2H), 4.71- 4.20 (m, 4H). |
| 27 | 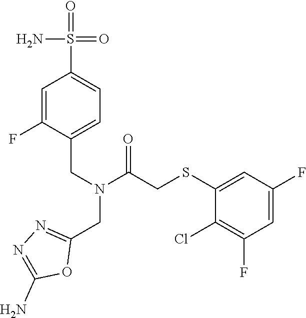 [SMILES: Nc1nnc(CN(Cc2ccc(S(N)(=O)=O)cc2F)C(=O)CSc2cc(F)cc(F)c2Cl)o1] | 522.0 [M + H]+ | (400 MHz, DMSO-d6) δ 7.38 (m, 5H), 7.36-7.18 (m, 2H), 7.14-6.92 (m, 2H), 4.99-4.79 (m, 2H), 4.67- 4.48 (m, 2H), 4.48-4.29 (m, 2H) |

### Example 6—Synthesis of N-1[(5-Amino-1,3,4-oxadiazol-2-yl)methyl]-2-(2-chloro-3-fluoro-phenyl)sulfanyl-N-[(4-cyano-2-fluoro-phenyl)methyl]acetamide (Compound No. 28)

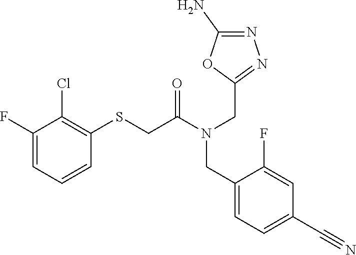 [SMILES: N#Cc1ccc(CN(Cc2nnc(N)o2)C(=O)CSc2cccc(F)c2Cl)c(F)c1]

### Methyl 2-(2-chloro-3-fluoro-phenyl)sulfanylacetate

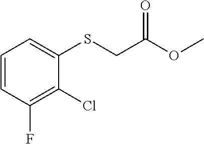

Methyl 2-(2-chloro-3-fluoro-phenyl)sulfanylacetate was prepared based on experimental procedure for methyl 2-(2-chloro-3,5-difluoro-phenyl)sulfanylacetate. LCMS Method B rt=1.22 min.

### 2-[[2-(2-Chloro-3-fluoro-phenyl)sulfanylacetyl]-[(4-cyano-2-fluoro-phenyl)methyl]amino]acetic acid

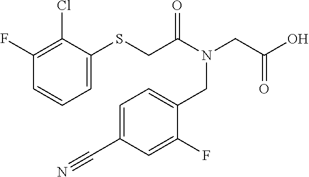 [SMILES: N#Cc1ccc(CN(CC(=O)O)C(=O)CSc2cccc(F)c2Cl)c(F)c1]

A solution of methyl 2-(2-chloro-3-fluoro-phenyl)sulfanylacetate (224 mg, 0.954 mmol) and hydroxylithium (114 mg, 4.77 mmol) in Water (0.96 mL)/Methanol (0.96 mL)/THF (0.96 mL) was stirred at RT over the weekend. The solvent was then evaporated and the resulting orange solid was macerated in water and filtered. The solid was then dissolved in basic water (pH=10) and the water phase was washed with Et₂O (3×5 mL). The pH was then adjusted to 2 adding HCl 1M solution and the product was extracted with Et₂O (3×5 mL) to obtain a pale orange solid.

The resulting pale orange solid was added to a solution of tert-butyl 2-([(4-cyano-2-fluoro-phenyl)methyl]amino)acetate (77 mg, 0.290 mmol), 2-(2-chloro-3-fluoro-phenyl)sulfanylacetic acid (64 mg, 0.290 mmol) and N-ethyl-N-isopropyl-propan-2-amine (0.10 mL, 0.580 mmol) in DMF-Anhydrous (3 mL) and the resulting mixture was stirred at RT for 2 h. The solvent was evaporated and the solid residue was dissolved in MeOH and passed through a 2 g SCX-2 column. The product was eluted with MeOH.

After solvent evaporation, the resulting material was suspended in DCM, the white precipitate was filtered off and trifluoroacetic acid (0.22 mL, 2.90 mmol) was added to the filtrate. The resulting solution was stirred for 18 h. After solvent evaporation, the solid was dissolved in basic water (pH=10) and the water phase was washed with Et₂O (3×5 mL). The pH was then adjusted to 2 adding HCl 1M solution and the product was extracted with Et₂O (3×5 mL) to obtain 2-[[2-(2-chloro-3-fluoro-phenyl)sulfanylacetyl]-[(4-cyano-2-fluoro-phenyl)methyl]amino]acetic acid (91 mg, 0.106 mmol, 37% Yield) as a pale orange solid. (ESI⁺)—LCMS Method B rt.=1.16 min (M+H)⁺ 411.1 and 413.1

### N-[(5-Amino-1,3,4-oxadiazol-2-yl)methyl]-2-(2-chloro-3-fluoro-phenyl)sulfanyl-N-[(4-cyano-2-fluoro-phenyl)methyl]acetamide (Compound No. 28)

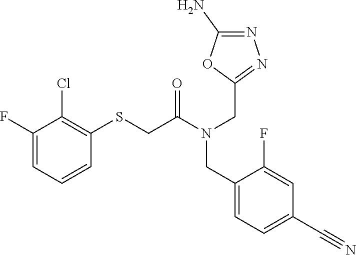 [SMILES: N#Cc1ccc(CN(Cc2nnc(N)o2)C(=O)CSc2cccc(F)c2Cl)c(F)c1]

1-[bis(dimethylamino)methylidene]-1H-[1,2,3]triazolo[4,5-b]pyridin-1-ium 3-oxide hexafluorophosphate (4.0 mL, 0.155 mmol) was added to a solution of 2-[[2-(2-chloro-3-fluoro-phenyl)sulfanylacetyl]-[(4-cyano-2-fluoro-phenyl)methyl]amino]acetic acid (50%, 91 mg, 0.111 mmol), tert-butyl N-aminocarbamate (20 mg, 0.155 mmol) and N-ethyl-N-isopropyl-propan-2-amine (0.039 mL, 0.222 mmol) in DMF-Anhydrous (2.4 mL) and the resulting mixture was stirred at RT for 20 h. The solvent was then evaporated and the crude was purified by Normal phase column chromatography Method 1.

After solvent evaporation, the BOC-intermediate was dissolved in a solution of HCl in dioxane (5 mL) and the resulting solution was stirred at RT for 24 h. The solvent was then evaporated and the oily residue was dissolved in MeOH and passed through a 2 g SCX-2 column. After rinsing with MeOH, the product was eluted with NH₃ 7 M methanolic solution to obtain 31 mg of the de-Boc intermediate, which was dissolved in 1,4-Dioxane-Anhydrous (2.4 mL) followed by the addition of sodium hydrogencarbonate (7.4 mg, 0.0886 mmol) and 1 M carbononitridic bromide (89 uL, 0.0886 mmol). The resulting suspension was then stirred for 18 h. The solid was filtered and the filtrate was purified Low pH preparative HPLC (Early elute method) to obtain N-[(5-amino-1,3,4-oxadiazol-2-yl)methyl]-2-(2-chloro-3-fluoro-phenyl)sulfanyl-N-[(4-cyano-2-fluoro-phenyl)methyl]acetamide (3.0 mg, 6.67 μmol, 6.0% Yield) as a white solid. ¹H-NMR (500 MHz, DMSO-d6) δ 7.94-7.77 (m, 1H), 7.73-7.62 (m, 1H), 7.50-7.30 (m, 2H), 7.27-7.18 (m, 2H), 7.03 (m, 2H), 4.90 (m, 2H), 4.60 (m, 2H), 4.32 (m, 2H). (ESI⁺) LCMS Method H rt.=3.80 min (M+H)⁺ 450.1 and 452.1.

The compounds in Table 6 below were prepared based on experimental procedures described herein above.

TABLE 6

| | | Mass | |
|---|---|---|---|
| Compound | | Spec. | |
| No. | Chemical Structure | (ES, m/z) | ¹H NMR Spectrum |
| 29 | 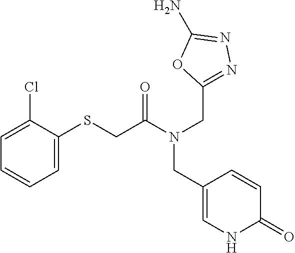 [SMILES: Nc1nnc(CN(Cc2ccc(=O)[nH]c2)C(=O)CSc2ccccc2Cl)o1] | 423.2 [M + NH4]+ | (400 MHz, DMSO-d6) δ 10.45 (s, 1H), 7.51-7.26 (m, 3H), 7.25-6.95 (m, 4H), 6.71-6.49 (m, 2H), 4.82-4.38 (m, 4H), 4.34- 4.22 (m, 2H). |
| 30 | 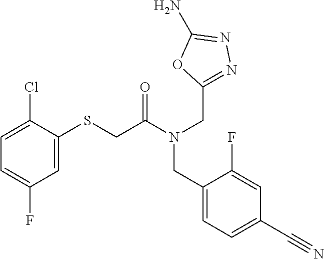 [SMILES: N#Cc1ccc(CN(Cc2nnc(N)o2)C(=O)CSc2cc(F)ccc2Cl)c(F)c1] | 450.1 [M + H]+ | (500 MHz, DMSO-d6) δ 7.92-7.75 (m, 1H), 7.72- 7.59 (m, 1H), 7.54-7.38 (m, 2H), 7.38-7.25 (m, 1H), 7.12-6.89 (m, 3H), 5.02-4.80 (m, 2H), 4.72- 4.48 (m, 2H), 4.45-4.22 (m, 2H). |

### Example 7—Synthesis of N-1[(5-Amino-1,3,4-oxadiazol-2-yl)methyl]-2-(2-chloro-3,5-difluoro-phenoxy)-N-1[(4-cyano-2-fluoro-phenyl)methyl]acetamide (Compound No. 31)

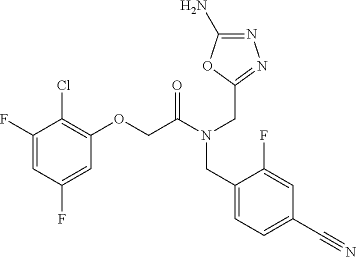 [SMILES: N#Cc1ccc(CN(Cc2nnc(N)o2)C(=O)COc2cc(F)cc(F)c2Cl)c(F)c1]

### 4-[([(5-Amino-1,3,4-oxadiazol-2-yl)methyl]amino)methyl]-3-fluoro-benzonitrile

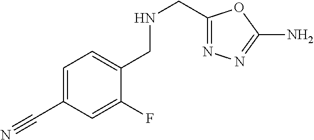 [SMILES: N#Cc1ccc(CNCc2nnc(N)o2)c(F)c1]

4-(Bromomethyl)-3-fluorobenzonitrile (0.60 g, 2.80 mmol) was added to a suspension of potassium; carbonate (3.10 g, 22.4 mmol), 5-(aminomethyl)-1,3,4-oxadiazol-2-amine dihydrochloride (1.05 g, 5.61 mmol) in Acetonitrile-Anhydrous (25 mL) and the resulting mixture was stirred at 80° C. for 24 h. The reaction mixture was cooled to room temperature, filtered through celite and concentrated to dryness. The crude material was then diluted in MeOH and purified by SCX-2 catch-release resin (5 g), washed with MeOH (3 CV) and eluting with 2M NH₃ in MeOH (3 CV). The eluent was collected and concentrated to dryness to provide 4-[([(5-amino-1,3,4-oxadiazol-2-yl)methyl]amino)methyl]-3-fluoro-benzonitrile (67.0%) (824 mg, 2.23 mmol, 80% Yield) as an orange. ¹H-NMR (500 MHz, Methanol-d4) δ 7.66 (t, J=7.6 Hz, 1H), 7.57-7.51 (m, 2H), 3.94 (s, 2H), 3.86 (s, 2H), 3.83 (s, 2H), 3.36 (s, 1H). (ESI⁺)—LCMS Method B rt=1.57 min (M+H)⁺ 248.2.

### N-[(5-Amino-1,3,4-oxadiazol-2-yl)methyl]-2-(2-chloro-3,5-difluoro-phenoxy)-N-[(4-cyano-2-fluoro-phenyl)methyl]acetamide (Compound No. 31)

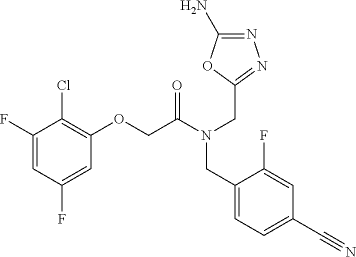 [SMILES: N#Cc1ccc(CN(Cc2nnc(N)o2)C(=O)COc2cc(F)cc(F)c2Cl)c(F)c1]

A solution 2-(2-chloro-3,5-difluoro-phenoxy)acetic acid (36 mg, 0.162 mmol), 1-[bis(dimethylamino)methylidene]-1H-[1,2,3]triazolo[4,5-b]pyridin-1-ium 3-oxide hexafluorophosphate (74 mg, 0.194 mmol), DMF Anhydrous (0.77 mL), N-ethyl-N-isopropyl-propan-2-amine (0.057 mL, 0.324 mmol) and 4-[([(5-amino-1,3,4-oxadiazol-2-yl)methyl]amino)methyl]-3-fluoro-benzonitrile (40 mg, 0.162 mmol) was prepared without stepwise addition. The reaction was then stirred 16 hours before assessing reaction progress by LCMS. After 16 hours the reaction had consumed the amine starting material and the crude reaction mixture was directed purified by Low pH preparative HPLC (Standard method). The desired fractions were concentrated to provide N-[(5-amino-1,3,4-oxadiazol-2-yl)methyl]-2-(2-chloro-3,5-difluoro-phenoxy)-N-[(4-cyano-2-fluoro-phenyl)methyl]acetamide (100.0%) (10 mg, 0.0221 mmol, 14% Yield) as a white solid. ¹H-NMR (400 MHz, Methanol-d₄) δ 7.64-7.40 (m, 3H), 6.84-6.66 (m, 2H), 5.24-5.07 (m, 2H), 4.93-4.72 (m, 2H), 4.69-4.50 (m, 2H). (ESI⁺)—LCMS Method A rt=2.85 min (M+H)⁺ 452.1 and 454.1.

The compounds in Table 7 below were prepared based on experimental procedures described herein above.

TABLE 7

| | | Mass | |
|---|---|---|---|
| Compound | | Spec. | |
| No. | Chemical Structure | (ES, m/z) | ¹H NMR Spectrum |
| 32 | 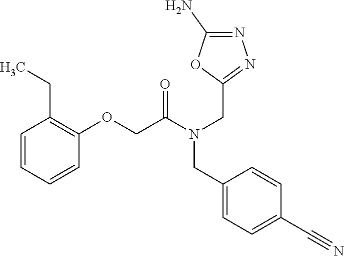 [SMILES: CCc1ccccc1OCC(=O)N(Cc1ccc(C#N)cc1)Cc1nnc(N)o1] | 392.0 [M + H]+ | (500 MHz, DMSO-d6) δ 7.87-7.73 (m, 2H), 7.50- 7.37 (m, 2H), 7.19-6.95 (m, 4H), 6.92-6.73 (m, 2H), 5.12-4.84 (m, 2H), 4.84-4.49 (m, 4H), 2.66- 2.56 (m, 2H), 1.19-1.02 (m, 3H). |
| 33 | 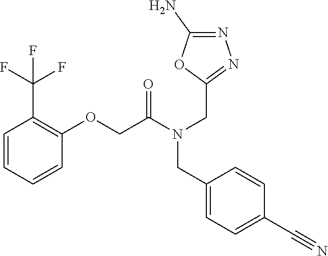 [SMILES: N#Cc1ccc(CN(Cc2nnc(N)o2)C(=O)COc2ccccc2C(F)(F)F)cc1] | 432.0 [M + H]+ | (500 MHz, DMSO-d6) δ 7.91-7.69 (m, 2H), 7.67- 7.54 (m, 2H), 7.53-7.35 (m, 2H), 7.20-6.92 (m, 4H), 5.16 (m, 2H), 4.85- 4.49 (m, 4H) |
| 34 | 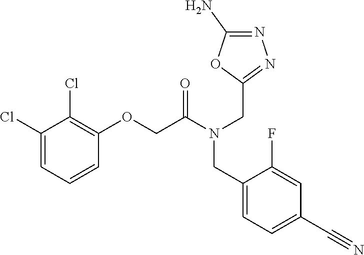 [SMILES: N#Cc1ccc(CN(Cc2nnc(N)o2)C(=O)COc2cccc(Cl)c2Cl)c(F)c1] | 450.0 [M + H]+ | (500 MHz, Methanol-d4) δ 7.61-7.33 (m, 3H), 7.26- 7.19 (m, 1H), 7.18-7.11 (m, 1H), 7.03-6.95 (m, 1H), 5.20-5.07 (m, 2H), 4.95-4.52 (m, 4H). |
| 35 | 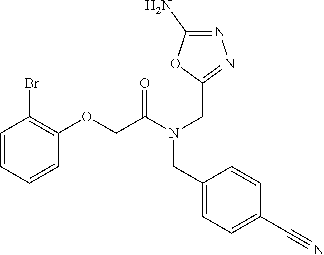 [SMILES: N#Cc1ccc(CN(Cc2nnc(N)o2)C(=O)COc2ccccc2Br)cc1] | 444.2 [M + H]+ | (400 MHz, DMSO-d6) δ 7.92-7.77 (m, 2H), 7.64- 7.44 (m, 2H), 7.44-7.27 (m, 2H), 7.14-6.79 (m, 4H), 5.23-4.94 (m, 2H), 4.86-4.47 (m, 4H). |
| 36 | 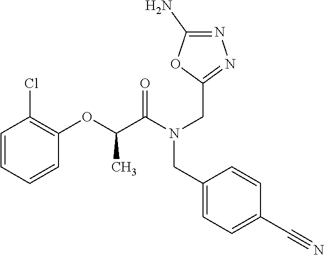 [SMILES: C[C@@H](Oc1ccccc1Cl)C(=O)N(Cc1ccc(C#N)cc1)Cc1nnc(N)o1] | 411.1 [M + H]+ | (400 MHz, Methanol-d4) δ 7.67-7.55 (m, 2H), 7.42- 7.17 (m, 4H), 7.07-6.90 (m, 2H), 5.46-5.18 (m, 1H), 1.77-1.47 (m, 3H). |
| 37 | 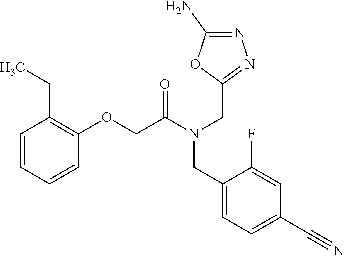 [SMILES: CCc1ccccc1OCC(=O)N(Cc1nnc(N)o1)Cc1ccc(C#N)cc1F] | 410.3 [M + H]+ | (500 MHz, Methanol-d4) δ 7.61-7.33 (m, 3H), 7.24- 6.77 (m, 4H), 5.07-4.60 (m, 6H), 2.72-2.45 (m, 2H), 1.23-1.05 (m, 3H). |
| 38 | 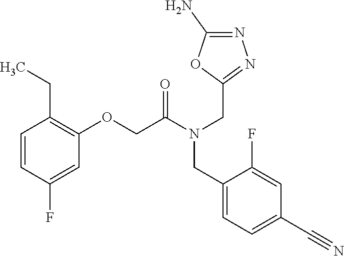 [SMILES: CCc1ccc(F)cc1OCC(=O)N(Cc1nnc(N)o1)Cc1ccc(C#N)cc1F] | 428.5 [M + H]+ | (400 MHz, Methanol-d4) δ 7.69-7.38 (m, 3H), 7.23- 7.05 (m, 1H), 6.78-6.55 (m, 2H), 5.13-4.65 (m, 6H), 2.77-2.45 (m, 2H), 1.25-1.09 (m, 3H). |
| 39 | 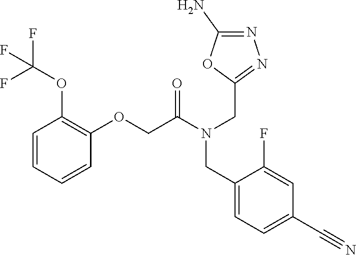 [SMILES: N#Cc1ccc(CN(Cc2nnc(N)o2)C(=O)COc2ccccc2OC(F)(F)F)c(F)c1] | 466.2 [M + H]+ | (400 MHz, Methanol-d4) δ 7.67-7.41 (m, 3H), 7.37- 7.25 (m, 2H), 7.18-7.01 (m, 2H), 5.23-5.05 (m, 2H), 4.81-4.67 (m, 2H). |
| 40 | 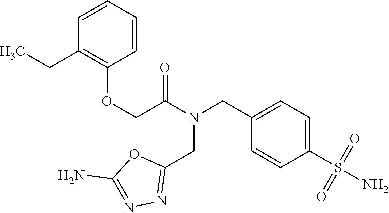 | 466.2 [M + H ]+ | (500 MHz, Methanol-d4) δ 7.92-7.77 (m, 2H), 7.47- 7.34 (m, 2H), 7.22-7.05 (m, 2H), 6.99-6.78 (m, 2H), 5.12-4.57 (m, 6H), 2.73-2.44 (m, 2H), 1.31- 1.04 (m, 3H). |
| 41 | 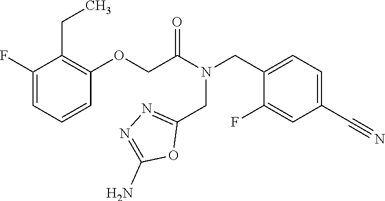 [SMILES: CCc1c(F)cccc1OCC(=O)N(Cc1nnc(N)o1)Cc1ccc(C#N)cc1F] | 428.2 [M + H]+ | (500 MHz, Methanol-d4) δ 7.63-7.39 (m, 3H), 7.17- 7.07 (m, 1H), 6.76-6.61 (m, 2H), 5.14-4.61 (m, 6H), 2.79-2.50 (m, 2H), 1.20-1.00 (m, 3H). |
| 42 | 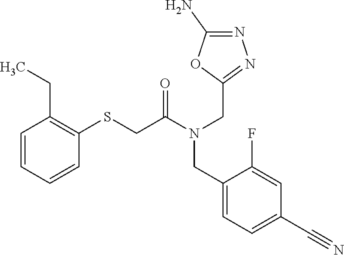 [SMILES: CCc1ccccc1SCC(=O)N(Cc1nnc(N)o1)Cc1ccc(C#N)cc1F] | 428.5 [M + H]+ | (500 MHz, Methanol-d4) δ 7.71-6.99 (m, 7H), 4.82- 4.54 (m, 4H), 4.06-3.82 (m, 2H), 2.84-2.72 (m, 2H), 1.23-1.12 (m, 3H). |
| 43 | 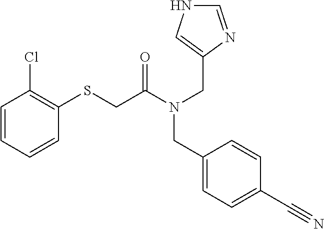 [SMILES: N#Cc1ccc(CN(Cc2c[nH]cn2)C(=O)CSc2ccccc2Cl)cc1] | 397.2 [M + H]+ | (500 MHz, Methanol-d4) δ 7.71-7.57 (m, 3H), 7.51- 7.18 (m, 6H), 7.05-6.88 (m, 1H), 4.83-4.49 (m, 4H), 4.26-3.87 (m, 2H). |
| 44 | 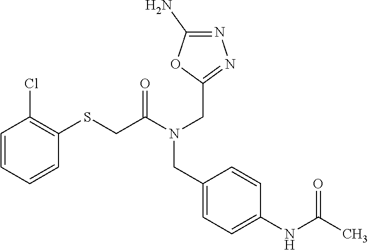 [SMILES: CC(=O)Nc1ccc(CN(Cc2nnc(N)o2)C(=O)CSc2ccccc2Cl)cc1] | 446.0 [M + H]+ | (500 MHz, DMSO-d6) δ 10.07-9.77 (m, 1H), 7.68- 6.91 (m, 11H), 4.69 (s, 2H), 4.53-4.45 (m, 2H), 4.33- 4.13 (m, 2H), 2.06-1.97 (m, 3H). |
| 45 | 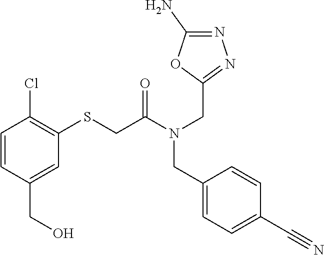 [SMILES: N#Cc1ccc(CN(Cc2nnc(N)o2)C(=O)CSc2cc(CO)ccc2Cl)cc1] | 444.2 [M + H]+ | (400 MHz, DMSO-d6) δ 7.88-7.73 (m, 2H), 7.54- 6.96 (m, 6H), 5.37-5.27 (m, 1H), 4.94-4.76 (m, 2H), 4.70-4.52 (m, 2H), 4.51-4.43 (m, 2H), 4.35- 4.07 (m, 2H). |
| 46 | 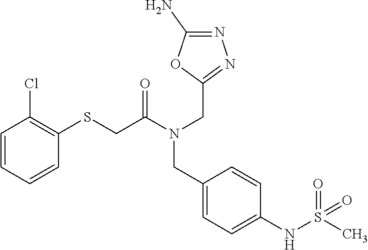 [SMILES: CS(=O)(=O)Nc1ccc(CN(Cc2nnc(N)o2)C(=O)CSc2ccccc2Cl)cc1] | 482.1 & 484.1 [M + H]+ | (400 MHz, DMSO-d6) δ 9.66 (br. s, 1H), 7.49-7.11 (m, 8H), 7.08-6.94 (m, 2H), 4.80-4.43 (m, 4H), 4.33-4.10 (m, 2H), 2.99- 2.91 (m, 3H). |
| 47 | 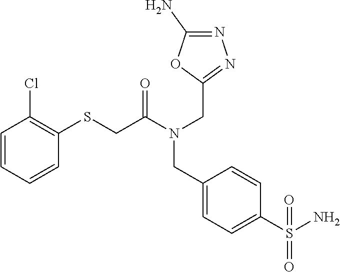 [SMILES: Nc1nnc(CN(Cc2ccc(S(N)(=O)=O)cc2)C(=O)CSc2ccccc2Cl)o1] | 468.1 [M + H]+ | (500 MHz, DMSO-d6) δ 7.87-7.71 (m, 1H), 7.50- 7.15 (m, 4H), 7.12-6.99 (m, 1H), 4.92-4.73 (m, 1H), 4.66-4.50 (m, 1H), 4.39-3.96 (m, 1H). |
| 48 | 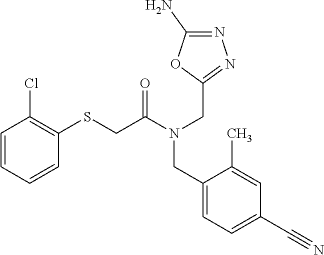 [SMILES: Cc1cc(C#N)ccc1CN(Cc1nnc(N)o1)C(=O)CSc1ccccc1Cl] | 428.2 [M + H]+ | (400 MHz, DMSO-d6) δ 7.74-7.56 (m, 2H), 7.50- 7.14 (m, 5H), 7.11-6.95 (m, 2H), 4.92-4.72 (m, 2H), 4.64-4.53 (m, 2H), 4.43-3.98 (m, 2H), 2.33- 2.22 (m, 3H). |
| 49 | 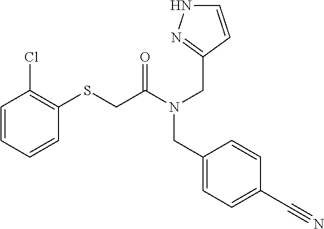 [SMILES: N#Cc1ccc(CN(Cc2cc[nH]n2)C(=O)CSc2ccccc2Cl)cc1] | 397.2 [M + H]+ | (400 MHz, Methanol-d4) δ 7.72-7.54 (m, 3H), 7.47- 7.31 (m, 4H), 7.28-7.16 (m, 2H), 6.27-6.17 (m, 1H), 4.81-4.55 (m, 4H), 4.23-3.89 (m, 2H). |
| 50 | 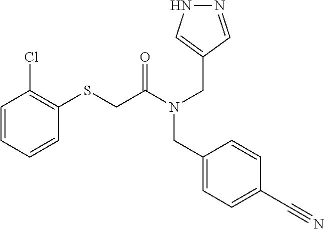 [SMILES: N#Cc1ccc(CN(Cc2cn[nH]c2)C(=O)CSc2ccccc2Cl)cc1] | 397.2 [M + H]+ | (400 MHz, Methanol-d4) δ 7.76-7.59 (m, 2H), 7.56 (s, 1H), 7.52-7.35 (m, 4H), 7.34-7.18 (m, 3H), 4.79- 4.38 (m, 4H), 4.16-3.86 (m, 2H). |
| 51 | 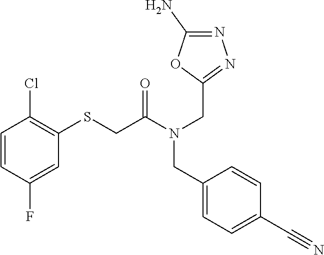 [SMILES: N#Cc1ccc(CN(Cc2nnc(N)o2)C(=O)CSc2cc(F)ccc2Cl)cc1] | 432.2 [M + H]+ | (400 MHz, DMSO-d6) δ 7.88-7.71 (m, 2H), 7.54- 7.26 (m, 4H), 7.09-6.94 (m, 3H), 4.96-4.77 (m, 2H), 4.69-4.48 (m, 2H), 4.43-4.20 (m, 2H). |
| 52 | 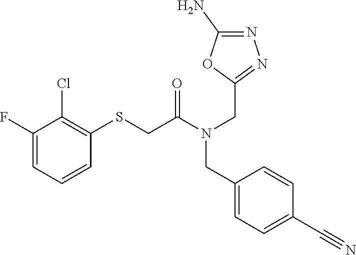 [SMILES: N#Cc1ccc(CN(Cc2nnc(N)o2)C(=O)CSc2cccc(F)c2Cl)cc1] | 432.1 [M + H]+ | (500 MHz, DMSO-d6) δ 7.90-7.71 (m, 2H), 7.53- 7.13 (m, 5H), 7.11-6.97 (m, 2H), 4.94-4.74 (m, 2H), 4.68-4.49 (m, 2H), 4.43-4.14 (m, 2H). |
| 53 | 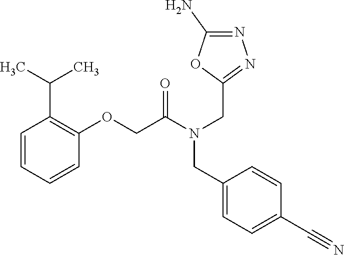 [SMILES: CC(C)c1ccccc1OCC(=O)N(Cc1ccc(C#N)cc1)Cc1nnc(N)o1] | 406.2 [M + H]+ | (500 MHz, DMSO-d6) δ 7.91-7.72 (m, 2H), 7.53- 7.37 (m, 2H), 7.25-6.97 (m, 4H), 6.95-6.74 (m, 2H), 5.15-4.48 (m, 6H), 1.32-0.87 (m, 6H) |
| 54 | 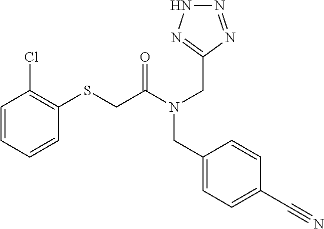 [SMILES: N#Cc1ccc(CN(Cc2nn[nH]n2)C(=O)CSc2ccccc2Cl)cc1] | 399.1 [M + H]+ | (500 MHz, DMSO-d6) δ 7.82 (m, 2H), 7.57-7.14 (m, 6H), 5.00 (m, 2H), 4.70 (m, 2H), 4.27 (m, 2H). |
| 55 | 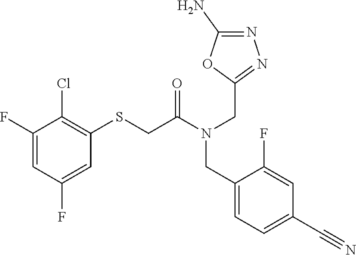 [SMILES: N#Cc1ccc(CN(Cc2nnc(N)o2)C(=O)CSc2cc(F)cc(F)c2Cl)c(F)c1] | 468.2 [M + H]+ | (500 MHz, DMSO-d6) δ 7.92-7.76 (m, 1H), 7.72- 7.61 (m, 1H), 7.53-7.40 (m, 1H), 7.37-7.19 (m, 2H), 7.14-6.91 (m, 2H), 5.00-4.80 (m, 2H), 4.72- 4.49 (m, 2H), 4.47-4.26 (m, 2H). |
| 56 | 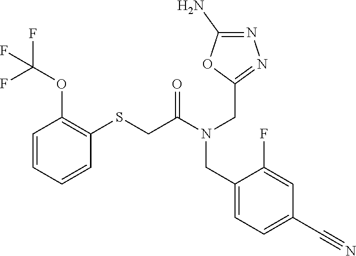 [SMILES: N#Cc1ccc(CN(Cc2nnc(N)o2)C(=O)CSc2ccccc2OC(F)(F)F)c(F)c1] | 482.1 [M + H]+ | (400 MHz, DMSO-d6) δ 7.91-7.77 (m, 1H), 7.72- 7.60 (m, 1H), 7.52-7.28 (m, 5H), 7.09-6.94 (m, 2H), 5.01-4.76 (m, 2H), 2.70-4.48 (m, 2H), 4.38- 4.12 (m, 2H). |
| 57 | 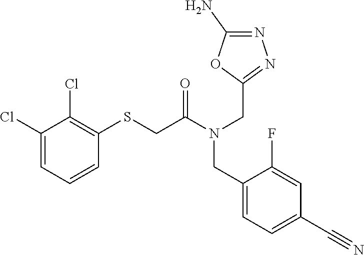 [SMILES: N#Cc1ccc(CN(Cc2nnc(N)o2)C(=O)CSc2cccc(Cl)c2Cl)c(F)c1] | 466.0 [M + H]+ | (400 MHz, Methanol-d4) δ 7.66-7.17 (m, 8H), 5.00- 4.89 (m, 2H), 4.78-4.64 (m, 2H), 4.29-4.07 (m, 2H). |
| 58 | 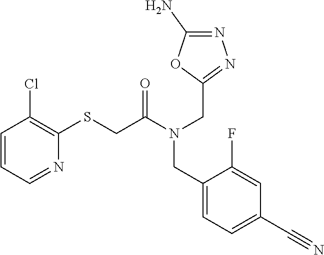 [SMILES: N#Cc1ccc(CN(Cc2nnc(N)o2)C(=O)CSc2ncccc2Cl)c(F)c1] | 433.1 [M + H]+ | (400 MHz, Methanol-d4) δ 8.37-8.28 (m, 1H), 7.75- 7.44 (m, 4H), 7.17-7.09 (m, 1H), 5.11-4.98 (m, 2H), 4.85-4.55 (m, 4H), 4.39-4.18 (m, 2H). |
| 59 | 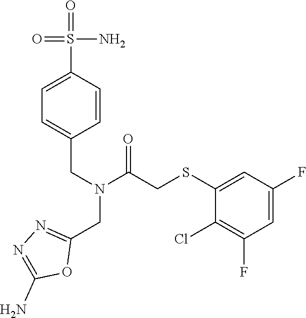 [SMILES: Nc1nnc(CN(Cc2ccc(S(N)(=O)=O)cc2)C(=O)CSc2cc(F)cc(F)c2Cl)o1] | 504.0 [M + H]+ | (500 MHz, Methanol-d4) δ 7.97-7.78 (m, 2H), 7.52- 7.35 (m, 2H), 7.20-6.92 (m, 2H), 4.99-4.11 (m, 6H). |

### Example 8—Synthesis of 2-[5-(2-Aminoethoxy)-2-chloro-phenyl]sulfanyl-N-[(5-amino-1,3,4-oxadiazol-2-yl)methyl]-N-[(4-cyano-2-fluoro-phenyl)methyl]acetamide (Compound No. 60)

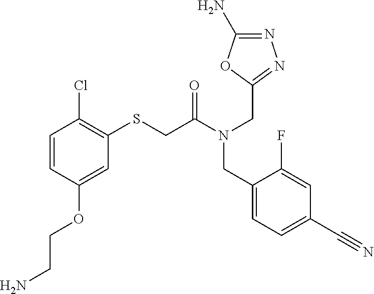 [SMILES: N#Cc1ccc(CN(Cc2nnc(N)o2)C(=O)CSc2cc(OCCN)ccc2Cl)c(F)c1]

### tert-Butyl N-[2-(3-bromo-4-chloro-phenoxy)ethyl]carbamate

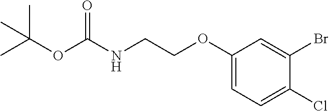 [SMILES: CC(C)(C)OC(=O)NCCOc1ccc(Cl)c(Br)c1]

tert-Butyl (2-hydroxyethyl)carbamate (2285 mg, 14.2 mmol) and triphenylphosphane (4213 mg, 16.1 mmol) were added to a stirred solution of 3-bromo-4-chlorophenol (98%, 2.00 g, 9.45 mmol) in THF-Anhydrous (94 mL). Finally isopropyl (N{E})-N-isopropoxycarbonyliminocarbamate (287 mg, 14.2 mmol) was added dropwise to the stirred solution at 0° C. The solution was raised to room temperature after 30 mins and left to stir for 24 hours. At this point the crude material was Normal phase column chromatography Method 1 to provide tert-butyl N-[2-(3-bromo-4-chloro-phenoxy)ethyl]carbamate (3.30 g, 9.41 mmol, 100% Yield) as a colourless oil. ¹H-NMR (500 MHz, DMSO-d₆) δ 7.51 (d, J=8.9 Hz, 1H), 7.34 (d, J=2.9 Hz, 1H), 7.00 (dd, J=8.9, 2.9 Hz, 2H), 4.01-3.95 (m, 2H), 3.30-3.22 (m, 2H), 1.38 (s, 9H). (ESI⁺) LCMS Method A rt=4.00 min (M+H)⁺ 293.4.

### Methyl 2-[5-[2-(tert-butoxycarbonylamino)ethoxy]-2-chloro-phenyl]sulfanylacetate

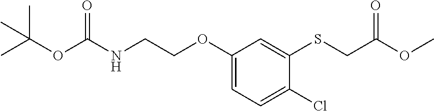 [SMILES: COC(=O)CSc1cc(OCCNC(=O)OC(C)(C)C)ccc1Cl]

To a pressure vial was added tert-butyl N-[2-(3-bromo-4-chloro-phenoxy)ethyl]carbamate (95%, 2.00 g, 5.42 mmol), methyl sulfanylacetate (0.59 mL, 6.50 mmol), disodium;carbonate (1436 mg, 13.5 mmol) and 1,4-Dioxane (16.42 mL). The reaction vial was purged with nitrogen for 5 minutes and then XantPhos Pd G3 (514 mg, 0.542 mmol) was added and the reaction was purged with nitrogen for a further minute. The reaction was placed in a pre-heated hot plate at 100° C. and stirred for 24 h. The crude was then purified Normal phase column chromatography Method 1 to provide methyl 2-[5-[2-(tert-butoxycarbonylamino)ethoxy]-2-chloro-phenyl]sulfanylacetate (1.14 g, 2.48 mmol, 46% Yield) as a yellow oil. ¹H-NMR (400 MHz, DMSO-d₆) δ 7.35 (d, J=8.8 Hz, 1H), 7.01 (t, J=5.4 Hz, 1H), 6.89 (d, J=2.8 Hz, 1H), 6.79 (dd, J=8.8, 2.8 Hz, 1H), 4.05 (d, J=2.5 Hz, 2H), 3.97 (t, J=5.8 Hz, 2H), 3.67 (s, 3H), 3.28 (q, J=5.9 Hz, 2H), 1.39 (s, 9H). (ESI⁺) LCMS Method A rt=3.71 min (M+H)⁺ 376.2.

### Lithium 2-[5-[2-(tert-butoxycarbonylamino)ethoxy]-2-chloro-phenyl]sulfanylacetate

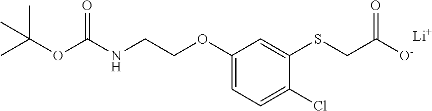 [SMILES: CC(C)(C)OC(=O)NCCOc1ccc(Cl)c(SCC(=O)[O-])c1 ; [Li+]]

A solution of methyl 2-[5-[2-(tert-butoxycarbonylamino)ethoxy]-2-chloro-phenyl]sulfanylacetate (1.14 g, 3.03 mmol) and hydroxylithium (80 mg, 3.33 mmol) in Water (3.0 mL)/Methanol (3.0 mL)/THF (3.0 mL) was stirred at RT for 16 h. The solvent was then evaporated to dryness then taken into the next step as the crude product lithium 2-[5-[2-(tert-butoxycarbonylamino)ethoxy]-2-chloro-phenyl]sulfanylacetate (1.09 g, 2.37 mmol, 78% Yield) as a white solid. ¹H-NMR (500 MHz, DMSO-d₆) δ 7.24 (d, J=8.7 Hz, 1H), 7.06 (t, J=5.4 Hz, 1H), 6.89 (d, J=2.8 Hz, 1H), 6.63 (dd, J=8.7, 2.8 Hz, 1H), 3.94 (t, J=5.8 Hz, 2H), 3.36 (s, 2H), 3.27 (q, J=5.8 Hz, 2H), 1.38 (s, 9H). (ESI⁺) LCMS Method B rt.=1.41 min (M−Boc−Li+H)⁺ 261.85 and 263.80

### 2-[5-(2-Aminoethoxy)-2-chloro-phenyl]sulfanyl-N-[(5-amino-1,3,4-oxadiazol-2-yl)methyl]-N-[(4-cyano-2-fluoro-phenyl)methyl]acetamide (Compound No. 60)

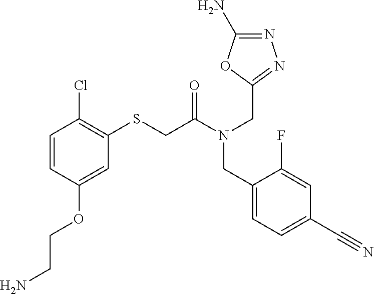 [SMILES: N#Cc1ccc(CN(Cc2nnc(N)o2)C(=O)CSc2cc(OCCN)ccc2Cl)c(F)c1]

A stirred solution of lithium 2-[5-[2-(tert-butoxycarbonylamino)ethoxy]-2-chloro-phenyl]sulfanylacetate (60%, 89 mg, 0.146 mmol), 1-[bis(dimethylamino)methylidene]-1H-[1,2,3]triazolo[4,5-b]pyridin-1-ium 3-oxide hexafluorophosphate (66 mg, 0.175 mmol), N-ethyl-N-isopropyl-propan-2-amine (0.051 mL, 0.291 mmol) and 4-[([(5-amino-1,3,4-oxadiazol-2-yl)methyl]amino)methyl]-3-fluoro-benzonitrile (60%, 60 mg, 0.146 mmol) in DMF-Anhydrous (0.7 mL) was stirred for 2 hours. After the reaction had consumed the amine starting material and the crude reaction mixture was directly purified by High pH preparative HPLC to provide tert-butyl N-[2-[3-[2-[(5-amino-1,3,4-oxadiazol-2-yl)methyl-[(4-cyano-2-fluoro-phenyl)methyl]amino]-2-oxo-ethyl]sulfanyl-4-chloro-phenoxy]ethyl]carbamate (18 mg, 0.0280 mmol, 19% Yield) as a white foam. Then 4 M HCl in ethanol (0.11 mL, 0.437 mmol) was added and the solution stirred for 16 hours then the suspension was concentrated and taken up in MeOH purified by SCX-2 column, eluting with 2M NH3 in MeOH. The eluted material was concentrated to provide 2-[5-(2-aminoethoxy)-2-chloro-phenyl]sulfanyl-N-[(5-amino-1,3,4-oxadiazol-2-yl)methyl]-N-[(4-cyano-2-fluoro-phenyl)methyl]acetamide (10 mg, 0.0204 mmol, 14% Yield) as a white foam. ¹H-NMR (400 MHz, Methanol-d4) δ 7.62-7.31 (m, 3H), 7.31-7.24 (m, 1H), 7.09-7.04 (m, 1H), 6.84-6.76 (m, 1H), 4.92 (d, J=38.7 Hz, 2H), 4.69 (d, J=49.7 Hz, 2H), 4.09-3.98 (m, 2H), 3.11-2.93 (m, 2H). (ESI⁺)—LCMS Method H rt=2.66 min (M+H)⁺ 491.3.

### Example 9—Synthesis of N-[[4-(Aminomethyl)phenyl]methyl]-N-[(5-amino-1,3,4-oxadiazol-2-yl)methyl]-2-(2-chlorophenyl)sulfanyl-acetamide (Compound No. 61)

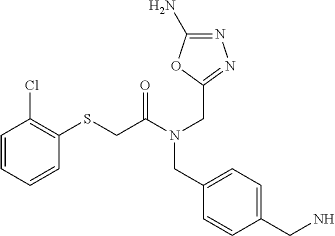 [SMILES: NCc1ccc(CN(Cc2nnc(N)o2)C(=O)CSc2ccccc2Cl)cc1]

### tert-Butyl 2-(2-chlorophenyl)sulfanylacetate

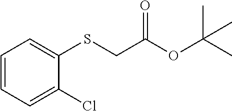 [SMILES: CC(C)(C)OC(=O)CSc1ccccc1Cl]

To a sealed tube was added 2-chlorobenzenethiol (0.91 mL, 8.00 mmol), tert-butyl 2-chloroacetate (1.7 mL, 12.0 mmol), dipotassium;carbonate (2.21 g, 16.0 mmol) and DMF (27 mL). The reaction was sealed and heated to 80° C. The reaction was cooled to room temperature, diluted with H2O (20 mL) and extracted with EtOAc (2×50 mL) and the combined organic extracts were dried (MgSO4) and concentrated in vacuo. The product was purified by Normal phase column chromatography Method 1. The desired fractions were concentrated in vacuo to afford tert-butyl 2-(2-chlorophenyl)sulfanylacetate (2.07 g, 7.68 mmol, 96% Yield) as a colourless liquid. ¹H-NMR (400 MHz, Chloroform-d) δ 7.39 (m, 2H), 7.22 (td, J=7.6, 1.5 Hz, 1H), 7.15 (td, J=7.6, 1.6 Hz, 1H), 3.61 (s, 2H), 1.41 (s, 9H). (ESI⁺)—LCMS Method A rt.=1.06 min

### 2-(2-Chlorophenyl)sulfanylacetic acid

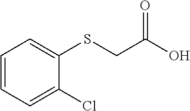 [SMILES: O=C(O)CSc1ccccc1Cl]

To a solution of tert-butyl 2-(2-chlorophenyl)sulfanylacetate (2.07 g, 7.20 mmol) in DCM (48 mL) was added 2,2,2-trifluoroacetic acid (2.7 mL, 36.0 mmol) and the reaction was stirred at room temperature for 18 h. The reaction was concentrated in vacuo to afford 2-(2-chlorophenyl)sulfanylacetic acid (1.52 g, 6.53 mmol, 91% Yield) as a pale pink solid. ¹H NMR (500 MHz, DMSO-d6) δ 12.92 (s, 1H), 7.46 (d, J=7.8 Hz, 1H), 7.35-7.32 (m, 2H), 7.22-7.17 (m, 1H), 3.91 (s, 2H). (ESI⁺)—LCMS Method A rt.=0.44 min (ESI⁺) 334.3

### tert-Butyl N-[[4-[([(5-amino-1,3,4-oxadiazol-2-yl)methyl]amino)methyl]phenyl]methyl]carbamate

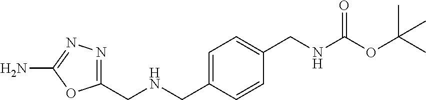 [SMILES: CC(C)(C)OC(=O)NCc1ccc(CNCc2nnc(N)o2)cc1]

5-(Aminomethyl)-1,3,4-oxadiazol-2-amine dihydrochloride (61 mg, 0.327 mmol) was dissolved in MeOH loaded on to a MeOH washed SCX-2 column (2 g). The column was eluted with MeOH followed by 2N NH₃/MeOH. The NH3/MeOH fraction was concentrated to give the amine free base which was dissolved in IPA (1.4876 mL). pyrrolidine (5.0 uL, 0.0595 mmol) and tert-butyl N-[(4-formylphenyl)methyl]carbamate (70 mg, 0.298 mmol) were added and the resultant mixture was stirred at 60° C. for 1 hour. Upon cooling sodium borohydride (46 mg, 1.2 mmol) was added and the reaction mixture was stirred at RT. The reaction quenched with a small amount of water and taken up in MeOH and loaded on to a MeOH washed SCX-2 column. The column was eluted with MeOH followed by 2N NH₃/MeOH. The NH₃/MeOH fractions were combined and the solvent was removed under reduced pressure to give tert-butyl N-[[4-[([(5-amino-1,3,4-oxadiazol-2-yl)methyl]amino)methyl]phenyl]methyl]carbamate (107 mg, 0.231 mmol, 78% yield) as a yellow gum. 1H NMR (400 MHz, Chloroform-d) δ=7.42-6.97 (m, 4H), 4.97 (m, 4H), 4.30 (m, 2H), 3.90-3.79 (m, 4H), 1.46 (s, 9H). (ESI⁺)—LCMS Method H rt.=0.44 min (ESI+) 334.3.

### tert-Butyl N-[[4-[[[(5-amino-1,3,4-oxadiazol-2-yl)methyl]-N-[2-(2-chlorophenyl)sulfanylacetyl] amino]methyl]phenyl]methyl]carbamate

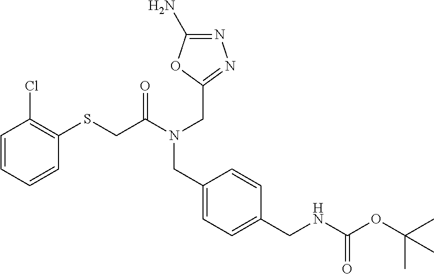 [SMILES: CC(C)(C)OC(=O)NCc1ccc(CN(Cc2nnc(N)o2)C(=O)CSc2ccccc2Cl)cc1]

tert-Butyl N-[[4-[([(5-amino-1,3,4-oxadiazol-2-yl)methyl]amino)methyl]phenyl]methyl]carbamate (72%, 107 mg, 0.231 mmol) was solubilized in DMF anhydrous (1.1554 mL). 2-(2-chlorophenyl)sulfanylacetic acid (47 mg, 0.231 mmol) and N-ethyl-N-isopropyl-propan-2-amine (0.12 mL, 0.693 mmol) were added to the solution. 1-[bis(dimethylamino) methylidene]-1H-[1,2,3]triazolo[4,5-b]pyridin-1-ium 3-oxide hexafluorophosphate (97 mg, 0.254 mmol) was added and the reaction was stirred at RT overnight. The reaction solution was purified by High pH preparative HPLC (Standard method) to give tert-butyl N-[[4-[[[(5-amino-1,3,4-oxadiazol-2-yl)methyl]-N-[2-(2-chlorophenyl)sulfanylacetyl]amino]methyl]phenyl] methyl]carbamate (54 mg, 0.104 mmol, 45% Yield) as a white gum. ¹H-NMR (500 MHz, Methanol-d4) δ=7.53-6.95 (m, 8H), 4.81-4.55 (m, 4H), 4.28-3.94 (m, 4H), 1.45 (s, 9H). (ESI⁺) LCMS Method H rt.=0.60 min (ESI+) 518.4.

### N-[[4-(Aminomethyl)phenyl]methyl]-N-[(5-amino-1,3,4-oxadiazol-2-yl)methyl]-2-(2-chlorophenyl)sulfanyl-acetamide (Compound No. 61)

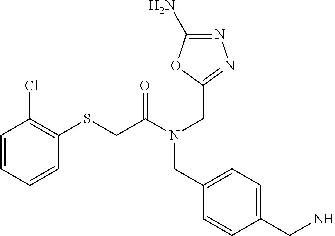 [SMILES: NCc1ccc(CN(Cc2nnc(N)o2)C(=O)CSc2ccccc2Cl)cc1]

4 M hydrogen chloride in dioxane (0.52 mL, 2.08 mmol) was added to tert-butyl N-[[4-[[[(5-amino-1,3,4-oxadiazol-2-yl)methyl]-N-[2-(2-chlorophenyl)sulfanylacetyl]amino]methyl]phenyl]methyl]carbamate (100%, 54 mg, 0.104 mmol) and the reaction was stirred at RT overnight. The solvent was removed under reduced pressure and the residue was taken up in MeOH and loaded on to a MeOH washed SCX-2 column (2 g). The column was eluted with MeOH followed by 7N NH₃/MeOH. The NH₃/MeOH fraction was collected and the solvent was removed to give N-[[4-(aminomethyl)phenyl]methyl]-N-[(5-amino-1,3,4-oxadiazol-2-yl)methyl]-2-(2-chlorophenyl)sulfanyl-acetamide (28 mg, 0.0672 mmol, 65% Yield) as a yellow gum. ¹H-NMR (400 MHz, DMSO-d6) δ=7.55-6.95 (m, 10H), 4.74 (m, 2H), 4.54 (s, 2H), 4.24 (m, 2H), 3.71 (m, 2H). (ESI⁺)—LCMS Method H rt.=3.50 min (ESI+) 418.10 & 420.10.

### Example 10—Synthesis of N-[(5-Amino-1,3,4-oxadiazol-2-yl)methyl]-2-[2-chloro-5-(1H-imidazol-4-yl)phenyl]sulfanyl-N-[(4-cyano-2-fluoro-phenyl)methyl]acetamide (Compound No. 62)

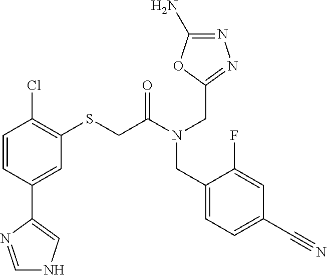 [SMILES: N#Cc1ccc(CN(Cc2nnc(N)o2)C(=O)CSc2cc(-c3c[nH]cn3)ccc2Cl)c(F)c1]

### 4-(3-Bromo-4-chloro-phenyl)-1H-imidazole

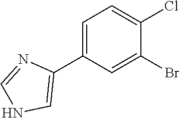 [SMILES: Clc1ccc(-c2c[nH]cn2)cc1Br]

1-(3-Bromo-4-chlorophenyl)ethan-1-one (2.00 g, 8.57 mmol) was dissolved in Et₂O (20 mL) and the solution was cooled to 0° C. under nitrogen. Bromine (0.49 mL, 9.42 mmol) was then added dropwise and the reaction left to stir at 0-5° C. for 1 h. The reaction was then quenched with water (20 mL) and the aqueous layer that formed was washed (2×20 mL). The organic layers were collected and dried with MgSO₄, filtered then concentrated to dryness to provide 2-bromo-1-(3-bromo-4-chloro-phenyl)ethanone as a crude product.

The crude material was diluted with formamide (14 mL, 0.343 mol) and the mixture stirred and heated to 160° C. for 24 h. The resultant crude reaction mixture was cooled to room temperature and diluted with sat. NaHCO3 (80 mL) and then the crude product was extracted with EtOAc (3×80 mL). The combined organics were then dried with MgSO4, filtered, concentrated in vacuo to afford the crude product as a brown oil. The crude was then purified using Normal phase column chromatography Method 1 to provide an impure product that was dissolved in EtOAc (50 mL) and washed with 1M HCl aq. (2×50 mL), the aqueous layers were combined and washed again with EtOAc (50 mL). The aqueous layers were then basified with solid NaHCO3 until the pH reached 8. The basic aqueous layer was extracted with EtOAc (2×50 mL). The two combined basic extraction organic layer was dried with MgSO4 solids, filtered and concentrated to provide 4-(3-bromo-4-chloro-phenyl)-1H-imidazole (492 mg, 1.91 mmol, 22% Yield) as a white solid. ¹H-NMR (400 MHz, DMSO-d₆) δ 12.29 (s, 1H), 8.14 (d, J=2.0 Hz, 1H), 7.86-7.70 (m, 3H), 7.58 (d, J=8.4 Hz, 1H). (ESI⁺)—LCMS Method H r.t.=3.62 min (M+H)⁺ 257.1, 259.1, 261.1 and 262.

### Methyl 2-[2-chloro-5-(1H-imidazol-4-yl)phenyl]sulfanylacetate

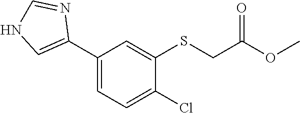 [SMILES: COC(=O)CSc1cc(-c2c[nH]cn2)ccc1Cl]

To a pressure vial was added 4-(3-bromo-4-chloro-phenyl)-1H-imidazole (0.49 g, 1.91 mmol), methyl sulfanylacetate (0.21 mL, 2.29 mmol), disodium carbonate (506 mg, 4.78 mmol) and 1,4-Dioxane (5.7897 mL). The reaction vial was purged with nitrogen for 5 minutes and then XantPhos Pd G3 (181 mg, 0.191 mmol) was added and the reaction was purged with nitrogen for a further minute. The reaction was placed in a pre-heated hot plate at 100° C. and stirred for 5 h before LCMS showed consumption of SM. The crude reaction mixture was concentrated directly onto silica to dryness. The crude was then purified by Normal phase column chromatograph Method 2 to provide methyl 2-[2-chloro-5-(1H-imidazol-4-yl)phenyl]sulfanylacetate (390 mg, 1.17 mmol, 61% Yield) as a green foam. ¹H-NMR (400 MHz, DMSO-d₆) δ 12.24 (s, 1H), 7.80-7.57 (m, 4H), 7.46-7.39 (m, 1H), 4.07-4.01 (m, 2H), 3.66 (s, 3H). (ESI⁺)—LCMS Method A rt=1.56 min (M+H)⁺ 225.0.

### Lithium 2-[2-chloro-5-(1H-imidazol-4-yl)phenyl]sulfanylacetate

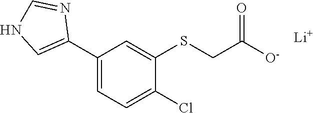 [SMILES: O=C([O-])CSc1cc(-c2c[nH]cn2)ccc1Cl ; [Li+]]

A solution of methyl 2-[2-chloro-5-(1H-imidazol-4-yl)phenyl]sulfanylacetate (390 mg, 1.38 mmol) and hydroxylithium monohydrate (64 mg, 1.52 mmol) in Water (1.4 mL)/Methanol (1.4 mL)/THF (1.4 mL) was stirred at RT for 16 h. The solvent was then evaporated and the reaction mixture was taken into the next step as the crude product lithium 2-[2-chloro-5-(1H-imidazol-4-yl)phenyl]sulfanylacetate (378 mg, 0.963 mmol, 70% Yield) as a yellow solid. ¹H-NMR (500 MHz, DMSO-d6) δ 7.69 (d, J=1.9 Hz, 1H), 7.64 (s, 1H), 7.54 (s, 1H), 7.43 (dd, J=8.2, 1.9 Hz, 1H), 7.30 (d, J=8.3 Hz, 1H), 3.48 (s, 2H), 3.35 (s, 1H). (ESI⁺)—LCMS Method B rt=0.86 min (M+H)⁺ 268.85 and 270.80.

### N-[(5-Amino-1,3,4-oxadiazol-2-yl)methyl]-2-[2-chloro-5-(1H-imidazol-4-yl)phenyl]sulfanyl-N-[(4-cyano-2-fluoro-phenyl)methyl]acetamide (Compound 62)

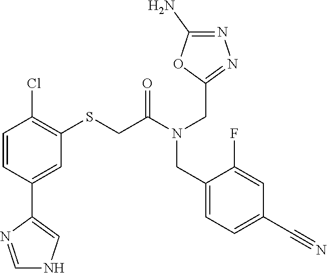 [SMILES: N#Cc1ccc(CN(Cc2nnc(N)o2)C(=O)CSc2cc(-c3c[nH]cn3)ccc2Cl)c(F)c1]

A solution of lithium 2-[2-chloro-5-(1H-imidazol-4-yl)phenyl]sulfanylacetate (71 mg, 0.258 mmol), 1-[bis(dimethylamino)methylidene]-1H-[1,2,3]triazolo[4,5-b]pyridin-1-ium 3-oxide hexafluorophosphate (107 mg, 0.282 mmol), DMF-Anhydrous (1.1187 mL), N-ethyl-N-isopropyl-propan-2-amine (0.082 mL, 0.469 mmol) and 4-[([(5-amino-1,3,4-oxadiazol-2-yl)methyl]amino)methyl]-3-fluoro-benzonitrile (58 mg, 0.235 mmol) was prepared without stepwise addition. The reaction was then stirred 2 hours followed by purification by High pH preparative HPLC (Early Elute method) to provide N-[(5-amino-1,3,4-oxadiazol-2-yl)methyl]-2-[2-chloro-5-(1H-imidazol-4-yl)phenyl]sulfanyl-N-[(4-cyano-2-fluoro-phenyl)methyl]acetamide (30 mg, 0.0602 mmol, 26% Yield) as an orange-glass powder. ¹H-NMR (500 MHz, DMSO-d₆) δ 12.29 (s, 1H), 8.02-7.26 (m, 8H), 7.16-6.94 (m, 2H), 5.02-4.81 (m, 2H), 4.73-4.50 (m, 2H), 4.43-4.18 (m, 2H). (ESI⁺)—LCMS Method A r.t.=0.71 min (M+H)⁺ 498.0.

### Example 11—Synthesis of N-[(5-Amino-1,3,4-oxadiazol-2-yl)methyl]-2-[2-chloro-5-(2-hydroxyethyl)phenyl]sulfanyl-N-[(4-cyano-2-fluoro-phenyl)methyl]acetamide (Compound No. 63)

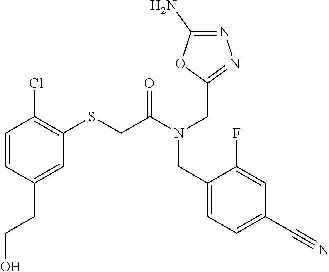 [SMILES: N#Cc1ccc(CN(Cc2nnc(N)o2)C(=O)CSc2cc(CCO)ccc2Cl)c(F)c1]

### 2-(3-Bromo-4-chlorophenyl)ethoxy-tert-butyldimethylsilane

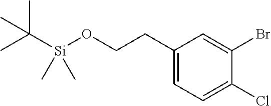 [SMILES: CC(C)(C)[Si](C)(C)OCCc1ccc(Cl)c(Br)c1]

To a stirred solution of 2-(3-bromo-4-chloro-phenyl)ethanol (444 mg, 1.89 mmol) in anhydrous THE (9.4 mL), was added 1H-imidazole (308 mg, 4.53 mmol) and tert-butyl(chloro)dimethylsilane (341 mg, 2.26 mmol). The mixture was left stirring at RT for 2 hours. The mixture was then concentrated in vacuo and the residue was purified by Normal phase column chromatography Method 1 to obtain 2-(3-bromo-4-chlorophenyl)ethoxy-tert-butyldimethylsilane (398 mg, 1.14 mmol, 60% yield) as a colourless oil.

### Methyl 2-[5-[2-[tert-butyl(dimethyl)silyl]oxyethyl]-2-chloro-phenyl]sulfanylacetate

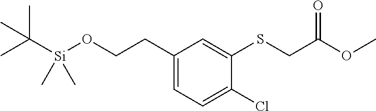 [SMILES: COC(=O)CSc1cc(CCO[Si](C)(C)C(C)(C)C)ccc1Cl]

To a pressure vial was added 2-(3-bromo-4-chloro-phenyl)ethoxy-tert-butyl-dimethyl-silane (398 mg, 1.14 mmol), methyl sulfanylacetate (0.12 mL, 1.37 mmol), disodium carbonate (302 mg, 2.84 mmol) and 1,4-Dioxane (3.4 mL). The reaction vial was purged with nitrogen for 5 minutes and then XantPhos Pd G3 (108 mg, 0.114 mmol) was added and the reaction was purged with nitrogen for 1 further minute. The reaction was placed in a pre-heated hot plate at 100° C. and stirred for 5 hours. The mixture was then diluted with water (30 mL), extracted with ethyl acetate (2×30 mL). The organic extracts were dried over Na₂SO₄, filtered and the resulting residue was then purified by Normal phase column chromatography Method 1 to obtain methyl 2-[5-[2-[tert-butyl(dimethyl)silyl]oxyethyl]-2-chloro-phenyl]sulfanylacetate (60 mg, 0.160 mmol, 14% Yield) as a colourless oil. ¹H NMR (500 MHz, DMSO-d6) δ 7.64 (d, J=2.0 Hz, 1H), 7.52 (d, J=8.2 Hz, 1H), 7.26 (dd, J=8.2, 2.0 Hz, 1H), 3.76 (t, J=6.3 Hz, 2H), 2.74 (t, J=6.3 Hz, 2H), 0.81 (s, 9H), −0.07 (s, 6H).

### 2-[5-[2-[tert-Butyl(dimethyl)silyl]oxyethyl]-2-chloro-phenyl]sulfanylacetic acid

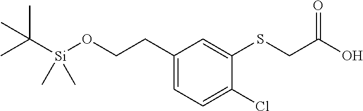 [SMILES: CC(C)(C)[Si](C)(C)OCCc1ccc(Cl)c(SCC(=O)O)c1]

To methyl 2-[5-[2-[tert-butyl(dimethyl)silyl]oxyethyl]-2-chloro-phenyl]sulfanylacetate (55 mg, 0.147 mmol) in Methanol (1 mL) and Water (0.3 mL), was added 2 M lithium hydroxide (0.11 mL, 0.220 mmol). The mixture was left stirring at RT for 1 hour. The mixture was then acidified using 2M HCl (1 ml), the mixture was then diluted with water (30 ml), extracted with ethyl acetate (2×30 ml). The combined organic extracts were dried over Na₂SO₄, filtered and concentrated to afford the title compound 2-[5-[2-[tert-butyl(dimethyl)silyl]oxyethyl]-2-chloro-phenyl]sulfanylacetic acid (27 mg, 0.0748 mmol, 51% Yield) as a white powder. (ESI⁺)—LCMS Method B rt.=1.45 min (ESI+) 459.1.

### N-[(5-Amino-1,3,4-oxadiazol-2-yl)methyl]-2-[5-[2-[tert-butyl(dimethyl)silyl]oxyethyl]-2-chloro-phenyl]sulfanyl-N-[(4-cyano-2-fluoro-phenyl)methyl]acetamide

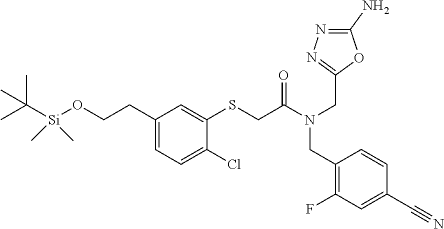 [SMILES: CC(C)(C)[Si](C)(C)OCCc1ccc(Cl)c(SCC(=O)N(Cc2nnc(N)o2)Cc2ccc(C#N)cc2F)c1]

To a solution of 2-[5-[2-[tert-butyl(dimethyl)silyl]oxyethyl]-2-chloro-phenyl]sulfanylacetic acid (27 mg, 0.0748 mmol) and N-ethyl-N-isopropyl-propan-2-amine (0.026 mL, 0.150 mmol) in DMF (0.3 mL) was added 4-[([(5-amino-1,3,4-oxadiazol-2-yl)methyl]amino)methyl]-3-fluoro-benzonitrile (19 mg, 0.0785 mmol) and 1-[bis(dimethylamino)methylidene]-1H-[1,2,3]triazolo[4,5-b]pyridin-1-ium 3-oxide hexafluorophosphate (34 mg, 0.0898 mmol) and the reaction stirred at room temperature for 2 h. The mixture was then diluted with water (30 mL), extracted with ethyl acetate (2×30 mL). The combined organic extracts were dried over Na₂SO₄, filtered and concentrated to obtain N-[(5-amino-1,3,4-oxadiazol-2-yl)methyl]-2-[5-[2-[tert-butyl(dimethyl)silyl]oxyethyl]-2-chloro-phenyl]sulfanyl-N-[(4-cyano-2-fluoro-phenyl)methyl]acetamide (35 mg, 0.0593 mmol, 79% Yield) as a yellow oil. (ESI⁺)—LCMS Method B rt.=1.48 min (ESI+) 590.3.

### N-[(5-Amino-1,3,4-oxadiazol-2-yl)methyl]-2-[2-chloro-5-(2-hydroxyethyl)phenyl]sulfanyl-N-[(4-cyano-2-fluoro-phenyl)methyl]acetamide (Compound No. 63)

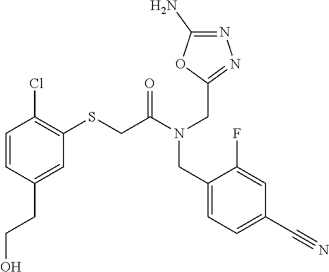 [SMILES: N#Cc1ccc(CN(Cc2nnc(N)o2)C(=O)CSc2cc(CCO)ccc2Cl)c(F)c1]

To N-[(5-amino-1,3,4-oxadiazol-2-yl)methyl]-2-[5-[2-[tert-butyl(dimethyl)silyl]oxyethyl]-2-chloro-phenyl]sulfanyl-N-[(4-cyano-2-fluoro-phenyl)methyl]acetamide (35 mg, 0.0593 mmol) in THE anhydrous (1 mL), was added 1 M N,N,N-tributylbutan-1-aminium fluoride in THE (0.071 mL, 0.0712 mmol). The mixture was stirred at RT for 3 h. The mixture was then concentrated in vacuo and purified by Low pH preparative HPLC (Standard method) to obtain N-[(5-amino-1,3,4-oxadiazol-2-yl)methyl]-2-[2-chloro-5-(2-hydroxyethyl)phenyl]sulfanyl-N-[(4-cyano-2-fluoro-phenyl)methyl]acetamide (15 mg, 0.0321 mmol, 54% Yield) as an off-white powder. ¹⁻NMR (500 MHz, DMSO-d6) δ 7.92-7.78 (m, 1H), 7.72-7.61 (m, 1H), 7.54-7.37 (m, 1H), 7.36-7.31 (m, 1H), 7.26-7.19 (m, 1H), 7.13-6.96 (m, 3H), 5.00-4.83 (m, 2H), 4.73-4.54 (m, 3H), 4.34-4.13 (m, 2H), 3.63-3.54 (m, 2H), 2.74-2.66 (m, 2H). (ESI⁺)—LCMS Method H rt.=1.41 min (ESI+) 476.2.

### Example 12—Synthesis of N-[(5-Amino-1,3,4-oxadiazol-2-yl)methyl]-N-[(4-cyano-2-fluoro-phenyl)methyl]-2-(2-ethyl-3,5-difluoro-phenoxy)acetamide (Compound No. 64)

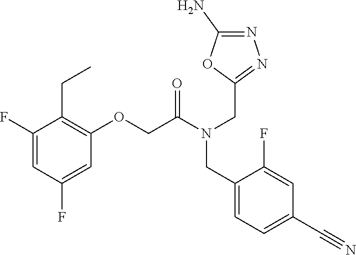 [SMILES: CCc1c(F)cc(F)cc1OCC(=O)N(Cc1nnc(N)o1)Cc1ccc(C#N)cc1F]

### Methyl 2-(2-bromo-3,5-difluoro-phenoxy)acetate

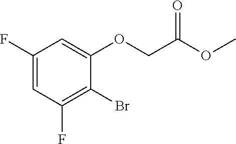

Methyl bromoacetate (0.34 mL, 3.59 mmol) was added to a suspension of 2-bromo-3,5-difluorophenol (500 mg, 2.39 mmol) and potassium dicarbonate (660 mg, 4.35 mmol) in DMF (5 mL) and stirred at 80° C. for 1.5 hours. The reaction was cooled to room temperature and diluted with water (20 mL). The product was then extracted with EtOAc (2×20 mL). The organic layers were combined, washed with brine, dried with MgSO₄ and filtered. The filtrate was then concentrated in vacuo to give methyl 2-(2-bromo-3,5-difluorophenoxy)acetate (90%) (580 mg, 2.06 mmol, 78% Yield). ¹H-NMR (400 MHz, DMSO-d6) δ 7.07 (td J=9.1, 2.7 Hz, 1H), 7.00 (dt, J=10.9, 2.2 Hz, 1H), 5.00 (s, 2H), 3.71 (s, 3H). (ESI⁺)—LCMS Method B rt=1.21.

### Methyl 2-(3,5-difluoro-2-vinyl-phenoxy)acetate

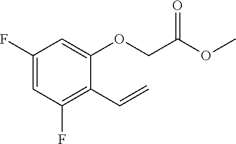 [SMILES: C=Cc1c(F)cc(F)cc1OCC(=O)OC]

[1,1-Bis(diphenylphosphino)ferrocene]palladium(II) chloride dichloromethane (36 mg, 0.04 mmol) was added to a solution of methyl 2-(2-bromo-3,5-difluorophenoxy)acetate (250 mg, 0.89 mmol), potassium trifluoro(vinyl)borate (169 mg, 1.78 mmol) and potassium dicarbonate (307 mg, 2.22 mmol) in 1,2-dimethoxyethane (1.75 mL) and water (0.59 mL) under nitrogen the stirred and heated in a microwave for 1 hour. The reaction was cooled, diluted with water (15 mL) and EtOAc (15 mL) and filtered. The layers were separated and the product was then extracted with EtOAc (2×15 mL). The organic layers were combined, washed with brine (3×15 mL), dried with MgSO₄ and filtered. The solvent was evaporated to give a dark brown oily residue. The crude product was purified by Normal phase column chromatography Method 1. The desired fractions were combined and concentrated in vacuo to give methyl 2-(2-ethenyl-3,5-difluorophenoxy)acetate (95%). (41 mg, 0.17 mmol, 19% Yield). ¹H-NMR (400 MHz, Methanol-d4) δ 6.80 (dd, J=18.1, 12.1 Hz, 1H), 6.64-6.56 (m, 2H), 6.05 (dt, J=18.1, 1.6 Hz, 1H), 5.47 (dt, J=12.1, 1.6 Hz, 1H), 4.80 (s, 2H), 3.81 (s, 3H). (ESI⁺)—LCMS Method B rt.=1.27 (ESI⁺) 228.90.

### Methyl 2-(2-ethyl-3,5-difluorophenoxy)acetate

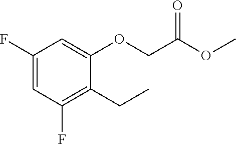 [SMILES: CCc1c(F)cc(F)cc1OCC(=O)OC]

Palladium on activated carbon (10% palladium) (5.1 mg, 0.05 mmol) was added to a solution of methyl 2-(2-ethenyl-3,5-difluorophenoxy)acetate (55 mg, 0.24 mmol) in EtOH (7.33 mL) under nitrogen and was stirred under a hydrogen atmosphere at room temperature for 3.5 hours. The reaction was filtered and the filtrate concentrated in vacuo to give methyl 2-(2-ethyl-3,5-difluorophenoxy)acetate (90%) (39 mg, 0.15 mmol, 63% Yield). ¹H-NMR (400 MHz, Chloroform-d) 6 (td, J=9.3, 2.3 Hz, 1H), 6.30-3.23 (m, 1H), 4.63 (s, 2H), 3.81 (s, 3H) 2.71-2.62 (m, 2H), 1.14 (t, J=7.5 Hz, 3H). (ESI⁺)—LCMS Method B rt.=1.30.

### 2-(2-Ethyl-3,5-difluorophenoxy)acetic acid

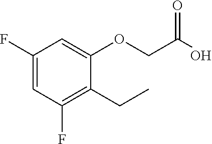

A solution of methyl 2-(2-ethyl-3,5-difluorophenoxy)acetate (38 mg, 0.15 mmol) and 2M lithium hydroxide (0.09 mL, 0.18 mmol) in water (0.18 mL), THF (0.18 mL) and MeOH (0.18 mL) was stirred at room temperature for 5 hours. The solvent was evaporated and the solid dissolved in water (15 mL). The pH was then adjusted to pH 1 with 6M HCl and the product extracted with EtOAc (3×20 mL). The organic layers were combined, washed with brine (2×15 mL), dried with MgSO₄ and filtered. The filtrated was concentrated in vacuo to give 2-(2-ethyl-3,5-difluorophenoxy)acetic acid (90%) (23 mg, 0.11 mmol, 71% Yield). ¹H-NMR (500 MHz, Chloroform-d) δ 6.48 (td, J=9.3, 2.3 Hz, 1H), 6.33-6.25 (m, 1H), 4.68 (s, 2H), 2.70-2.63 (m, 2H), 1.14 (t, J=7.5 Hz, 3H). (ESI⁻)—LCMS Method B rt.=1.15 (ESI−) 215.15.

### N-[(5-Amino-1,3,4-oxadiazol-2-yl)methyl]-N-[(4-cyano-2-fluoro-phenyl)methyl]-2-(2-ethyl-3,5-difluoro-phenoxy)acetamide (Compound No. 64)

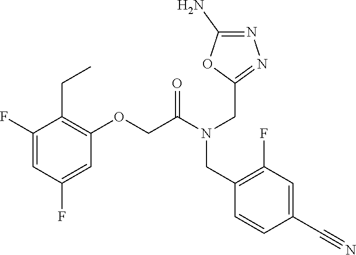 [SMILES: CCc1c(F)cc(F)cc1OCC(=O)N(Cc1nnc(N)o1)Cc1ccc(C#N)cc1F]

N-ethyl-N-isopropyl-propan-2-amine (0.03 mL, 0.19 mmol) was added to a solution of 2-(2-ethyl-3,5-difluorophenoxy)acetic acid (20 mg, 0.09 mmol), 4-[([(5-amino-1,3,4-oxadiazol-2-yl)methyl]amino)methyl]-3-fluoro-benzonitrile (23 mg, 0.09 mmol) and 1-[bis(dimethylamino)methylidene]-1H-[1,2,3]triazolo[4,5-b]pyridin-1-ium 3-oxide hexafluorophosphate (39 mg, 0.10 mmol) in anhydrous DMF (2 mL) and stirred at room temperature for 19 hours. The solvent was evaporated and the crude product was purified by Low pH preparative HPLC (Early elute method). The desired fractions were combined and concentrated in vacuo to give N-[(5-amino-1,3,4-oxadiazol-2-yl)methyl]-N-[(4-cyano-2-fluoro-phenyl)methyl]-2-(2-ethyl-3,5-difluoro-phenoxy)acetamide (98%) (4.5 mg, 9.81 μmol, 11% Yield). ¹H-NMR (400 MHz, Methanol-d4) δ 7.68-7.45 (m, 3H), 6.66-6.48 (m, 2H), 5.11 (d, J=34.1 Hz, 2H), 4.93 (s, 1H), 4.84 (s, 1H), 4.75 (d, J=39.8 Hz, 2H), 2.65 (dq, J=41.0, 7.2 Hz, 2H), 1.13 (dt, J=23.2, 7.5 Hz, 3H). (ESI⁺)—LCMS Method H rt.=4.05 (ESI⁺) 446.1.

### Example 13—Synthesis of 2-(2-Chlorophenyl)sulfanyl-N-[(4-cyanophenyl)methyl]-N-[(5-methyl-1,3,4-oxadiazol-2-yl)methyl]acetamide (Compound No. 65)

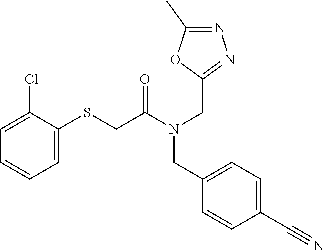 [SMILES: Cc1nnc(CN(Cc2ccc(C#N)cc2)C(=O)CSc2ccccc2Cl)o1]

### 4-[([(5-Methyl-1,3,4-oxadiazol-2-yl)methyl]amino)methyl]benzonitrile

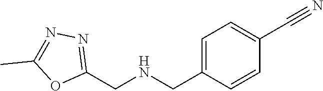 [SMILES: Cc1nnc(CNCc2ccc(C#N)cc2)o1]

To a solution of (5-methyl-1,3,4-oxadiazol-2-yl)methanamine hydrochloride (299 mg, 2.00 mmol) in DCM (10 mL) was added N-ethyl-N-isopropyl-propan-2-amine (1.0 mL, 6.00 mmol), 4-(bromomethyl)benzonitrile (0.38 mL, 2.00 mmol) and N,N-dimethylpyridin-4-amine (244 mg, 2.00 mmol). The resultant mixture was stirred at room temperature for 4 mins. The reaction was diluted with Na₂CO₃ (20 mL) and extracted with a 3:1 solution of CHCl₃:IPA (3×20 mL). The organic fractions were dried over Na₂SO₄, filtered and concentrated in vacuo. The product was purified by Normal phase column chromatography Method 2 to afford the title compound (26 mg, 0.0945 mmol, 4.7% yield) as a white solid. ¹H-NMR (500 MHz, Chloroform-d) δ 7.63-7.58 (m, 2H), 7.46 (d, J=8.4 Hz, 2H), 3.97 (s, 2H), 3.91 (s, 2H), 2.51 (s, 3H). (ESI⁺)—LCMS Method D, rt=1.19 mins, [M+H]⁺=229.1.

### 2-(2-Chlorophenyl)sulfanyl-N-[(4-cyanophenyl)methyl]-N-[(5-methyl-1,3,4-oxadiazol-2-yl)methyl]acetamide (Compound No. 65)

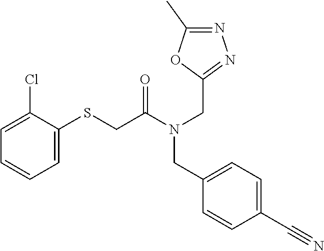 [SMILES: Cc1nnc(CN(Cc2ccc(C#N)cc2)C(=O)CSc2ccccc2Cl)o1]

2-(2-Chlorophenyl)sulfanylacetic acid (23 mg, 0.114 mmol), 4-[([(5-methyl-1,3,4-oxadiazol-2-yl)methyl]amino)methyl]benzonitrile (26 mg, 0.114 mmol) and N-ethyl-N-isopropyl-propan-2-amine (0.022 mL, 0.125 mmol) were dissolved in anhydrous DMF (0.50 mL). Without delay, 1-[bis(dimethylamino)methylidene]-1H-[1,2,3]triazolo[4,5-b]pyridin-1-ium 3-oxide hexafluorophosphate (48 mg, 0.125 mmol) was added and the reaction was stirred at room temperature for 2 hours. The reaction was purified by High pH preparative HPLC (Early Elute method) to afford the title compound (31 mg, 0.0751 mmol, 66% yield) as a white solid. ¹H-NMR (500 MHz, Methanol-d4) δ 7.73 (d, J=8.3 Hz, 1H), 7.64 (d, J=8.3 Hz, 1H), 7.52-7.21 (m, 6H), 4.97 (s, 2H), 4.74 (d, J=5.8 Hz, 2H), 4.19-3.98 (m, 2H), 2.47 (s, 3H). (ESI⁺)—LCMS Method A, rt=3.05 mins, [M+H]⁺=394.2.

### Example 14—Synthesis of 2-(2-Chlorophenyl)sulfanyl-N-[(4-cyanophenyl)methyl]-N-[(5-hydroxy-1,3,4-oxadiazol-2-yl)methyl]acetamide (Compound No. 66)

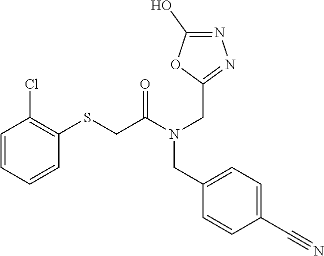 [SMILES: N#Cc1ccc(CN(Cc2nnc(O)o2)C(=O)CSc2ccccc2Cl)cc1]

2-(2-Chlorophenyl)sulfanyl-N-[(4-cyanophenyl)methyl]-N-(2-hydrazino-2-oxo-ethyl)acetamide was synthesised from 2-[[2-(2-chlorophenyl)sulfanylacetyl]-[(4-cyanophenyl)methyl]amino]acetic acid by analogous methods as described in Example 2.

To a solution of 2-(2-chlorophenyl)sulfanyl-N-[(4-cyanophenyl)methyl]-N-(2-hydrazino-2-oxo-ethyl)acetamide (35 mg, 0.0900 mmol) in anhydrous 1,4-Dioxane (1.70 mL) was added bis(trichloromethyl) carbonate (40 mg, 0.135 mmol) and the reaction stirred for 2 hours. The reaction was purified by High pH preparative HPLC (Early Elute method) to afford the title compound (8.0 mg, 0.0193 mmol, 21% yield) as a white solid. ¹H-NMR (400 MHz, Methanol-d4) δ 7.69 (dd, J=34.6, 8.3 Hz, 2H), 7.52-7.34 (m, 4H), 7.25 (dddd, J=15.1, 13.5, 6.7, 2.7 Hz, 2H), 4.93 (s, 1H), 4.72 (s, 1H), 4.58 (d, J=80.9 Hz, 2H), 4.06 (d, J=59.9 Hz, 2H). (ESI⁺)—LCMS Method H, rt=2.67 mins, [M+H]⁺=415.1.

### Example 15—Synthesis of N-[[5-(Aminomethyl)-1,3,4-oxadiazol-2-yl]methyl]-2-(2-chlorophenyl)sulfanyl-N-[(4-cyanophenyl)methyl]acetamide (Compound No. 67)

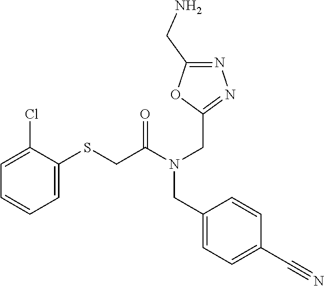 [SMILES: N#Cc1ccc(CN(Cc2nnc(CN)o2)C(=O)CSc2ccccc2Cl)cc1]

### tert-Butyl N-[2-[2-[2-[[2-(2-chlorophenyl)sulfanylacetyl]-[(4-cyanophenyl)methyl]amino]acetyl]hydrazino]-2-oxo-ethyl]carbamate

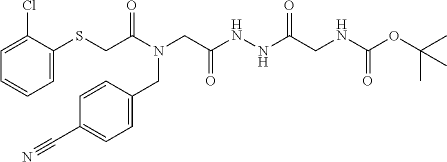 [SMILES: CC(C)(C)OC(=O)NCC(=O)NNC(=O)CN(Cc1ccc(C#N)cc1)C(=O)CSc1ccccc1Cl]

To a solution of 2-[[2-(2-chlorophenyl)sulfanylacetyl]-[(4-cyanophenyl)methyl]amino]acetic acid (93%, 54 mg, 0.134 mmol) and N-ethyl-N-isopropyl-propan-2-amine (0.047 mL, 0.268 mmol) in DMF (0.65 mL) was added tert-butyl N-(2-hydrazino-2-oxo-ethyl)carbamate (30 mg, 0.161 mmol) and 1-[bis(dimethylamino)methylidene]-1H-[1,2,3]triazolo[4,5-b]pyridin-1-ium 3-oxide hexafluorophosphate (56 mg, 0.147 mmol) and the reaction stirred for 18 hours. The reaction was purified by Normal phase column chromatography Method 2. The reaction was concentrated in vacuo and the crude residue was dissolved in MeOH (10 mL) and purified by SCX-2 cartridge (5 g), eluting with MeOH (10 mL) and then 2M NH₃ in MeOH (15 mL). The ammonia solution was concentrated in vacuo to afford the title compound (84 mg, 0.134 mmol, 100% yield) as a white solid. (ESI⁺)—LCMS Method D, rt=1.46 mins, [M+H]⁺=546.3.

### N-[[5-(Aminomethyl)-1,3,4-oxadiazol-2-yl]methyl]-2-(2-chlorophenyl)sulfanyl-N-[(4-cyanophenyl)methyl]acetamide (Compound No. 67)

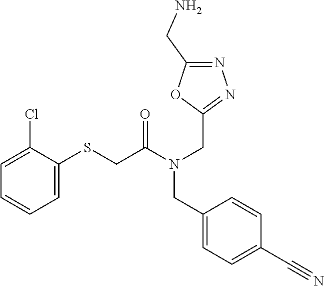 [SMILES: N#Cc1ccc(CN(Cc2nnc(CN)o2)C(=O)CSc2ccccc2Cl)cc1]

To a solution of tert-butyl N-[2-[2-[2-[[2-(2-chlorophenyl)sulfanylacetyl]-[(4-cyanophenyl)methyl]amino]acetyl]hydrazino]-2-oxo-ethyl]carbamate (87%, 84 mg, 0.134 mmol) in DCM (1.70 mL) was added methoxycarbonyl-(triethylammonio)sulfonyl-azanide (48 mg, 0.201 mmol) and the reaction stirred for 18 hours. The reaction was purified by High pH preparative HPLC (Early Elute method) to afford an intermediate, which was dissolved in DCM (5 mL) and TFA (1 mL) was added. The reaction was stirred for 1 h, and volatiles removed under reduced pressure. The residue was dissolved in MeOH (5 mL) and put on an SCX-2 cartridge (5 g), the cartridge was washed with MeOH (20 mL) the product was released using MeOH/NH₃ (20 mL). Solvents were removed under reduced pressure to afford the title compound (87%, 84 mg, 0.134 mmol) as a white solid. ¹H-NMR (500 MHz, Methanol-d4) δ 7.73 (d, J=8.3 Hz, 1H), 7.65 (d, J=8.2 Hz, 1H), 7.54-7.33 (m, 4H), 7.33-7.17 (m, 2H), 4.99 (d, J=6.5 Hz, 2H), 4.75 (d, J=17.1 Hz, 2H), 4.10 (dd, J=75.3, 9.5 Hz, 1H), 3.96 (d, J=6.3 Hz, 2H), 3.35 (s, 1H). (ESI⁺)—LCMS Method H, rt=3.63 mins, [M+H]⁺=428.1.

### Example 16—Synthesis of 2-(2-Chlorophenyl)sulfanyl-N-[(4-cyanophenyl)methyl]-N-[(3-methyl-1,2,4-oxadiazol-5-yl)methyl]acetamide (Compound No. 68)

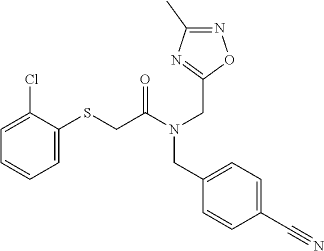 [SMILES: Cc1noc(CN(Cc2ccc(C#N)cc2)C(=O)CSc2ccccc2Cl)n1]

### 4-[([(3-Methyl-1,2,4-oxadiazol-5-yl)methyl]amino)methyl]benzonitrile

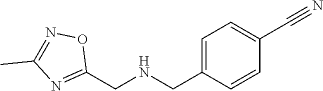 [SMILES: Cc1noc(CNCc2ccc(C#N)cc2)n1]

To a sealed tube was added 4-(aminomethyl)benzonitrile (0.078 mL, 0.757 mmol), 5-(chloromethyl)-3-methyl-1,2,4-oxadiazole (0.14 mL, 1.13 mmol), dipotassium carbonate (209 mg, 1.51 mmol) and DMF (2 mL). The reaction was sealed and heated to 80° C. and stirred overnight. The reaction was cooled to room temperature, diluted with H₂O (20 mL) and extracted with EtOAc (2×50 mL) and the combined organic extracts were dried (Na₂SO₄) and concentrated in vacuo to afford the crude residue, which was purified by High pH preparative HPLC to afford 4-[([(3-methyl-1,2,4-oxadiazol-5-yl)methyl]amino)methyl]benzonitrile (44 mg, 24% yield) as a colourless oil. ¹H-NMR (500 MHz, Chloroform-d) δ 7.65-7.61 (m, 2H), 7.48 (d, J=8.4 Hz, 2H), 4.02 (d, J=7.2 Hz, 2H), 3.94 (d, J=5.9 Hz, 2H), 2.41 (s, 3H). (ESI⁺)—LCMS Method D, rt=1.30 mins, [M+H]⁺=229.2.

### 2-(2-Chlorophenyl)sulfanyl-N-[(4-cyanophenyl)methyl]-N-[(3-methyl-1,2,4-oxadiazol-5-yl)methyl]acetamide (Compound No. 68)

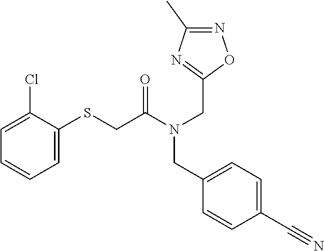 [SMILES: Cc1noc(CN(Cc2ccc(C#N)cc2)C(=O)CSc2ccccc2Cl)n1]

2-(2-Chlorophenyl)sulfanylacetic acid (32 mg, 0.158 mmol), N-ethyl-N-isopropyl-propan-2-amine (0.030 mL, 0.174 mmol) and 4-[([(3-methyl-1,2,4-oxadiazol-5-yl)methyl]amino)methyl]benzonitrile (42 mg, 0.174 mmol) were dissolved in DMF-Anhydrous (1.1429 mL). Without delay, 1-[bis(dimethylamino)methylidene]-1H-[1,2,3]triazolo[4,5-b]pyridin-1-ium 3-oxide hexafluorophosphate (66 mg, 0.174 mmol) was added and the reaction was stirred at room temperature for 2 hours. The reaction was purified by High pH preparative HPLC (Early Elute method) to afford 2-(2-chlorophenyl)sulfanyl-N-[(4-cyanophenyl)methyl]-N-[(3-methyl-1,2,4-oxadiazol-5-yl)methyl]acetamide (34 mg, 0.0815 mmol, 52% yield) as a colourless solid. ¹H-NMR (500 MHz, Methanol-d4) δ 9.32-9.17 (m, 2H), 9.09-8.76 (m, 6H), 6.60-6.53 (m, 2H), 6.32-6.27 (m, 2H), 5.72-5.55 (m, 2H), 3.90-3.87 (m, 3H). (ESI⁺)—LCMS Method C, rt=4.23 mins, [M+H]⁺=412.90.

### Example 17—Synthesis of N-[(5-Amino-1,3,4-thiadiazol-2-yl)methyl]-2-(2-chlorophenyl)sulfanyl-N-[(4-cyanophenyl)methyl]acetamide (Compound No. 69)

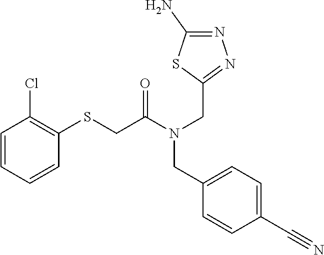 [SMILES: N#Cc1ccc(CN(Cc2nnc(N)s2)C(=O)CSc2ccccc2Cl)cc1]

2-[[2-(2-chlorophenyl)sulfanylacetyl]-[(4-cyanophenyl)methyl]amino]acetic acid (220 mg, 0.587 mmol) and hydrazinecarbothioamide (59 mg, 0.646 mmol) were dissolved in phosphoryl trichloride (0.55 mL, 5.87 mmol) and the reaction was stirred at 80° C. for 5 hours. The reaction was diluted with DCM, and cooled to 0° C. K₂CO₃ was carefully added with stirring until pH=10. The residue was extracted up in DCM (3×10 ml) and the organics washed with 2×10 ml water then 1×10 ml saturated brine solution. The organics were then separated and dried (MgSO₄) before concentration to dryness. The reaction was then purified by High pH preparative HPLC (Early Elute method) to afford the title compound (9.0 mg, 0.021 mmol, 3.6% Yield) as an off white solid. ¹H-NMR (400 MHz, Methanol-d4) δ 7.67 (dd, J=37.0, 8.3 Hz, 2H), 7.54-7.18 (m, 6H), 4.89 (d, J=15.3 Hz, 2H), 4.65 (d, J=23.9 Hz, 2H), 4.06 (d, J=72.4 Hz, 2H). (ESI⁺)—LCMS Method H, rt=3.80 mins, [M+H]⁺=430.0.

### Example 18—Synthesis of N-[(5-Amino-4H-1,2,4-triazol-3-yl)methyl]-N-[(4-cyanophenyl)methyl]-2-(2-ethylphenyl)sulfanyl-acetamide (Compound No. 70)

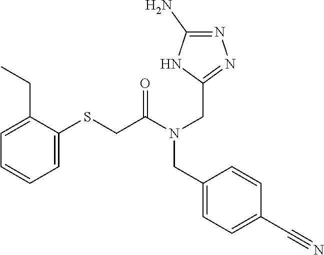 [SMILES: CCc1ccccc1SCC(=O)N(Cc1ccc(C#N)cc1)Cc1nnc(N)[nH]1]

### N-[(4-Cyanophenyl)methyl]-2-(2-ethylphenyl)sulfanyl-N-(2-hydrazino-2-oxo-ethyl)acetamide

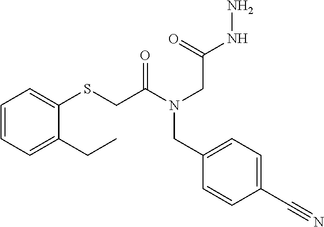 [SMILES: CCc1ccccc1SCC(=O)N(CC(=O)NN)Cc1ccc(C#N)cc1]

To a solution of 2-[(4-cyanophenyl)methyl-[2-(2-ethylphenyl)sulfanylacetyl]amino]acetic acid (175 mg, 0.475 mmol) and N-ethyl-N-isopropyl-propan-2-amine (0.17 mL, 0.950 mmol) in DMF (2.7891 mL) was added tert-butyl N-aminocarbamate (94 mg, 0.712 mmol) and 1-[bis(dimethylamino)methylidene]-1H-[1,2,3]triazolo[4,5-b]pyridin-1-ium 3-oxide hexafluorophosphate (217 mg, 0.570 mmol), and the reaction stirred for 3 hours. The reaction was purified by High pH preparative HPLC to afford the BOC protected intermediate, which was dissolved in DCM (2 mL) and 2,2,2-trifluoroacetic acid (0.35 mL, 4.75 mmol) was added and the reaction stirred at RT for 2 hours. The reaction was concentrated in vacuo and azeotroped with DCM (2×20 mL) to afford the title compound (160 mg, 0.251 mmol, 53% Yield) as a pale yellow oil. (ESI⁺)—LCMS Method D, rt=1.47 mins, [M+H]⁺=383.2.

### N-[(5-Amino-4H-1,2,4-triazol-3-yl)methyl]-N-[(4-cyanophenyl)methyl]-2-(2-ethylphenyl)sulfanyl-acetamide (Compound No. 70)

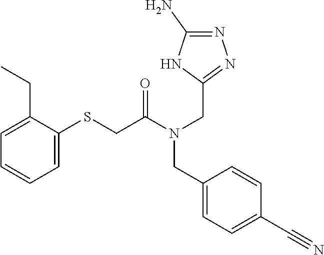 [SMILES: CCc1ccccc1SCC(=O)N(Cc1ccc(C#N)cc1)Cc1nnc(N)[nH]1]

Triethylamine (0.045 mL, 0.322 mmol) was added to a suspension of N-[(4-cyanophenyl)methyl]-2-(2-ethylphenyl)sulfanyl-N-(2-hydrazino-2-oxo-ethyl)acetamide (41 mg, 0.107 mmol), in IPA (535.98 uL) and 2-methylisothiourea;sulfurous acid (14 mg, 0.0536 mmol). The resulting mixture was stirred at 130° C. for 15 hours. The reaction was purified by High pH preparative HPLC (Early Elute method) to afford the title compound (4.5 mg, 0.0106 mmol, 9.9% Yield) as an off white solid. ¹H-NMR (400 MHz, Methanol-d4) δ 7.72-7.62 (m, 2H), 7.49-7.31 (m, 3H), 7.29-7.13 (m, 3H), 4.70 (s, 2H), 4.48 (d, J=23.5 Hz, 2H), 3.99 (d, J=103.5 Hz, 2H), 2.83 (q, J=7.5 Hz, 2H), 1.20 (dt, J=22.0, 7.5 Hz, 3H). (ESI⁺)—LCMS Method H, rt=3.68 mins, [M+H]⁺=407.3.

### Example 19—Synthesis of rel-(2R)—N-[(5-amino-1,3,4-oxadiazol-2-yl)methyl]-N-[(4-cyano-2-fluoro-phenyl)methyl]-2-(2-ethyl-3,5-difluoro-phenoxy)propanamide (Compound No. 100)

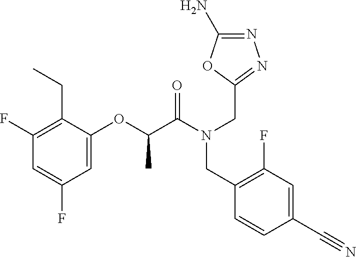 [SMILES: CCc1c(F)cc(F)cc1O[C@H](C)C(=O)N(Cc1nnc(N)o1)Cc1ccc(C#N)cc1F]

### Ethyl 2-(2-bromo-3,5-difluoro-phenoxy)propanoate

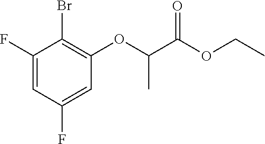 [SMILES: CCOC(=O)C(C)Oc1cc(F)cc(F)c1Br]

2-Bromo-3,5-difluoro-phenol (1.00 g, 4.78 mmol), ethyl 2-bromopropanoate (0.80 mL, 7.18 mmol), dipotassium carbonate (1.32 g, 9.57 mmol) and DMF (25 mL) were stirred at RT for 1.5 hours. The reaction was diluted with water (20 mL), then extracted with TBME (2×20 mL). The organic phases were combined, washed with brine (10 mL), dried and concentrated under vacuum to yield ethyl 2-(2-bromo-3,5-difluoro-phenoxy)propanoate (1.54 g, 4.63 mmol, 97% Yield) as an off white solid. ¹H NMR (400 MHz, Chloroform-d) δ 6.58 (td, J=8.5, 2.6 Hz, 1H), 6.36 (dt, J=10.1, 2.2 Hz, 1H), 4.72 (q, J=6.8 Hz, 1H), 4.24 (q, J=7.1 Hz, 2H), 1.70 (d, J=6.8 Hz, 3H), 1.27 (t, J=7.1 Hz, 3H).

### Ethyl 2-(3,5-difluoro-2-vinyl-phenoxy)propanoate

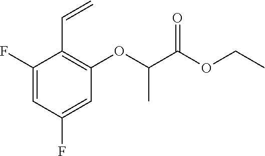 [SMILES: C=Cc1c(F)cc(F)cc1OC(C)C(=O)OCC]

Ethyl 2-(2-bromo-3,5-difluoro-phenoxy)propanoate (65%, 700 mg, 1.47 mmol), potassium vinyltrifluoroborate (296 mg, 2.21 mmol) and Cs₂CO₃ (959 mg, 2.94 mmol) were stirred in 1,4-dioxane (10 mL) and water (2 mL) and degassed with N₂. Bis(triphenylphosphine) palladium(II) dichloride (52 mg, 0.0736 mmol) was added to the reaction solution was further degassed with N₂, then sealed and heated to 80° C. for 3 hours. potassium vinyltrifluoroborate (150 mg, 1.1 mmol) was added and the reaction degassed with N₂, then sealed and heated to 80° C. for a further 13 hours. The reaction was diluted with water (5 mL) and extracted into EtOAc (3×20 mL). Combined organics were washed with brine (10 mL), dried over MgSO₄, filtered and concentrated under vacuum. Purification by Normal phase column chromatography General Method 1 afforded ethyl 2-(3,5-difluoro-2-vinyl-phenoxy)propanoate (56% purity, 573 mg, 1.25 mmol, 74% Yield). ¹H NMR (500 MHz, Chloroform-d) δ 6.79 (dd, J=18.1, 12.1 Hz, 1H), 6.51-6.36 (m, 1H), 6.28 (dt, J=10.4, 2.0 Hz, 1H), 6.00 (dt, J=18.1, 1.6 Hz, 1H), 5.48 (dt, J=12.0, 1.6 Hz, 1H), 4.76-4.63 (m, 1H), 4.27-4.19 (m, 2H), 1.65 (d, J=6.8 Hz, 3H), 1.30-1.22 (m, 3H). LCMS Method A, m/z: 257.1 [M+H]+, (ESI+), RT=4.06.

### Ethyl 2-(2-ethyl-3,5-difluoro-phenoxy)propanoate

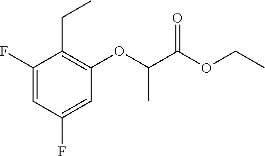 [SMILES: CCOC(=O)C(C)Oc1cc(F)cc(F)c1CC]

Ethyl 2-(3,5-difluoro-2-vinyl-phenoxy)propanoate (56%, 570 mg, 1.25 mmol) was stirred in ethanol (6 mL) and the vessel was evacuated and filled with nitrogen×3. Palladium (10% on carbon, wet basis) (10%, 28 mg, 0.0267 mmol) was added and the vessel evacuated and filled with nitrogen×3, then hydrogen×3. The reaction was stirred under hydrogen for 29 hours. The reaction mixture was filtered through celite washing with ethanol and EtOAc, the filtrate was concentrated under vacuum to yield ethyl 2-(2-ethyl-3,5-difluoro-phenoxy)propanoate (48% purity, 510 mg, 0.948 mmol, 76% Yield). ¹H NMR (400 MHz, Chloroform-d) δ 6.49-6.18 (m, 2H), 4.74-4.63 (m, 1H), 4.28-4.14 (m, 2H), 2.66 (pd, J=7.4, 1.6 Hz, 2H), 1.67-1.58 (m, 3H), 1.31-1.21 (m, 3H), 1.14 (t, J=7.5 Hz, 3H). LCMS Method A: m/z: 259.1 [M+H]+, (ESI+), RT=4.25.

### 2-(2-Ethyl-3,5-difluoro-phenoxy)propanoic acid

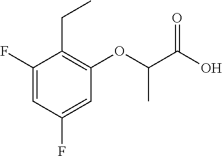 [SMILES: CCc1c(F)cc(F)cc1OC(C)C(=O)O]

Ethyl 2-(2-ethyl-3,5-difluoro-phenoxy)propanoate (56%, 510 mg, 1.11 mmol) was stirred in Acetonitrile (3 mL), 2 M lithium hydroxide (2.5 mL, 5.00 mmol) was added and the reaction stirred for 1 hr at 50° C., then stirred at room temperature for 18 h. The reaction was concentrated under vacuum to remove MeCN. The aqueous remainder was acidified with 2M HCl (3 mL), extracted with EtOAc (4×5 mL), and the organic layers were dried and concentrated. Purification by Normal phase column chromatography General Method 2 afforded 2-(2-ethyl-3,5-difluoro-phenoxy)propanoic acid (176 mg, 0.765 mmol, 69% Yield). ¹H NMR (400 MHz, Chloroform-d) δ 6.45 (td, J=9.3, 2.3 Hz, 1H), 6.28 (dt, J=10.4, 1.9 Hz, 1H), 4.75 (q, J=6.8 Hz, 1H), 2.65 (tq, J=11.6, 5.8 Hz, 2H), 1.69 (d, J=6.8 Hz, 3H), 1.13 (t, J=7.5 Hz, 3H). LCMS Method E: m/z: 229.1 [M−H]−, (ESI−), RT=0.90.

### Ethyl 2-[(4-cyano-2-fluoro-phenyl)methylamino]acetate

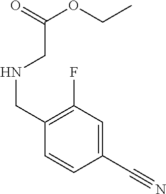 [SMILES: CCOC(=O)CNCc1ccc(C#N)cc1F]

Glycine ethyl ester hydrochloride (6.52 g, 46.7 mmol) and dipotassium carbonate (6.46 g, 46.7 mmol) were suspended in MeCN (100 mL) with stirring then a solution of 4-(bromomethyl)-3-fluorobenzonitrile (5.00 g, 23.4 mmol) in MeCN (75 mL) was added dropwise over 45 mins. The reaction was stirred at room temperature for 18 hours, then filtered, washing with further MeCN, then the filtrate was concentrated and the residue was partitioned between EtOAc (50 mL) and water (50 mL), stirred for 5 mins, then the organics were separated, washed with water (50 mL), dried and concentrated to afford 5.4 g (77% purity, 75% yield). ¹H NMR (400 MHz, Chloroform-d) δ 7.57 (t, J=7.5 Hz, 1H), 7.44 (dd, J=7.9, 1.4 Hz, 1H), 7.33 (dd, J=9.3, 1.5 Hz, 1H), 4.19 (q, J=7.1 Hz, 2H), 4.12 (q, J=7.1 Hz, 1H), 3.92 (s, 2H), 3.41 (s, 2H), 2.04 (s, 1H), 1.26 (dt, J=8.6, 7.2 Hz, 5H). LCMS Method D: m/z: 237 [M+H]+, (ESI+), RT=0.49.

### Ethyl 2-[(4-cyano-2-fluoro-phenyl)methyl-[2-(2-ethyl-3,5-difluoro-phenoxy)propanoyl]amino]acetate

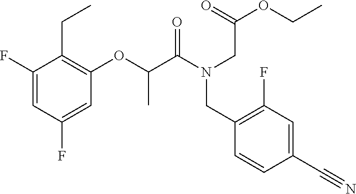 [SMILES: CCOC(=O)CN(Cc1ccc(C#N)cc1F)C(=O)C(C)Oc1cc(F)cc(F)c1CC]

2-(2-Ethyl-3,5-difluoro-phenoxy)propanoic acid (176 mg, 0.765 mmol) and ethyl 2-[(4-cyano-2-fluoro-phenyl)methylamino]acetate (77%, 260 mg, 0.847 mmol) were stirred in DCM (5 mL). N-ethyl-N-isopropyl-propan-2-amine (0.40 mL, 2.29 mmol) and 1-[bis(dimethylamino)methylidene]-1H-[1,2,3]triazolo[4,5-b]pyridin-1-ium 3-oxide hexafluorophosphate (320 mg, 0.842 mmol) were added and the reaction stirred at room temp for 1 hour. The reaction was retreated with 1-[bis(dimethylamino)methylidene]-1H-[1,2,3]triazolo[4,5-b]pyridin-1-ium 3-oxide hexafluorophosphate (60 mg, 0.158 mmol) and stirred for 2 hours. The reaction mixture was diluted with DCM (5 mL) washed with water (2×5 mL), organics dried and concentrated. Purification by Normal phase column chromatography General Method 1 afforded ethyl 2-[(4-cyano-2-fluoro-phenyl)methyl-[2-(2-ethyl-3,5-difluoro-phenoxy)propanoyl]amino]acetate (260 mg, 0.49 mmol, 59% Yield). ¹H NMR (400 MHz, DMSO-d6) δ 7.90-7.73 (m, 1H), 7.64 (dd, J=12.4, 6.8 Hz, 1H), 7.46 (dt, J=38.6, 7.8 Hz, 1H), 6.59 (dd, J=88.8, 11.2 Hz, 2H), 5.49-5.29 (m, 1H), 5.00-4.78 (m, 1H), 4.71-4.51 (m, 1H), 4.49-4.29 (m, 1H), 4.13-4.01 (m, 3H), 2.55 (s, 2H), 1.44 (d, J=6.4 Hz, 3H), 1.14 (t, J=7.1 Hz, 3H), 1.11-0.98 (m, 3H). LCMS Method E: m/z: 449 [M+H]+, (ESI+), RT=1.07.

### 2-[(4-Cyano-2-fluoro-phenyl)methyl-[2-(2-ethyl-3,5-difluoro-phenoxy)propanoyl]amino]acetic acid

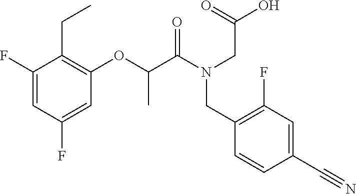 [SMILES: CCc1c(F)cc(F)cc1OC(C)C(=O)N(CC(=O)O)Cc1ccc(C#N)cc1F]

To a stirred solution of ethyl 2-[(4-cyano-2-fluoro-phenyl)methyl-[2-(2-ethyl-3,5-difluoro-phenoxy)propanoyl]amino]acetate (260 mg, 0.45 mmol) in MeCN (1.5 mL) was added 2 M lithium hydroxide (1.5 mL, 3.00 mmol) and the reaction stirred at 50° C. for 2 hours. The reaction mixture was concentrated under vacuum to remove MeCN. The aqueous residue was acidified by addition of 2M HCl (3 mL, 6 mmol) and product extracted with EtOAc (3×5 mL), filtered through MgSO₄ and concentrated under vacuum to yield 2-[(4-cyano-2-fluoro-phenyl)methyl-[2-(2-ethyl-3,5-difluoro-phenoxy)propanoyl]amino]acetic acid (255 mg, 0.491 mmol, 100% Yield). LCMS Method E: m/z: 421 [M+H]+, (ESI+), RT=0.94.

### tert-Butyl N-[[2-[(4-cyano-2-fluoro-phenyl)methyl-[2-(2-ethyl-3,5-difluoro-phenoxy)propanoyl]amino]acetyl]amino]carbamate

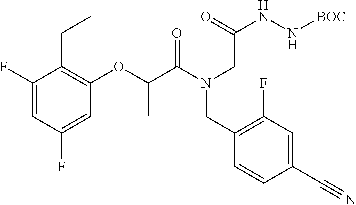 [SMILES: CCc1c(F)cc(F)cc1OC(C)C(=O)N(CC(=O)NNC(=O)OC(C)(C)C)Cc1ccc(C#N)cc1F]

2-[(4-Cyano-2-fluoro-phenyl)methyl-[2-(2-ethyl-3,5-difluoro-phenoxy)propanoyl]amino]acetic acid (81%, 200 mg, 0.385 mmol) and tert-butyl hydrazinecarboxylate (75 mg, 0.578 mmol) were stirred in DCM (2 mL) and DMF (0.5 mL). DIPEA (206 uL, 1.16 mmol) and HATU (160 mg, 0.424 mmol) were added and the reaction stirred at room temperature for 18 h. The reaction was treated with tert-butyl hydrazinecarboxylate (75 mg, 0.578 mmol), HATU (160 mg, 0.424 mmol) and DIPEA (206 uL, 1.16 mmol) and stirred for 1 hour. The reaction was diluted with water (2 mL) and extracted with EtOAc (3×5 mL), organics were washed with brine (5 mL), dried over MgSO₄, filtered and concentrated under vacuum. Purification by Normal phase column chromatography General Method 2 afforded tert-butyl N-[[2-[(4-cyano-2-fluoro-phenyl)methyl-[2-(2-ethyl-3,5-difluoro-phenoxy)propanoyl]amino]acetyl]amino]carbamate (195 mg, 0.339 mmol, 88% Yield). LCMS Method E: m/z: 533.3 [M−H]−, (ESI−), RT=1.01.

### N-[(4-Cyano-2-fluoro-phenyl)methyl]-2-(2-ethyl-3,5-difluoro-phenoxy)-N-(2-hydrazino-2-oxo-ethyl)propanamide

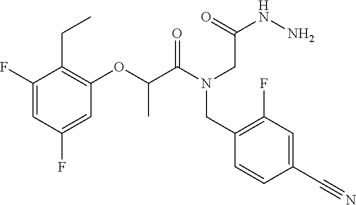 [SMILES: CCc1c(F)cc(F)cc1OC(C)C(=O)N(CC(=O)NN)Cc1ccc(C#N)cc1F]

To a stirred solution of tert-butyl N-[[2-[(4-cyano-2-fluoro-phenyl)methyl-[2-(2-ethyl-3,5-difluoro-phenoxy)propanoyl]amino]acetyl]amino]carbamate (88%, 195 mg, 0.321 mmol) in DCM (2 mL), was added trifluoroacetic acid (123 uL, 1.61 mmol) and the reaction was stirred for 18 h, then concentrated under vacuum and loaded in MeOH onto a conditioned SCX-2 cartridge. Residual TFA was washed off with MeOH, and the product eluted in 2M methanolic ammonia. Product fractions were concentrated to yield N-[(4-cyano-2-fluoro-phenyl)methyl]-2-(2-ethyl-3,5-difluoro-phenoxy)-N-(2-hydrazino-2-oxo-ethyl)propanamide (160 mg, 0.324 mmol, 100% Yield). LCMS Method E: m/z: 435.3 [M+H]+, (ESI+), RT=0.85.

### rel-(2R)—N-[(5-Amino-1,3,4-oxadiazol-2-yl)methyl]-N-[(4-cyano-2-fluoro-phenyl)methyl]-2-(2-ethyl-3,5-difluoro-phenoxy)propanamide (Compound No. 100)

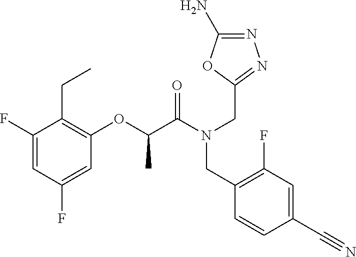 [SMILES: CCc1c(F)cc(F)cc1O[C@H](C)C(=O)N(Cc1nnc(N)o1)Cc1ccc(C#N)cc1F]

To a solution of carbononitridic bromide (27 mg, 0.256 mmol) and N-[(4-cyano-2-fluoro-phenyl)methyl]-2-(2-ethyl-3,5-difluoro-phenoxy)-N-(2-hydrazino-2-oxo-ethyl)propanamide (63%, 160 mg, 0.232 mmol) in 1,4-Dioxane-Anhydrous (4.34 mL) was added sodium hydrogen carbonate (21 mg, 0.255 mmol) and the reaction was stirred at room temperature for 16 h. The reaction mixture was diluted with water (4 mL) and extracted with EtOAc (3×5 mL). The organic extracts were concentrated in vacuo and purified by High pH reverse phase column chromatography to yield N-[(5-amino-1,3,4-oxadiazol-2-yl)methyl]-N-[(4-cyano-2-fluoro-phenyl)methyl]-2-(2-ethyl-3,5-difluoro-phenoxy)propanamide 53 mg containing a mixture of stereoisomers. Chiral separation using: Column: Chiralcel OD-H, 20×250 mm, 5 μm. Mobile phase: 90:10 Heptane:Ethanol, Flow rate: 18 mL/min afforded the title compound at retention time 13.98 mins: rel-(2R)—N-[(5-amino-1,3,4-oxadiazol-2-yl)methyl]-N-[(4-cyano-2-fluoro-phenyl)methyl]-2-(2-ethyl-3,5-difluoro-phenoxy)propanamide (21 mg, 0.0457 mmol, 20% Yield). ¹H NMR (400 MHz, DMSO-d6) δ 7.92-7.76 (m, 1H), 7.68-7.55 (m, 1H), 7.47-7.27 (m, 1H), 7.12-6.94 (m, 2H), 6.80-6.47 (m, 2H), 5.57-5.43 (m, 1H), 5.02-4.78 (m, 2H), 4.70-4.41 (m, 2H), 4.38-4.29 (m, 1H), 3.83-3.71 (m, 1H), 1.58-1.40 (m, 3H), 1.10-0.99 (m, 3H). LCMS Method H: m/z: 460.3 [M+H]+, (ESI+), RT=3.39.

### Example 20—Synthesis of N-[(5-amino-1,3,4-oxadiazol-2-yl)methyl]-N-[(4-cyano-2-fluoro-phenyl)methyl]-2,2-dideuterio-2-[3,5-difluoro-2-(1,1,2,2,2-pentadeuterioethyl)phenoxy]acetamide (Compound No. 77)

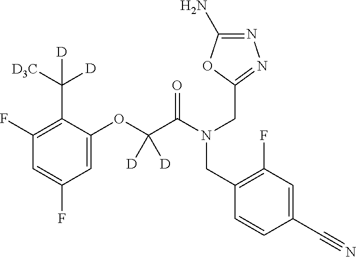 [SMILES: [2H]C([2H])(Oc1cc(F)cc(F)c1C([2H])([2H])C([2H])([2H])[2H])C(=O)N(Cc1nnc(N)o1)Cc1ccc(C#N)cc1F]

### (3,5-Difluorophenyl) N-isopropylcarbamate

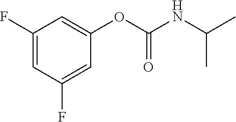

To a cooled (0° C.) solution of 3,5-difluorophenol (200 mg, 1.54 mmol) and N,N-dimethylpyridin-4-amine (19 mg, 0.154 mmol) in dry THE (0.75 mL), 2-isocyanatopropane (0.23 mL, 2.31 mmol) was added. The reaction mixture was stirred at 60° C. for 2 h. The mixture was then cooled to rt, and treated with 2 M HCl (1.5 ml). After addition of Et2O (5 ml), the aqueous layer was separated, extracted with Et₂O (2×5 ml), and the combined organic extracts were washed with saturated NaHCO₃ solution (5 ml), dried, and concentrated in vacuo. The crude product was purified by Normal phase column chromatography General Method 2 (296 mg, 1.35 mmol, 88% Yield) as off white needles. ¹H-NMR (500 MHz, Chloroform-d) δ 6.73 (qd, J=6.8, 2.9 Hz, 2H), 6.65 (tt, J=8.9, 2.2 Hz, 1H), 4.89 (s, 1H), 3.96-3.79 (m, 1H), 1.24 (s, 3H), 1.23 (s, 3H). LCMS Method E: m/z: 216.2 [M+H]+, (ESI+), RT=0.81.

### (3,5-Difluoro-2-iodo-phenyl) N-isopropylcarbamate

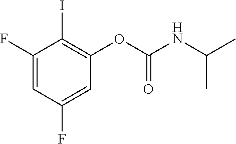 [SMILES: CC(C)NC(=O)Oc1cc(F)cc(F)c1I]

A stirred solution of (3,5-difluorophenyl) N-isopropylcarbamate (2.00 g, 9.29 mmol) and N,N,N′,N′-tetramethylethane-1,2-diamine (1.5 mL, 10.2 mmol) in Diethyl ether (100 mL) at room temperature was treated dropwise with trimethylsilyl trifluoromethanesulfonate (1.9 mL, 9.76 mmol). After 30 mins the mixture was cooled to −78° C. and TMEDA (2.7 mL, 18.6 mmol) and 1.6 M butyllithium (12 mL, 18.6 mmol) were added. The mixture was stirred for 1 hour before molecular iodine (5.90 g, 23.2 mmol) dissolved in Diethyl ether (30 mL) was added. After 1 hour MeOH (0.92 mL) and 2M HCl (55 mL) were added dropwise, and the mixture allowed to warm to room temperature. The aqueous layer was extracted with Et2O (3×50 ml). The combined organics were washed with NaHCO₃ (50 mL), dried (MgSO₄) and concentrated under a stream of nitrogen. The crude product was purified by Normal phase column chromatography General Method 2 to afford (3,5-difluoro-2-iodo-phenyl) N-isopropylcarbamate (2.07 g, 4.13 mmol, 44% Yield) as a yellow oil. ¹H-NMR (400 MHz, Chloroform-d) δ 6.80 (d, J=9.1 Hz, 1H), 6.74-6.61 (m, 1H), 4.94 (s, 1H), 3.91-3.74 (m, 1H), 1.21 (s, 3H), 1.19 (s, 3H). LCMS Method E: m/z: 342.0 [M+H]+, (ESI+), RT=0.87.

### 3,5-Difluoro-2-iodo-phenol

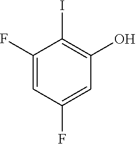

To a solution of (3,5-difluoro-2-iodo-phenyl) N-isopropylcarbamate (50%, 3.48 g, 5.10 mmol) in Ethanol (50 mL) was added 2 M Sodium Hydroxide (7.7 mL, 15.3 mmol) and the resulting mixture was stirred at room temperature for 2 h. 2M aq HCl (16 mL) and Diethylether (40 ml) were added. The organic layer was separated and the aqueous washed with further diethyl ether (2×40 ml). The combined organic phases were washed with brine (40 ml), dried (MgSO₄) and concentrated under reduced pressure to afford 3,5-difluoro-2-iodo-phenol (50% purity, 1.85 g, 3.61 mmol, 71% Yield) as a yellow oil. The product was taken forward to the next reaction without further purification. ¹H-NMR (500 MHz, CDCl3) δ 6.54-6.49 (m, 1H), 6.41-6.36 (m, 1H), 4.57 (s, 1H). LCMS Method E: m/z: 510.9, (ESI−), RT=0.80.

### 3,5-Difluoro-2-(2-trimethylsilylethynyl)phenol

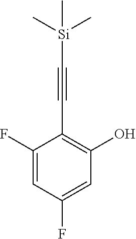

To an oven dried pressure vial containing a magnetic stirrer was added 3,5-difluoro-2-iodo-phenol (70%, 1.85 g, 5.06 mmol), palladium(2+) chloride-triphenylphosphane (1:2:2) (178 mg, 0.253 mmol) and copper(1) iodide (48 mg, 0.253 mmol). The lid was closed and the vessel flushed with nitrogen for 5 mins. Triethylamine (10 mL, 71.7 mmol) was added followed by dropwise addition of ethynyl(trimethyl)silane (0.70 mL, 5.06 mmol). The reaction stirred at room temperature for 2 hours, and heated to 45° C. for a further 2 hours. The solvent was removed under reduced pressure and the resulting black residue purified by Normal phase column chromatography General Method 1 to afford 3,5-difluoro-2-(2-trimethylsilylethynyl)phenol (49% purity, 1.94 g, 4.20 mmol, 83% Yield) as a brown oil. The product was taken forward to reaction without further purification. LCMS Method E: m/z: 225.1 [M−H]−, (ESI−), RT=0.98.

### Methyl 2-[3,5-difluoro-2-(2-trimethylsilylethynyl)phenoxy]acetate

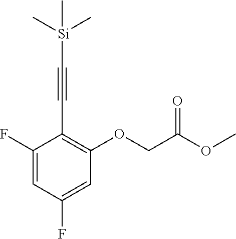 [SMILES: COC(=O)COc1cc(F)cc(F)c1C#C[Si](C)(C)C]

3,5-Difluoro-2-(2-trimethylsilylethynyl)phenol (87%, 2.43 g, 9.34 mmol), methyl bromoacetate (1.3 mL, 14.0 mmol), dipotassium carbonate (2.582 g, 18.7 mmol) and Acetonitrile (20 mL) were added to a pressure vial and stirred at RT for 3 hours. The reaction solvent was then removed under reduced pressure. The residue was resuspended in water (20 mL) and EtOAc (20 ml). The organic layer was removed and the aqueous extracted with further EtOAc (2×20 mL). The organics were combined, washed with brine (20 mL), dried, and concentrated under reduced pressure. The crude product was purified by Normal phase column chromatography General Method 1 to afford methyl 2-[3,5-difluoro-2-(2-trimethylsilylethynyl)phenoxy]acetate (2.24 g, 6.16 mmol, 66% Yield) as a brown solid. ¹H-NMR (500 MHz, CDCl3) δ 6.51 (td, J=8.9, 2.3 Hz, 1H), 6.31 (dt, J=10.1, 1.9 Hz, 1H), 4.70 (s, 2H), 3.82 (s, 3H), 0.27 (s, 9H). LCMS Method E: m/z: 619.2 [2M+Na]+, (ESI+), RT=1.08.

### 2,2-Dideuterio-2-[2-(2-deuterioethynyl)-3,5-difluoro-phenoxy]acetic acid

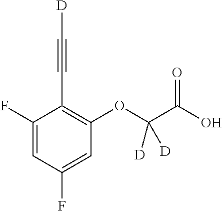 [SMILES: [2H]C#Cc1c(F)cc(F)cc1OC([2H])([2H])C(=O)O]

Methyl 2-[3,5-difluoro-2-(2-trimethylsilylethynyl)phenoxy]acetate (82%, 2.24 g, 6.16 mmol) and dipotassium carbonate (1.70 g, 12.3 mmol) were dissolved in trideuterio(deuteriooxy)methane (10 mL, 6.16 mmol). The reaction was stirred at room temperature for 18 hours. Lithium hydroxide (154 mg, 6.16 mmol) dissolved in D₂O (5 mL) and THF (5 ml) were added to the reaction mixture, which was stirred at room temperature for 1 hour, then concentrated under reduced pressure. The residue was resuspended between D₂O (40 ml) and Et₂O (40 ml). The organic layer was separated and the aqueous was acidified to ˜pH 1 with 4M HCl and extracted with EtOAc (3×40 ml) to isolate the acid product. The extracts were combined, dried, and concentrated under reduced pressure to afford 2,2-dideuterio-2-[2-(2-deuterioethynyl)-3,5-difluoro-phenoxy]acetic acid (1.51 g, 5.68 mmol, 92% Yield) as a pale brown solid. ¹H-NMR (500 MHz, CDCl3) δ 6.57 (td, J=8.8, 2.3 Hz, 1H), 6.37 (ddd, J=9.7, 2.2, 1.4 Hz, 1H). LCMS Method D: m/z: 214.2 [M−H]−, (ESI−), RT=0.27.

### 2,2-Dideuterio-2-[3,5-difluoro-2-(1,1,2,2,2-pentadeuterioethyl)phenoxy]acetic acid

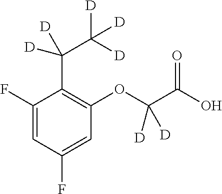 [SMILES: [2H]C([2H])(Oc1cc(F)cc(F)c1C([2H])([2H])C([2H])([2H])[2H])C(=O)O]

A stirred solution of 2,2-dideuterio-2-[2-(2-deuterioethynyl)-3,5-difluoro-phenoxy]acetic acid (90%, 1.34 g, 5.61 mmol) in Ethyl acetate (64 mL) was purged with N₂ (×3). Palladium on carbon 10% (119 mg, 1.12 mmol) was then added, the mixture was purged with N₂ (×3), then with D₂ (×3). The mixture was stirred at RT for 6 hours. The D₂ was then evacuated from the flask and the mixture purged with N₂ (×3). The mixture was then filtered through celite to remove Pd/C, and concentrated to obtain 2,2-dideuterio-2-[3,5-difluoro-2-(1,1,2,2,2-pentadeuterioethyl)phenoxy]acetic acid (1.46 g, 5.17 mmol, 92% Yield) as a brown solid. ¹H-NMR (400 MHz, CDCl3) δ 7.58 (s, 1H), 6.52-6.43 (m, 1H), 6.29 (dt, J=10.2, 2.0 Hz, 1H). LCMS Method D: m/z: 222.4 [M−H]−, (ESI−), RT=0.34.

### Ethyl 2-[(4-cyano-2-fluoro-phenyl)methyl-[2,2-dideuterio-2-[3,5-difluoro-2-(1,1,2,2,2-pentadeuterioethyl)phenoxy]acetyl]amino]acetate

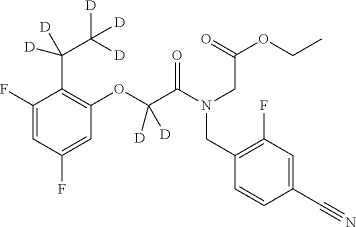 [SMILES: [2H]C([2H])(Oc1cc(F)cc(F)c1C([2H])([2H])C([2H])([2H])[2H])C(=O)N(CC(=O)OCC)Cc1ccc(C#N)cc1F]

1-[Bis(dimethylamino)methylidene]-1H-[1,2,3]triazolo[4,5-b]pyridin-1-ium 3-oxide hexafluorophosphate (295 mg, 0.775 mmol) was added to a solution of deuterio 2,2-dideuterio-2-[3,5-difluoro-2-(1,1,2,2,2-pentadeuterioethyl)phenoxy]acetate (79%, 200 mg, 0.705 mmol), ethyl 2-[(4-cyano-2-fluoro-phenyl)methylamino]acetate (as prepared in Example 19, above; 77%, 195 mg, 0.634 mmol) and N-ethyl-N-isopropyl-propan-2-amine (0.25 mL, 1.41 mmol) in DMF-Anhydrous (3.4143 mL) and the resulting mixture was stirred at RT for 2 h. The reaction mixture was then diluted with water (20 ml) and extracted with EtOAc (3×20 ml). The organics were combined, dried, and concentrated under reduced pressure before being purified by Normal phase column chromatography General Method 1 to afford ethyl 2-[(4-cyano-2-fluoro-phenyl)methyl-[2,2-dideuterio-2-[3,5-difluoro-2-(1,1,2,2,2-pentadeuterioethyl)phenoxy]acetyl] amino]acetate (263 mg, 0.477 mmol, 68% Yield) as a pale yellow oil. ¹H-NMR (400 MHz, DMSO) δ 7.76 (s, 1H), 7.70-7.50 (m, 2H), 6.74-6.60 (m, 2H), 4.91-4.59 (m, 2H), 4.39-3.99 (m, 4H), 1.20 (t, J=7.1 Hz, 3H). LCMS Method D: m/z: 459.4 [M+NH4]+, (ESI+), RT=0.71.

### tert-Butyl N-[[2-[(4-cyano-2-fluoro-phenyl)methyl-[2,2-dideuterio-2-[3,5-difluoro-2-(1,1,2,2,2-pentadeuterioethyl)phenoxy]acetyl]amino]acetyl]amino]carbamate

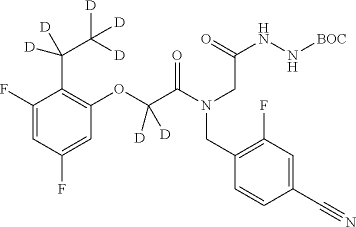 [SMILES: [2H]C([2H])(Oc1cc(F)cc(F)c1C([2H])([2H])C([2H])([2H])[2H])C(=O)N(CC(=O)NNC(=O)OC(C)(C)C)Cc1ccc(C#N)cc1F]

Ethyl 2-[(4-cyano-2-fluoro-phenyl)methyl-[2,2-dideuterio-2-[3,5-difluoro-2-(1,1,2,2,2-pentadeuterioethyl)phenoxy]acetyl]amino]acetate (85%, 888 mg, 1.68 mmol) was dissolved in 1,4-Dioxane (11.4 mL), then 2 M LiOH (aq) (1.68 mL, 3.36 mmol) was added and the reaction was stirred for 1 hour at ambient temperature, concentrated under reduced pressure, re-suspended in water (15 ml), and extracted with diethyl ether (15 ml). The aqueous was then acidified to ˜pH 1 by the addition of 4M HCl. The aqueous was extracted with EtOAc (3×15 ml). The combined organics were dried and concentrated in vacuo to afford 2-[(4-cyano-2-fluoro-phenyl)methyl-[2,2-dideuterio-2-[3,5-difluoro-2-(1,1,2,2,2-pentadeuterioethyl)phenoxy] acetyl]amino]acetic acid (752 mg, 1.60 mmol, 95% Yield). ¹H-NMR (400 MHz, DMSO) δ 7.84-7.69 (m, 1H), 7.69-7.47 (m, 2H), 6.74-6.58 (m, 2H), 4.88-4.57 (m, 2H), 4.30-3.92 (m, 2H). LCMS Method D: m/z: 412.6 [M−H]−, (ESI−), RT=0.42.

2-[(4-Cyano-2-fluoro-phenyl)methyl-[2,2-dideuterio-2-[3,5-difluoro-2-(1,1,2,2,2-pentadeuterioethyl)phenoxy]acetyl]amino]acetic acid (88%, 752 mg, 1.60 mmol) was stirred in DCM (16 mL). To the colourless solution was added tert-butyl hydrazinecarboxylate (317 mg, 2.40 mmol), DIPEA (854 uL, 4.80 mmol) and then HATU (730 mg, 1.92 mmol) and the reaction was stirred at room temperature for 1.5 hours, then diluted with water (15 mL) and extracted with DCM (3×20 mL). The organics were combined, dried, and concentrated under vacuum before being purified by Normal phase column chromatography General Method 1 to afford tert-butyl N-[[2-[(4-cyano-2-fluoro-phenyl)methyl-[2,2-dideuterio-2-[3,5-difluoro-2-(1,1,2,2,2-pentadeuterioethyl)phenoxy]acetyl]amino]acetyl]amino]carbamate (464 mg, 0.809 mmol, 51% Yield). ¹H-NMR (400 MHz, DMSO) δ 9.74 (s, 1H), 8.56 (s, 1H), 7.74 (d, J=9.0 Hz, 1H), 7.62 (d, J=7.0 Hz, 1H), 7.54 (t, J=7.5 Hz, 1H), 6.77-6.57 (m, 2H), 4.90-4.48 (m, 2H), 4.23-3.99 (m, 3H), 1.40 (s, 9H). LCMS Method D: m/z: 526.6 [M−H]−, (ESI−), RT=0.67.

### N-[(5-Amino-1,3,4-oxadiazol-2-yl)methyl]-N-[(4-cyano-2-fluoro-phenyl)methyl]-2,2-dideuterio-2-[3,5-difluoro-2-(1,1,2,2,2-pentadeuterioethyl)phenoxy]acetamide (Compound No. 77)

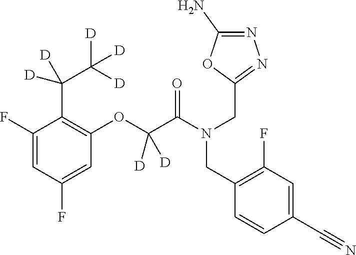 [SMILES: [2H]C([2H])(Oc1cc(F)cc(F)c1C([2H])([2H])C([2H])([2H])[2H])C(=O)N(Cc1nnc(N)o1)Cc1ccc(C#N)cc1F]

To a solution of tert-butyl N-[[2-[(4-cyano-2-fluoro-phenyl)methyl-[2,2-dideuterio-2-[3,5-difluoro-2-(1,1,2,2,2-pentadeuterioethyl)phenoxy]acetyl]amino]acetyl]amino]carbamate (92%, 464 mg, 0.809 mmol) in DCM (10 mL), was added trifluoroacetic acid (0.62 mL, 8.09 mmol). The mixture stirred at RT for 1 hour, then concentrated. The residue was re-suspended in methanol and passed through an SCX column (5 g), the column was washed using methanol, then the product was eluted using ˜2M NH3 in methanol to afford N-[(4-cyano-2-fluoro-phenyl)methyl]-2,2-dideuterio-2-[3,5-difluoro-2-(1,1,2,2,2-pentadeuterioethyl)phenoxy]-N-(2-hydrazino-2-oxo-ethyl)acetamide (318 mg, 0.475 mmol, 59% Yield) as a colourless solid. ¹H NMR (400 MHz, DMSO) δ 9.14 (s, 1H), 7.75 (d, J=9.1 Hz, 1H), 7.68-7.44 (m, 2H), 6.82-6.57 (m, 2H), 4.60 (s, 2H), 4.22 (s, 2H), 4.01 (s, 2H). LCMS Method D: m/z: 426.5 [M−H]−, (ESI−), RT=0.57.

To a solution of N-[(4-cyano-2-fluoro-phenyl)methyl]-2,2-dideuterio-2-[3,5-difluoro-2-(1,1,2,2,2-pentadeuterioethyl)phenoxy]-N-(2-hydrazino-2-oxo-ethyl)acetamide (66%, 424 mg, 0.664 mmol) and sodium hydrogen carbonate (61 mg, 0.731 mmol) in 1,4-Dioxane-Anhydrous (15 mL) was added carbononitridic bromide (77 mg, 0.731 mmol) and the reaction stirred at room temperature for 16 h. The reaction mixture was concentrated in vacuo and purified by High pH reverse phase column chromatography to afford N-[(5-amino-1,3,4-oxadiazol-2-yl)methyl]-N-[(4-cyano-2-fluoro-phenyl)methyl]-2,2-dideuterio-2-[3,5-difluoro-2-(1,1,2,2,2-pentadeuterioethyl)phenoxy]acetamide (272 mg, 0.595 mmol, 90% Yield) as a colourless powder. ¹H NMR (400 MHz, DMSO) δ 7.75 (d, J=9.9 Hz, 1H), 7.62 (d, J=7.8 Hz, 1H), 7.49 (t, J=7.7 Hz, 1H), 6.77 (s, 2H), 6.69 (t, J=9.5 Hz, 2H), 4.84-4.51 (m, 4H).

LCMS Method H:

m/z: 451.6 [M−H]−, (ESI−), RT=3.29.

### Example 21—Synthesis of N-[(5-Amino-1,3,4-oxadiazol-2-yl)methyl]-2-(2-ethyl-3,5-difluoro-phenoxy)-N-[(2-fluoro-4-sulfamoyl-phenyl)methyl]acetamide (Compound No. 79)

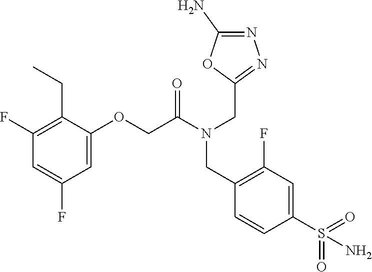 [SMILES: CCc1c(F)cc(F)cc1OCC(=O)N(Cc1nnc(N)o1)Cc1ccc(S(N)(=O)=O)cc1F]

### 2-Fluoro-4-sulfamoyl-benzoic acid

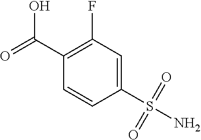 [SMILES: NS(=O)(=O)c1ccc(C(=O)O)c(F)c1]

3-Fluoro-4-methyl-benzenesulfonamide (5.00 g, 26.4 mmol) was suspended in Water (250 mL) then stirred vigorously and heated to reflux. Potassium permanganate (16.70 g, 0.106 mol) was added in eight equal portions every 15 mins over two hours, the reaction was left at reflux for 1 hour before being cooled to ambient temperature and left standing overnight. The reaction was filtered through celite then the filtrate was acidified with the addition of 1M HCl (aq) (100 mL) then the solution was extracted with EtOAc (100 mL×2) which were combined and dried over Na₂SO₄, then filtered and concentrated to afford 2-fluoro-4-sulfamoyl-benzoic acid (4.18 g, 18.7 mmol, 71% Yield) as a white crystalline solid. ¹H-NMR (500 MHz, DMSO) δ 13.67 (s, 1H), 8.08-8.03 (m, 1H), 7.73 (dd, J=8.1, 1.7 Hz, 1H), 7.68 (dd, J=10.2, 1.6 Hz, 1H), 7.65 (s, 2H). LCMS Method E: m/z: 218 [M−H]−, (ESI−), RT=0.32.

### 3-Fluoro-4-(hydroxymethyl)benzenesulfonamide

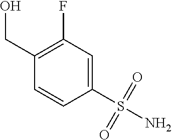 [SMILES: NS(=O)(=O)c1ccc(CO)c(F)c1]

2-Fluoro-4-sulfamoyl-benzoic acid (4.18 g, 19.1 mmol) was dissolved in anhydrous THF (84 mL) then 10 M (methylsulfanyl)methane-borane (1:1) (7.6 mL, 76.3 mmol) was gradually added to the reaction, then the reaction was stirred at room temperature 18 hours to result in a suspension. The reaction was quenched with the slow addition of MeOH (10 mL). Once effervescence had abated, further MeOH was added until a pale yellow solution had resulted. The mixture was then concentrated and the residue dissolved in MeOH and concentrated to afford 3-fluoro-4-(hydroxymethyl)benzenesulfonamide (3.95 g, 18.7 mmol, 98% Yield) as a cream powdery solid. ¹H-NMR (400 MHz, DMSO) δ 7.70-7.62 (m, 2H), 7.56-7.50 (m, 1H), 7.44 (s, 2H), 5.45 (t, J=5.7 Hz, 1H), 4.60 (d, J=5.7 Hz, 2H). LCMS Method D: m/z: 206 [M+H]+, (ESI+), RT=0.22.

### Ethyl 2-[(2-fluoro-4-sulfamoyl-phenyl)methylamino]acetate

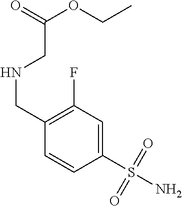 [SMILES: CCOC(=O)CNCc1ccc(S(N)(=O)=O)cc1F]

Triphenylphosphine (2.96 g, 11.3 mmol), 1H-imidazole (0.77 g, 11.3 mmol) and iodine (2.86 g, 11.3 mmol) were dissolved in DCM (30 mL) then the reaction was stirred for 10 mins. 3-fluoro-4-(hydroxymethyl)benzenesulfonamide (97%, 1.99 g, 9.41 mmol) was added then the reaction was stirred for 1 hour at room temperature. The reaction was diluted with further DCM (20 mL) then 1:1 water/NaHCO₃ (sat aq) (90 mL) was added and the biphasic suspension was stirred for 10 minutes. The organics were separated, and the aqueous layer extracted with DCM (4×30 mL). The organics were combined, dried, and concentrated under reduced pressure.

The residue was dissolved in MeCN (20 mL) then added to a pre-stirred solution of glycine ethyl ester hydrochloride (3.28 g, 23.5 mmol) and DIPEA (6.6 mL, 37.6 mmol) in MeCN (20 mL). The reaction was then heated to 45° C. for 3 hours. The reaction was concentrated, then the residue was partitioned between DCM (30 mL) and 1:1 water/NaHCO₃ (sat aq) (30 mL) then the organics were separated, dried over Na₂SO₄, filtered and concentrated. The residue was purified by Normal phase column chromatography General Method 2 to afford ethyl 2-[(2-fluoro-4-sulfamoyl-phenyl)methylamino]acetate (1.61 g, 5.55 mmol, 59% Yield) as a yellow solid. ¹H-NMR, (500 MHz, DMSO) δ 7.65 (d, J=6.8 Hz, 1H), 7.62 (dd, J=8.0, 1.6 Hz, 1H), 7.54 (dd, J=9.7, 1.6 Hz, 1H), 7.45 (s, 2H), 4.08 (q, J=7.1 Hz, 2H), 3.82 (d, J=3.8 Hz, 2H), 3.33 (d, J=5.7 Hz, 2H), 2.64-2.58 (m, 1H), 1.19 (t, J=7.1 Hz, 3H). LCMS Method D: m/z: 291.2 [M+H]+, (ESI+), RT=0.37.

### Ethyl 2-[[2-(2-ethyl-3,5-difluoro-phenoxy)acetyl]-[(2-fluoro-4-sulfamoyl-phenyl)methyl]amino]acetate

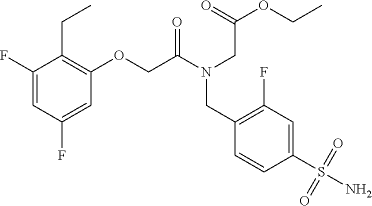 [SMILES: CCOC(=O)CN(Cc1ccc(S(N)(=O)=O)cc1F)C(=O)COc1cc(F)cc(F)c1CC]

Ethyl 2-[(2-fluoro-4-sulfamoyl-phenyl)methylamino]acetate (100%, 336 mg, 1.16 mmol) and 2-(2-ethyl-3,5-difluoro-phenoxy)acetic acid (95%, 263 mg, 1.16 mmol; prepared, for example, as described in Example 12) were dissolved in DMF (4.4 mL) then DIPEA (0.24 mL, 1.39 mmol) was added followed by the addition of T3P (50% in EtOAc) (50%, 1.4 mL, 2.31 mmol) then the reaction was stirred at room temperature for 4 hours. The reaction mixture was diluted with water (15 mL) and extracted with EtOAc (3×20 mL). The organics were washed with brine (15 mL), dried over MgSO₄, filtered and concentrated under reduced pressure. The residue was purified by Normal phase column chromatography General Method 1 to afford ethyl 2-[[2-(2-ethyl-3,5-difluoro-phenoxy)acetyl]-[(2-fluoro-4-sulfamoyl-phenyl)methyl]amino]acetate (502 mg, 0.812 mmol, 70% Yield) as a white solid. ¹H-NMR, (400 MHz, CDCl3) δ 7.68-7.64 (m, 1H), 7.61 (dd, J=9.3, 1.7 Hz, 1H), 7.57-7.50 (m, 1H), 6.46 (ddd, J=9.2, 7.2, 2.4 Hz, 1H), 6.43-6.35 (m, 1H), 4.90-4.81 (m, 4H), 4.71 (s, 2H), 4.19 (qd, J=7.0, 2.2 Hz, 3H), 4.05 (s, 1H), 2.62 (q, J=7.5 Hz, 1H), 2.43 (q, J=7.5 Hz, 1H), 1.28-1.24 (m, 3H), 1.12 (t, J=7.5 Hz, 2H), 1.00 (t, J=7.5 Hz, 1H). LCMS Method E: m/z: 489.2 [M+H]+, (ESI+), RT=0.91.

### tert-Butyl N-[[2-[[2-(2-ethyl-3,5-difluoro-phenoxy)acetyl]-[(2-fluoro-4-sulfamoyl-phenyl)methyl]amino]acetyl]amino]carbamate

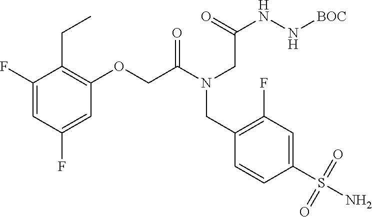 [SMILES: CCc1c(F)cc(F)cc1OCC(=O)N(CC(=O)NNC(=O)OC(C)(C)C)Cc1ccc(S(N)(=O)=O)cc1F]

Ethyl 2-[[2-(2-ethyl-3,5-difluoro-phenoxy)acetyl]-[(2-fluoro-4-sulfamoyl-phenyl)methyl]amino]acetate (79%, 502 mg, 0.812 mmol) dissolved in 1,4-Dioxane (6 mL) then 2 M LiOH (aq) (0.81 mL, 1.62 mmol) was added and left to stir for 2.5 h at room temperature. The reaction was concentrated under reduced pressure, the residue was re-suspended in water (15 ml) and extracted with diethyl ether (15 ml). The aqueous was then acidified to ˜pH 1 by the addition of 4M HCl (˜1 mL) and extracted with EtOAc (3×15 ml). The combined organics were dried and concentrated to afford 2-[[2-(2-ethyl-3,5-difluoro-phenoxy)acetyl]-[(2-fluoro-4-sulfamoyl-phenyl)methyl]amino]acetic acid (428 mg, 0.799 mmol, 98% Yield) as a colourless oil. ¹H-NMR, (400 MHz, DMSO) δ 12.58 (s, 1H), 7.60 (s, 3H), 7.27 (s, 2H), 6.71-6.63 (m, 2H), 4.96 (d, J=32.3 Hz, 2H), 4.80 (s, 1H), 4.64 (s, 1H), 4.21 (s, 1H), 3.97 (s, 1H), 2.59 (s, 2H), 1.10 (t, J=5.3 Hz, 3H). LCMS Method D: m/z: 459.6 [M−H]−, (ESI−), RT=0.37.

2-[[2-(2-Ethyl-3,5-difluoro-phenoxy)acetyl]-[(2-fluoro-4-sulfamoyl-phenyl)methyl]amino]acetic acid (86%, 428 mg, 0.799 mmol) and tert-butyl hydrazinecarboxylate (127 mg, 0.959 mmol) were dissolved in 1,4-Dioxane (4 mL), then DIPEA (0.17 mL, 0.959 mmol) followed by the addition of T3P (50% in EtOAc) (50%, 0.95 mL, 1.60 mmol) and the reaction left to stir at ambient temperature for 1 h. The reaction was partitioned between EtOAc (15 mL) and water (15 mL), the organic phase was separated and the aqueous was extracted with EtOAc (2×15 mL). The combined organics were washed with brine (15 mL), dried over MgSO₄, filtered and concentrated under reduced pressure. The residue was purified by Normal phase column chromatography General Method 1 to afford ethyl 2-[[2,2-dideuterio-2-[3,5-difluoro-2-(1,1,2,2,2-pentadeuterioethyl)phenoxy]acetyl]-[(2-fluoro-4-sulfamoyl-phenyl)methyl]amino]acetate (981 mg, 1.43 mmol, 67% Yield) as a white solid. ¹H-NMR, (400 MHz, DMSO) δ 9.30 (s, 1H), 8.55 (s, 1H), 7.59 (d, J=10.2 Hz, 2H), 7.56-7.03 (m, 3H), 6.73 (d, J=10.7 Hz, 1H), 6.66 (td, J=9.7, 2.3 Hz, 1H), 4.99 (s, 2H), 4.83 (s, 1H), 4.61 (s, 2H), 4.10 (s, 1H), 2.60 (d, J=7.2 Hz, 2H), 1.41 (s, 9H), 1.10 (t, J=7.4 Hz, 3H). LCMS Method E: m/z: 573.2 [M−H]−, (ESI−), RT=0.90.

### N-[(5-Amino-1,3,4-oxadiazol-2-yl)methyl]-2-(2-ethyl-3,5-difluoro-phenoxy)-N-[(2-fluoro-4-sulfamoyl-phenyl)methyl]acetamide (Compound No. 79)

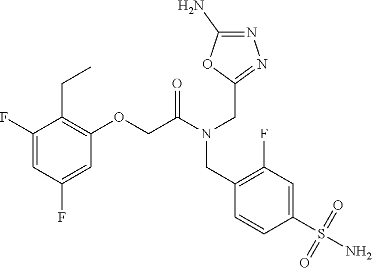 [SMILES: CCc1c(F)cc(F)cc1OCC(=O)N(Cc1nnc(N)o1)Cc1ccc(S(N)(=O)=O)cc1F]

To a solution of tert-butyl N-[[2-[[2-(2-ethyl-3,5-difluoro-phenoxy)acetyl]-[(2-fluoro-4-sulfamoyl-phenyl)methyl]amino]acetyl]amino]carbamate (98%, 475 mg, 0.810 mmol) in DCM (10 mL), was added trifluoroacetic acid (2.0 mL, 26.1 mmol). The mixture stirred at RT for 1 hour. The mixture was then concentrated and the residue was re-suspended in methanol and passed through an SCX column (2 g), the column was washed using methanol, then the product was eluted using ˜2M NH3 in methanol to afford 2-(2-ethyl-3,5-difluoro-phenoxy)-N-[(2-fluoro-4-sulfamoyl-phenyl)methyl]-N-(2-hydrazino-2-oxo-ethyl)acetamide (41% purity, 336 mg, 0.290 mmol, 36% Yield) as a white solid. ¹H-NMR, (400 MHz, DMSO) δ 10.22 (s, 1H), 7.58 (d, J=16.2 Hz, 4H), 7.25 (s, 2H), 6.75 (d, J=9.1 Hz, 1H), 6.70-6.61 (m, 1H), 5.01 (s, 1H), 4.94 (s, 1H), 4.61 (s, 2H), 4.22 (s, 1H), 4.00 (s, 1H), 2.60 (d, J=6.5 Hz, 2H), 1.10 (t, J=7.2 Hz, 3H). LCMS Method E: m/z: 475.2 [M+H]+, (ESI+), RT=0.73.

To a solution of 2-(2-ethyl-3,5-difluoro-phenoxy)-N-[(2-fluoro-4-sulfamoyl-phenyl)methyl]-N-(2-hydrazino-2-oxo-ethyl)acetamide (41%, 336 mg, 0.290 mmol) and sodium hydrogen carbonate (27 mg, 0.319 mmol) in 1,4-Dioxane-Anhydrous (3.5 mL) was added carbononitridic bromide (34 mg, 0.319 mmol) and the mixture left to stir at room temperature for 18 h. The reaction mixture was concentrated and purified by High pH reverse phase column chromatography followed by High pH preparative HPLC (Standard method) to afford N-[(5-amino-1,3,4-oxadiazol-2-yl)methyl]-2-(2-ethyl-3,5-difluoro-phenoxy)-N-[(2-fluoro-4-sulfamoyl-phenyl)methyl]acetamide (29 mg, 0.0575 mmol, 20% Yield) as a white solid. ¹H-NMR, (400 MHz, DMSO) δ 8.56 (s, 1H), 8.22 (d, J=8.3 Hz, 1H), 8.09 (d, J=8.0 Hz, 1H), 6.77 (s, 2H), 6.72-6.60 (m, 2H), 5.06 (s, 2H), 4.84-4.70 (m, 4H), 1.82-1.72 (m, 1H), 0.91-0.82 (m, 4H). LCMS Method A: m/z: 500.2 [M+H]+, (ESI+), RT=2.73.

### Example 22—Synthesis of N-[(5-Amino-1,3,4-oxadiazol-2-yl)methyl]-2,2-dideuterio-2-[3,5-difluoro-2-(1,1,2,2,2-pentadeuterioethyl)phenoxy]-N-[(2-fluoro-4-sulfamoyl-phenyl)methyl]acetamide (Compound No. 111)

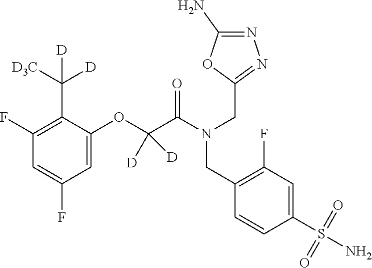 [SMILES: [2H]C([2H])(Oc1cc(F)cc(F)c1C([2H])([2H])C([2H])([2H])[2H])C(=O)N(Cc1nnc(N)o1)Cc1ccc(S(N)(=O)=O)cc1F]

### Ethyl 2-[[2,2-dideuterio-2-[3,5-difluoro-2-(1,1,2,2,2-pentadeuterioethyl)phenoxy]acetyl]-[(2-fluoro-4-sulfamoyl-phenyl)methyl]amino]acetate

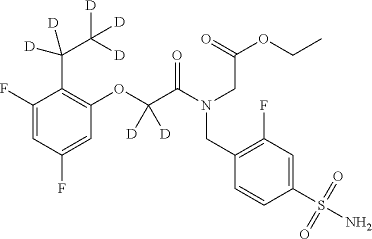 [SMILES: [2H]C([2H])(Oc1cc(F)cc(F)c1C([2H])([2H])C([2H])([2H])[2H])C(=O)N(CC(=O)OCC)Cc1ccc(S(N)(=O)=O)cc1F]

Ethyl 2-[(2-fluoro-4-sulfamoyl-phenyl)methylamino]acetate (100%, 614 mg, 2.11 mmol; prepared, for example, as described in Example 21) and deuterio 2,2-dideuterio-2-[3,5-difluoro-2-(1,1,2,2,2-pentadeuterioethyl)phenoxy]acetate (79%, 600 mg, 2.11 mmol; prepared, for example, as described in Example 20) were dissolved in DMF (8 mL) then DIPEA (0.44 mL, 2.53 mmol) was added followed by the addition of T3P (50% in EtOAc) (50%, 2.5 mL, 4.22 mmol) then the reaction was stirred at room temperature for 4 hours. The reaction was partitioned between EtOAc (15 mL) and water (15 mL), the organic phase was separated and the aqueous was extracted with EtOAc (2×15 mL). The combined organics were washed with brine (15 mL), dried over MgSO₄, filtered and concentrated. The residue was purified by Normal phase column chromatography General Method 1 to afford ethyl 2-[[2,2-dideuterio-2-[3,5-difluoro-2-(1,1,2,2,2-pentadeuterioethyl)phenoxy]acetyl]-[(2-fluoro-4-sulfamoyl-phenyl)methyl]amino]acetate (981 mg, 1.43 mmol, 67% Yield) as a pale yellow solid. ¹H-NMR, (400 MHz, CDCl3) δ 7.68-7.63 (m, 1H), 7.60 (dd, J=9.2, 1.6 Hz, 1H), 7.53 (t, J=7.5 Hz, 1H), 6.50-6.42 (m, 1H), 6.39 (ddd, J=10.0, 8.1, 1.9 Hz, 1H), 4.98-4.91 (m, 2H), 4.83 (s, 1H), 4.71 (s, 1H), 4.23-4.14 (m, 3H), 4.05 (s, 1H), 1.25 (td, J=7.1, 2.8 Hz, 3H). LCMS Method E: m/z: 496.3 [M+H]+, (ESI−), RT=0.90. tert-butyl N-[[2-[[2,2-dideuterio-2-[3,5-difluoro-2-(1,1,2,2,2-pentadeuterioethyl)phenoxy]acetyl]-[(2-fluoro-4-sulfamoyl-phenyl)methyl]amino]acetyl]amino]carbamate

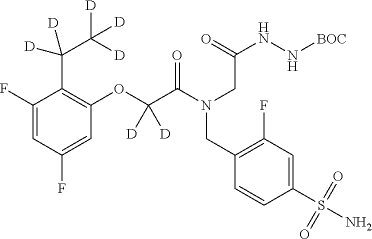 [SMILES: [2H]C([2H])(Oc1cc(F)cc(F)c1C([2H])([2H])C([2H])([2H])[2H])C(=O)N(CC(=O)NNC(=O)OC(C)(C)C)Cc1ccc(S(N)(=O)=O)cc1F]

Ethyl 2-[[2,2-dideuterio-2-[3,5-difluoro-2-(1,1,2,2,2-pentadeuterioethyl)phenoxy]acetyl]-[(2-fluoro-4-sulfamoyl-phenyl)methyl]amino]acetate (72%, 981 mg, 1.43 mmol) was dissolved in 1,4-Dioxane (10 mL) then 2 M LiOH (aq) (1.4 mL, 2.85 mmol) was added and left to stir for 2.5 h at room temperature. The reaction was concentrated under reduced pressure. The residue was re-suspended in water (15 ml) which was extracted with diethyl ether (15 ml). The aqueous was then acidified to ˜pH 1 by the addition of 4M HCl (˜1 mL), and extracted with EtOAc (3×15 ml). The combined organics were dried and concentrated to afford 2-[[2,2-dideuterio-2-[3,5-difluoro-2-(1,1,2,2,2-pentadeuterioethyl)phenoxy]acetyl]-[(2-fluoro-4-sulfamoyl-phenyl)methyl]amino]acetic acid (878 mg, 1.43 mmol, 100% Yield) as a colourless oil. ¹H-NMR, (400 MHz, DMSO) δ 7.69-7.49 (m, 3H), 7.27 (s, 2H), 6.66 (ddd, J=9.3, 8.1, 2.5 Hz, 2H), 4.77 (d, J=26.6 Hz, 1H), 4.64 (s, 1H), 4.20 (s, 1H), 3.96 (s, 1H). LCMS Method E: m/z: 468.2 [M+H]+, (ESI+), RT=0.77.

2-[[2,2-Dideuterio-2-[3,5-difluoro-2-(1,1,2,2,2-pentadeuterioethyl)phenoxy]acetyl]-[(2-fluoro-4-sulfamoyl-phenyl)methyl]amino]acetic acid (79%, 878 mg, 1.48 mmol) and tert-butyl hydrazinecarboxylate (235 mg, 1.78 mmol) were dissolved in 1,4-Dioxane (7.5 mL), then DIPEA (0.31 mL, 1.78 mmol) followed by the addition of T3P (50% in EtOAc) (50%, 1.8 mL, 2.97 mmol) and the reaction left to stir at room temperature for 2.5 h. The reaction was partitioned between EtOAc (15 mL) and water (15 mL), the organic phase was separated and the aqueous was extracted with EtOAc (2×15 mL). The combined organics were washed with brine (15 mL), dried over MgSO₄, filtered and concentrated. The residue was purified by Normal phase column chromatography General Method 1 to afford tert-butyl N-[[2-[[2,2-dideuterio-2-[3,5-difluoro-2-(1,1,2,2,2-pentadeuterioethyl)phenoxy]acetyl]-[(2-fluoro-4-sulfamoyl-phenyl)methyl]amino]acetyl]amino]carbamate (625 mg, 0.871 mmol, 59% Yield) as a white solid. ¹H-NMR, (400 MHz, DMSO) δ 8.50 (s, 1H), 7.59 (d, J=10.8 Hz, 2H), 7.56-6.95 (m, 3H), 6.73 (d, J=11.0 Hz, 1H), 6.66 (td, J=9.7, 2.3 Hz, 1H), 4.68 (s, 1H), 4.61 (s, 2H), 4.09 (s, 2H), 1.41 (s, 9H). LCMS Method E: m/z: 580.2 [M−H]−, (ESI−), RT=0.89.

### N-[(5-amino-1,3,4-oxadiazol-2-yl)methyl]-2,2-dideuterio-2-[3,5-difluoro-2-(1,1,2,2,2-pentadeuterioethyl)phenoxy]-N-[(2-fluoro-4-sulfamoyl-phenyl)methyl]acetamide (Compound No. 111)

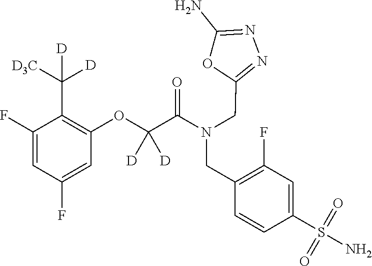 [SMILES: [2H]C([2H])(Oc1cc(F)cc(F)c1C([2H])([2H])C([2H])([2H])[2H])C(=O)N(Cc1nnc(N)o1)Cc1ccc(S(N)(=O)=O)cc1F]

To a solution of tert-butyl N-[[2-[[2,2-dideuterio-2-[3,5-difluoro-2-(1,1,2,2,2-pentadeuterioethyl)phenoxy]acetyl]-[(2-fluoro-4-sulfamoyl-phenyl)methyl]amino]acetyl]amino]carbamate (70%, 625 mg, 0.871 mmol) in DCM (7.5 mL), was added trifluoroacetic acid (1.9 mL, 24.5 mmol). The mixture stirred at RT for 1.5 hour. The mixture was then concentrated in vacuo, re-suspended in methanol and passed through an SCX column (2 g), the column was washed using methanol, the product was eluted using ˜2M NH3 in methanol to afford 2,2-dideuterio-2-[3,5-difluoro-2-(1,1,2,2,2-pentadeuterioethyl)phenoxy]-N-[(2-fluoro-4-sulfamoyl-phenyl)methyl]-N-(2-hydrazino-2-oxo-ethyl)acetamide (410 mg, 0.792 mmol, 91% Yield) as a white solid. ¹H-NMR, (400 MHz, DMSO) δ 9.14 (s, 1H), 7.58 (d, J=11.1 Hz, 2H), 7.50 (s, 1H), 7.27 (s, 2H), 6.76 (s, 1H), 6.66 (td, J=9.7, 2.4 Hz, 1H), 4.67 (d, J=67.8 Hz, 2H), 4.22 (s, 2H), 4.00 (s, 2H). LCMS Method E: m/z: 482.3 [M+H]+, (ESI+), RT=0.73.

To a solution of 2,2-dideuterio-2-[3,5-difluoro-2-(1,1,2,2,2-pentadeuterioethyl)phenoxy]-N-[(2-fluoro-4-sulfamoyl-phenyl)methyl]-N-(2-hydrazino-2-oxo-ethyl)acetamide (93%, 380 mg, 0.734 mmol) and sodium hydrogen carbonate (68 mg, 0.807 mmol) in 1,4-Dioxane-Anhydrous (8 mL) was added carbononitridic bromide (86 mg, 0.807 mmol) and the mixture left to stir at room temperature for 18 h. The reaction mixture was concentrated in vacuo and purified by High pH reverse phase column chromatography, then purified by High pH preparative HPLC (Standard method), and then Low pH preparative HPLC (Standard method) to afford N-[(5-amino-1,3,4-oxadiazol-2-yl)methyl]-2,2-dideuterio-2-[3,5-difluoro-2-(1,1,2,2,2-pentadeuterioethyl)phenoxy]-N-[(2-fluoro-4-sulfamoyl-phenyl)methyl]acetamide (185 mg, 0.362 mmol, 49% Yield) as a white solid. ¹H-NMR (400 MHz, DMSO) δ 7.68-7.56 (m, 2H), 7.48 (t, J=7.2 Hz, 1H), 6.84 (s, 2H), 6.68 (ddd, J=14.6, 8.7, 4.0 Hz, 2H), 4.71 (s, 4H). LCMS Method H: m/z: 505.6 [M−H]−, (ESI−), RT=2.68.

### Example 23—Synthesis of Additional Arylthioether Acetamides and Related Compounds

The compounds in Table 8 below were prepared using experimental procedures described in the Examples and Detailed Description.

TABLE 8

| Compound | |
|---|---|
| No. | Chemical Structure |
| 71 | 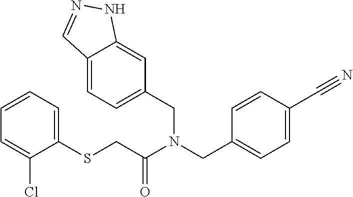 [SMILES: N#Cc1ccc(CN(Cc2ccc3cn[nH]c3c2)C(=O)CSc2ccccc2Cl)cc1] |
| 72 | 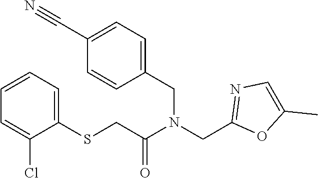 [SMILES: Cc1cnc(CN(Cc2ccc(C#N)cc2)C(=O)CSc2ccccc2Cl)o1] |
| 73 | 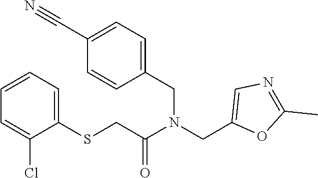 [SMILES: Cc1ncc(CN(Cc2ccc(C#N)cc2)C(=O)CSc2ccccc2Cl)o1] |
| 74 | 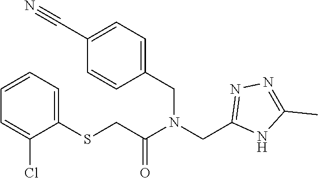 [SMILES: Cc1nnc(CN(Cc2ccc(C#N)cc2)C(=O)CSc2ccccc2Cl)[nH]1] |
| 75 | 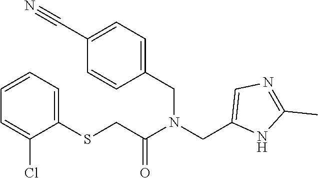 [SMILES: Cc1ncc(CN(Cc2ccc(C#N)cc2)C(=O)CSc2ccccc2Cl)[nH]1] |
| 76 | 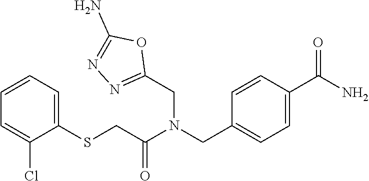 [SMILES: NC(=O)c1ccc(CN(Cc2nnc(N)o2)C(=O)CSc2ccccc2Cl)cc1] |
| 78 | 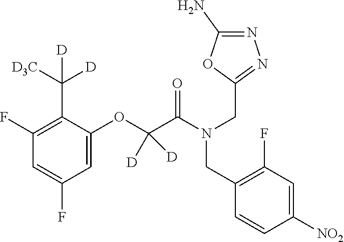 [SMILES: [2H]C([2H])(Oc1cc(F)cc(F)c1C([2H])([2H])C([2H])([2H])[2H])C(=O)N(Cc1nnc(N)o1)Cc1ccc([N+](=O)[O-])cc1F] |
| 80 | 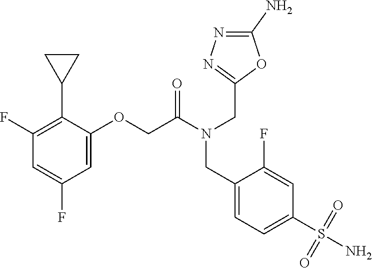 [SMILES: Nc1nnc(CN(Cc2ccc(S(N)(=O)=O)cc2F)C(=O)COc2cc(F)cc(F)c2C2CC2)o1] |
| 81 | 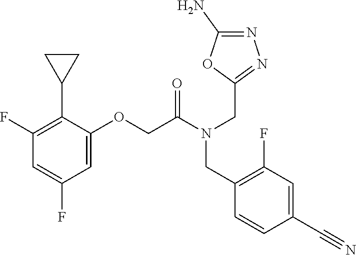 [SMILES: N#Cc1ccc(CN(Cc2nnc(N)o2)C(=O)COc2cc(F)cc(F)c2C2CC2)c(F)c1] |
| 82 | 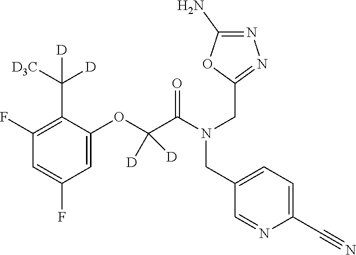 [SMILES: [2H]C([2H])(Oc1cc(F)cc(F)c1C([2H])([2H])C([2H])([2H])[2H])C(=O)N(Cc1ccc(C#N)nc1)Cc1nnc(N)o1] |
| 83 | 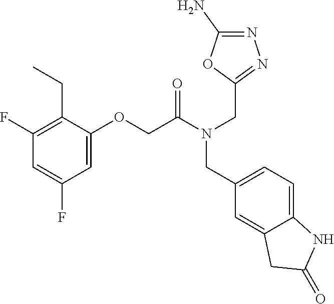 [SMILES: CCc1c(F)cc(F)cc1OCC(=O)N(Cc1ccc2c(c1)CC(=O)N2)Cc1nnc(N)o1] |
| 84 | 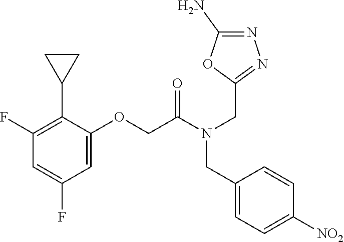 [SMILES: Nc1nnc(CN(Cc2ccc([N+](=O)[O-])cc2)C(=O)COc2cc(F)cc(F)c2C2CC2)o1] |
| 85 | 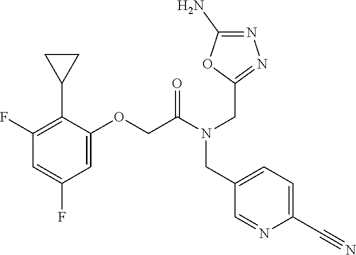 [SMILES: N#Cc1ccc(CN(Cc2nnc(N)o2)C(=O)COc2cc(F)cc(F)c2C2CC2)cn1] |
| 86 | 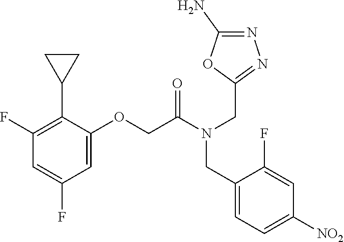 [SMILES: Nc1nnc(CN(Cc2ccc([N+](=O)[O-])cc2F)C(=O)COc2cc(F)cc(F)c2C2CC2)o1] |
| 87 | 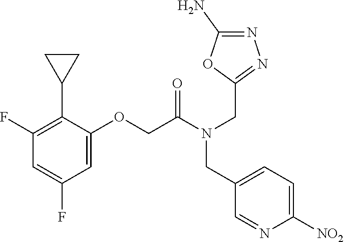 [SMILES: Nc1nnc(CN(Cc2ccc([N+](=O)[O-])nc2)C(=O)COc2cc(F)cc(F)c2C2CC2)o1] |
| 88 | 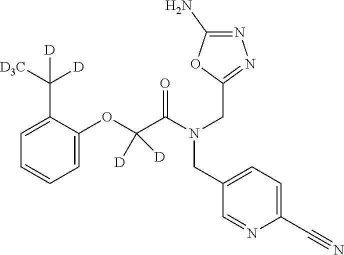 [SMILES: [2H]C([2H])(Oc1ccccc1C([2H])([2H])C([2H])([2H])[2H])C(=O)N(Cc1ccc(C#N)nc1)Cc1nnc(N)o1] |
| 89 | 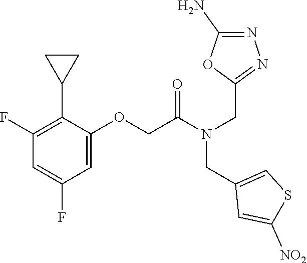 [SMILES: Nc1nnc(CN(Cc2csc([N+](=O)[O-])c2)C(=O)COc2cc(F)cc(F)c2C2CC2)o1] |
| 90 | 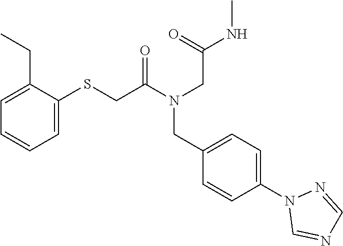 [SMILES: CCc1ccccc1SCC(=O)N(CC(=O)NC)Cc1ccc(-n2cncn2)cc1] |
| 91 | 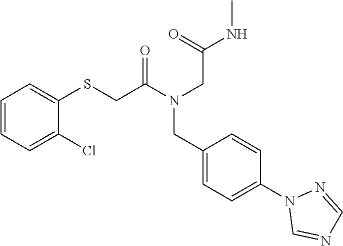 [SMILES: CNC(=O)CN(Cc1ccc(-n2cncn2)cc1)C(=O)CSc1ccccc1Cl] |
| 92 | 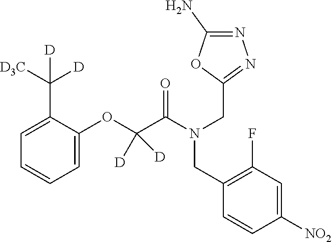 [SMILES: [2H]C([2H])(Oc1ccccc1C([2H])([2H])C([2H])([2H])[2H])C(=O)N(Cc1nnc(N)o1)Cc1ccc([N+](=O)[O-])cc1F] |
| 93 | 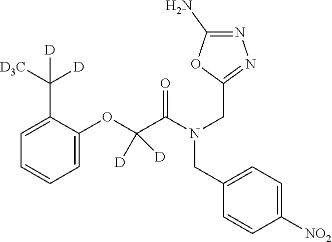 [SMILES: [2H]C([2H])(Oc1ccccc1C([2H])([2H])C([2H])([2H])[2H])C(=O)N(Cc1ccc([N+](=O)[O-])cc1)Cc1nnc(N)o1] |
| 95 | 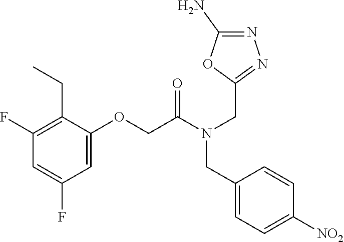 [SMILES: CCc1c(F)cc(F)cc1OCC(=O)N(Cc1ccc([N+](=O)[O-])cc1)Cc1nnc(N)o1] |
| 96 | 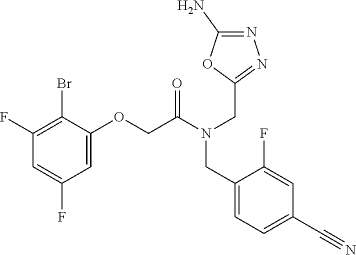 [SMILES: N#Cc1ccc(CN(Cc2nnc(N)o2)C(=O)COc2cc(F)cc(F)c2Br)c(F)c1] |
| 97 | 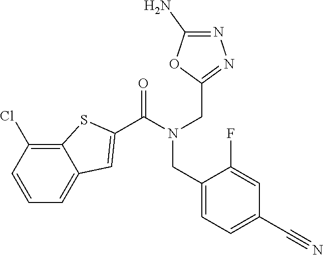 [SMILES: N#Cc1ccc(CN(Cc2nnc(N)o2)C(=O)c2cc3cccc(Cl)c3s2)c(F)c1] |
| 98 | 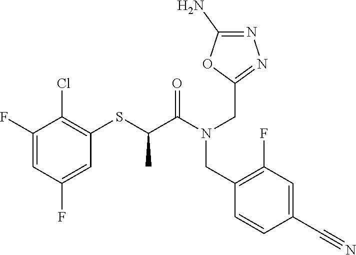 [SMILES: C[C@@H](Sc1cc(F)cc(F)c1Cl)C(=O)N(Cc1nnc(N)o1)Cc1ccc(C#N)cc1F] |
| 99 | 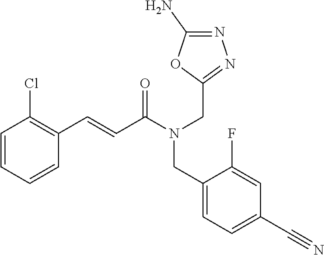 [SMILES: N#Cc1ccc(CN(Cc2nnc(N)o2)C(=O)/C=C/c2ccccc2Cl)c(F)c1] |
| 101 | 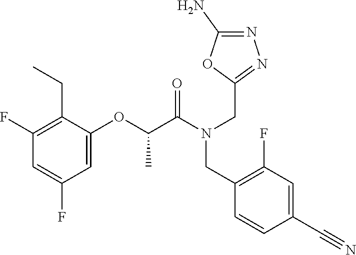 [SMILES: CCc1c(F)cc(F)cc1O[C@@H](C)C(=O)N(Cc1nnc(N)o1)Cc1ccc(C#N)cc1F] |
| 103 | 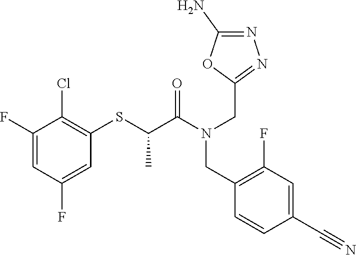 [SMILES: C[C@H](Sc1cc(F)cc(F)c1Cl)C(=O)N(Cc1nnc(N)o1)Cc1ccc(C#N)cc1F] |
| 106 | 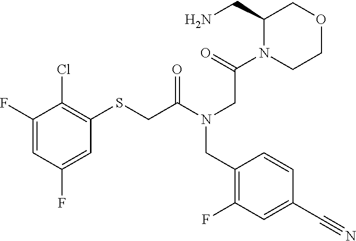 [SMILES: N#Cc1ccc(CN(CC(=O)N2CCOC[C@@H]2CN)C(=O)CSc2cc(F)cc(F)c2Cl)c(F)c1] |
| 107 | 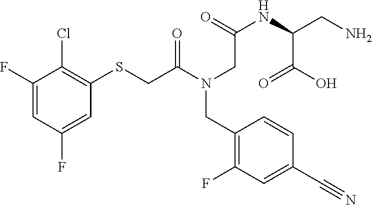 [SMILES: N#Cc1ccc(CN(CC(=O)N[C@@H](CN)C(=O)O)C(=O)CSc2cc(F)cc(F)c2Cl)c(F)c1] |
| 110 | 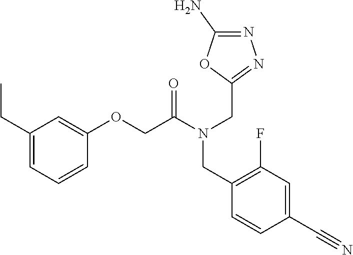 [SMILES: CCc1cccc(OCC(=O)N(Cc2nnc(N)o2)Cc2ccc(C#N)cc2F)c1] |
| 112 | 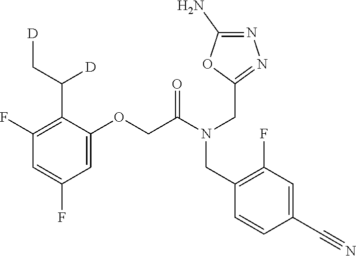 [SMILES: [2H]CC([2H])c1c(F)cc(F)cc1OCC(=O)N(Cc1nnc(N)o1)Cc1ccc(C#N)cc1F] |
| 113 | 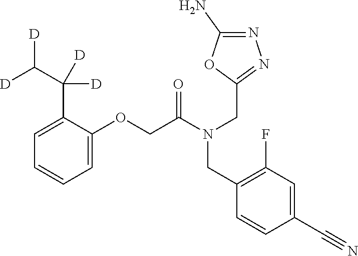 [SMILES: [2H]C([2H])C([2H])([2H])c1ccccc1OCC(=O)N(Cc1nnc(N)o1)Cc1ccc(C#N)cc1F] |
| 114 | 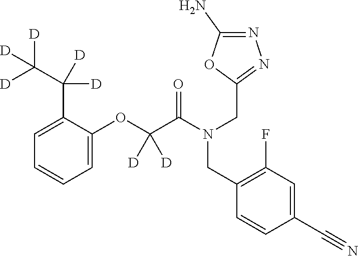 [SMILES: [2H]C([2H])(Oc1ccccc1C([2H])([2H])C([2H])([2H])[2H])C(=O)N(Cc1nnc(N)o1)Cc1ccc(C#N)cc1F] |
| 115 | 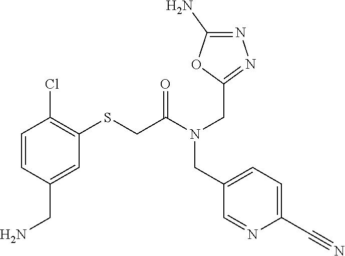 [SMILES: N#Cc1ccc(CN(Cc2nnc(N)o2)C(=O)CSc2cc(CN)ccc2Cl)cn1] |
| 116 | 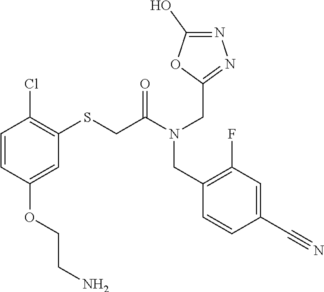 [SMILES: N#Cc1ccc(CN(Cc2nnc(O)o2)C(=O)CSc2cc(OCCN)ccc2Cl)c(F)c1] |
| 117 | 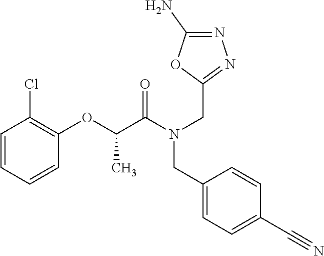 [SMILES: C[C@H](Oc1ccccc1Cl)C(=O)N(Cc1ccc(C#N)cc1)Cc1nnc(N)o1] |
| 118 | 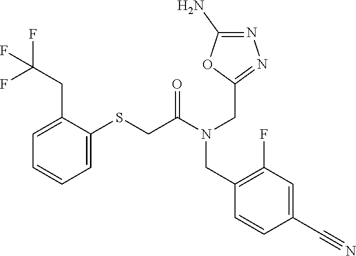 [SMILES: N#Cc1ccc(CN(Cc2nnc(N)o2)C(=O)CSc2ccccc2CC(F)(F)F)c(F)c1] |
| 119 | 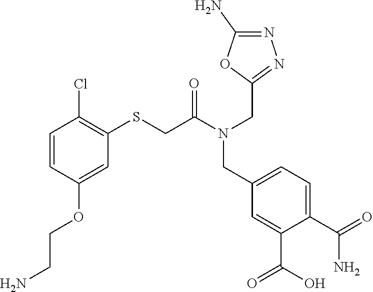 [SMILES: NCCOc1ccc(Cl)c(SCC(=O)N(Cc2ccc(C(N)=O)c(C(=O)O)c2)Cc2nnc(N)o2)c1] |
| 120 | 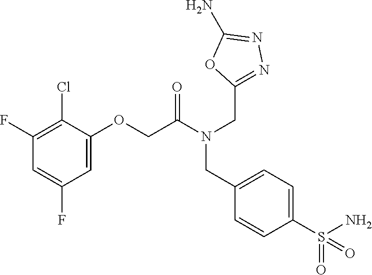 [SMILES: Nc1nnc(CN(Cc2ccc(S(N)(=O)=O)cc2)C(=O)COc2cc(F)cc(F)c2Cl)o1] |
| 121 | 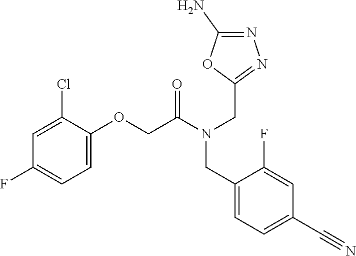 [SMILES: N#Cc1ccc(CN(Cc2nnc(N)o2)C(=O)COc2ccc(F)cc2Cl)c(F)c1] |

### Example 24—Biochemical Assay for Inhibition of LpxA

Exemplary compounds from the above Examples were tested for ability to inhibit LpxA activity using an in vitro substrate acylation assay with RapidFire™ MS analysis. Assay procedures and results are described below.

### Part I—Procedures for In Vitro Substrate Acylation Assay

Reactions were carried out in a final volume of 30 μL with 25 mM HEPES (pH 7.3), 150 mM NaCl, 2 mM DTT, 0.01% BSA, 5 μM UDP-GlcNac, recombinant LpxA enzyme (0.5 nM P. aeruginosa or 1.0 nM E. coli, both produced at Evotec), 0.5 μM acyl-transfer reagent (decanoyl P. aeruginosa acyl-carrier protein [PaACP-C₁₀] or myristoyl E. coli acyl-carrier protein [EcACP-C₁₋₄], both produced at Evotec), and 1% DMSO containing test compound. After 30 min incubation at room temperature, reactions were stopped by addition of 30 μL of 10% TCA in water.

Samples were centrifuged (4350 rpm, 4° C., 5 min), and then subjected to RapidFire™ MS analysis on an Agilent RapidFire 300 coupled to an Agilent 6460 triple-quadrupole mass spectrometer. Analytes were retained on a C4 (type A) cartridge using the following solvents and flow rates:
Pump 1—H₂O with 5 mM NH₄OAc at 2.0 mL/min,
Pump 2—MeOH/H₂O (90/10) at 1.5 mL/min,
Pump 3—MeOH/H₂O (90/10) at 0.5 mL/min, and
Pump 4—ACN (organic)/H₂O (aqueous).
   Substrate and product of the reaction were detected in negative mode ESI-MS/MS by MRM (multiple reaction monitoring) of transitions 606.0/385.0 (UDP-GlcNAc substrate) and 776.2/385.0 (UDP-3-O—(R-3-hydroxydecanoyl)-GlcNAc product, for P. aeruginosa LpxA assay) or 832.2/385.0 (UDP-3-O—(R-3-hydroxymyristoyl)-GlcNAc product, for E. coli LpxA assay).

Total activity (0% inhibition) was obtained from reactions containing no compound, and 100% inhibition is defined as reactions in absence of acyl-transfer reagent. For IC₅₀ determinations, peak areas of the substrate and product were compared to compute the proportion of product formed by the LpxA enzyme at each compound concentration. The fractional activity was converted to percentage inhibition and plotted against compound concentration in Evotec's proprietary platform Aplus and fit using a four-parameter logistic using non-linear regression to yield the IC₅₀ value.

### Part II—Results

Experimental results are provided in Table 9 below. For E. coli, the symbol “++++” indicates an IC₅₀ less than 0.5 μM. The symbol “+++” indicates an IC₅₀ in the range of 0.5 μM to 5.0 μM. The symbol “++” indicates an IC₅₀ in the range of greater than 5.0 μM to 20 μM. The symbol “+” indicates an IC₅₀ greater than 20 μM. The symbol “N/A” indicates that no data was available.

For P. aeruginosa, the symbol “****” indicates an IC₅₀ less than 20 nM. The symbol “***” indicates an IC₅₀ in the range of 20 nM to 100 nM. The symbol “**” indicates an IC₅₀ in the range of greater than 100 nM to 1.0 μM. The symbol “*” indicates an IC₅₀ greater than 1.0 μM. The symbol “N/A” indicates that no data was available.

TABLE 9

| Compound | E. coli | P. aeruginosa |
|---|---|---|
| No. | IC₅₀ | IC₅₀ |
| 1 | N/A | ** |
| 2 | + | * |
| 3 | + | * |
| 4 | N/A | ** |
| 5 | N/A | ** |
| 6 | N/A | * |
| 7 | N/A | **** |
| 8 | + | **** |
| 9 | + | **** |
| 10 | N/A | **** |
| 11 | + | ** |
| 12 | N/A | *** |
| 13 | N/A | *** |
| 14 | + | **** |
| 15 | N/A | ** |
| 16 | + | ** |
| 17 | N/A | *** |
| 18 | N/A | *** |
| 19 | N/A | *** |
| 20 | N/A | * |
| 21 | N/A | **** |
| 22 | N/A | *** |
| 23 | N/A | *** |
| 24 | N/A | *** |
| 25 | N/A | **** |
| 26 | N/A | **** |
| 27 | + | **** |
| 28 | N/A | **** |
| 29 | N/A | ** |
| 30 | N/A | **** |
| 31 | N/A | *** |
| 32 | N/A | **** |
| 33 | N/A | *** |
| 34 | N/A | *** |
| 35 | N/A | *** |
| 36 | N/A | ** |
| 37 | N/A | *** |
| 38 | N/A | *** |
| 39 | N/A | ** |
| 40 | N/A | *** |
| 41 | N/A | **** |
| 42 | N/A | **** |
| 43 | N/A | *** |
| 44 | + | ** |
| 45 | N/A | *** |
| 46 | + | *** |
| 47 | N/A | *** |
| 48 | N/A | *** |
| 49 | N/A | ** |
| 50 | N/A | ** |
| 51 | N/A | **** |
| 52 | N/A | **** |
| 53 | N/A | *** |
| 54 | + | *** |
| 55 | N/A | **** |
| 56 | N/A | ** |
| 57 | N/A | *** |
| 58 | N/A | *** |
| 59 | + | **** |
| 60 | N/A | ** |
| 61 | N/A | * |
| 62 | ++ | ** |
| 63 | N/A | *** |
| 64 | N/A | **** |
| 65 | N/A | ** |
| 66 | N/A | ** |
| 67 | N/A | ** |
| 68 | N/A | *** |
| 69 | N/A | *** |
| 70 | N/A | *** |
| 71 | N/A | * |
| 72 | N/A | ** |
| 73 | + | ** |
| 74 | N/A | ** |
| 75 | N/A | ** |
| 76 | + | ** |
| 77 | N/A | **** |
| 78 | N/A | **** |
| 79 | N/A | **** |
| 80 | N/A | **** |
| 81 | N/A | **** |
| 82 | N/A | **** |
| 83 | N/A | **** |
| 84 | N/A | **** |
| 85 | N/A | **** |
| 86 | N/A | **** |
| 87 | N/A | *** |
| 88 | N/A | *** |
| 89 | N/A | *** |
| 90 | N/A | ** |
| 91 | N/A | ** |
| 92 | N/A | *** |
| 93 | N/A | *** |
| 95 | N/A | **** |
| 96 | N/A | **** |
| 97 | N/A | * |
| 98 | N/A | **** |
| 99 | N/A | * |
| 100 | N/A | **** |
| 101 | N/A | ** |
| 103 | N/A | ** |
| 106 | N/A | * |
| 107 | N/A | ** |
| 110 | N/A | ** |
| 111 | N/A | **** |
| 112 | N/A | **** |
| 113 | N/A | *** |
| 114 | N/A | *** |
| 115 | N/A | N/A |
| 116 | N/A | * |
| 117 | N/A | * |
| 118 | N/A | * |
| 119 | N/A | * |
| 120 | N/A | N/A |
| 121 | N/A | * |

### Example 25—Biochemical Assay for Inhibition of LpxD

Exemplary compounds from the above Examples were tested for ability to inhibit LpxD activity using an in vitro substrate acylation assay with RapidFire™ MS analysis. Assay procedures and results are described below.

### Part I—Procedures for In Vitro Substrate Acylation Assay

Reactions were carried out in a final volume of 30 μL with 25 mM HEPES (pH 7.3), 150 mM NaCl, 2 mM DTT, 0.01% BSA, 5 μM UDP-3-O[R-3-Hydroxymyristoyl]-glucosamine (produced at Charles River according to literature procedures, and partially purified at Evotec), 10 nM recombinant LpxD enzyme (P. aeruginosa or E. coli, produced at Evotec), 1 μM decanoyl E. coli acyl-carrier protein (EcACP-C₁₀, produced at Evotec), and 1% DMSO containing test compound. After 120 min incubation at room temperature, reactions were stopped by addition of 30 μL of 10% TCA in water.

Samples were centrifuged (4350 rpm, 4° C., 5 min), and then subjected to RapidFire™ MS analysis on an Agilent RapidFire 300 coupled to an Agilent 6460 triple-quadrupole mass spectrometer. Analytes were retained on a C4 (type A) cartridge using the following solvents and flow rates:
Pump 1—H₂O with 5 mM NH₄OAc at 2.0 mL/min,
Pump 2—MeOH/H₂O (90/10) at 1.5 mL/min,
Pump 3—MeOH/H₂O (90/10) at 0.5 mL/min, and
Pump 4—ACN (organic)/H₂O (aqueous).
   Substrate and product of the reaction were detected in negative mode ESI-MS/MS by MRM (multiple reaction monitoring) of transitions 790.3/385.0 (UDP-3-O[R-3-Hydroxymyristoyl]-glucosamine substrate) and 960.4/385.0 (UDP-3-O—[R-3-Hydroxymyristoyl]-N—[R-3-hydroxydecanoyl]-glucosamine product).

Total activity (0% inhibition) was obtained from reactions containing no compound, and 100% inhibition is defined as reactions in absence of acyl-transfer reagent. For IC₅₀ determinations, peak areas of the substrate and product were compared to compute the proportion of product formed by the LpxD enzyme at each compound concentration. The fractional activity was converted to percentage inhibition and plotted against compound concentration in Evotec's proprietary platform Aplus and fit using a four-parameter logistic using non-linear regression to yield the IC₅₀ value.

### Part II—Results

Experimental results are provided in Table 10 below. For E. coli, the symbol “++++” indicates an IC₅₀ less than 0.5 μM. The symbol “+++” indicates an IC₅₀ in the range of 0.5 μM to 5.0 μM. The symbol “++” indicates an IC₅₀ in the range of greater than 5.0 μM to 20 μM. The symbol “+” indicates an IC₅₀ greater than 20 μM. The symbol “N/A” indicates that no data was available.

For P. aeruginosa, the symbol “****” indicates an IC₅₀ less than 0.5 μM. The symbol “***” indicates an IC₅₀ in the range of 0.5 μM to 5.0 μM. The symbol “**” indicates an IC₅₀ in the range of greater than 5.0 μM to 20 μM. The symbol “*” indicates an IC₅₀ greater than 20 μM. The symbol “N/A” indicates that no data was available.

TABLE 10

| Compound | E. coli | P. aeruginosa |
|---|---|---|
| No. | IC₅₀ | IC₅₀ |
| 2 | N/A | * |
| 5 | N/A | * |
| 6 | N/A | * |

### Example 26—Broth Microdilution Minimum Inhibition Concentration Assay

Exemplary compounds from the above Examples were tested to determine their minimum inhibitory concentration (MIC) for exemplary strains of Escherichia and Pseudomonas bacteria using broth microdilution methods. Assay procedures and results are described below.

### Part I—Procedures for Broth Microdilution Minimum Inhibition Concentration Assay

Broth microdilution assays were conducted according to Clinical and Laboratory Standards Institute guidelines (CLSI, M07-A¹¹: Clinical and Laboratory Standards Institute. Methods for Dilution Antimicrobial Susceptibility Tests for Bacteria that Grow Aerobically; Approved Standard-11th Edition. Wayne, PA: CLSI, 2018). Briefly, bacterial suspensions were adjusted to a density equivalent to a 0.5 McFarland standard and diluted in sterile, cation-adjusted Mueller-Hinton broth (Beckton Dickinson) to yield a final inoculum between 2×10⁵ and 8×10⁵ colony-forming units (CFU)/mL. An inoculum volume of 98 μL was added to wells containing 2 μL of 2-fold serial dilutions of drug (prepared in DMSO). All inoculated microdilution trays were incubated in ambient air at 37° C. for 18 h. Following incubation, the lowest concentration to completely inhibit all visible growth to the unaided eye was determined as the MIC. Performance of the assay was monitored by the use of laboratory quality-control strains (S. aureus ATCC 29213 and P. aeruginosa ATCC 27853) and levofloxacin, a compound with a defined MIC spectrum, in accordance with CLSI guidelines (CLSI, M100Ed29: Clinical and Laboratory Standards Institute. Performance Standards for Antimicrobial Susceptibility Testing, 29th Edition. Wayne, PA: CLSI, 2018).

Multiple enterobacteriaceae strains were tested, including both wild-type strains and multi-drug resistance pump deficient mutants. Specific strains tested included the following:
Wild-type P. aeruginosa PAO1,
P. aeruginosa PAO1 ΔmexABoprM—Disabled multi-drug efflux system Δ(MexAB-OprM),
P. aeruginosa PAO397—PAO1Δ(mexAB-oprM) Δ(mexCD-oprJ) Δ(mexEF-oprN) Δ(mexJK) Δ(mexXY) ΔopmH,
P. aeruginosa LESB58—Clinical isolate; colistin-resistant, isolated in the United Kingdom in 1988 from a patient with cystic fibrosis
E. coli JW0452-3 (BW25113 JacrA::kan)—Disabled multi-drug efflux system (AcrAB-TolC).

### Part II—Results

Experimental results are provided in Table 11, which provides MIC values for wild-type P. aeruginosa PAO1 (“PAO1 WT MIC”), P. aeruginosa PAO ΔmexABoprM (“PAO1 KO MIC”), P. aeruginosa PAO397 (“PAO397 KO MIC”), P. aeruginosa LESB58 (“LESB58”), and E. coli JW0452-3 (BW25113 JacrA::kan) (“E. coli KO MIC”).

The symbol “++++” indicates a MIC less than or equal to 1 μg/mL. The symbol “+++” indicates a MIC in the range of greater than 1 μg/mL to 16 μg/mL. The symbol “++” indicates a MIC in the range of greater than 16 μg/mL to 64 μg/mL. The symbol “+” indicates a MIC greater than 64 μg/mL. The symbol “N/A” indicates that no data was available.

TABLE 11

| Compound | PAO1 WT | PAO1 KO | PAO397 KO | LESB58 | E. coli KO |
|---|---|---|---|---|---|
| No. | MIC | MIC | MIC | MIC | MIC |
| 1 | N/A | N/A | +++ | N/A | N/A |
| 2 | + | N/A | ++ | N/A | N/A |
| 3 | + | N/A | ++ | N/A | N/A |
| 4 | + | N/A | ++ | N/A | N/A |
| 5 | + | N/A | ++ | N/A | ++ |
| 6 | + | N/A | +++ | N/A | N/A |
| 7 | + | +++ | ++++ | ++ | N/A |
| 8 | + | +++ | ++++ | N/A | N/A |
| 9 | ++ | +++ | ++++ | N/A | N/A |
| 10 | + | +++ | ++++ | N/A | N/A |
| 11 | N/A | +++ | ++++ | N/A | N/A |
| 12 | + | +++ | ++++ | N/A | N/A |
| 13 | ++ | +++ | ++++ | +++ | N/A |
| 14 | + | +++ | ++++ | ++ | N/A |
| 15 | + | +++ | +++ | N/A | N/A |
| 16 | + | ++ | +++ | N/A | N/A |
| 17 | + | N/A | +++ | N/A | N/A |
| 18 | + | +++ | +++ | + | N/A |
| 19 | + | +++ | ++++ | ++ | N/A |
| 20 | + | ++ | +++ | + | N/A |
| 21 | ++ | +++ | ++++ | ++ | N/A |
| 22 | + | N/A | +++ | N/A | N/A |
| 23 | + | N/A | +++ | N/A | N/A |
| 24 | + | +++ | ++++ | ++ | N/A |
| 25 | +++ | +++ | ++++ | +++ | N/A |
| 26 | + | ++++ | ++++ | +++ | N/A |
| 27 | + | ++++ | ++++ | +++ | N/A |
| 28 | ++ | +++ | ++++ | N/A | N/A |
| 29 | + | + | +++ | N/A | N/A |
| 30 | ++ | +++ | ++++ | N/A | N/A |
| 31 | + | +++ | ++++ | +++ | N/A |
| 32 | + | +++ | ++++ | N/A | N/A |
| 33 | + | ++ | ++++ | N/A | N/A |
| 34 | + | ++ | ++++ | N/A | N/A |
| 35 | + | +++ | ++++ | N/A | N/A |
| 36 | + | ++ | ++++ | + | N/A |
| 37 | + | +++ | ++++ | ++ | N/A |
| 38 | ++ | +++ | ++++ | ++ | N/A |
| 39 | + | ++ | +++ | + | N/A |
| 40 | + | ++ | ++++ | + | N/A |
| 41 | + | ++++ | ++++ | +++ | N/A |
| 42 | ++ | +++ | ++++ | +++ | N/A |
| 43 | + | + | +++ | N/A | N/A |
| 44 | + | N/A | ++ | N/A | N/A |
| 45 | + | +++ | ++++ | N/A | N/A |
| 46 | + | + | +++ | N/A | ++ |
| 47 | + | ++ | ++++ | N/A | N/A |
| 48 | + | ++ | ++++ | N/A | N/A |
| 49 | + | N/A | +++ | N/A | N/A |
| 50 | + | N/A | +++ | N/A | N/A |
| 51 | + | +++ | ++++ | +++ | N/A |
| 52 | + | +++ | ++++ | +++ | N/A |
| 53 | + | N/A | +++ | N/A | N/A |
| 54 | + | N/A | ++ | N/A | N/A |
| 55 | +++ | ++++ | ++++ | +++ | N/A |
| 56 | + | ++ | ++++ | + | N/A |
| 57 | + | +++ | ++++ | ++ | N/A |
| 58 | + | +++ | ++++ | + | N/A |
| 59 | + | +++ | ++++ | ++ | N/A |
| 60 | + | +++ | ++++ | N/A | N/A |
| 61 | + | N/A | ++ | N/A | N/A |
| 62 | + | N/A | +++ | N/A | ++ |
| 63 | + | +++ | ++++ | ++ | N/A |
| 64 | ++ | ++++ | ++++ | +++ | N/A |
| 65 | + | + | +++ | N/A | N/A |
| 66 | + | +++ | ++++ | N/A | N/A |
| 67 | + | ++ | +++ | N/A | N/A |
| 68 | + | ++ | +++ | N/A | N/A |
| 69 | + | +++ | +++ | N/A | N/A |
| 70 | + | +++ | ++++ | N/A | N/A |
| 71 | + | N/A | N/A | N/A | N/A |
| 72 | + | N/A | +++ | N/A | N/A |
| 73 | + | N/A | +++ | N/A | N/A |
| 74 | + | ++ | +++ | N/A | N/A |
| 75 | + | N/A | +++ | N/A | N/A |
| 76 | + | ++ | +++ | N/A | N/A |
| 77 | +++ | N/A | N/A | +++ | N/A |
| 78 | +++ | N/A | N/A | +++ | N/A |
| 79 | +++ | N/A | N/A | +++ | N/A |
| 80 | ++ | N/A | N/A | +++ | N/A |
| 81 | ++ | N/A | N/A | +++ | N/A |
| 82 | + | N/A | N/A | ++ | N/A |
| 83 | + | N/A | N/A | ++ | N/A |
| 84 | ++ | N/A | N/A | +++ | N/A |
| 85 | + | N/A | N/A | ++ | N/A |
| 86 | ++ | N/A | N/A | +++ | N/A |
| 87 | + | N/A | N/A | + | N/A |
| 88 | + | N/A | N/A | + | N/A |
| 89 | + | N/A | N/A | + | N/A |
| 90 | + | N/A | N/A | N/A | N/A |
| 91 | + | N/A | N/A | N/A | N/A |
| 92 | + | N/A | N/A | ++ | N/A |
| 93 | + | N/A | N/A | + | N/A |
| 95 | ++ | N/A | N/A | +++ | N/A |
| 96 | ++ | N/A | N/A | +++ | N/A |
| 97 | + | N/A | N/A | + | N/A |
| 98 | + | N/A | N/A | + | N/A |
| 99 | + | N/A | N/A | + | N/A |
| 100 | +++ | N/A | N/A | +++ | N/A |
| 101 | + | N/A | N/A | + | N/A |
| 103 | + | N/A | N/A | + | N/A |
| 106 | + | N/A | N/A | + | N/A |
| 107 | + | N/A | N/A | + | N/A |
| 110 | + | N/A | N/A | + | N/A |
| 111 | ++ | N/A | N/A | +++ | N/A |
| 112 | +++ | N/A | N/A | +++ | N/A |
| 113 | + | N/A | N/A | ++ | N/A |
| 114 | + | N/A | N/A | ++ | N/A |
| 115 | + | N/A | N/A | N/A | N/A |
| 116 | + | N/A | N/A | + | N/A |
| 117 | N/A | N/A | N/A | N/A | N/A |
| 118 | + | N/A | N/A | + | N/A |
| 119 | N/A | N/A | N/A | N/A | N/A |
| 120 | + | N/A | N/A | ++ | N/A |
| 121 | + | N/A | N/A | + | N/A |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound represented by Formula I: 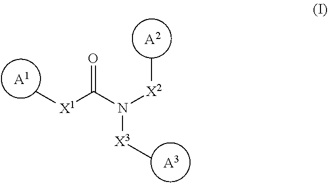
or a pharmaceutically acceptable salt thereof, wherein:
A¹ is a phenyl, 6-10 membered aza-heterocyclyl, or C₃₋₇ cycloalkyl, each of which is substituted with (i) one occurrence of R¹ and (ii) 0, 1, 2, or 3 occurrences of R²;
A² is one of the following:
a 5-membered heteroaryl containing at least two ring heteroatoms independently selected from the group consisting of nitrogen, oxygen, and sulfur, wherein at least one ring heteroatom is nitrogen, and said heteroaryl is substituted with 0, 1, 2, or 3 occurrences of R³; or
a 9-10 membered bicyclic aza-heteroaryl or 4-7 membered heterocycloalkyl, each of which is substituted with 0, 1, 2, or 3 occurrences of R³;
A³ is a 3-10 membered carbocyclyl or 3-10 membered heterocyclyl;
X¹ is 2-4 membered heteroalkylene or C₁₋₄ alkylene, each of which is substituted by 0, 1, or 2 occurrences of R⁴ and optionally one or more occurrences of halogen or deuterium;
X² is C₁₋₄ alkylene, —(C₁₋₃ alkylene)-C(O)-ψ, or —(C₁₋₄ alkylene)-C(O)N(R⁷)-ψ, each of which substituted by 0, 1, or 2 occurrences of R⁵ and optionally one or more occurrences of halogen; wherein ψ is a bond to A²; or 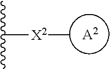
is —(C₁₋₄ alkylene)-C(O)N(R⁷)-(5-membered heteroaryl);
X³ is C₁₋₄ alkylene, C₂₋₄ alkenylene, or C₂₋₄ alkynylene; each of which substituted by 0, 1, or 2 occurrences of R⁶ and optionally one or more occurrences of halogen;
R¹ is halogen, cyano, C₁₋₄ alkyl, C₁₋₄ haloalkyl, C₁₋₄ deuteroalkyl, —O—C₁₋₄ alkyl, —O—C₁₋₄ haloalkyl, —S—C₁₋₄ alkyl, or —S—C₁₋₄ haloalkyl;
R² represents independently for each occurrence halogen, cyano, hydroxyl, —N(R⁷)₂, —CON(R⁷)₂, —SOₜN(R⁷)₂, C₁₋₆ alkyl, —O—C₁₋₆ alkyl, —S—C₁₋₆ alkyl, C₂₋₆ alkenyl, C₂₋₆ alkynyl, C₃₋₆ cycloalkyl, —O—C₃₋₆ cycloalkyl, or 3-6 membered heterocyclyl; wherein the C₃₋₆ cycloalkyl, —O—C₃₋₆ cycloalkyl, and 3-6 membered heterocyclyl are each optionally substituted with 1 or 2 substituents independently selected from C₁₋₆ alkyl, hydroxyl, oxo, —N(R⁴)₂, —CON(R⁴)₂, —SOₜN(R⁶)₂, and halogen; wherein each of the foregoing C₁₋₆ alkyl groups is optionally substituted with —OR⁷, —N(R⁷)₂, —CON(R⁷)₂, —SOₜN(R⁷)₂, or 1-3 halogens;
R³ represents independently for each occurrence —(C₀₋₄ alkylene)-N(R⁷)₂, —(C₀₋₄ alkylene)-OR⁷, C₁₋₄ alkyl, C₁₋₄ haloalkyl, or halogen;
R⁴, R⁵, and R⁶ each represent independently for each occurrence —(C₀₋₄ alkylene)-N(R⁷)₂ or —(C₀₋₄ alkylene)-OR⁷; or two occurrences of R⁴ are taken together with the carbon atom or atoms to which they are attached to form X⁴, two occurrences of R⁵ are taken together with the carbon atom or atoms to which they are attached to form X⁴, and/or two occurrences of R⁶ are taken together with the carbon atom or atoms to which they are attached to form X⁴; wherein X⁴ is a 3-7 membered ring optionally substituted with 1 or 2 substituents independently selected from halogen, C₁₋₄ alkyl, C₁₋₄ haloalkyl, —(C₀₋₄ alkylene)-N(R⁷)₂, and —(C₀₋₄ alkylene)-OR⁷;
R⁷ represents independently for each occurrence hydrogen, C₁₋₆ alkyl, C₁₋₆ haloalkyl, or —(C₀₋₆ alkylene)-(C₃₋₇ cycloalkyl); or two R⁷ attached to the same nitrogen atom are taken together with the nitrogen atom to which they are attached to form a 3-7 membered heterocyclic ring; and
t represents independently for each occurrence 1 or 2.

2. The compound of claim 1, wherein the compound is represented by Formula I.

3. The compound of claim 1 or 2, wherein A¹ is phenyl or 6-membered aza-heteroaryl, each of which is substituted with (i) one occurrence of R¹ and (ii) 0, 1, 2, or 3 occurrences of R².

4. The compound of claim 1 or 2, wherein A¹ is phenyl, pyridinyl, or pyrimidinyl; each of which is substituted with (i) one occurrence of R¹ and (ii) 0, 1, 2, or 3 occurrences of R².

5. The compound of claim 1 or 2, wherein A¹ is 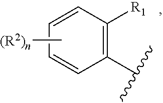
wherein n is 0, 1 or 2.

6. The compound of claim 1 or 2, wherein A¹ is

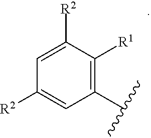

7. The compound of claim 1 or 2, wherein A¹ is 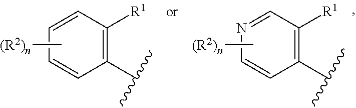
wherein n is 0, 1 or 2.

8. The compound of any one of claims 1-7, wherein A² is a 5-membered heteroaryl containing at least two ring heteroatoms independently selected from the group consisting of nitrogen, oxygen, and sulfur, wherein at least one ring heteroatom is nitrogen, and said heteroaryl is substituted with 0, 1, 2, or 3 occurrences of R³.

9. The compound of any one of claims 1-8, wherein X¹ is 2-4 membered heteroalkylene or C₁₋₄ alkylene, each of which is substituted by 0, 1, or 2 occurrences of R⁴.

10. The compound of any one of claims 1-9, wherein X² is C₁₋₄ alkylene substituted by 0, 1, or 2 occurrences of R⁵.

11. The compound of any one of claims 1-10, wherein X³ is C₁₋₄ alkylene substituted by 0, 1, or 2 occurrences of R⁶.

12. The compound of claim 1, wherein the compound is represented by Formula I-A: 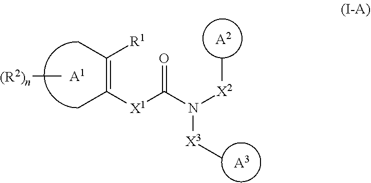
or a pharmaceutically acceptable salt thereof, wherein:
A¹ is phenyl or 6-membered aza-heteroaryl;
A² is a 5-membered heteroaryl containing at least two ring heteroatoms independently selected from the group consisting of nitrogen, oxygen, and sulfur, wherein at least one ring heteroatom is nitrogen, and said heteroaryl is substituted with 0, 1, 2, or 3 occurrences of R³;
A³ is 3-10 membered carbocyclyl or 3-10 membered heterocyclyl;
X¹ is —(C₁₋₂ alkylene)-S-ϕ, —(C₁₋₂ alkylene)-O-ϕ, —(C₁₋₂ alkylene)-SOₜ-ϕ, or C₁₋₃ alkylene; each of which is substituted by 0, 1, or 2 occurrences of R⁴; wherein 4 is a bond to A′;
X² is C₁₋₃ alkylene optionally substituted with 1 or 2 occurrences of R⁵;
X³ is C₁₋₃ alkylene or C₂₋₃ alkenylene, each of which is substituted by 0, 1, or 2 occurrences of R⁶;
R¹ is halogen, cyano, C₁₋₄ alkyl, C₁₋₄ haloalkyl, —O—C₁₋₄ alkyl, —O—C₁₋₄ haloalkyl, —S—C₁₋₄ alkyl, or —S—C₁₋₄ haloalkyl;
R² represents independently for each occurrence halogen, cyano, hydroxyl, —N(R⁷)₂, —CON(R⁷)₂, —SOₜN(R⁷)₂, C₁₋₆ alkyl, —O—C₁₋₆ alkyl, —S—C₁₋₆ alkyl, C₂₋₆ alkenyl, C₂₋₆ alkynyl, C₃₋₆ cycloalkyl, —O—C₃₋₆ cycloalkyl, or 3-6 membered heterocyclyl; wherein the C₃₋₆ cycloalkyl, —O—C₃₋₆ cycloalkyl, and 3-6 membered heterocyclyl are each optionally substituted with 1 or 2 substituents independently selected from C₁₋₆ alkyl, hydroxyl, oxo, —N(R⁴)₂, —CON(R⁴)₂, —SOₜN(R⁴)₂, and halogen; wherein each of the foregoing C₁₋₆ alkyl groups is optionally substituted with —OR⁷, —N(R⁷)₂, —CON(R⁷)₂, —SOₜN(R⁷)₂, or 1-3 halogens;
R³ represents independently for each occurrence —(C₀₋₄ alkylene)-N(R⁷)₂, —(C₀₋₄ alkylene)-OR⁷, C₁₋₄ alkyl, C₁₋₄ haloalkyl, or halogen;
R⁴, R⁵, and R⁶ each represent independently for each occurrence —(C₀₋₄ alkylene)-N(R⁷)₂ or —(C₀₋₄ alkylene)-OR⁷; or two occurrences of R⁴ are taken together with the carbon atom or atoms to which they are attached to form X⁴, two occurrences of R⁵ are taken together with the carbon atom or atoms to which they are attached to form X⁴, and/or two occurrences of R⁶ are taken together with the carbon atom or atoms to which they are attached to form X⁴; wherein X⁴ is a 3-7 membered ring optionally substituted with 1 or 2 substituents independently selected from halogen, C₁₋₄ alkyl, C₁₋₄ haloalkyl, —(C₀₋₄ alkylene)-N(R⁷)₂, and —(C₀₋₄ alkylene)-OR⁷;
R⁷ represents independently for each occurrence hydrogen, C₁₋₆ alkyl, C₁₋₆ haloalkyl, or —(C₀₋₆ alkylene)-(C₃₋₇ cycloalkyl);
n is 0, 1, or 2; and
t represents independently for each occurrence 1 or 2.

13. The compound of claim 12, wherein the compound is represented by Formula I-A.

14. The compound of claim 12 or 13, wherein

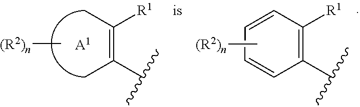

15. The compound of claim 12 or 13, wherein

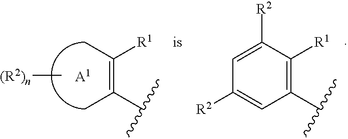

16. The compound of claim 12 or 13, wherein

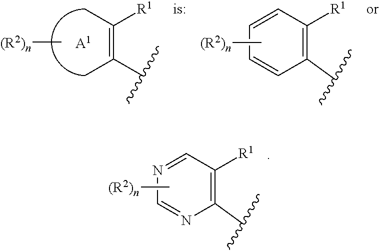

17. The compound of any one of claims 1-16, wherein R¹ is halogen, C₁₋₄ alkyl, C₁₋₄ haloalkyl, —O—C₁₋₄ alkyl, or —O—C₁₋₄ haloalkyl.

18. The compound of any one of claims 1-16, wherein R¹ is fluoro, chloro, methyl, or ethyl.

19. The compound of any one of claims 1-18, wherein R² represents independently for each occurrence halogen, cyano, hydroxyl, —N(R⁷)₂, —CON(R⁷)₂, —SOₜN(R⁷)₂, C₁₋₆ alkyl, or —O—C₁₋₆ alkyl; wherein each of the foregoing C₁₋₆ alkyl groups is optionally substituted 1-3 halogens.

20. The compound of any one of claims 1-18, wherein R² represents independently for each occurrence halogen, C₁₋₆ alkyl, or —O—C₁₋₆ alkyl; wherein said C₁₋₆ alkyl and —O—C₁₋₆ alkyl are each optionally substituted with —OR⁷, —N(R⁷)₂, or 1-3 halogens.

21. The compound of any one of claims 1-18, wherein R² represents independently for each occurrence fluoro, chloro, methyl, or ethyl.

22. The compound of any one of claims 1-18, wherein R² represents independently for each occurrence C₃₋₆ cycloalkyl, —O—C₃₋₆ cycloalkyl, or 3-6 membered heterocyclyl; each of which are optionally substituted with 1 or 2 substituents independently selected from C₁₋₆ alkyl, hydroxyl, oxo, —N(R⁷)₂, —CON(R⁷)₂, —SOₜN(R⁷)₂, and halogen; wherein said C₁₋₆ alkyl is optionally substituted with —OR⁷, —N(R⁷)₂, —CON(R⁷)₂, —SOₜN(R⁷)₂, or 1-3 halogens.

23. The compound of any one of claims 1-22, wherein R³ represents independently for each occurrence —(C₀₋₄ alkylene)-N(R⁷)₂.

24. The compound of any one of claims 1-22, wherein R³ is —N(R⁷)₂.

25. The compound of any one of claims 1-24, wherein two occurrences of R⁴ are taken together with the carbon atom or atoms to which they are attached to form a 3-7 membered ring optionally substituted with 1 or 2 substituents independently selected from halogen, C₁₋₄ alkyl, C₁₋₄ haloalkyl, —(C₀₋₄ alkylene)-N(R⁷)₂, and —(C₀₋₄ alkylene)-OR⁷.

26. The compound of any one of claims 1-25, wherein R⁵ represents independently for each occurrence —(C₀₋₄ alkylene)-N(R⁷)₂ or —(C₀₋₄ alkylene)-OR⁷.

27. The compound of any one of claims 1-25, wherein two occurrences of R⁵ are taken together with the carbon atom or atoms to which they are attached to form a 3-7 membered ring optionally substituted with 1 or 2 substituents independently selected from halogen, C₁₋₄ alkyl, C₁₋₄ haloalkyl, —(C₀₋₄ alkylene)-N(R⁷)₂, and —(C₀₋₄ alkylene)-OR⁷.

28. The compound of any one of claims 1-27, wherein two occurrences of R⁶ are taken together with the carbon atom or atoms to which they are attached to form a 3-7 membered ring optionally substituted with 1 or 2 substituents independently selected from halogen, C₁₋₄ alkyl, C₁₋₄ haloalkyl, —(C₀₋₄ alkylene)-N(R⁷)₂, and —(C₀₋₄ alkylene)-OR⁷.

29. The compound of any one of claims 1-28, wherein R⁷ represents independently for each occurrence hydrogen or C₁₋₄ alkyl.

30. The compound of any one of claims 1-28, wherein R⁷ represents independently for each occurrence hydrogen or methyl.

31. The compound of any one of claims 1-30, wherein A² is a 5-membered heteroaryl containing two or three ring heteroatoms each independently selected from the group consisting of nitrogen, oxygen, and sulfur, wherein at least two ring heteroatoms are nitrogen and said heteroaryl is substituted with 0, 1, 2, or 3 occurrences of R³.

32. The compound of any one of claims 1-30, wherein A² is oxadiazolyl, thiadiazolyl, oxazolyl, triazolyl, imidazolyl, or pyrazolyl, each of which is optionally substituted with 0, 1, 2, or 3 occurrences of R³.

33. The compound of any one of claims 1-30, wherein A² is oxadiazolyl or thiadiazolyl, each of which is optionally substituted with one occurrence of R³.

34. The compound of any one of claims 1-30, wherein A² is

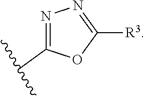

35. The compound of any one of claims 1-30, wherein A² is

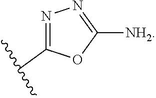 [SMILES: *c1nnc(N)o1]

36. The compound of any one of claims 1-35, wherein A³ is a 3-10 membered carbocyclyl or 5-6 membered heteroaryl, each of which is substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, oxo, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

37. The compound of any one of claims 1-35, wherein, A³ is a 3-10 membered carbocyclyl substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, oxo, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

38. The compound of any one of claims 1-35, wherein A³ is phenyl or a 5-6 membered heteroaryl, each of which is substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

39. The compound of any one of claims 1-35, wherein A³ is phenyl substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

40. The compound of any one of claims 1-35, wherein A³ is phenyl substituted with (i) one substituent selected from the group consisting of halogen, cyano, —SOₜN(H)R⁷, —CON(H)R⁷, —N(H)SOₜR⁸, —N(H)COR⁷, and 3-6 membered heterocyclyl, and (ii) 0, 1, 2, or 3 substituents independent selected from the group consisting of halogen, hydroxyl, cyano, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

41. The compound of any one of claims 1-35, wherein A³ is a 5-6 membered heteroaryl substituted by (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

42. The compound of any one of claims 1-35, wherein A³ is 5-6 membered heteroaryl substituted with (i) one substituent selected from the group consisting of halogen, cyano, —SOₜN(H)R⁷, —CON(H)R⁷, —N(H)SOₜR⁸, —N(H)COR⁷, and 3-6 membered heterocyclyl, and (ii) 0, 1, 2, or 3 substituents independent selected from the group consisting of halogen, hydroxyl, cyano, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

43. The compound of any one of claims 1-35, wherein A³ is 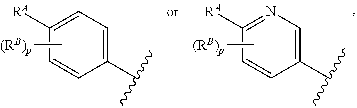
wherein R^{A} is halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), or —N(R⁷)SOₜ-(3-6 membered heterocyclyl); and R^{B} is halogen, hydroxyl, oxo, cyano, —N(R⁷)₂, C₁₋₆ alkyl, C₁₋₆ haloalkyl, and C₁₋₆ hydroxyalkyl, and p is 0, 1, or 2; and R⁸ is C₁₋₆ alkyl, C₁₋₆ haloalkyl, or —(C₀₋₆ alkylene)-(C₃₋₇ cycloalkyl).

44. The compound of any one of claims 1-35, wherein A³ is a 3-10 membered monocyclic saturated or partially unsaturated heterocyclyl or 5-10 membered bicyclic heterocyclyl, each of which is substituted with (i) one substituent selected from the group consisting of halogen, cyano, —SOₜN(H)R⁷, —CON(H)R⁷, —N(H)SOₜR⁸, —N(H)COR⁷, —O—C₁₋₆ alkyl, hydroxyl, —(C₀₋₆ alkylene)-N(H)R⁷, —SOₜN(H)—(C₁₋₆ alkylene)-N(H)R⁷, 3-6 membered heterocyclyl, —O-(3-6 membered heterocyclyl), —N(R⁷)-(3-6 membered heterocyclyl), and —N(R⁷)SOₜ-(3-6 membered heterocyclyl) and (ii) 0, 1, 2, or 3 substituents independent selected from the group consisting of halogen, hydroxyl, oxo, cyano, —N(R⁷)₂, C₁₋₆ alkyl, C₁₋₆ haloalkyl, and C₁₋₆ hydroxyalkyl; and R⁸ is C₁₋₆ alkyl, C₁₋₆ haloalkyl, or —(C₀₋₆ alkylene)-(C₃₋₇ cycloalkyl).

45. The compound of any one of claims 1-44, wherein X¹ is —(C₁₋₂ alkylene)-S-ϕ or —(C₁₋₂ alkylene)-O-ϕ, wherein the alkylene portion of each is optionally substituted with 1 or 2 occurrences of R⁴; wherein is a bond to A′.

46. The compound of any one of claims 1-44, wherein X¹ is —(C₁₋₂ alkylene)-S-ϕ or —(C₁₋₂ alkylene)-O-ϕ; wherein ϕ is a bond to A′.

47. The compound of any one of claims 1-44, wherein X¹ is C₁₋₃ alkylene optionally substituted with 1 or 2 occurrences of R⁴.

48. The compound of any one of claims 1-44, wherein X² is C₁₋₃ alkylene.

49. The compound of any one of claims 1-48, wherein X³ is C₁₋₃ alkylene optionally substituted with 1 or 2 occurrences of R⁶.

50. The compound of any one of claims 1-48, wherein X³ is C₁₋₃ alkylene.

51. The compound of any one of claims 1-48, wherein X³ is C₂₋₃ alkenylene optionally substituted with 1 or 2 occurrences of R⁶.

52. A compound in Table 1 or 9 herein, or a pharmaceutically acceptable salt thereof.

53. A pharmaceutical composition comprising a compound of any one of claims 1-52 and a pharmaceutically acceptable carrier.

54. A method of treating a bacterial infection in a patient, comprising administering to a patient in need thereof a therapeutically effective amount of a compound of any one of claims 1-52 to treat the bacterial infection.

55. The method of claim 54, wherein the bacterial infection is an infection by a gram-negative bacteria.

56. The method of claim 54, wherein the bacterial infection is an infection by a gram-positive bacteria.

57. The method of claim 54, wherein the bacterial infection is an infection by a Staphylococcus, Streptococcus, Enterococcus, Pseudomonas, Escherichia, Fusobacterium, Klebsiella, Haemophilus, Bordetella, Serratia, Proteus, Enterobacter, Campylobacter, Citrobacter, Vibrio, Morganella, Salmonella, Shigella, Acinetobacter, Legionella, Bacteroides, Neisseria, Moraxella, Chlamydia, Helicobacter, Prevotella, Porphyromonas, Veillonella, Bilophila, Centipeda, Leptotrichia, Selenomonas, or Sutterella bacterium, or a combination thereof.

58. The method of claim 54, wherein the bacterial infection is an infection by Staphylococcus aureus, Staphylococcus epidermidis, Streptococcus pyogenes, Streptococcus agalactiae, Streptococcus pneumoniae, Enterococcus faecalis, Enterococcus faecium, Pseudomonas aeruginosa, Escherichia coli, Fusobacterium nucleatum, Fusobacterium polymorphum, Fusobacterium vincentii, Fusobacterium animalis, Fusobacterium fusiforme, Fusobacterium canifelium, Fusobacterium necrophorum, Fusobacterium funduliforme, Fusobacterium ulcerans, Fusobacterium gonidiaformans, Fusobacterium mortiferum, Fusobacterium naviforme, Fusobacterium necrogenes, Fusobacterium russii, Fusobacterium varium, Klebsiella pneumoniae, Klebsiella oxytoca, Haemophilus influenzae, Haemophilus parainfluenzae, Bordetella pertussis, Bordetella bronchiseptica, Serratia marcescens, Proteus mirabilis, Enterobacter cloacae, Campylobacter jejuni, Vibrio parahemolyticus, Vibrio cholerae, Morganella morganii, Salmonella typhi, Salmonella paratyphi, Shigella dysenteriae, Acinetobacter baumannii, Acinetobacter calcoaceticus, Legionella pneumophila, Bacteroides fragilis, Bacteroides thetaiotaomicron, Bacteroides vulgatus, Bacteroides distasonis, Bacteroides ovatus, Bacteroides uniformis, Bacteroides caccae, Neisseria gonorrhoeae, Neisseria meningitides, Moraxella catarrhalis, Chlamydia trachomatis, Chlamydia psittaci, Helicobacter pylori, Prevotella intermedia, Prevotella melaninogenica, Prevotella bivia, Prevotella nigrescens, Prevotella disiens, Porphyromonas gingivalis, Veillonella atypica, Veillonella caviae, Veillonella criceti, Veillonella denticariosi, Veillonella dispar, Veillonella magna, Veillonella montpellierensis, Veillonella parvula, Veillonella ratti, Veillonella rodentium, Veillonella rogosae, Veillonella seminalis, Veillonella tobetsuensis Bilophila wadsworthia, Centipeda periodontii, Leptotrichia buccalis, Leptotrichia goodfellowii, Leptotrichia hofstadii, Leptotrichia honkongensis, Leptotrichia shahii, Leptotrichia trevisanii, Leptotrichia wadei, Selenomonas sputigena, Sutterella wadsworthensis, Sutterella parvirubra, Sutterella stercoricanis, or a combination thereof.

59. The method of claim 54, wherein the bacterial infection is an infection by Staphylococcus aureus, Staphylococcus epidermidis, Streptococcus pyogenes, Streptococcus agalactiae, Streptococcus pneumoniae, Enterococcus faecalis, Enterococcus faecium, Pseudomonas aeruginosa, Escherichia coli, Fusobacterium nucleatum, Klebsiella pneumoniae, Klebsiella oxytoca, Haemophilus influenzae, Haemophilus parainfluenzae, Bordetella pertussis, Bordetella bronchiseptica, Serratia marcescens, Proteus mirabilis, Enterobacter cloacae, Campylobacter jejuni, Vibrio parahemolyticus, Vibrio cholerae, Morganella morganii, Salmonella typhi, Salmonella paratyphi, Shigella dysenteriae, Acinetobacter baumannii, Acinetobacter calcoaceticus, Legionella pneumophila, Bacteroides fragilis, Neisseria gonorrhoeae, Neisseria meningitides, Moraxella catarrhalis, Chlamydia trachomatis, Chlamydia psittaci, Helicobacter pylori, or a combination thereof.

60. The method of claim 54, wherein the bacterial infection is an infection by Pseudomonas aeruginosa, Escherichia coli, or a combination thereof.

61. The method of any one of claims 54-60, wherein the patient is a human.

62. A method of inducing death of a bacterial cell, comprising exposing a bacterial cell to an effective amount of a compound of any one of claims 1-52 to induce death of the bacterial cell.

63. The method of claim 62, wherein the bacterial cell is a gram-negative bacteria.

64. The method of claim 62, wherein the bacterial cell is a gram-positive bacteria.

65. The method of claim 62, wherein the bacterial cell is a Staphylococcus, Streptococcus, Enterococcus, Pseudomonas, Escherichia, Fusobacterium, Klebsiella, Haemophilus, Bordetella, Serratia, Proteus, Enterobacter, Campylobacter, Citrobacter, Vibrio, Morganella, Salmonella, Shigella, Acinetobacter, Legionella, Bacteroides, Neisseria, Moraxella, Chlamydia, Helicobacter, Prevotella, Porphyromonas, Veillonella, Bilophila, Centipeda, Leptotrichia, Selenomonas, or Sutterella bacterium, or a combination thereof.

66. The method of claim 62, wherein the bacterial cell is Staphylococcus aureus, Staphylococcus epidermidis, Streptococcus pyogenes, Streptococcus agalactiae, Streptococcus pneumoniae, Enterococcus faecalis, Enterococcus faecium, Pseudomonas aeruginosa, Escherichia coli, Fusobacterium nucleatum, Fusobacterium polymorphum, Fusobacterium vincentii, Fusobacterium animalis, Fusobacterium fusiforme, Fusobacterium canifelium, Fusobacterium necrophorum, Fusobacterium funduliforme, Fusobacterium ulcerans, Fusobacterium gonidiaformans, Fusobacterium mortiferum, Fusobacterium naviforme, Fusobacterium necrogenes, Fusobacterium russii, Fusobacterium varium, Klebsiella pneumoniae, Klebsiella oxytoca, Haemophilus influenzae, Haemophilus parainfluenzae, Bordetella pertussis, Bordetella bronchiseptica, Serratia marcescens, Proteus mirabilis, Enterobacter cloacae, Campylobacter jejuni, Vibrio parahemolyticus, Vibrio cholerae, Morganella morganii, Salmonella typhi, Salmonella paratyphi, Shigella dysenteriae, Acinetobacter baumannii, Acinetobacter calcoaceticus, Legionella pneumophila, Bacteroides fragilis, Bacteroides thetaiotaomicron, Bacteroides vulgatus, Bacteroides distasonis, Bacteroides ovatus, Bacteroides uniformis, Bacteroides caccae, Neisseria gonorrhoeae, Neisseria meningitides, Moraxella catarrhalis, Chlamydia trachomatis, Chlamydia psittaci, Helicobacter pylori, Prevotella intermedia, Prevotella melaninogenica, Prevotella bivia, Prevotella nigrescens, Prevotella disiens, Porphyromonas gingivalis, Veillonella atypica, Veillonella caviae, Veillonella criceti, Veillonella denticariosi, Veillonella dispar, Veillonella magna, Veillonella montpellierensis, Veillonella parvula, Veillonella ratti, Veillonella rodentium, Veillonella rogosae, Veillonella seminalis, Veillonella tobetsuensis Bilophila wadsworthia, Centipeda periodontii, Leptotrichia buccalis, Leptotrichia goodfellowii, Leptotrichia hofstadii, Leptotrichia honkongensis, Leptotrichia shahii, Leptotrichia trevisanii, Leptotrichia wadei, Selenomonas sputigena, Sutterella wadsworthensis, Sutterella parvirubra, Sutterella stercoricanis, or a combination thereof.

67. The method of claim 62, wherein the bacterial cell is Staphylococcus aureus, Staphylococcus epidermidis, Streptococcus pyogenes, Streptococcus agalactiae, Streptococcus pneumoniae, Enterococcus faecalis, Enterococcus faecium, Pseudomonas aeruginosa, Escherichia coli, Fusobacterium nucleatum, Klebsiella pneumoniae, Klebsiella oxytoca, Haemophilus influenzae, Haemophilus parainfluenzae, Bordetella pertussis, Bordetella bronchiseptica, Serratia marcescens, Proteus mirabilis, Enterobacter cloacae, Campylobacter jejuni, Vibrio parahemolyticus, Vibrio cholerae, Morganella morganii, Salmonella typhi, Salmonella paratyphi, Shigella dysenteriae, Acinetobacter baumannii, Acinetobacter calcoaceticus, Legionella pneumophila, Bacteroides fragilis, Neisseria gonorrhoeae, Neisseria meningitides, Moraxella catarrhalis, Chlamydia trachomatis, Chlamydia psittaci, Helicobacter pylori, or a combination thereof.

68. The method of claim 62, wherein the bacterial cell is a Pseudomonas aeruginosa or Escherichia coli bacterium.

69. A method of inhibiting the activity of LpxA, comprising exposing an LpxA to an effective amount of a compound of any one of claims 1-52 to inhibit the activity of LpxA.
